(19) United States
(12) Patent Application Publication
(10) Pub. No.: US 2025/0188465 A1
(43) Pub. Date: Jun. 12, 2025

(54) POLYNUCLEIC ACID MOLECULES TARGETING APOC3 AND USES THEREOF

(71) Applicant: Sirius Therapeutics, Inc., San Diego, CA (US)

(72) Inventors: Curt BRADSHAW, San Diego, CA (US); Anthony NICHOLAS, Oregon, WI (US)

(21) Appl. No.: 19/048,758

(22) Filed: Feb. 7, 2025

Related U.S. Application Data
(63) Continuation of application No. PCT/US2023/030020, filed on Aug. 10, 2023.
(60) Provisional application No. 63/371,326, filed on Aug. 12, 2022.

(51) Int. Cl. C12N 15/113; A61P 3/06

(52) U.S. Cl. CPC C12N 15/113; A61P 3/06; C12N 2310/11; C12N 2310/14; C12N 2310/315; C12N 2310/321; C12N 2310/322; C12N 2310/351

(57) ABSTRACT

Disclosed herein are polynucleic acids, pharmaceutical compositions, and methods for suppressing the expression of apolipoprotein C3 (APOC3).

### CROSS-REFERENCE

This application is a continuation of International Application No. PCT/US2023/030020, filed Aug. 10, 2023, which claims the benefit of U.S. Provisional Application No. 63/371,326, filed on Aug. 12, 2022, all of which are incorporated herein by reference in their entirety.

### SEQUENCE LISTING

The instant application contains a Sequence Listing which has been submitted electronically in XML format and is hereby incorporated by reference in its entirety. Said XML copy, created on Feb. 6, 2025, is named 61382-713.301_SL.xml and is 2,267,336 bytes in size.

### BACKGROUND OF THE DISCLOSURE

Apolipoprotein C3 (APOC3) is a protein encoded by the APOC3 gene. APOC3 may be secreted by the liver as well as the small intestine, and may play an important role in inhibiting hepatic uptake of triglyceride-rich particles. Therefore, high plasma APOC3 correlates with high glyceride and an increased risk of developing cardiovascular diseases. Accordingly, there is a need for developing an effective APOC3 inhibitor without cytotoxicity. The polynucleic acid molecules, conjugates thereof, and methods described herein satisfy this need and provide related advantages.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### SUMMARY OF THE DISCLOSURE

To meet the need for a more effective APOC3 inhibitor, provided herein, in one aspect, is a polynucleic acid molecule for modulating expression of apolipoprotein C3 (APOC3) gene, comprising a sense strand and an antisense strand, wherein the antisense strand comprises a nucleic acid sequence that is at least 80%, at least 85%, at least 90%, at least 95% identical to a nucleic acid sequence selected from SEQ ID NOs: 1-108, 433-456, 529-534, and 553-563. In some cases, the antisense strand comprises a nucleic acid sequence comprising at least 14, 15, 16, 17, 18, 19, 20, 21, or 22 consecutive sequences of a nucleic acid sequence selected from SEQ ID NOs: 1-108, 433-456, 529-534, and 553-563 with no more than 1, 2, 3, or 4 mismatches. In some cases, the sense strand comprises a nucleic acid sequence that is at least 80%, at least 85%, at least 90%, at least 95% identical to a nucleic acid sequence selected from SEQ ID NOs: 217-324, 481-504, 541-546, and 575-585. In some cases, the sense strand comprises a nucleic acid sequence comprising at least 14, 15, 16, 17, 18, 19, or 20 consecutive sequences of a nucleic acid sequence selected from SEQ ID NOs: 217-324, 481-504, 541-546, and 575-585 with no more than 1, 2, 3, or 4 mismatches.

In some cases, the sense strand comprises a nucleic acid sequence of SEQ ID NOs: 217-324, 481-504, 541-546, and 575-585 and the antisense strand comprises a nucleic acid sequence selected from SEQ ID NOs: 1-108, 433-456, 529-534, and 553-563.

In some cases, the polynucleic acid molecule comprises (1) a 2′-fluoro modified nucleotides; (2) a 2′-O-methyl modified nucleotides; (3) 2′-deoxy modified nucleotides, or (4) a modified internucleotide linkage. In some cases, the polynucleic acid molecule comprise at least two consecutive modified internucleotide linkages at the 5′ end or at the 3′ end.

In some cases, the antisense strand comprises 5′-nNfnnnNfnNfNfnnnnNfnNfnnnnnnn-3′, 5′-nNfnnnNfnnnnnnnNfnNfnnnnnnnn-3′, 5′-nNfnnnnNfnnnnNfnNfnnnnnnnnn-3′, 5′-nNfnnnnNfnnnnNfnNfnNfnnnnnnn-3′, or 5′-nNfnnnnnnnnnNfnNfnNfnnnnnnn-3′, wherein “Nf” stands for a 2′-fluoro modified nucleotide, and wherein “n” stands for a 2′-O-methyl modified nucleotide. In some cases, the sense strand comprises 5′-nnnnnnNfnNfnNfnnnnnnnnn-3′, 5′-nnnnnnNfnNfNfNfnnnnnnnnn-3′, or 5′-nnnnnnnnNfNfNfnnnnnnnnnn-3′, wherein “NF” stands for a 2′-fluoro modified nucleotide, and wherein “n” stands for a 2′-O-methyl modified nucleotide. In some cases, the sense strand of the polynucleic acid molecule comprises 5′-NfnNfnNfnNfnNfNtNfnNfnNfnNfNfnNf-3′, the antisense strand of the polynucleic acid molecule comprises 5′-nNfnNfnNfnNfnNfnnnNfnNffnNfnNfnnn-3′; the sense strand of the polynucleic acid molecule comprises 5′-nnnnnnNfnNfNfNfnnnnnnnnnn-3′, the antisense strand of the polynucleic acid molecule comprises 5′-nNfnnnNfnNfNfnnnnNfnNfnnnnnnn-3′; the sense strand of the polynucleic acid molecule comprises 5′-nnnnnnnnNfnNfnnnnnnnnnn-3′, wherein the antisense strand of the polynucleic acid molecule comprises 5′-nNfnnnnnnnnnNfnNfnnnnnnnnn-3′; the sense strand of the polynucleic acid molecule comprises 5′-nnnnnnNfnNfnNfnnnnnnnnnn-3′, the antisense strand of the polynucleic acid molecule comprises 5′-nNfnnnnnnnnnNfnNfnNfnnnnnnn-3′; or the sense strand of the polynucleic acid molecule comprises 5′-nnnnnnNfnNfnNfnnnnnnnnnn-3′, the antisense strand of the polynucleic acid molecule comprises 5′_nNfnnnnNfnnnnNfnNfnNfnnnnnnn-3′, wherein “Nf” stands for a 2′-fluoro modified nucleotide, and wherein “n” stands for a 2′-O-methyl modified nucleotide.

In some cases, the modified internucleotide linkage is a phosphorothioate internucleotide linkage. In some cases, the modified internucleotide linkage comprises a stereochemically enriched phosphorothioate internucleotide linkage.

In some cases, the sense strand comprises a nucleic acid sequence that is at least 80%, at least 85%, at least 90%, at least 95% identical to a nucleic acid sequence selected from SEQ ID NOs: 325-432, 505-528, 547-552, and 586-596. In some cases, the antisense strand comprises a nucleic acid sequence that is at least 80%, at least 85%, at least 90%, at least 95% identical to a nucleic acid sequence selected from SEQ ID NOs: 109-216, 457-480, 535-540, and 564-574. In some cases, the sense strand comprises a nucleic acid sequence selected from SEQ ID NOs: 325-432, 505-528, 547-552, and 586-596, and the antisense strand comprises a nucleic acid sequence selected from SEQ ID NOs: 109-216, 457-480, 535-540, and 564-574.

In another aspect, provided herein is polynucleic acid molecule for modulating expression of apolipoprotein C3 (APOC3) gene, wherein polynucleic acid molecule comprises: (a) an antisense strand comprising the nucleotide sequence of UUUCAGGGAACUGAAGCCAUCGG (SEQ ID NO:529) and a sense strand comprising the nucleotide sequence of GAUGGCUUCAGUUCCCUGAAA (SEQ ID NO:541); (b) an antisense strand comprising the nucleotide sequence of UGAAUACUGUCCCUUUUAAGCAA (SEQ ID NO:530) and a sense strand comprising the nucleotide sequence of GCUUAAAAGGGACAGUAUUCA (SEQ ID NO:542); (c) an antisense strand comprising the nucleotide sequence of UAGAAUACUGUCCCUUUUAAGCA (SEQ ID NO:531) and a sense strand comprising the nucleotide sequence of CUUAAAAGGGACAGUAUUCUA (SEQ ID NO:543); (d) an antisense strand comprising the nucleotide sequence of UUGAGAAUACUGUCCCUUUUAAG (SEQ ID NO:532) and a sense strand comprising the nucleotide sequence of UAAAAGGGACAGUAUUCUCAA (SEQ ID NO:544); (e) an antisense strand comprising the nucleotide sequence of UCUGAGAAUACUGUCCCUUUUAA (SEQ ID NO:533) and a sense strand comprising the nucleotide sequence of AAAAGGGACAGUAUUCUCAGA (SEQ ID NO:545); (f) an antisense strand comprising the nucleotide sequence of UCACUGAGAAUACUGUCCCUUUU (SEQ ID NO:534) and a sense strand comprising the nucleotide sequence of AAGGGACAGUAUUCUCAGUGA (SEQ ID NO:546); (g) an antisense strand comprising the nucleotide sequence of UCUGAGAAUACUGUCCCCUUUUAA (SEQ ID NO: 554) and a sense strand comprising the nucleotide sequence of AAAAGGGACAGUAUUCUCAGA (SEQ ID NO: 576); (h) an antisense strand comprising the nucleotide sequence of UCUGAGAAUACUGUCCCUUUCAA (SEQ ID NO: 555) and a sense strand comprising the nucleotide sequence of GAAAGGGACAGUAUUCUCAGA (SEQ ID NO. 577); (i) an antisense strand comprising the nucleotide sequence of UCUGAGAAUACUGUCCCUUUGAA (SEQ ID NO: 556) and a sense strand comprising the nucleotide sequence of CAAAGGGACAGUAUUCUCAGA (SEQ ID NO: 578); (j) an antisense strand comprising the nucleotide sequence of UCUGAGAAUACUGUCCCUUGCAA (SEQ ID NO: 557) and a sense strand comprising the nucleotide sequence of GCAAGGGACAGUAUUCUCAGA (SEQ ID NO: 579); (k) an antisense strand comprising the nucleotide sequence of UCUGAGAAUACUGUCCCUUCGAA (SEQ ID NO: 558) and a sense strand comprising the nucleotide sequence of CGAAGGGACAGUAUUCUCAGA (SEQ ID NO: 580); (l) an antisense strand comprising the nucleotide sequence of UCUGAGAAUACUGUCCCUUUUAA (SEQ ID NO: 559) and a sense strand comprising the nucleotide sequence of AAAAGGGACAGUAUUCUCAGA (SEQ ID NO: 581); (m) an antisense strand comprising the nucleotide sequence of UCUGAGAAUACUGUCCCUUUGAA (SEQ ID NO: 560) and a sense strand comprising the nucleotide sequence of CAAAGGGACAGUAUUCUCAGA (SEQ ID NO: 582); (n) an antisense strand comprising the nucleotide sequence of UCUGAGAAUACUGUCCCUUUUAA (SEQ ID NO: 561) and a sense strand comprising the nucleotide sequence of AAAAGGGACAGUAUUCUCAGA (SEQ ID NO: 583); (o) an antisense strand comprising the nucleotide sequence of UCUGAGAAUACUGUCCCUUUUAA (SEQ ID NO: 562) and a sense strand comprising the nucleotide sequence of AAAAGGGACAGUAUUCUCAGA (SEQ ID NO: 584); or (p) an antisense strand comprising the nucleotide sequence of UCUGAGAAUACUGUCCCUUUUAA (SEQ ID NO: 563) and a sense strand comprising the nucleotide sequence of AAAAGGGACAGUAUUCUCAGA (SEQ ID NO: 585).

In another aspect, provided herein is polynucleic acid molecule for modulating expression of apolipoprotein C3 (APOC3) gene, wherein polynucleic acid molecule comprises: (a) an antisense strand comprising the nucleotide sequence of usUfsucagGfgaacUfgAfaGfccaucsgsg (SEQ ID NO:535) and a sense strand comprising the nucleotide sequence of gsasuggcUfuCfaGfuucccugaaa (SEQ ID NO:547); (b) an antisense strand comprising the nucleotide sequence of usGfsaauaCfugucCfcUfuUfuaagcsasa (SEQ ID NO:536) and a sense strand comprising the nucleotide sequence of gscsuuaaAfaGfgGfacaguauuca (SEQ ID NO:548); (c) an antisense strand comprising the nucleotide sequence of usAfsgaauAfcuguCfcCfuUfuuaagscsa (SEQ ID NO:537) and a sense strand comprising the nucleotide sequence of csusuaaaAfgGfgAfcaguauucua (SEQ ID NO:549); (d) an antisense strand comprising the nucleotide sequence of usUfsgagaAfuacuGfuCfcCfuuuuasasg (SEQ ID NO:538) and sense strand comprising the nucleotide sequence of usasaaagGfgAfcAfguauucucaa (SEQ ID NO:550); (e) an antisense strand comprising the nucleotide sequence of usCfsugagAfauacUfgUfcCfcuuuusasa (SEQ ID NO:539) and a sense strand comprising the nucleotide sequence of asasaaggGfaCfaGfuauucucaga (SEQ ID NO:551); (f) an antisense strand comprising the nucleotide sequence of usCfsacugAfgaauAfcUfgUfcccuususu (SEQ ID NO:540) and a sense strand comprising the nucleotide sequence of asascgggaCfaGfuAfuucucaguga (SEQ ID NO:552); (g) an antisense strand comprising the nucleotide sequence of vpusCfsugagAfauacUfgUfcCfcuuuusasa (SEQ ID NO: 565) and a sense strand comprising the nucleotide sequence of asasaaggGfaCfaGfuauucucaga (SEQ ID NO: 587); (h) an antisense strand comprising the nucleotide sequence of usCfsugagAfauacUfgUfcCfauuucsasa (SEQ ID NO: 566) and a sense strand comprising the nucleotide sequence of gsasaaggGfaCfaGfuauucucaga (SEQ ID NO: 588); (i) an antisense strand comprising the nucleotide sequence of usCfsugagAfauacUfgUfcCfcuuugsasa (SEQ ID NO: 567) and a sense strand comprising the nucleotide sequence of csasaaggGfaCfaGfuauucucaga (SEQ ID NO: 589); (j) an antisense strand comprising the nucleotide sequence of usCfsugagAfauacUfgUfcCfcuugcsasa (SEQ ID NO: 568) and a sense strand comprising the nucleotide sequence of gscsaaggGfaCfaGfuauucucaga (SEQ ID NO: 590); (k) an antisense strand comprising the nucleotide sequence of usCfsugagAfauacUfgUfcCfcuucgsasa (SEQ ID NO: 569) and a sense strand comprising the nucleotide sequence of csgsaaggGfaCfaGfuauucucaga (SEQ ID NO: 591); (l) an antisense strand comprising the nucleotide sequence of usCfsugagAfauacUfgUfcCfcuuuusasa (SEQ ID NO: 570) and a sense strand comprising the nucleotide sequence of (invAb)asasaaggGfaCfaGfuauucucaga (SEQ ID NO: 592); (m) an antisense strand comprising the nucleotide sequence of usCfsugagAfauacUfgUfcCfcuuugsasa (SEQ ID NO: 571) and a sense strand comprising the nucleotide sequence of (invAb)csasaaggGfaCfaGfuauucucaga (SEQ ID NO: 593); (n) an antisense strand comprising the nucleotide sequence of usCfsugdAgdAauacUfgUfcCfcuuuusasa (SEQ ID NO: 572) and a sense strand comprising the nucleotide sequence of asasaaggGfaCfaGfuauucucaga (SEQ ID NO: 594); (o) an antisense strand comprising the nucleotide sequence of usCfsugagAfauacUfgUfscCfcuuuusasa (SEQ ID NO: 573) and a sense strand comprising the nucleotide sequence of asasaaggGfaCfaGfuauucucaga (SEQ ID NO: 595); or (p) an antisense strand comprising the nucleotide sequence of usCfsugagAfauacUfgUfcCfcsuuuusasa (SEQ ID NO: 574) and a sense strand comprising the nucleotide sequence of asasaaggGfaCfaGfuauucucaga (SEQ ID NO: 596), wherein “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide.

In another aspect, provided herein is a polynucleic acid molecule conjugate for modulating expression of apolipoprotein C3 (APOC3) gene, wherein the polynucleic acid molecule conjugate comprises a polynucleic acid molecule described herein and an asialoglycoprotein receptor targeting moiety. In some cases, the asialoglycoprotein receptor targeting moiety comprises N-Acetylgalactosamine (GalNAc) or galactose.

In some cases, the polynucleic acid molecule and the asialoglycoprotein receptor targeting moiety is coupled via a linker. In some cases, the linker comprises formula (IV) below,

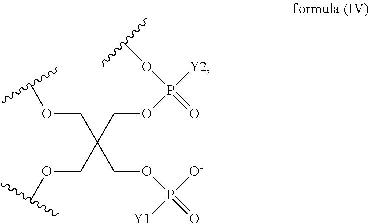

wherein at least one of Y1 and Y2 is a nucleotide in the polynucleic acid molecule. In some cases, the Y1 is the last nucleotide on the 3′-terminus of the sense strand of the polynucleic acid molecule or the Y1 and Y2 are two consecutive nucleotides in the polynucleic acid molecule. In some instances, the linker and the asialoglycoprotein receptor targeting moiety with the last nucleotide on the 3′-terminus of the sense strand of the polynucleic acid molecule are shown in Formula (V′):

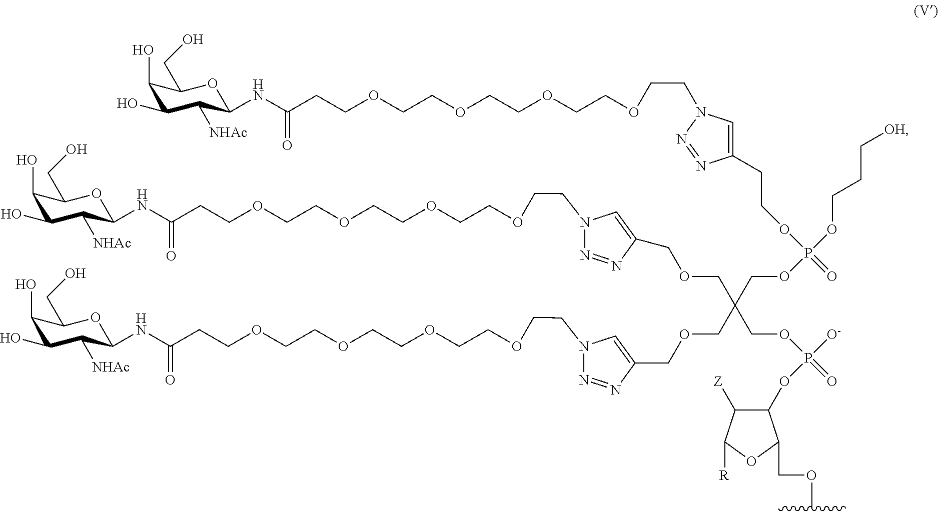

wherein Z in formula (V′) is —H, —OH, —O-Methyl, —F, or —O-methoxyethyl and R in formula (V′) is adenine, uracil, guanine, cytosine, thymine, abasic, or others.

In some instances, the linker and the asialoglycoprotein receptor targeting moiety with the last nucleotide on the 3′-terminus of the sense strand of the polynucleic acid molecule are shown in Formula (V″″):

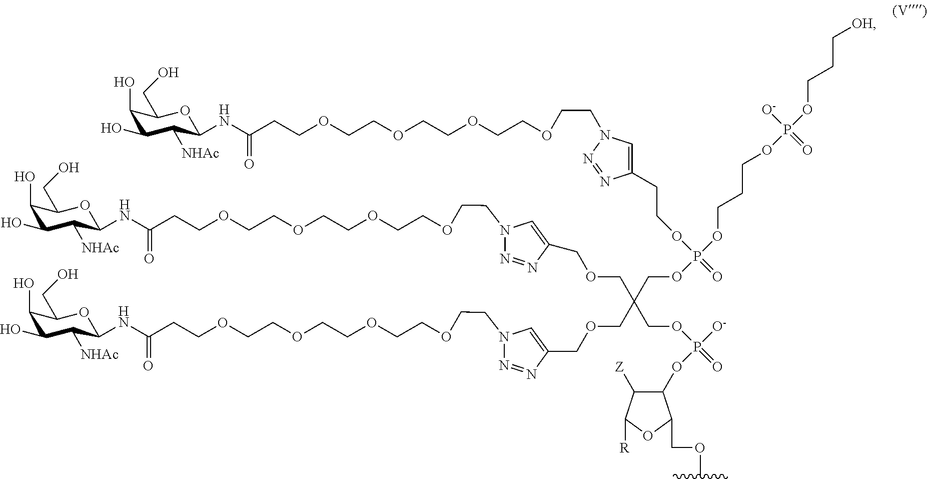

wherein Z in formula (V″″) is a moiety that corresponds to one of the sugar modifications described herein (e.g., —H, —OH, —O-Methyl, —F, or —O-methoxyethyl) and R in formula (V″″) is adenine, uracil, guanine, cytosine, thymine, abasic, or others.

In some instances, the linker and the asialoglycoprotein receptor targeting moiety with the last nucleotide on the 3′-terminus of the sense strand of the polynucleic acid molecule are shown in Formula (V′″″):

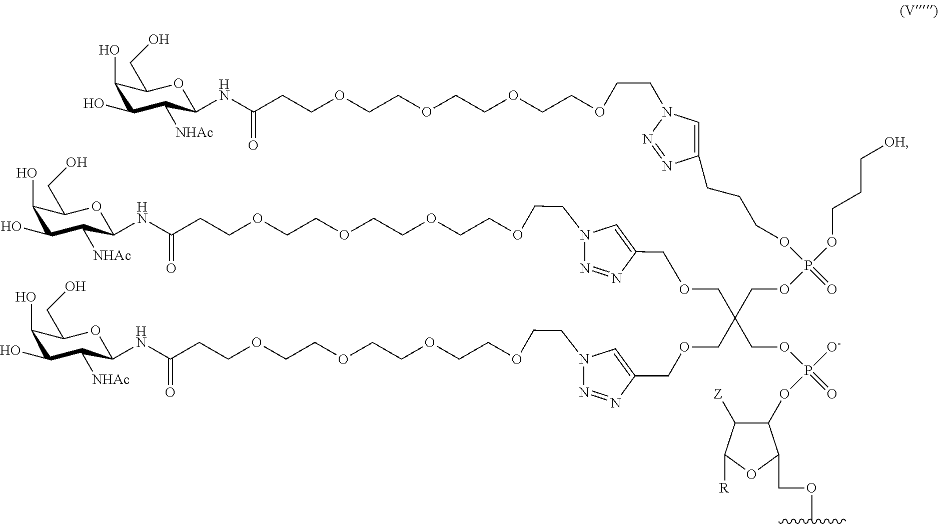

wherein Z in formula (V′″″) is a moiety that corresponds to one of the sugar modifications described herein (e.g., —H, —OH, —O-Methyl, —F, or —O-methoxyethyl) and R in formula (V′″″) is adenine, uracil, guanine, cytosine, thymine, abasic, or others.

In some instances, the linker and the asialoglycoprotein receptor targeting moiety with the last nucleotide on the 3′-terminus of the sense strand of the polynucleic acid molecule are shown in Formula (V″″″):

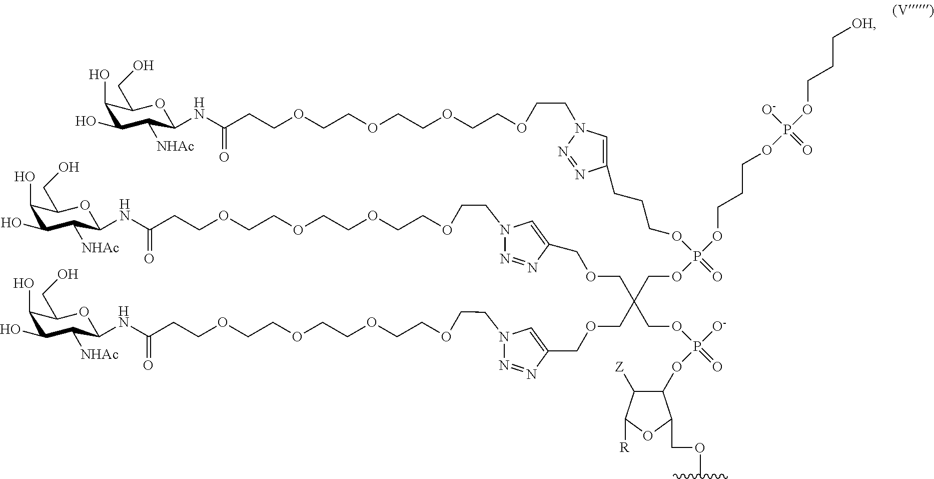

wherein Z in formula (V″″″) is a moiety that corresponds to one of the sugar modifications described herein (e.g., —H, —OH, —O-Methyl, —F, or —O-methoxyethyl) and R in formula (V″″″) is adenine, uracil, guanine, cytosine, thymine, abasic, or others.

In another aspect, provided herein is a pharmaceutical composition comprising a polynucleic acid molecule described herein or a polynucleic acid molecule conjugate described herein, and a pharmaceutically acceptable excipient.

In some cases, the pharmaceutical composition is formulated as a nanoparticle formulation. In some cases, the pharmaceutical composition is formulated for parenteral, oral, intranasal, buccal, rectal, transdermal, intravenous, subcutaneous, or intrathecal administration.

In another aspect, provided herein is a method of modulating expression of apolipoprotein C3 (APOC3) gene in a subject, comprising: administering to the subject a polynucleic acid molecule described herein or a polynucleic acid molecule conjugate described herein, or a pharmaceutical composition described herein, thereby modulating the expression of APOC3 gene in the subject. In some cases, the subject in need thereof is diagnosed of, suffers from, or having a symptom of cardiovascular disease or hypertriglyceridemia.

In another aspect, provided herein is a method of modulating triglyceride level in a subject in need thereof, comprising: administering to the subject a polynucleic acid molecule described herein or a polynucleic acid molecule conjugate described herein, or a pharmaceutical composition described herein, thereby modulating triglyceride level in the subject. In some cases, the subject in need thereof is diagnosed of, suffers from, or having a symptom of cardiovascular disease or hypertriglyceridemia.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings below.

FIGS. 1A-1X illustrate drug response curves for selected siRNAs for suppressing APOC3 in primary hepatocytes. FIG. 1A shows a drug response curve for SRS-000665. FIG. 1B shows a drug response curve for SRS-000770. FIG. 1C shows a drug response curve for SRS-000667. FIG. 1D shows a drug response curve for SRS-000771. FIG. 1E shows a drug response curve for SRS-000712. FIG. 1F shows a drug response curve for SRS-000772. FIG. 1G shows a drug response curve for SRS-000713. FIG. 1H shows a drug response curve for SRS-000773. FIG. 1I shows a drug response curve for SRS-000717. FIG. 1J shows a drug response curve for SRS-000774. FIG. 1K shows a drug response curve for SRS-000723. FIG. 1L shows a drug response curve for SRS-000775. FIG. 1M shows a drug response curve for SRS-000753. FIG. 1N shows a drug response curve for SRS-000776. FIG. 1O shows a drug response curve for SRS-000754. FIG. 1P shows a drug response curve for SRS-000777. FIG. 1Q shows a drug response curve for SRS-000755. FIG. 1R shows a drug response curve for SRS-000778. FIG. 1S shows a drug response curve for SRS-000756. FIG. 1T shows a drug response curve for SRS-000779, FIG. 1U shows a drug response curve for SRS-000757. FIG. 1V shows a drug response curve for SRS-000780. FIG. 1W shows a drug response curve for SRS-000758. FIG. 1X shows a drug response curve for SRS-000781.

FIG. 2 shows plasma APOC3 protein expression relative to pre-dose levels following single dose administrations of siRNAs listed in Table 5 (SRS-000225 and SRS-000228 to SRS-000232) to cynomolgus monkeys.

FIGS. 3A-3C show liver APOC3 mRNA expression relative to pre-dose levels following single dose administrations of siRNAs listed in Table 5 (SRS-000225 and SRS-000228 to SRS-000232) to cynomolgus monkeys. FIG. 3A shows liver APOC3 mRNA expression relative to pre-dose levels normalized to ACTB. FIG. 3B shows liver APOC3 mRNA expression relative to pre-dose levels normalized to ARL1. FIG. 3C shows liver APOC3 mRNA expression relative to pre-dose levels normalized to PPIA.

### DETAILED DESCRIPTION OF THE DISCLOSURE

APOC3 is a small protein with 79 amino acid residues that contains two amphipathic helices (see e.g., Gangabadage et al., J Biol Chem. 2008; 283(25):17416-17427). APOC3 is found to be on circulating lipoproteins including high-density lipoproteins (HDL), low-density lipoprotein (LDL), and triglyceride-rich lipoproteins (TRLs) such as chylomicrons (CM) and very low density lipoprotein (VLDL).

APOC3 is an important regulator in lipid (e.g., triglyceride) metabolism and cardiovascular diseases (pathological processes involved in atherosclerosis). Specifically, studies have shown that impaired catabolism of TRLs is linked to increased levels of plasma APOC3 (see e.g., Boren et al., Arterioscler Thromb Vasc Biol. 2015; 35(10):2218-2224). In addition to effects on lipid metabolism, APOC3 has been shown to directly influence development of atherosclerosis.

A lifelong deficiency of APOC3 may be cardioprotective. A reduction of plasma triglycerides by inhibition of APOC3 might be a promising strategy in management of severe hypertriglyceridemia or those with elevated plasma triglyceride.

Described herein is a polynucleic acid molecule for modulating expression of APOC3 gene. In some aspects, the polynucleic acid molecule is a single-stranded nucleic acid molecule. In some aspects, the polynucleic acid molecule comprises a sense strand and an antisense strand, and wherein the polynucleic acid molecule comprises a nucleic acid sequence in Table 1, Table 3, Table 5, and Table 12. Accordingly, provided herein are various target regions of human APOC3 mRNA the polynucleic acid molecule described herein hybridizes to. In some cases, provided herein is the sequences of the polynucleic acid molecule described herein. In some cases, provided herein is the possible modifications of the polynucleic acid molecule described herein. In some cases, provided herein is the possible conjugates of the polynucleic acid molecule described herein.

Also described herein is a method of modulating expression of APOC3 gene in a subject. Described further herein is a method of modulating triglyceride in a subject in need thereof.

### Definitions

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about,” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

“Percent (%) sequence identity” or “Percent (%) identity” with respect to the nucleic acid sequences identified herein is defined as the percentage of nucleic acid in a candidate sequence that are identical with the nucleic acid sequence being compared, after aligning the sequences considering any conservative substitutions as part of the sequence identity.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and the like. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Unless defined otherwise, all technical and scientific terms used herein have the same meaning as commonly understood by one of ordinary skill in the art to which the polynucleic acid molecules, the polynucleic acid molecule conjugates, the pharmaceutical compositions, the methods and other aspects belong.

As used herein, the term “complementary” indicates a sufficient degree of complementarity between two nucleic acid molecules that bind stably and specifically to avoid nonspecific binding.

As used herein, the term “polynucleic acid” and the term “polynucleotide” are interchangeably used to refer a chain of nucleotides. The term “nucleotide” includes a sequence “G,” “C,” “A,” “T” and “U” each generally stand for a nucleotide that contains guanine, cytosine, adenine, thymidine and uracil as a base. In some instances, the “nucleotide” can refer to a modified nucleotide (e.g., with modified sugar moiety, modified base, modified internucleotide linkage, or combination thereof, including, but not limited to 2′-modified nucleotide, LNA, ENA, BNA, UNA, GNA etc.) In some instances, the “nucleotide” can refer to a modified nucleotide with a non-canonical base (e.g. including, but not limited to, 2-thiouridine, 2-thiothymidine, inosine, 2-aminopurine, 2,6-diaminopurine, dihydrouridine, 4-thiouridine, 4-thiothymidine, 2-thiocytidine).

As used herein, a “subject” can be any mammal, including a human and a non-human primate.

The term “condition.” as used herein, includes diseases, disorders, and susceptibilities. In some cases, the condition is an APOC3 related disorder or symptoms thereof.

As used herein, the term “treat,” “treating” or “treatment” of any disease or disorder refers, in one instance, to ameliorating the disease or disorder (i.e., slowing or arresting or reducing the development of the disease or at least one of the clinical symptoms thereof). In another instance, “treat”, “treating” or “treatment” refers to alleviating or ameliorating at least one physical parameter including those which may not be discernible by the patient. In yet another instance, “treat”, “treating” or “treatment” refers to modulating the disease or disorder, either physically, (e.g., stabilization of a discernible symptom), physiologically, (e.g., stabilization of a physical parameter), or both.

The terms “prevent,” “preventing,” and “prevention,” as used herein, refer to a decrease in the occurrence of pathology of a condition in a subject, who does not have, but is at risk of or susceptible to developing a disease or condition. The prevention may be complete, e.g., the total absence of pathology of a condition in a subject. The prevention may also be partial, such that the occurrence of pathology of a condition in a subject is less than that which would have occurred without the present disclosure.

“Administering” and its grammatical equivalents as used herein can refer to providing pharmaceutical compositions described herein to a subject or a patient. Conventional methods, known to those of ordinary skill in the art of medicine, can be used to administer the composition to the subject, depending upon the type of disease to be treated or the site of the disease. For example, the composition can be administered, e.g., orally, parenterally, by inhalation spray, topically, rectally, nasally, buccally, vaginally, via an implanted reservoir, or via infusion. One or more such routes can be employed.

The terms “pharmaceutical composition” and its grammatical equivalents as used herein can refer to a mixture or solution comprising a therapeutically effective amount of an active pharmaceutical ingredient together with one or more pharmaceutically acceptable excipients, carriers, and/or a therapeutic agent to be administered to a subject, e.g., a human in need thereof.

The term “pharmaceutically acceptable” and its grammatical equivalents as used herein can refer to an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer a material, such as a carrier or diluent, which does not abrogate the biological activity or properties of the compound, and is relatively nontoxic, i.e., the material may be administered to a subject without causing undesirable biological effects or interacting in a deleterious manner with any of the components of the pharmaceutical composition in which it is contained.

A “pharmaceutically acceptable excipient” refers to an excipient that can be administered to a subject, together with an agent, and which does not destroy the pharmacological activity thereof and is nontoxic when administered in doses sufficient to deliver a therapeutic amount of the agent.

The term “therapeutic agent” can refer to any agent that, when administered to a subject, has a therapeutic, diagnostic, and/or prophylactic effect and/or elicits a desired biological and/or pharmacological effect. Therapeutic agents can also be referred to as “actives” or “active agents.” Such agents include, but are not limited to, cytotoxins, radioactive ions, chemotherapeutic agents, small molecule drugs, proteins, and nucleic acids.

It is appreciated that certain features of the polynucleic acid molecules, and/or polynucleic acid molecule conjugates, pharmaceutical composition comprising the polynucleic acid molecules or the polynucleic acid molecule conjugates, methods and other aspects, which are, for clarity, described in the context of separate embodiments, may also be provided in combination in a single embodiment. Conversely, various features of the polynucleic acid molecules, and/or polynucleic acid molecule conjugates, pharmaceutical composition comprising the polynucleic acid molecules or the polynucleic acid molecule conjugates, methods and other aspects, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present polynucleic acid molecules, and/or polynucleic acid molecule conjugates, pharmaceutical composition comprising the polynucleic acid molecules or the polynucleic acid molecule conjugates, methods and other aspects and are disclosed herein just as if each and every such sub-combination was individually and explicitly disclosed herein.

As used herein, the term “sense strand” can be interchangeably used with the term “passenger strand”, and the term “antisense strand” can be interchangeably used with the term “guide strand”.

As used herein, the term “consecutive sequence” refers to a sequence contains a number of consecutive nucleotides from a reference sequence. For example, if a reference sequence is N₁N₂N₃N₄N₅N₆N₇, a consecutive sequence can be N₁N₂N₃N₄ or N₃N₄N₅N₆, but a sequence of N₁N₃N₄N₅ or N₃N₄N₇ cannot be a consecutive sequence.

As used herein, the term “negative control” refers to a subject or a cell receiving no treatment or placebo.

### Polynucleic Acid Molecules

### Target Regions of Polynucleic Acid Molecules

Described herein is a polynucleic acid molecule for modulating expression of APOC3 gene. In some instances, the polynucleic acid molecule comprises a single-stranded nucleic acid molecule that hybridizes to certain regions of mRNA, In some instances, the polynucleic acid molecule is a double-stranded nucleic acid molecule. Also described herein is a polynucleic acid molecule for modulating expression of APOC3 gene, wherein the polynucleic acid molecule is a double-stranded nucleic acid molecule, which comprises a sense strand and an antisense strand, and the antisense strand hybridizes to certain regions of APOC3 mRNA.

In some aspects, the polynucleic acid molecule described herein hybridizes to certain regions of human APOC3 mRNA. In some instances, the human APOC3 mRNA is NM_000040.3. In some aspects, the polynucleic acid molecule described herein hybridizes to certain regions of non-human APOC3 mRNA.

In some aspects, the polynucleic acid molecule described herein hybridizes to the 5′ UTR region of human APOC3 mRNA. In some aspects, the polynucleic acid molecule described herein hybridizes to the coding region of human APOC3 mRNA. In some aspects, the polynucleic acid molecule described herein hybridizes to a portion of exon 1 of human APOC3 mRNA. In some aspects, the polynucleic acid molecule described herein hybridizes to a portion of exon 2 of human APOC3 mRNA. In some aspects, the polynucleic acid molecule described herein hybridizes to a portion of exon 3 of human APOC3 mRNA. In some aspects, the polynucleic acid molecule described herein hybridizes to a portion of exon 4 of human APOC3 mRNA. In some aspects, the polynucleic acid molecule described herein hybridizes to the 3′ UTR region of human APOC3 mRNA.

In some aspects, the target region that the polynucleic acid molecule described herein hybridizes to is determined by APOC3 silencing effectiveness and possible off-target effects. In some instances, the start of the target region fall between positions 1-10, 11-20, 21-30, 31-40, 41-50, 51-60, 61-70, 71-80, 81-90, or 91-100 of NM 000040.3. In some instances, the start of the target region fall between positions 101-110, 111-120, 121-130, 131-140, 141-150, 151-160, 161-170, 171-180, 181-190, or 191-200 of NM 000040.3. In some instances, the start of the target region fall between positions of 201-210, 211-220, 221-230, 231-240, 241-250, 251-260, 261-270, 271-280, 281-290, or 291-300 of NM_000040.3. In some instances, the start of the target region fall between positions 301-310, 311-320, 321-330, 331-340, 341-350, 351-360, 361-370, 371-380, 381-390, or 391-400 of NM_000040.3. In some instances, the start of the target region fall between positions 401-410, 411-420, 421-430, 431-440, 441-450, 451-460, 461-470, 471-480, 481-490, or 491-500 of NM 000040.3. In some instances, the start of the target region fall between positions 501-510, 511-520, 521-530, or 531-535 of NM 000040.3.

### Structure of Polynucleic Acid Molecules

### Single-Stranded Nucleic Acid Molecule

Described herein is a polynucleic acid molecule for modulating expression of APOC3 gene, wherein the polynucleic acid molecule comprises a single-stranded nucleic acid molecule that is reverse complementary to the target region of APOC3 mRNA as described above.

In some aspects, the polynucleic acid molecule described herein is not 100% complementary to the target region of APOC3 mRNA. Accordingly, in some instances, the polynucleic acid molecule described herein is about 95% complementary to the target region of APOC3 mRNA. In some instances, the polynucleic acid molecule described herein is about 90% complementary to the target region of APOC3 mRNA. In some instances, the polynucleic acid molecule described herein is about 85% complementary to the target region of APOC3 mRNA. In some instances, the polynucleic acid molecule described herein is about 80% complementary to the target region of APOC3 mRNA. In some in stances, the polynucleic acid molecule described herein is about 75% complementary to the target region of APOC3 mRNA. In some instances, the polynucleic acid molecule described herein is about 70% complementary to the target region of APOC3 mRNA.

In some aspects, the polynucleic acid molecule described herein comprises a nucleic acid sequence in Table 1, Table 3, Table 5, and Table 12. In some instances, the polynucleic acid molecule described herein comprises a nucleic acid sequence that is at least 80%, at least 85%, at least 90%, or at least 95% complementary to a sequence in Table 1, Table 3, Table 5, and Table 12. In some instances, the polynucleic acid molecule described herein comprises a nucleic acid sequence that is at least 80%, at least 85%, at least 90%, or at least 95% complementary to a nucleic acid sequence selected from SEQ ID NOs: 217-324, 481-504, 541-546, and 575-585. In some instances, the polynucleic acid molecule described herein comprises a nucleic acid sequence that is at least 80%, at least 85%, at least 90%, or at least 95% complementary to a nucleic acid sequence selected from SEQ ID NOs: 541-546.

In some instances, the polynucleic acid molecule described herein comprises a nucleic acid sequence that is at least 80%, at least 85%, at least 90%, or at least 95% complementary to a sequence in Table 1, Table 3, Table 5, and Table 12, excluding overhangs. In some instances, the polynucleic acid molecule described herein comprises a nucleic acid sequence that is at least 80%, at least 85%, at least 90%, or at least 95% complementary to a nucleic acid sequence selected from SEQ ID NOs: 217-324, 481-504, 541-546, and 575-585, excluding overhangs. In some instances, the polynucleic acid molecule described herein comprises a nucleic acid sequence that is at least 80%, at least 85%, at least 90%, or at least 95% complementary to a nucleic acid sequence selected from SEQ ID NOs: 541-546, excluding overhangs.

In yet other aspects, the polynucleic acid molecule described herein comprises a nucleic acid sequence comprising 14 consecutive nucleotides that are complementary to a sequence in Table 1, Table 3, Table 5, and Table 12 with no more than 1, 2, 3, or 4 mismatches. In some aspects, the polynucleic acid molecule described herein comprises a nucleic acid sequence comprising 14 consecutive nucleotides that are complementary to a nucleic acid sequence of SEQ ID NOs: 217-324, 481-504, 541-546, and 575-585 with no more than 1, 2, 3, or 4 mismatches. In yet other aspects, the polynucleic acid molecule described herein comprises a nucleic acid sequence comprising 15 consecutive nucleotides that are complementary to a sequence in Table 1, Table 3, Table 5, and Table 12 with no more than 1, 2, 3, or 4 mismatches. In some aspects, the polynucleic acid molecule described herein comprises a nucleic acid sequence comprising 15 consecutive nucleotides that are complementary to a nucleic acid sequence of SEQ ID NOs: 217-324, 481-504, 541-546, and 575-585 with no more than 1, 2, 3, or 4 mismatches. In yet other aspects, the polynucleic acid molecule described herein comprises a nucleic acid sequence comprising 16 consecutive nucleotides that are complementary to a sequence in Table 1, Table 3, Table 5, and Table 12 with no more than 1, 2, 3, or 4 mismatches. In some aspects, the polynucleic acid molecule described herein comprises a nucleic acid sequence comprising 16 consecutive nucleotides that are complementary to a nucleic acid sequence of SEQ ID NOs: 217-324, 481-504, 541-546, and 575-585 with no more than 1, 2, 3, or 4 mismatches. In yet other aspects, the polynucleic acid molecule described herein comprises a nucleic acid sequence comprising 17 consecutive nucleotides that are complementary to a sequence in Table 1, Table 3, Table 5, and Table 12 with no more than 1, 2, 3, or 4 mismatches. In some aspects, the polynucleic acid molecule described herein comprises a nucleic acid sequence comprising 17 consecutive nucleotides that are complementary to a nucleic acid sequence of SEQ ID NOs: 217-324, 481-504, 541-546, and 575-585 with no more than 1, 2, 3, or 4 mismatches. In yet other aspects, the polynucleic acid molecule described herein comprises a nucleic acid sequence comprising 18 consecutive nucleotides that are complementary to a sequence in Table 1, Table 3, Table 5, and Table 12 with no more than 1, 2, 3, or 4 mismatches. In some aspects, the polynucleic acid molecule described herein comprises a nucleic acid sequence comprising 18 consecutive nucleotides that are complementary to a nucleic acid sequence of SEQ ID NOs: 217-324, 481-504, 541-546, and 575-585 with no more than 1, 2, 3, or 4 mismatches. In yet other aspects, the polynucleic acid molecule described herein comprises a nucleic acid sequence comprising 19 consecutive nucleotides that are complementary to a sequence in Table 1, Table 3, Table 5, and Table 12 with no more than 1, 2, 3, or 4 mismatches. In some aspects, the polynucleic acid molecule described herein comprises a nucleic acid sequence comprising 19 consecutive nucleotides that are complementary to a nucleic acid sequence of SEQ ID NOs: 217-324, 481-504, 541-546, and 575-585 with no more than 1, 2, 3, or 4 mismatches. In yet other aspects, the polynucleic acid molecule described herein comprises a nucleic acid sequence comprising 20 consecutive nucleotides that are complementary to a sequence in Table 1, Table 3, Table 5, and Table 12 with no more than 1, 2, 3, or 4 mismatches. In some aspects, the polynucleic acid molecule described herein comprises a nucleic acid sequence comprising 20 consecutive nucleotides that are complementary to a nucleic acid sequence of SEQ ID NOs: 217-324, 481-504, 541-546, and 575-585 with no more than 1, 2, 3, or 4 mismatches. In yet other aspects, the polynucleic acid molecule described herein comprises a nucleic acid sequence comprising 21 consecutive nucleotides that are complementary to a sequence in Table 1, Table 3, Table 5, and Table 12 with no more than 1, 2, 3, or 4 mismatches. In some aspects, the polynucleic acid molecule described herein comprises a nucleic acid sequence comprising 21 consecutive nucleotides that are complementary to a nucleic acid sequence of SEQ ID NOs: 217-324, 481-504, 541-546, and 575-585 with no more than 1, 2, 3, or 4 mismatches. In yet other aspects, the polynucleic acid molecule described herein comprises a nucleic acid sequence comprising 22 consecutive nucleotides that are complementary to a sequence in Table 1, Table 3, Table 5, and Table 12 with no more than 1, 2, 3, or 4 mismatches. In some aspects, the polynucleic acid molecule described herein comprises a nucleic acid sequence comprising 22 consecutive nucleotides that are complementary to a nucleic acid sequence of SEQ ID NOs: 217-324, 481-504, 541-546, and 575-585 with no more than 1, 2, 3, or 4 mismatches.

In some aspects, the polynucleic acid molecule described herein comprises a nucleic acid sequence comprising 14 consecutive nucleotides that are complementary to a nucleic acid sequence of SEQ ID NOs: 541-546 with no more than 1, 2, 3, or 4 mismatches. In some aspects, the polynucleic acid molecule described herein comprises a nucleic acid sequence comprising 15 consecutive nucleotides that are complementary to a nucleic acid sequence of SEQ ID NOs: 541-546 with no more than 1, 2, 3, or 4 mismatches. In some aspects, the polynucleic acid molecule described herein comprises a nucleic acid sequence comprising 16 consecutive nucleotides that are complementary to a nucleic acid sequence of SEQ ID) NOs: 541-546 with no more than 1, 2, 3, or 4 mismatches. In some aspects, the polynucleic acid molecule described herein comprises a nucleic acid sequence comprising 17 consecutive nucleotides that are complementary to a nucleic acid sequence of SEQ ID NOs: 541-546 with no more than 1, 2, 3, or 4 mismatches. In some aspects, the polynucleic acid molecule described herein comprises a nucleic acid sequence comprising 18 consecutive nucleotides that are complementary to a nucleic acid sequence of SEQ ID NOs: 541-546 with no more than 1, 2, 3, or 4 mismatches. In some aspects, the polynucleic acid molecule described herein comprises a nucleic acid sequence comprising 19 consecutive nucleotides that are complementary to a nucleic acid sequence of SEQ ID NOs: 541-546 with no more than 1, 2, 3, or 4 mismatches. In some aspects, the polynucleic acid molecule described herein comprises a nucleic acid sequence comprising 20 consecutive nucleotides that are complementary to a nucleic acid sequence of SEQ ID NOs: 541-546 with no more than 1, 2, 3, or 4 mismatches. In some aspects, the polynucleic acid molecule described herein comprises a nucleic acid sequence comprising 21 consecutive nucleotides that are complementary to a nucleic acid sequence of SEQ ID NOs: 541-546 with no more than 1, 2, 3, or 4 mismatches. In some aspects, the polynucleic acid molecule described herein comprises a nucleic acid sequence comprising 22 consecutive nucleotides that are complementary to a nucleic acid sequence of SEQ ID NOs: 541-546 with no more than 1, 2, 3, or 4 mismatches.

In yet other aspects, the polynucleic acid molecule described herein comprises a nucleic acid sequence comprising 14, 15, 16, 17, 18, 19, 20, 21, or 22 consecutive nucleotides that are complementary to a sequence in Table 1, Table 3, Table 5, and Table 12 without overhangs with no more than 1, 2, 3, or 4 mismatches. In some aspects, the polynucleic acid molecule described herein comprises a nucleic acid sequence comprising 14, 15, 16, 17, 18, 19, 20, 21, or 22 consecutive nucleotides that are complementary to a nucleic acid sequence of SEQ ID NOs: 217-324, 481-504, 541-546, and 575-585 without overhangs with no more than 1, 2, 3, or 4 mismatches. In some aspects, the polynucleic acid molecule described herein comprises a nucleic acid sequence comprising 14, 15, 16, 17, 18, 19, 20, 21, or 22 consecutive nucleotides that are complementary to a nucleic acid sequence of SEQ ID NOs: 541-546 without overhangs with no more than 1, 2, 3, or 4 mismatches.

In some aspects, the polynucleic acid molecule described herein comprises a strand of at least 10, 11, 12, 13, 14, or 15 nucleotides in length. In some aspects, the polynucleic acid molecule described herein comprises a strand of about 15-40, 16-30, 17-30, 18-30, 18-27, 18-25, 18-23, 19-23, 20-23, or 21-23 nucleotides in length. In some aspects, the polynucleic acid molecule described herein comprises a strand of about 15, 16, 17, 18, 19, 20 nucleotides long. In some aspects, the polynucleic acid molecule described herein comprises a strand of about 21, 22, 23, 24, 25 nucleotides long. In some aspects, the polynucleic acid molecule described herein comprises a strand of about 26, 27, 28, 29, 30 nucleotides long.

In some aspects, the polynucleic acid molecule described herein comprises a single-stranded nucleic acid of at least 10, 11, 12, 13, 14, or 15 nucleotides in length. In some aspects, the polynucleic acid molecule described herein comprises a single-stranded nucleic acid of about 15-30, 16-30, 17-30, 18-30, 18-27, 18-25, 18-23, 19-23, 20-23, or 21-23 nucleotides in length. In some aspects, the polynucleic acid molecule described herein comprises a single-stranded nucleic acid of about 15, 16, 17, 18, 19, 20 nucleotides long. In some aspects, the polynucleic acid molecule described herein comprises a single-stranded nucleic acid of about 21, 22, 23, 24, 25 nucleotides long. In some aspects, the polynucleic acid molecule described herein comprises a single-stranded nucleic acid of about 26, 27, 28, 29, 30 nucleotides long. In some aspects, the polynucleic acid molecule described herein comprises a single-stranded nucleic acid of about 21 nucleotides long. In some aspects, the polynucleic acid molecule described herein comprises a single-stranded nucleic acid of about 23 nucleotides long.

### Double-Stranded Nucleic Acid Molecule

Further described herein is a polynucleic acid molecule for modulating expression of APOC3 gene, wherein the polynucleic acid molecule is a double-stranded molecule that comprises a sense strand and an antisense strand, and the antisense strand is reverse complementary to the target region of APOC3 mRNA as described above.

In some aspects, the antisense strand described herein is 100% complementary to the target region of APOC3 mRNA. In other aspects, the antisense strand described herein is not 100% complementary to the target region of APOC3 mRNA. Accordingly, in some instances, the antisense strand described herein is about 95% complementary to the target region of APOC3 mRNA. In some aspects, the antisense strand described herein is about 90% complementary to the target region of APOC3 mRNA. In some aspects, the antisense strand described herein is about 85% complementary to the target region of APOC3 mRNA. In some aspects, the antisense strand described herein is about 80% complementary to the target region of APOC3 mRNA. In some aspects, the antisense strand described herein is about 75% complementary to the target region of APOC3 mRNA. In some aspects, the antisense strand described herein is about 70% complementary to the target region of APOC3 mRNA.

In some aspects, the polynucleic acid molecule described herein comprises a nucleic acid sequence in Table 1, Table 3, Table 5, and Table 12. In other aspects, the polynucleic acid molecule described herein comprises a nucleic acid sequence that is at least 80%, at least 85%, at least 90%, or at least 95% identical to a sequence in Table 1, Table 3, Table 5, and Table 12. In some instances, the sense strand described herein comprises a nucleic acid sequence that is at least 80%, at least 85%, at least 90%, or at least 95% identical to a nucleic acid sequence selected from SEQ ID NOs:217-324, 481-504, 541-546, and 575-585. In some instances, the antisense strand described herein comprises a nucleic acid sequence that is at least 80%, at least 85%, at least 90%, or at least 95% identical to a nucleic acid sequence selected from SEQ ID NOs:1-108, 433-456, 529-534, and 553-563. In some instances, the sense strand described herein comprises a nucleic acid sequence that is at least 80%, at least 85%, at least 90%, or at least 95% identical to a nucleic acid sequence selected from SEQ ID NOs: 541-546. In some instances, the antisense strand described herein comprises a nucleic acid sequence that is at least 80%, at least 85%, at least 90%, or at least 95% identical to a nucleic acid sequence selected from SEQ ID NOs: 529-534.

In yet other aspects, the polynucleic acid molecule described herein comprises a nucleic acid sequence comprising 14 consecutive sequences out of the sequences in Table 1, Table 3, Table 5, and Table 12 with no more than 1, 2, 3 or 4 mismatches. In some aspects, the sense strand described herein comprises a nucleic acid sequence comprising 14 consecutive sequences of SEQ ID NOs: 217-324, 481-504, 541-546, and 575-585 with no more than 1, 2, 3, or 4, mismatches. In some aspects, the antisense strand described herein comprises a nucleic acid sequence comprising 14 consecutive sequences of SEQ ID NOs: 1-108, 433-456, 529-534, and 553-563 with no more than 1, 2, 3, or 4 mismatches. In yet other aspects, the polynucleic acid molecule described herein comprises a nucleic acid sequence comprising 15 consecutive sequences out of the sequences in Table 1, Table 3, Table 5, and Table 12 with no more than 1, 2, 3, or 4 mismatches. In some aspects, the sense strand described herein comprises a nucleic acid sequence comprising 15 consecutive sequences of SEQ ID NOs: 217-324, 481-504, 541-546, and 575-585 with no more than 1, 2, 3, or 4 mismatches. In some aspects, the antisense strand described herein comprises a nucleic acid sequence comprising 15 consecutive sequences of SEQ ID NOs: 1-108, 433-456, 529-534, and 553-563 with no more than 1, 2, 3, or 4 mismatches. In yet still other aspects, the polynucleic acid molecule described herein comprises a nucleic acid sequence comprising 16 consecutive sequences out of the sequences in Table 1, Table 3, Table 5, and Table 12 with no more than 1, 2, 3, or 4 mismatches. In some aspects, the sense strand described herein comprises a nucleic acid sequence comprising 16 consecutive sequences of SEQ ID NOs: 217-324, 481-504, 541-546, and 575-585 with no more than 1, 2, 3, or 4 mismatches. In some aspects, the antisense strand described herein comprises a nucleic acid sequence comprising 16 consecutive sequences of SEQ ID NOs: 1-108, 433-456, 529-534, and 553-563 with no more than 1, 2, 3, or 4 mismatches. In yet still other aspects, the polynucleic acid molecule described herein comprises a nucleic acid sequence comprising 17 consecutive sequences out of the sequences in Table 1, Table 3, Table 5, and Table 12 with no more than 1, 2, 3, or 4 mismatches. In some aspects, the sense strand described herein comprises a nucleic acid sequence comprising 17 consecutive sequences of SEQ ID NOs: 217-324, 481-504, 541-546, and 575-585 with no more than 1, 2, 3, or 4 mismatches. In some aspects, the antisense strand described herein comprises a nucleic acid sequence comprising 17 consecutive sequences of SEQ ID NOs: 1-108, 433-456, 529-534, and 553-563 with no more than 1, 2, 3, or 4 mismatches. In yet other aspects, the polynucleic acid molecule described herein comprises a nucleic acid sequence comprising 18 consecutive sequences out of the sequences in Table 1, Table 3, Table 5, and Table 12 with no more than 1, 2, 3, or 4 mismatches. In some aspects, the sense strand described herein comprises a nucleic acid sequence comprising 18 consecutive sequences of SEQ ID NOs: 217-324, 481-504, 541-546, and 575-585 with no more than 1, 2, 3, or 4 mismatches. In some aspects, the antisense strand described herein comprises a nucleic acid sequence comprising 18 consecutive sequences of SEQ ID NOs: 1-108, 433-456, 529-534, and 553-563 with no more than 1, 2, 3, or 4 mismatches. In yet other aspects, the polynucleic acid molecule described herein comprises a nucleic acid sequence comprising 19 consecutive sequences out of the sequences in Table 1, Table 3, Table 5, and Table 12 with no more than 1, 2, 3, or 4 mismatches. In some aspects, the sense strand described herein comprises a nucleic acid sequence comprising 19 consecutive sequences of SEQ ID NOs: 217-324, 481-504, 541-546, and 575-585 with no more than 1, 2, 3, or 4 mismatches. In some aspects, the antisense strand described herein comprises a nucleic acid sequence comprising 19 consecutive sequences of SEQ ID NOs: 1-108, 433-456, 529-534, and 553-563 with no more than 1, 2, 3, or 4 mismatches. In yet other aspects, the polynucleic acid molecule described herein comprises a nucleic acid sequence comprising 20 consecutive sequences out of the sequences in Table 1, Table 3, Table 5, and Table 12 with no more than 1, 2, 3, or 4 mismatches. In some aspects, the sense strand described herein comprises a nucleic acid sequence comprising 20 consecutive sequences of SEQ ID NOs: 217-324, 481-504, 541-546, and 575-585 with no more than 1, 2, 3, or 4 mismatches. In some aspects, the antisense strand described herein comprises a nucleic acid sequence comprising 20 consecutive sequences of SEQ ID NOs: 1-108, 433-456, 529-534, and 553-563 with no more than 1, 2, 3, or 4 mismatches. In yet other aspects, the polynucleic acid molecule described herein comprises a nucleic acid sequence comprising 21 consecutive sequences out of the sequences in Table 1, Table 3, Table 5, and Table 12 with no more than 1, 2, 3, or 4 mismatches. In some aspects, the sense strand described herein comprises a nucleic acid sequence comprising 21 consecutive sequences of SEQ ID NOs: 217-324, 481-504, 541-546, and 575-585 with no more than 1, 2, 3, or 4 mismatches. In some aspects, the antisense strand described herein comprises a nucleic acid sequence comprising 21 consecutive sequences of SEQ ID NOs: 1-108, 433-456, 529-534, and 553-563 with no more than 1, 2, 3, or 4 mismatches. In yet other aspects, the polynucleic acid molecule described herein comprises a nucleic acid sequence comprising 22 consecutive sequences out of the sequences in Table 1, Table 3, Table 5, and Table 12 with no more than 1, 2, 3, or 4 mismatches. In some aspects, the sense strand described herein comprises a nucleic acid sequence comprising 22 consecutive sequences of SEQ ID NOs: 217-324, 481-504, 541-546, and 575-585 with no more than 1, 2, 3, or 4 mismatches. In some aspects, the antisense strand described herein comprises a nucleic acid sequence comprising 22 consecutive sequences of SEQ ID NOs: 1-108, 433-456, 529-534, and 553-563 with no more than 1, 2, 3, or 4 mismatches.

In some aspects, the sense strand described herein comprises a nucleic acid sequence comprising 15 consecutive sequences of SEQ ID NOs: 541-546 with no more than 1, 2, 3, or 4 mismatches. In some aspects, the antisense strand described herein comprises a nucleic acid sequence comprising 15 consecutive sequences of SEQ ID NOs: 529-534 with no more than 1, 2, 3, or 4 mismatches. In some aspects, the sense strand described herein comprises a nucleic acid sequence comprising 16 consecutive sequences of SEQ ID NOs: 541-546 with no more than 1, 2, 3, or 4 mismatches. In some aspects, the antisense strand described herein comprises a nucleic acid sequence comprising 16 consecutive sequences of SEQ ID NOs: 529-534 with no more than 1, 2, 3, or 4 mismatches. In some aspects, the sense strand described herein comprises a nucleic acid sequence comprising 17 consecutive sequences of SEQ ID NOs: 541-546 with no more than 1, 2, 3, or 4 mismatches. In some aspects, the antisense strand described herein comprises a nucleic acid sequence comprising 17 consecutive sequences of SEQ ID NOs: 529-534 with no more than 1, 2, 3, or 4 mismatches. In some aspects, the sense strand described herein comprises a nucleic acid sequence comprising 18 consecutive sequences of SEQ ID NOs: 541-546 with no more than 1, 2, 3, or 4 mismatches. In some aspects, the antisense strand described herein comprises a nucleic acid sequence comprising 18 consecutive sequences of SEQ ID NOs: 529-534 with no more than 1, 2, 3, or 4 mismatches. In some aspects, the sense strand described herein comprises a nucleic acid sequence comprising 19 consecutive sequences of SEQ ID NOs: 541-546 with no more than 1, 2, 3, or 4 mismatches. In some aspects, the antisense strand described herein comprises a nucleic acid sequence comprising 19 consecutive sequences of SEQ ID NOs: 529-534 with no more than 1, 2, 3, or 4 mismatches. In some aspects, the sense strand described herein comprises a nucleic acid sequence comprising 20 consecutive sequences of SEQ ID NOs: 541-546 with no more than 1, 2, 3, or 4 mismatches. In some aspects, the antisense strand described herein comprises a nucleic acid sequence comprising 20 consecutive sequences of SEQ ID NOs: 529-534 with no more than 1, 2, 3, or 4 mismatches. In some aspects, the sense strand described herein comprises a nucleic acid sequence comprising 21 consecutive sequences of SEQ ID NOs: 541-546 with no more than 1, 2, 3, or 4 mismatches. In some aspects, the antisense strand described herein comprises a nucleic acid sequence comprising 2l consecutive sequences of SEQ ID NOs: 529-534 with no more than 1, 2, 3, or 4 mismatches. In some aspects, the sense strand described herein comprises a nucleic acid sequence comprising 22 consecutive sequences of SEQ ID NOs: 541-546 with no more than 1, 2, 3, or 4 mismatches. In some aspects, the antisense strand described herein comprises a nucleic acid sequence comprising 22 consecutive sequences of SEQ ID NOs: 529-534 with no more than 1, 2, 3, or 4 mismatches.

In some aspects, the polynucleic acid molecule described herein comprises a sense and an antisense strand of at least 10, 11, 12, 13, 14, or 15 nucleotides in length. In some aspects, the polynucleic acid molecule described herein comprises a sense and an antisense strand of about 15-30, 16-30, 17-30, 18-30, 18-27, 18-25, 18-23, 19-23, 20-23, or 21-23 nucleotides in length. In some aspects, the polynucleic acid molecule described herein comprises a sense and an antisense strand of about 15, 16, 17, 18, 19, 20 nucleotides long. In some aspects, the polynucleic acid molecule described herein comprises a sense and an antisense strand of about 21, 22, 23, 24, 25 nucleotides long. In some aspects, the polynucleic acid molecule described herein comprises a sense and an antisense strand of about 26, 27, 28, 29, 30 nucleotides long. In some aspects, the polynucleic acid molecule described herein comprises a sense strand of 19 nucleotides long, and an antisense strand of about 21 nucleotides long. In some aspects, the polynucleic acid molecule described herein comprises a sense strand of 21 nucleotides long, and an antisense strand of about 23 nucleotides long.

In some aspects, the sense strand and the antisense strand described herein are reverse complementary to each other and form a duplex with a 3′ overhang on the antisense strand. In some aspects, the sense strand and the antisense strand described herein are reverse complementary to each other and form a duplex with a 5′ overhang on the antisense strand. In some aspects, the sense strand and the antisense strand described herein are reverse complementary to each other and form a duplex with a 3′ overhang on the sense strand. In some aspects, the sense strand and the antisense strand described herein are reverse complementary to each other and form a duplex with a 5′ overhang on the sense strand.

### Modifications of Polynucleic Acid Molecules

In some aspects, described herein is the polynucleic acid molecule described herein with modifications. In some aspects, the modifications described herein occurs one or more different structures of the polynucleotide molecule described herein (e.g., modifications on sugar ring(s), backbone(s), base(s)). In some aspects, the modifications described herein comprise substitutions of one or more nucleotide in the polynucleic acid molecule described herein. In some aspects, different percentages of the polynucleic acid molecule described herein comprise the modifications described herein. In some aspects, different positions of the polynucleic acid molecule described herein comprise the modifications described herein. WO/2018/035380 is herein incorporated by reference in its entirety.

### Types of Modifications

In some aspects, the polynucleotide molecule described herein comprises one or more sugar-modified nucleotide. In some aspects, the sugar-modified nucleotide is a 2′-fluoro modified nucleotide. In some instances, the sugar-modified nucleotide includes a modification at a 2′ hydroxyl group of the ribose moiety. In some instances, the sugar-modified nucleotide includes modification with an H, OR, R, halo, SH, SR, NH2, NHR, NR2, or CN, wherein R is an alkyl moiety. In some aspects, the sugar-modified nucleotide is a 2′-O-methyl modified nucleotide or 2′-alkoxy modified nucleotide (e.g., 2′-methoxy modified nucleotide). In some instances, 2′ hydroxyl group modification includes 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In some instances, 2′ hydroxyl group of the ribose moiety includes a locked or bridged ribose modification (e.g., LNA), an unlocked ribose modification (e.g., UNA), or ethylene nucleic acids (ENA). In some instances, the alkyl moiety comprises a hetero substitution. In some instances, the carbon of the heterocyclic group is substituted by a nitrogen, oxygen or sulfur. In some aspects, the sugar-modified nucleotide is a 2′-amino modified nucleotide. In some aspects, the sugar-modified nucleotide is a 2′-azido modified nucleotide. In some aspects, the sugar-modified nucleotide is a 2′-deoxy modified nucleotide. In some aspects, the sugar-modified nucleotide is a 2′-O-methoxythyl (2′-MOE). In some aspects, the sugar-modified nucleotide is a locked nucleic acid (LNA). In some aspects, the sugar-modified nucleotide is an ethylene-bridged nucleic acid (ENA). In some aspects, the sugar-modified nucleotide is a (S)-constrained ethyl (cEt). In some aspects, the sugar-modified nucleotide is a tricyclo-DNA (tcDNA). In some aspects, the sugar-modified nucleotide is a 2′-NH₂ nucleic acid.

In some aspects, the polynucleotide molecule described herein comprises one or more sugarphosphate-modified nucleotide. In some aspects, the modified sugarphosphate is phosphorodiamidate morpholino (PMO). In some aspects, the modified sugarphosphate is phosphoramidate. In some instances, the heterocyclic substitution includes imidazole, and pyrrolidino. In some aspects, the modified sugarphosphate is thiophosphoramidate. In some aspects, the modified sugarphosphate is peptide nucleic acid (PNA).

In some aspects, the polynucleotide molecule described herein comprises one or more backbone-modified nucleotide. In some aspects, the modified backbone is a methylphosphonate. In some aspects, the modified backbone is phosphorothioate. In some aspects, the modified backbone is a guanidinopropyl phosphoramidate. In some aspects, the modified backbone is a mesyl-phosphoramidate (MsPA) linkages. In some instances, the modified backbone comprises one or more of phosphorodithioates, methylphosphonates, 5′-alkylenephosphonates, 5′-methylphosphonate, 3′-alkylene phosphonates, borontrifluoridates, borano phosphate esters and selenophosphates of 3′-5′ linkage or 2′-5′ linkage, phosphotriesters, thionoalkylphosphotriesters, hydrogen phosphonate linkages, alkyl phosphonates, alkylphosphonothioates, arylphosphonothioates, phosphoroselenoates, phosphoramidates.

In some aspects, the modified nucleotide comprises a modified guanine (e.g., inosine) or one or more of any types of unnatural nucleic acids.

In some aspects, the modified backbone is phosphorothioate, and the phosphorothioate is a stereochemically enriched phosphorothioate. In certain aspects, the strand contains at least one stereochemically enriched phosphorothioate. In some aspects, the strand comprises at least 1, 2, 3 stereochemically enriched phosphorothioates. In some aspects, the strand comprises only 1, 2, 3, or 4 stereochemically enriched phosphorothioates. In further aspects, at least one (e.g., one or two) stereochemically enriched phosphorothioate is disposed between two consecutive nucleosides that are two of six 5′-terminal nucleosides of the strand. In yet further aspects, at least one (e.g., one or two) stereochemically enriched phosphorothioate is disposed between two consecutive nucleosides that are two of six 3′-terminal nucleosides of the strand. In still further aspects, one stereochemically enriched phosphorothioate is covalently bonded to the first nucleoside and the second nucleoside from the 5′-end within the strand. In some aspects, one stereochemically enriched phosphorothioate is covalently bonded to the twenty first nucleoside and the twenty second nucleoside from the 5′-end within the strand. In certain aspects, one stereochemically enriched phosphorothioate is covalently bonded to the twenty second nucleoside and the twenty third nucleoside from the 5′-end within the strand. In particular aspects, the stereochemically enriched phosphorothioate has Rₚ stereochemical identity. In certain aspects, the stereochemically enriched phosphorothioate has Sₚ stereochemical identity.

In some aspects, the polynucleotide molecules described herein comprises one or more (e.g., from 1 to 20, from 1 to 10, or from 1 to 5) stereochemically enriched (e.g., internucleoside) phosphorothioates (e.g., having diastereomeric excess of at least 10%, at least 50%, at least 60%, at least 70%, at least 80%, or at least 90%, e.g., up to about 99%, for the P-stereogenic center). The polynucleotide molecules described herein comprises one or more (e.g., from 1 to 20, from 1 to 10, or from 1 to 5; e.g., internucleoside) phosphorodithioates. The phosphorodithioates may be non-P-stereogenic in the polynucleotide molecules described herein. Phosphorothioates aid phosphorodithioates may enhance the stability of the polynucleotide molecules described herein to exonuclease activity of serum. Non-P-stereogenic phosphorodithioates may simplify the synthesis of the polynucleotide molecule described herein by reducing the number of possible diastereomers. Typically, the phosphorothioate or phosphorodithioate may connect two contiguous nucleosides within the six 3′-terminal nucleosides and the six 5′-terminal nucleosides of the polynucleotide molecules described herein. In some aspects, the stereochemically enriched phosphorothioate (e.g., Rₚ-enriched phosphorothioate) may be covalently bonded to the first nucleoside (e.g., the 3′-carbon atom of the first nucleoside) and the second nucleoside (e.g., the 5′-carbon atom of the second nucleoside) from the 5′-end of the antisense strand. Additionally or alternatively, the stereochemically enriched phosphorothioate (e.g., Sp-enriched phosphorothioate) may be covalently bonded to the 21^{st} nucleoside (e.g., the 3′-carbon atom of the 21^{st}′ nucleoside) from the 5′-end and the 22^{nd} nucleoside (e.g., the 5′-carbon atom of the 22^{nd} nucleoside) of the antisense strand. Further, additionally or alternatively, the stereochemically enriched phosphorothioate (e.g., Sₚ-enriched phosphorothioate or Rp-enriched phosphorothioate) may be covalently bonded to the 22^{nd} nucleoside (e.g., the 3′-carbon atom of the 22^{nd}-nucleoside) and the 23^{rd} nucleoside (e.g., the 5′-carbon atom of the 23^{rd} nucleoside) from the 5′-end of the antisense strand.

Combinations of a 5′ Rₚ-enriched phosphorothioate (e g., Rₚ-enriched phosphorothioate covalently bonded to the first nucleoside (e.g., the 3′-carbon atom of the first nucleoside) and the second nucleoside (e.g., the 5′-carbon atom of the second nucleoside) from the 5′-end and a 3′ Sₚ-enriched phosphorothioate (e.g., Sₚ-enriched phosphorothioate covalently bonded to the 21^{st} nucleoside (e.g., the 3′-carbon atom of the 21^{st} nucleoside) and the 22^{nd} nucleoside (e.g., the 5′-carbon atom of the 22^{nd} nucleoside) from the 5′-end in an antisense strand can produce superior efficacy and/or duration of action, e.g., as measured by the reduction in the activity of the target relative to a reference guide strand that lacks the combination of a 5′ Rₚ-enriched phosphorothioate and a 3′ Sₚ-enriched phosphorothioate, or a 5′ Rₚ-enriched phosphorothioate and a 3′ Sp and Rp-enriched phosphorothioate. In some embodiments, the stereochemically enriched phosphorothioate may comprise RₚRₚSₚSₚ (RₚRₚ at the positions 1 and 2 of the guide strand and SₚSₚ at the positions 21 and 22 of the guide strand) or RₚRₚSₚRₚ (RₚRₚ at the positions 1 and 2 of the guide strand and SₚRₚ at the positions 21 and 22 of the guide strand). In some aspects, the polynucleotide molecules described herein comprises four stereochemically enriched phosphorothioates: (1) a Rp-enriched phosphorothioate covalently bonded to the 1^{st} nucleoside (e.g., the 3′-carbon atom of the 1 nucleoside) and the 2^{nd} nucleoside (e.g., the 5′-carbon atom of the 2^{nd} nucleoside) from the 5′-end of the antisense strand; (2) a Rp-enriched phosphorothioate covalently bonded to the 2^{nd} nucleoside (e.g., the 3′-carbon atom of the 2^{nd} nucleoside) and the 3^{rd} nucleoside (e.g., the 5′-carbon atom of the 3^{rd} nucleoside) from the 5′-end of the antisense strand; (3) a Sp-enriched phosphorothioate covalently bonded to the 21^{st} nucleoside (e.g., the 3′-carbon atom of the 21^{st} nucleoside) and the 22^{th} nucleoside (e.g., the 5′-carbon atom of the 22^{th} nucleoside) from the 5′-end of the antisense strand; and (4) a Sp-enriched phosphorothioate covalently bonded to the 22^{th} nucleoside (e.g., the 3′-carbon atom of the 22^{th} nucleoside) and the 23^{rd} nucleoside (e.g., the 5′-carbon atom of the 23^{rd} nucleoside) from the 5′-end of the antisense strand. In some aspects, the polynucleotide molecules described herein comprises four stereochemically enriched phosphorothioates: (1) a Rp-enriched phosphorothioate covalently bonded to the 1^{st} nucleoside (e.g., the 3′-carbon atom of the 1^{st} nucleoside) and the 2^{nd} nucleoside (e.g., the 5′-carbon atom of the 2^{nd} nucleoside) from the 5′-end of the antisense strand; (2) a Rp-enriched phosphorothioate covalently bonded to the 2^{nd} nucleoside (e.g., the 3-carbon atom of the 2nd nucleoside) and the 3^{rd} nucleoside (e.g., the 5′-carbon atom of the 3^{rd} nucleoside) from the 5′-end of the antisense strand; (3) a Sp-enriched phosphorothioate covalently bonded to the 21^{st} nucleoside (e.g., the 3′-carbon atom of the 21^{st} nucleoside) and the 22 nucleoside (e.g., the 5-carbon atom of the 22^{th} nucleoside) from the 5′-end of the antisense strand; and (4) a Rp-enriched phosphorothioate covalently bonded to the 22^{th} nucleoside (e.g., the 3′-carbon atom of the 22^{nd} nucleoside) and the 23^{rd} nucleoside (e.g., the 5′-carbon atom of the 23^{rd} nucleoside) from the 5′-end of the antisense strand.

In some aspects, the polynucleotide molecule described herein comprises one or more purine modification. In some aspects, the purine modification described herein is 2,6-diaminopurine. In some aspects, the purine modification described herein is 3-deaza-adenine. In some aspects, the purine modification described herein is 7-deaza-guanine. In some aspects, the purine modification described herein is 8-azido-adenine.

In some aspects, the polynucleotide molecule described herein comprises one or more pyrimidine modification. In some aspects, the pyrimidine modification described herein is 2-thiothymidine. In some aspects, the pyrimidine modification described herein is 5-carboxamide-uracil. In some aspects, the pyrimidine modification described herein is 5-methyl-cytosine. In some aspects, the pyrimidine modification described herein is 5-ethynyl uracil.

In some case, the polynucleic acid molecule described herein comprises an abasic substitution. In those cases where a hybridized polynucleotide construct is contemplated for use as siRNA, a reduction of miRNA-like off-target effects is desirable. The inclusion of one or more (e.g., one or two) abasic substitutions in the hybridized polynucleotide constructs may reduce or even eliminate miRNA-like off-target effects, as the abasic substitutions lack nucleobases that are capable of engaging in base-pairing interactions and alleviate steric hindrance. Thus, the polynucleotide molecule disclosed herein may include one or more (e.g., one or two) abasic substitutions. In some aspects, abasic substitution is at the 5^{th} nucleotide from the 5′ end of the antisense strand described herein. In some aspects, abasic substitution is at the 7^{th} nucleotide from the 5′ end of the antisense strand described herein.

When the polynucleotide molecule disclosed herein includes two or more of the abasic substitutions, their structures may be same or different. In certain aspects, a sense strand contains one abasic substitution (e.g., an antisense strand may be free of abasic substitutions). In other aspects, an antisense strand contains one abasic substitution (e.g., a sense strand may be free of abasic substitutions). In yet other aspects, an antisense strand contains one abasic substitution, and a sense strand contains one abasic substitution. In further aspects, a sense strand includes an abasic substitution between a nucleoside number (x) and a nucleoside number (x+1), where x is an integer from 2 to 7. In yet further aspects, an antisense strand includes an abasic substitution between a nucleoside number (x) and a nucleoside number (x+1), where x is an integer from 2 to 7.

The abasic substitution may be of formula (III):

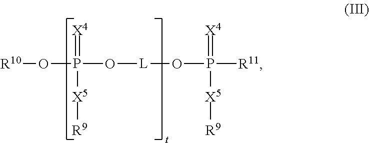

where
L is a sugar analogue, or is substituted with a heteroacyl from A, U, C, G, or is any other substituted nucleic acid (e.g., locked or unlocked nucleic acid, glycol nucleic acid, etc.);
each X⁴ is independently O or S;
each X⁵ is independently O, S, NH, or a bond;
each R⁹ is independently H, optionally substituted C₁₋₆ alkyl, optionally substituted C₂₋₆ alkenyl, optionally substituted C₂₋₆ alkynyl, optionally substituted (C₁₋₉ heterocyclyl)-C₁₋₆-alkyl, optionally substituted (C₆₋₁₀ aryl)-C₁₋₆-alkyl, optionally substituted (C₃₋₈ cycloalkyl)-C₁₋₆-alkyl, -LinkA(-T)ₚ, or a conjugation moiety;
each LinkA is independently a multivalent linker (e.g., including —C(O)—N(H)—);
each T is independently an auxiliary moiety;
R¹⁰ is a bond to a 3′-carbon atom of a nucleoside (x) in the strand;
R¹¹ is a bond to a 5′-oxygen atom of a nucleoside (x+1) in the strand;
p is an integer from 1 to 6; and
t is an integer from 1 to 6.

In some aspects, the abasic substitution described herein is attached to the antisense strand of the polynucleic acid molecule described herein. In particular aspects, an abasic substitution (e.g., an internucleotide, abasic spacer of formula (III) in which t is 1) may be included in the antisense strand described herein (e.g., within the seed region of the guide strand). In some aspects, an abasic substitution (e.g., an internucleotide, abasic spacer of formula (III) in which t is 1) may be bonded to the 3′ carbon atom of the second, third, fourth, or fifth nucleoside from the 5′-end of the antisense strand described herein. In certain aspects, an abasic substitution (e.g., an internucleotide, abasic spacer of formula (III) in which t is 1) may be bonded to the 3′ carbon atom of the thirteenth, fourteenth, fifteenth, or sixteenth nucleoside from the 5′-end of the antisense strand described herein. In some aspects, an abasic substitution fourth, fifth, sixth, seventh, eighth, and/or ninth nucleoside from the 5′-end of the antisense strand described herein.

The polynucleotide molecule described herein may contain a strand including a seed region including a hypoxanthine nucleobase-containing nucleoside (e.g., inosine).

In certain aspects, the hypoxanthine nucleobase-containing nucleoside is a second nucleoside from the 5′-end in the strand. In further aspects, the hypoxanthine nucleobase-containing nucleoside is a third nucleoside from the 5′-end in the strand. In yet further aspects, the hypoxanthine nucleobase-containing nucleoside is a fourth nucleoside from the 5′-end in the strand. In still further aspects, the hypoxanthine nucleobase-containing nucleoside is a fifth nucleoside from the 5′-end in the strand. In particular aspects, the hypoxanthine nucleobase-containing nucleoside is a sixth nucleoside in the strand. In particular aspects, the hypoxanthine nucleobase-containing nucleoside is a seventh nucleoside in the strand.

### The Amount and Location of Modifications

In some aspects, the polynucleotide molecule described herein comprises one or more type of modifications as described above. Accordingly, in some aspects, about 10% of the nucleotides from the polynucleotide molecule described herein are modified with one or more type of modifications as described above. In other aspects, about 20% of the nucleotides from the polynucleotide molecule described herein are modified with one or more type of modifications as described above. In other aspects, about 30% of the nucleotides from the polynucleotide molecule described herein are modified with one or more type of modifications as described above. In other aspects, about 40% of the nucleotides from the polynucleotide molecule described herein are modified with one or more type of modifications as described above. In other aspects, about 50% of the nucleotides from the polynucleotide molecule described herein are modified with one or more type of modifications as described above. In other aspects, about 60% of the nucleotides from the polynucleotide molecule described herein are modified with one or more type of modifications as described above. In other aspects, about 70% of the nucleotides from the polynucleotide molecule described herein are modified with one or more type of modifications as described above. In other aspects, about 80% of the nucleotides from the polynucleotide molecule described herein are modified with one or more type of modifications as described above. In other aspects, about 90% of the nucleotides from the polynucleotide molecule described herein are modified with one or more type of modifications as described above. In other aspects, 100% of the nucleotides from the polynucleotide molecule described herein are modified with one or more type of modifications as described above.

In some aspects, the one or more types of modifications described herein occurs at different positions within the polynucleotide molecule described herein. In some aspects, the one or more types of modifications described herein occurs in the seed region within the polynucleotide molecule described herein. In some aspects, the one or more types of modifications described herein occurs at 3′ terminal of the polynucleotide molecule described herein. In some aspects, the one or more types of modifications described herein occurs at 5′ terminal of the polynucleotide molecule described herein. In some aspects, the one or more types of modifications described herein occurs dispersedly within the polynucleotide molecule described herein. In some aspects, the one or more types of modifications described herein occurs in clusters within the polynucleotide molecule described herein.

### Specific Modification Patterns

In some aspects, described herein is a specific modification pattern for the polynucleic acid molecule which is a double-stranded nucleic acid molecule comprising a sense strand and an antisense strand. In some aspects, the antisense strand comprises a 2′-fluoro modified nucleotide in position 2 from the 5′ end. In some aspects, the antisense strand comprises a 2′-fluoro modified nucleotide in position 14 from the 5′ end. In some aspects, the antisense strand comprises 2′-fluoro modified nucleotides in positions 2 and 14 from the 5′ end. In some aspects, the antisense strand comprises a 2′-fluoro modified nucleotide in position 12 from the 5′ end. In some aspects, the antisense strand comprises a 2′-fluoro modified nucleotide in position 16 from the 5′ end. In other aspects, the antisense strand comprises a 2′-fluoro modified nucleotide in position 6 from the 5′ end. In other aspects, the antisense strand comprises a 2′-fluoro modified nucleotide in position 7 from the 5′ end. In other aspects, the antisense strand comprises a 2′-fluoro modified nucleotide in position 8 from the 5′ end. In other aspects, the antisense strand comprises a 2′-fluoro modified nucleotide in position 9 from the 5′ end. In other aspects, the antisense strand comprises a 2′-fluoro modified nucleotide in position 4 from the 5′ end.

In some aspects, described herein is a specific modification pattern for the polynucleic acid molecule which is a double-stranded nucleic acid molecule comprising a sense strand and an antisense strand. In some aspects, the sense strand comprises a 2′-fluoro modified nucleotide in position 9 from the 5′ end. In some aspects, the sense strand comprises a 2′-fluoro modified nucleotide in position 11 from the 5′ end. In some aspects, the sense strand comprises 2′-fluoro modified nucleotides in positions 9 and 11 from the 5′ end. In some aspects, the sense strand comprises a 2′-fluoro modified nucleotide in position 7 from the 5′ end. In some aspects, the sense strand comprises a 2′-fluoro modified nucleotide in position 10 from the 5′ end. In some aspects, the sense strand comprises 2′-fluoro modified nucleotides in positions 9, 11, and 10 from the 5′ end. the sense strand comprises 2′-fluoro modified nucleotides in positions 9 and 11, and 10 from the 5′ end. the sense strand comprises 2′-fluoro modified nucleotides in positions 9 and 7 from the 5′ end. the sense strand comprises 2′-fluoro modified nucleotides in positions 9 and 10 from the 5′ end. the sense strand comprises 2′-fluoro modified nucleotides in positions 9, 11, 7, and 10 from the 5′ end. In other aspects, the sense strand comprises a 2′-fluoro modified nucleotide in position 8 from the 5′ end. In other aspects, the sense strand comprises a 2′-fluoro modified nucleotide in position 12 from the 5′ end. In other aspects, the sense strand comprises a 2′-fluoro modified nucleotide in position 16 from the 5′ end.

In some aspects, the sense and antisense strand of the polynucleic acid molecule comprises any combination of two or more 2′-fluoro modified nucleotides at the positions described in the above two paragraphs.

In some aspects, the antisense strand comprises 5′-nNfnnnNfnNfNfnnnnNfnNfnnnnnnn-3′. In some aspects, the antisense strand comprises 5′-nNfnnnNfnnnnnnnNfnNfnnnnnnn-3′. In some aspects, the antisense strand comprises 5′-nNfnnnnNfnnnnNfnNfnnnnnnnnn-3′, In the modification patterns described above, “Nf” stands for a 2′-fluoro modified nucleotide, and wherein “n” stands for a 2′-O-methyl modified nucleotide.

In some aspects, the sense strand comprises 5′-nnnnnnNfnNfnNfnnnnnnnnnn-3′. In some aspects, the sense strand comprises 5′-nnnnnnNfnNfNfNfnnnnnnnnnn-3′. In some aspects, the sense strand comprises 5′-nnnnnnnnNfNfNfnnnnnnnnnn-3′. In the modification patterns described above, “NT” stands for a 2′-fluoro modified nucleotide, and wherein “n” stands for a 2′-O-methyl modified nucleotide.

In some aspects, described herein is a specific modification pattern for the polynucleic acid molecule which is a double-stranded nucleic acid molecule comprising a sense strand and an antisense strand, wherein the sense strand comprises about twelve 2′-fluoro modified nucleotides and about nine 2′-O-methyl modified nucleotides, and wherein the antisense strand comprises about nine 2′-fluoro modified nucleotides and about fourteen 2′-O-methyl modified nucleotides.

In some aspects, described herein is a specific modification pattern, wherein the sense strand is fully modified and comprises twelve 2′-fluoro modified nucleotides, nine 2′-O-methyl modified nucleotides, and wherein the antisense strand is fully modified and comprises nine 2′-fluoro modified nucleotides and fourteen 2′-O-methyl modified nucleotides.

In some aspects, described herein is a specific modification pattern, wherein the sense strand comprises 5′-NfnNfnNfnNfnNfNfNfnNfnNfnNfnNfnNf-3′, wherein the antisense strand comprises 5′-nNfnNfnNfnNfnNfnnnNfnNfnNfnNfnnn-3′, wherein “Nf” stands for a 2′-fluoro modified nucleotide, and wherein “n” stands for a 2′-O-methyl modified nucleotide.

In some aspects, described herein is a specific modification pattern, wherein the sense strand comprises 5′-NfnNfnNfnNfnNfNfNfnNfnNfnNfnNfnNf-3′, wherein the antisense strand comprises 5′-nNfnNfnNfnNfnNfnnnNfnNfnNfnNfnnn-3′, wherein the sense and/or antisense strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the sense strand comprises 5′-NfnNfnNfnNfnNfNfNfnNfnNfnNfnNfnNf-3′, wherein the antisense strand comprises 5′-nNfnNfnNfnNfnNfnnnNfnNfnNfnNfnnn-3′, wherein the sense comprises two phosphorothioate linkages, wherein the antisense comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the sense strand and/or antisense strand is modified as Type I in Table 19.

TABLE 19
Nucleotide Modification Patterns

| Pattern Name | Pattern |
|---|---|
| Type I for sense strand | 5′-NfsnsNfnNfnN |
| | fnNfNfNfnNfnNfn |
| | NfnNfnNf-3′ |
| Type I for antisense strand | 5′-nsNfsnNfnNfn |
| | NfnNfnnnNfnNfnN |
| | fnNfnsnsn-3′ |
| Type II for sense strand | 5′-nsnsnnnnNfnN |
| | fNfNfnnnnnnnnnn |
| | -3′ |
| Type II for antisense strand | 5′-nsNfsnnnNfnN |
| | fNfannnNfnNfnnn |
| | nnsnsn-3′ |
| Type III for sense strand | 5′-nsnsnnnnnnNf |
| | nNfnnnnnnnnnn- |
| | 3′ |
| Type III for antisense strand | 5′-nsNfsnnnnnnn |
| | nnNfnNfnnnnnnns |
| | nsn-3′ |
| Type IV for sense strand | 5′-nsnsnnnnNfnN |
| | fnNfnnnnnnnnnn- |
| | 3′ |
| Type IV for antisense strand | 5′-nsNfsnnnnnnn |
| | nnNfnNfnNfnnnnn |
| | snsn-3′ |
| Type V for sense strand | 5′-nsnsnnnnNfnN |
| | fnNfnnnnnnnnnn- |
| | 3′ |
| Type V for antisense strand | 5′-nsNfsnnnnNfn |
| | nnnNfnNfnNfnnnn |
| | nsnsn-3′ |
| Type VI for sense strand | 5′-nnnnnnNfnNfn |
| | Nfnnnnnnnnnn-in |
| | vdN-invdN-3′ |

Note:
“Nf” stands for a 2′-fluoro modified nucleotide, “n” stands for a 2′-O-methyl modified nucleotide, “s” stands for a 3′-phosphorothioate, “invdN” stands for an inverted deoxy-nucleotide.

In some aspects, the polynucleotide molecule provided herein comprises a sense strand comprising a nucleic acid sequence of SEQ ID NOs: 217-324, 481-504, 541-546, and 575-585 and an antisense strand comprises a nucleic acid sequence of SEQ ID NOs: 1-108, 433-456, 529-534, and 553-563. In other aspects, the polynucleotide molecule provided herein comprises a sense strand comprising a nucleic acid sequence of SEQ ID NOs: 217-324, 481-504, 541-546, and 575-585, an antisense strand comprises a nucleic acid sequence of SEQ ID NOs: 1-108, 433-456, 529-534, and 553-563, and wherein the sense and/or antisense strand is modified in Type I modification pattern specified in Table 19. In some aspects, the polynucleotide molecule provided herein comprises a sense strand comprising a nucleic acid sequence of SEQ ID NOs: 541-546 and an antisense strand comprises a nucleic acid sequence of SEQ ID NOs: 529-534. In other aspects, the polynucleotide molecule provided herein comprises a sense strand comprising a nucleic acid sequence of SEQ ID NOs: 541-546, an antisense strand comprises a nucleic acid sequence of SEQ ID NOs: 529-534, and wherein the sense and/or antisense strand is modified in Type I modification pattern specified in Table 19.

In some aspects, described herein is a specific modification pattern, wherein the sense strand comprises about four 2′-fluoro modified nucleotides and about seventeen 2′-O-methyl modified nucleotides, and wherein the antisense strand comprises about six 2′-fluoro modified nucleotides and about seventeen 2′-O-methyl modified nucleotides.

In some aspects, described herein is a specific modification pattern, wherein the sense strand is fully modified and comprises four 2′-fluoro modified nucleotides, seventeen 2′-O-methyl modified nucleotides, and wherein the antisense strand is fully modified and comprises six 2′-fluoro modified nucleotides and seventeen 2′-O-methyl modified nucleotides.

In some aspects, described herein is a specific modification pattern, wherein the sense strand comprises 5′-nnnnnnNfnNfNfNfnnnnnnnnnn-3′, wherein the antisense strand comprises 5′-nNfnnnNfnNfNfnnnnNfnNfnnnnnnn-3′, wherein “Nf” stands for a 2′-fluoro modified nucleotide, and wherein “n” stands for a 2′-O-methyl modified nucleotide.

In some aspects, described herein is a specific modification pattern, wherein the sense strand comprises 5′-nnnnnnNfnNfNfNfnnnnnnnnnn-3′, wherein the antisense strand comprises 5′-nNfnnnNfnNfNfnnnnNfnNfnnnnnnn-3′, wherein the sense and/or antisense strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the sense strand comprises 5′-nnnnnnNfnNfNfNfnnnnnnnnn-3′, wherein the antisense strand comprises 5′-nNfnnnNfnNfNfnnnnNfnNfnnnnnnn-3′, wherein the sense comprises two phosphorothioate linkages, wherein the antisense comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the sense strand and/or antisense strand is modified as Type II in Table 19.

In some aspects, the polynucleotide molecule provided herein comprises a sense strand comprising a nucleic acid sequence of SEQ ID NOs: 217-324, 481-504, 541-546, and 575-585, and/or an antisense strand comprising a nucleic acid sequence of SEQ ID NOs: 1-108, 433-456, 529-534, and 553-563, and wherein the sense and/or antisense strand is modified in Type II modification pattern specified in Table 19. In other aspects, the polynucleotide molecule provided herein comprises a sense strand comprising a nucleic acid sequence of SEQ ID NOs: 541-546, and/or an antisense strand comprising a nucleic acid sequence of SEQ ID NOs: 529-534, and wherein the sense and/or antisense strand is modified in Type II modification pattern specified in Table 19.

In some aspects, described herein is a specific modification pattern, wherein the sense strand comprises about two 2′-fluoro modified nucleotides and about nineteen 2′-O-methyl modified nucleotides, and wherein the antisense strand comprises about three 2′-fluoro modified nucleotides and about twenty 2′-O-methyl modified nucleotides.

In some aspects, described herein is a specific modification pattern, wherein the sense strand is fully modified and comprises two 2′-fluoro modified nucleotides and nineteen 2′-O-methyl modified nucleotides, and wherein the antisense strand is fully modified and comprises three 2′-fluoro modified nucleotides and twenty 2′-O-methyl modified nucleotides.

In some aspects, described herein is a specific modification pattern, wherein the sense strand comprises 5′-nnnnnnnnnNfnNfnnnnnnnnnn-3′, wherein the antisense strand comprises 5′-nNfnnnnnnnnnNfnNfnnnnnnnnn-3′, wherein “Nf” stands for a 2′-fluoro modified nucleotide, and wherein “n” stands for a 2′-O-methyl modified nucleotide.

In some aspects, described herein is a specific modification pattern, wherein the sense strand comprises 5′-nnnnnnnnNfnNfnnnnnnnnnn-3′, wherein the antisense strand comprises 5′-nNfnnnnnnnnnNfnNfnnnnnnnnnn-3′, wherein the sense and/or antisense strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the sense strand comprises 5′-nnnnnnnnNfnNfnnnnnnnnnn-3′, wherein the antisense strand comprises 5′-nNfnnnnnnnnnNfnNfnnnnnnnnn-3′, wherein the sense comprises two phosphorothioate linkages, wherein the antisense comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the sense strand and/or antisense strand is modified as Type III in Table 19.

In some aspects, the polynucleotide molecule provided herein comprises a sense strand comprising a nucleic acid sequence of SEQ ID NOs: 217-324, 481-504, 541-546, and 575-585, and/or an antisense strand comprising a nucleic acid sequence of SEQ ID NOs: 1-108, 433-456, 529-534, and 553-563, and wherein the sense and/or antisense strand is modified in Type III modification pattern specified in Table 19. In other aspects, the polynucleotide molecule provided herein comprises a sense strand comprising a nucleic acid sequence of SEQ ID NOs: 541-546, and/or an antisense strand comprising a nucleic acid sequence of SEQ ID NOs: 529-534, and wherein the sense and/or antisense strand is modified in Type III modification pattern specified in Table 19.

In some aspects, described herein is a specific modification pattern, wherein the sense strand comprises about three 2′-fluoro modified nucleotides and about eighteen 2′-O-methyl modified nucleotides, and wherein the antisense strand comprises about four 2′-fluoro modified nucleotides and about nineteen 2′-O-methyl modified nucleotides.

In some aspects, described herein is a specific modification pattern, wherein the sense strand is fully modified and comprises three 2′-fluoro modified nucleotides, eighteen 2′-O-methyl modified nucleotides, and wherein the antisense strand is fully modified and comprises four 2′-fluoro modified nucleotides, nineteen 2′-O-methyl modified nucleotides.

In some aspects, described herein is a specific modification pattern, wherein the sense strand comprises 5′-nnnnnnNfnNfnNfnnnnnnnnnn-3′, wherein the antisense strand comprises 5′-nNfnnnnnnnnnNfnNfnNfnnnnnnn-3′, wherein “Nf” stands for a 2′-fluoro modified nucleotide, and wherein “n” stands for a 2′-O-methyl modified nucleotide.

In some aspects, described herein is a specific modification pattern, wherein the sense strand comprises 5′-nnnnnnNfnNfnNfnnnnnnnnnn-3′, wherein the antisense strand comprises 5′-nNfnnnnnnnnnNfnNfnNfnnnnnnn-3′, wherein the sense and/or antisense strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the sense strand comprises 5′-nnnnnnNfnNfnNfnnnnnnnnn-3′, wherein the antisense strand comprises 5′-nNfnnnnnnnnnNfnNfnNfnnnnnnn-3′, wherein the sense comprises two phosphorothioate linkages, wherein the antisense comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the sense strand and/or antisense strand is modified as Type IV in Table 19.

In some aspects, the polynucleotide molecule provided herein comprises a sense strand comprising a nucleic acid sequence of SEQ ID NOs: 217-324, 481-504, 541-546, and 575-585, and/or an antisense strand comprising a nucleic acid sequence of SEQ ID NOs: 1-108, 433-456, 529-534, and 553-563, and wherein the sense and/or antisense strand is modified in Type IV modification pattern specified in Table 19. In other aspects, the polynucleotide molecule provided herein comprises a sense strand comprising a nucleic acid sequence of SEQ ID NOs: 541-546, and/or an antisense strand comprising a nucleic acid sequence of SEQ ID NOs: 529-534, and wherein the sense and/or antisense strand is modified in Type IV modification pattern specified in Table 19.

In some aspects, described herein is a specific modification pattern, wherein the sense strand comprises about three 2′-fluoro modified nucleotides and about eighteen 2′-O-methyl modified nucleotides, and wherein the antisense strand comprises about five 2′-fluoro modified nucleotides and about eighteen 2′-O-methyl modified nucleotides.

In some aspects, described herein is a specific modification pattern, wherein the sense strand is fully modified and comprises three 2′-fluoro modified nucleotides and eighteen 2′-O-methyl modified nucleotides, and wherein the antisense strand is fully modified and comprises five 2′-fluoro modified nucleotides, eighteen 2′-O-methyl modified nucleotides.

In some aspects, described herein is a specific modification pattern, wherein the sense strand comprises 5′-nnnnnnNfnNfnNfnnnnnnnnnn-3′, wherein the antisense strand comprises 5′-nNfnnnnNfnnnnNfnNfnNfnnnnnnn-3′, wherein “Nf” stands for a 2′-fluoro modified nucleotide, and wherein “n” stands for a 2′-O-methyl modified nucleotide.

In some aspects, described herein is a specific modification pattern, wherein the sense strand comprises 5′-nnnnnnNfnNfnNfnnnnnnnnnn-3′, wherein the antisense strand comprises 5′-nNfnnnnNfnnnnNfnNfnNfnnnnnnn-3′, wherein the sense and/or antisense strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the sense strand comprises 5′-nnnnnnNfnNfnNfnnnnnnnnnn-3′, wherein the antisense strand comprises 5′-nNfnnnnNfnnnnNfnNfnNfnnnnnnn-3′, wherein the sense comprises two phosphorothioate linkages, wherein the antisense comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the sense strand and/or antisense strand is modified as Type V in Table 19.

In some aspects, the polynucleotide molecule provided herein comprises a sense strand comprising a nucleic acid sequence of SEQ ID NOs: 217-324, 481-504, 541-546, and 575-585, and/or an antisense strand comprising a nucleic acid sequence of SEQ ID NOs: 1-108, 433-456, 529-534, and 553-563, and wherein the sense and/or antisense strand is modified in Type V modification pattern specified in Table 19. In other aspects, the polynucleotide molecule provided herein comprises a sense strand comprising a nucleic acid sequence of SEQ ID NOs: 541-546, and/or an antisense strand comprising a nucleic acid sequence of SEQ ID NOs: 529-534, and wherein the sense and/or antisense strand is modified in Type V modification pattern specified in Table 19.

In some aspects, described herein is a specific modification pattern, wherein the sense strand comprises about three 2′-fluoro modified nucleotides and about eighteen 2′-O-methyl modified nucleotides, with one or more inverted deoxy-nucleotides on the 3′ end as an overhang.

In some aspects, described herein is a specific modification pattern, wherein the sense strand is fully modified and comprises three 2′-fluoro modified nucleotides and eighteen 2′TO-methyl modified nucleotides, with two inverted deoxy-nucleotides on the 3′ end as an overhang.

In some aspects, described herein is a specific modification pattern, wherein the sense strand comprises 5′-nnnnnnNfnNfnNfnnnnnnnnnn-invdN-invdN-3′, wherein “Nf” stands for a 2′-fluoro modified nucleotide, wherein “n” stands for a 2′-O-methyl modified nucleotide, and “invdN” stands for an inverted deoxy-nucleotide. In some instances, the invdN is an inverted deoxyl-thymine. In some aspects, the linker conjugated with one or more targeting moieties as shown in Formula (IV″) or (IV′″) is added to the first nucleic acid on the 5′ end. In some aspects, the linker conjugated with one or more GalNAc as shown in Formula (V″) or (V′″) is added to the first nucleic acid on the 5′ end. In some aspects, the modification pattern comprises one or more phosphorothioate linkages. In some aspects, the modification pattern is shown in Formula (VII). In some aspects, the 5′ end modification known in the art is applied to the one or more inverted nucleotides.

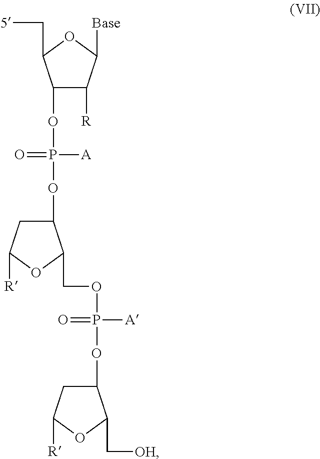

wherein R is a moiety that corresponds to the sugar modification described herein, in some instances, R is —O-methyl; wherein R′ is thymine, abasic, or others; wherein A is —O or —S; and wherein A′ is —O or —S.

In some aspects, the polynucleotide molecule provided herein comprises a sense strand comprising a nucleic acid sequence of SEQ ID NOs: 217-324, 481-504, 541-546, and 575-585, and/or an antisense strand comprising a nucleic acid sequence of SEQ ID NOs: 1-108, 433-456, 529-534, and 553-563, and wherein the sense strand is modified in Type VI modification pattern specified in Table 19 or as described in the preceding paragraph. In other aspects, the polynucleotide molecule provided herein comprises a sense strand comprising a nucleic acid sequence of SEQ ID NOs: 541-546, and/or an antisense strand comprising a nucleic acid sequence of SEQ ID NOs: 529-534, and wherein the sense strand is modified in Type VI modification pattern specified in Table 19 or as described in the preceding paragraph.

Described herein is a polynucleic acid molecule, whose sense strand comprises a nucleic acid sequence that is at least 80% identical to a nucleic acid sequence selected from SEQ ID NOs: 325-432, 505-528, 547-552, and 586-596. Described herein is a polynucleic acid molecule, whose sense strand comprises a nucleic acid sequence that is at least 85% identical to a nucleic acid sequence selected from SEQ ID NOs: 325-432, 505-528, 547-552, and 586-596. Described herein is a polynucleic acid molecule, whose sense strand comprises a nucleic acid sequence that is at least 90% identical to a nucleic acid sequence selected from SEQ ID NOs: 325-432, 505-528, 547-552, and 586-596. Described herein is a polynucleic acid molecule, whose sense strand comprises a nucleic acid sequence that is at least 95% identical to a nucleic acid sequence selected from SEQ ID NOs: 325-432, 505-528, 547-552, and 586-596.

Described herein is a polynucleic acid molecule, which antisense strand comprises a nucleic acid sequence that is at least 80% identical to a nucleic acid sequence selected from SEQ ID NOs: 109-216, 457-480, 535-540, and 564-574. Described herein is a polynucleic acid molecule, which antisense strand comprises a nucleic acid sequence that is at least 85% identical to a nucleic acid sequence selected from SEQ ID NOs: 109-216, 457-480, 535-540, and 564-574. Described herein is a polynucleic acid molecule, which antisense strand comprises a nucleic acid sequence that is at least 90% identical to a nucleic acid sequence selected from SEQ ID NOs: 109-216, 457-480, 535-540, and 564-574. Described herein is a polynucleic acid molecule, which antisense strand comprises a nucleic acid sequence that is at least 95% identical to a nucleic acid sequence selected from SEQ ID NOs: 109-216, 457-480, 535-540, and 564-574.

Further provided herein is a polynucleic acid molecule for modulating expression of APOC3 gene, wherein polynucleic acid molecule comprises an antisense strand comprising the nucleotide sequence of usUfsucagGfgaacUfgAfaGfccaucsgsg (SEQ ID NO:535) and a sense strand comprising the nucleotide sequence of gsasuggcUfuCfaGfuucccugaaa (SEQ ID NO:547), wherein “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide.

Further provided herein is a polynucleic acid molecule for modulating expression of APOC3 gene, wherein polynucleic acid molecule comprises an antisense strand comprising the nucleotide sequence of usGfsaauaCfugucCfcUfuUfuaagcsasa (SEQ ID NO:536) and a sense strand comprising the nucleotide sequence of gscsuuaaAfaGfgGfacaguauuca (SEQ ID NO:548), where “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide.

Further provided herein is a polynucleic acid molecule for modulating expression of APOC3 gene, wherein polynucleic acid molecule comprises an antisense strand comprising the nucleotide sequence of usAfsgaauAfcuguCfcCfuUfuuaagscsa (SEQ ID NO:537) and a sense strand comprising the nucleotide sequence of csusuaaaAfgGfgAfcaguauucua (SEQ ID NO:549), where “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide.

Further provided herein is a polynucleic acid molecule for modulating expression of APOC3 gene, wherein polynucleic acid molecule comprises an antisense strand comprising the nucleotide sequence of usUfsgagaAfuacuGfuCfcCfuuuuasasg (SEQ ID NO:538) and a sense strand comprising the nucleotide sequence of usasaaagGfgAfcAfguauucucaa (SEQ ID NO:550), where “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide.

Further provided herein is a polynucleic acid molecule for modulating expression of APOC3 gene, wherein polynucleic acid molecule comprises an antisense strand comprising the nucleotide sequence of usCfsugagAfauacUfgUfcCfcuuuusasa (SEQ ID NO:539) and a sense strand comprising the nucleotide sequence of asasaaggGfaCfaGfuauucucaga (SEQ ID NO:551), where “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “UT” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide.

Further provided herein is a polynucleic acid molecule for modulating expression of APOC3 gene, wherein polynucleic acid molecule comprises an antisense strand comprising the nucleotide sequence of usCfsacugAfgaauAfcUfgUfcccuususu (SEQ ID NO:540) and a sense strand comprising the nucleotide sequence of asasgggaCfaGfuAfuucucaguga (SEQ ID NO:552), where “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide.

Further provided herein is a polynucleic acid molecule for modulating expression of APOC3 gene, wherein polynucleic acid molecule comprises an antisense strand comprising the nucleotide sequence of vpusCfsugagAfauacUfgUfcCfcuuuusasa (SEQ ID NO: 565) and a sense strand comprising the nucleotide sequence of asasaaggGfaCfaGfuaucucaga (SEQ ID NO: 587), where “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate, “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide.

Further provided herein is a polynucleic acid molecule for modulating expression of APOC3 gene, wherein polynucleic acid molecule comprises an antisense strand comprising the nucleotide sequence of usCfsugagAfauacUfgUfcCfcuuucsasa (SEQ ID NO: 566) and a sense strand comprising the nucleotide sequence of gsasaaggGfaCfaGfuauucucaga (SEQ ID NO: 588), where “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide.

Further provided herein is a polynucleic acid molecule for modulating expression of APOC3 gene, wherein polynucleic acid molecule comprises an antisense strand comprising the nucleotide sequence of usCfsugagAfauacUfgUfcCfcuuugsasa (SEQ ID NO: 567) and a sense strand comprising the nucleotide sequence of csasaaggGfaCfaGfuauucucaga (SEQ ID NO: 589), where “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “If” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide.

Further provided herein is a polynucleic acid molecule for modulating expression of APOC3 gene, wherein polynucleic acid molecule comprises an antisense strand comprising the nucleotide sequence of usCfsugagAfauacUfgUfcCfcuugcsasa (SEQ ID NO: 568) and a sense strand comprising the nucleotide sequence of gscsaaggGfaCfaGfuauucucaga (SEQ ID NO: 590), where “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide.

Further provided herein is a polynucleic acid molecule for modulating expression of APOC3 gene, wherein polynucleic acid molecule comprises an antisense strand comprising the nucleotide sequence of usCfsugagAfauacUfgUfcCfcuucgsasa (SEQ ID NO: 569) and a sense strand comprising the nucleotide sequence of csgsaaggGfaCfaGfuauucucaga (SEQ ID NO: 591), where “A” refers to adenosine-3′-phosphate; “a” refers to 2′-O-methyladenosine-3′-phosphate; “Af” refers to 2′-fluoroadenosine-3′-phosphate, “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide.

Further provided herein is a polynucleic acid molecule for modulating expression of APOC3 gene, wherein polynucleic acid molecule comprises an antisense strand comprising the nucleotide sequence of usCfsugagAfauacUfgUfcCfcuuuusasa (SEQ ID NO: 570) and a sense strand comprising the nucleotide sequence of (invAb)asasaaggGfaCfaGfuauucucaga (SEQ ID NO: 592), where “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide.

Further provided herein is a polynucleic acid molecule for modulating expression of APOC3 gene, wherein polynucleic acid molecule comprises an antisense strand comprising the nucleotide sequence of usCfsugagAfauacUfgUfcCfcuuugsasa (SEQ ID NO: 571) and a sense strand comprising the nucleotide sequence of (invAb)csasaaggGfaCfaGfuauucucaga (SEQ ID NO: 593), where “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide.

Further provided herein is a polynucleic acid molecule for modulating expression of APOC3 gene, wherein polynucleic acid molecule comprises an antisense strand comprising the nucleotide sequence of usCfsugdAgdAauacUfgUfcCfcuuuusasa (SEQ ID NO: 572) and a sense strand comprising the nucleotide sequence of asasaaggGfaCfaGfuauucucaga (SEQ ID NO: 594), where “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “UT” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide.

Further provided herein is a polynucleic acid molecule for modulating expression of APOC3 gene, wherein polynucleic acid molecule comprises an antisense strand comprising the nucleotide sequence of usCfsugagAfauacUfgUfscCfcuuuusasa (SEQ ID NO: 573) and a sense strand comprising the nucleotide sequence of asasaaggGfaCfaGfuauucucaga (SEQ ID NO: 595) where “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide.

Further provided herein is a polynucleic acid molecule for modulating expression of APOC3 gene, wherein polynucleic acid molecule comprises an antisense strand comprising the nucleotide sequence of usCfsugagAfauacUfgUfcCfcsuuuusasa (SEQ ID NO: 574) and a sense strand comprising the nucleotide sequence of asasaaggGfaCfaGfuauucucaga (SEQ ID NO: 596), where “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide.

### Conjugation

### Targeting Moiety

In certain aspects, the polynucleotide molecule described herein is coupled or conjugated with one or more targeting moieties to form a polynucleotide-targeting moiety conjugate molecule. In some instances, a targeting moiety is selected based on its ability to target the conjugate molecule described herein to a desired cell population, tissue, or an organ selectively or preferably. In some instances, the targeting moiety targets the cell, tissue, or an organ that expresses the corresponding binding partner (e.g., either the corresponding receptor or ligand) of the targeting moiety. For example, the polynucleotide molecule conjugated with N-acetyl galactosamine (GalNAc) can target hepatocytes expressing asialoglycoprotein (ASGP-R). Any suitable GalNAc molecules that are known in the art to be used as a targeting moiety are contemplated. Exemplary GalNAc molecule includes a triantennary GalNAc (e.g., L96). A further example of the targeting moiety is galactose. The targeting moiety can also be a lipid, peptide, or small molecule.

A targeting moiety (i.e., an intracellular targeting moiety) that targets a desired site within the cell (e.g., endoplasmic reticulum, Golgi apparatus, nucleus, or mitochondria) may be included in the hybridized polynucleotide constructs disclosed herein. Non-limiting examples of the intracellular targeting moieties are provided in WO 2015/069932 and in WO 2015/188197; the disclosure of the intracellular targeting moieties in WO 2015/069932 and in WO 2015/188197 is incorporated herein by reference.

The polynucleotide molecule described herein, thus, may include one or more targeting moieties selected from the group consisting of intracellular targeting moieties, extracellular targeting moieties, and combinations thereof. Thus, the inclusion of one or more targeting moieties (e.g., extracellular targeting moieties including targeting moieties independently selected from the group consisting of folate, mannose, N-acetyl galactosamine, and prostate specific membrane antigen) and one or more intracellular targeting moiety (e.g., a moiety targeting endoplasmic reticulum, Golgi apparatus, nucleus, or mitochondria) in the polynucleotide molecule described herein can facilitate the delivery of the polynucleotides to a specific site within the specific cell population. In some aspects, the targeting moiety contains one or more mannose carbohydrates. Mannose targets the mannose receptor, which is a 175 KDa membrane-associated receptor that is expressed on sinusoidal liver cells and antigen presenting cells (e.g., macrophages and dendritic cells). It is a highly effective endocytotic/recycling receptor that binds and internalizes mannosylated pathogens and proteins (Lennartz et. al. J. Biol. Chem. 262:9942-9944, 1987; Taylor et. al. J. Biol. Chem. 265:12156-62, 1990).

Some of the targeting moieties are described herein. In some aspects, the targeting moiety contains or specifically binds to a protein selected from the group including insulin, insulin-like growth factor receptor 1 (IGF1R), IGF2R, insulin-like growth factor (IGF; e.g., IGF 1 or 2), mesenchymal epithelial transition factor receptor (c-met; also known as hepatocyte growth factor receptor (HGFR)), hepatocyte growth factor (HGF), epidermal growth factor receptor (EGFR), epidermal growth factor (EGF), heregulin, fibroblast growth factor receptor (FGFR), platelet-derived growth factor receptor (PDGFR), platelet-derived growth factor (PDGF), vascular endothelial growth factor receptor (VEGFR), vascular endothelial growth factor (VEGF), tumor necrosis factor receptor (TNFR), tumor necrosis factor alpha (TNF-α), TNF-β, folate receptor (FOLR), folate, transferrin, transferrin receptor (TfR), mesothelin, Fc receptor, c-kit receptor, c-kit, an integrin (e.g., an α4 integrin or a β-1 integrin), P-selectin, sphingosine-1-phosphate receptor-1 (S1PR), hyaluronate receptor, leukocyte function antigen-1 (LFA-1), CD4, CD11, CD18, CD20, CD25, CD27, CD52, CD70, CD80, CD85, CD95 (Fas receptor), CD106 (vascular cell adhesion molecule 1 (VCAM1), CD166 (activated leukocyte cell adhesion molecule (ALCAM)), CD178 (Fas ligand), CD253 (TNF-related apoptosis-inducing ligand (TRAIL), ICOS ligand, CCR2, CXCR3, CCR5, CXCL12 (stromal cell-derived factor 1 (SDF-1)), interleukin 1 (IL-1), IL-1ra, IL-2, IL-3, IL-4, IL-6, IL-7, IL-8, CTLA-4, MART-1, gp100, MAGE-1, ephrin (Eph) receptor, mucosal addressin cell adhesion molecule 1 (MAdCAM-1), carcinoembryonic antigen (CEA), LewisY, MUC-1, epithelial cell adhesion molecule (EpCAM), cancer antigen 125 (CA125), prostate specific membrane antigen (PSMA), TAG-72 antigen, and fragments thereof. In further aspects, the targeting moiety contains erythroblastic leukemia viral oncogene homolog (ErbB) receptor (e.g., ErbB1 receptor; ErbB2 receptor; ErbB3 receptor; and ErbB4 receptor). In some aspects, the targeting moiety contains one or more (e.g., from 1 to 6) N-acetyl galactosamines (GalNAc). In some aspects, the targeting moiety contains one or m ore (e.g., from 1 to 6) galactose. In certain aspects, the targeting moiety contains one or more (e.g., from 1 to 6) mannoses. In other aspects, the targeting moiety contains a folate ligand. The folate ligand has the structure:

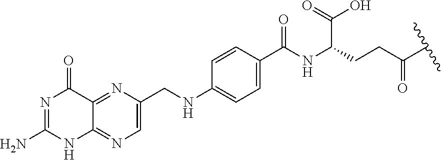 [SMILES: *C(=O)CC[C@H](NC(=O)c1ccc(NCc2cnc3[nH]c(N)nc(=O)c3n2)cc1)C(=O)O]

Certain targeting moieties may include bombesin, gastrin, gastrin-releasing peptide, tumor growth factors (TGF) (e.g., TGF-α or TGF-β), or vaccinia virus growth factor (VVGF). Non-peptidyl targeting moieties can also be used in the targeting moieties and may include, for example, steroids, carbohydrates, vitamins, and lectins. Some targeting moieties may include a polypeptide, such as somatostatin or somatostatin analog (e.g., octreotide or lanreotide), bombesin, or an antibody or antigen-binding fragment thereof. Antibodies may be of any recognized class or subclass, e.g., IgG, IgA, IgM, IgD, or IgE. Typical are those antibodies which fall within the IgG class. The antibodies can be derived from any species according to techniques known in the art. Typically, however, the antibody is of human, murine, or rabbit origin. In addition, the antibody may be polyclonal or monoclonal, but is typically monoclonal. Human or chimeric (e.g., humanized) antibodies may be used in targeting moieties. Targeting moieties may include an antigen-binding fragment of an antibody. Such antibody fragments may include, for example, the Fab′, F(ab′)2, Fv, or Fab fragments, single domain antibody, ScFv, or other antigen-binding fragments. Fe fragments may also be employed in targeting moieties. Such antibody fragments can be prepared, for example, by proteolytic enzyme digestion, for example, by pepsin or papain digestion, reductive alkylation, or recombinant techniques. The materials and methods for preparing antibody fragments are well-known to those skilled in the art. See, e.g., Parham, J. Immunology, 131:2895, 1983; Lamoyi et al., J. Immunological Methods, 56:235, 1983.

Other peptides for use as a targeting auxiliary moiety in polynucleotide molecule described herein can be selected from KiSS peptides and analogs, urotensin II peptides and analogs, GnRH I and II peptides and analogs, depreotide, vapreotide, vasoactive intestinal peptide (VIP), cholecystokinin (CCK), RGD-containing peptides, melanocyte-stimulating hormone (MSH) peptide, neurotensin, calcitonin, glutathione, YIGSR (leukocyte-avid peptides, e.g., P483H, which contains the heparin-binding region of platelet factor-4 (PF-4) and a lysine-rich sequence), atrial natriuretic peptide (ANP), β-amyloid peptides, delta-opioid antagonists (such as ITIPP(psi)), annexin-V, endothelin, leukotriene B4 (LTB4), chemotactic peptides (e.g., N-formyl-methionyl-leucyl-phenylalanine-lysine (fMLFK), GP IIb/IIIa receptor antagonists (e.g., DMP444), human neutrophil elastase inhibitor (EPI-HNE-2 and EPI-HNE-4), plasmin inhibitor, antimicrobial peptides, apticide (P280 and P274), thrombospondin receptor (including analogs such as TP-1300), bitistatin, pituitary adenylyl cyclase type I receptor (PAC1), fibrin α-chain, peptides derived from phage display libraries, and conservative substitutions thereof.

One or more (e.g., from 1 to 6) targeting moieties can be linked to MOIETY or to X2 in formula (V′, V″, V′″, V″″, V′″″, V″″″) through -LinkA-.

In some aspects, the targeting moiety includes one or more (e.g., from 1 to 6 or from 1 to 3) asialoglycoprotein receptor ligands (e.g., GalNAc). In some aspects, an asialoglycoprotein receptor ligand (e.g., GalNAc) is attached to -LinkA- through an anomeric carbon (e.g., where the anomeric carbon is the carbon atom in an acetal or a hemiaminal). In some aspects, an asialoglycoprotein receptor ligand (e.g., GalNAc) comprises an anomeric carbon bonded to trivalent, tetravalent linker, pentavalent, or hexavalent linker, wherein the anomeric carbon is part of a hemiaminal group. An asialoglycoprotein receptor ligand (e.g., GalNAc) attached to a linker through a hemiaminal may produce a hybridized polynucleotide construct having superior efficacy in gene silencing as compared to hybridized polynucleotide constructs having the asialoglycoprotein receptor ligand (e.g., GalNAc) attached to a linker through an acetal.

In some aspects, the linker and three asialoglycoprotein receptor targeting moieties, each of which comprises GalNAc, are as shown in Formula (V). In some instances, the conjugate described herein only comprises one asialoglycoprotein receptor targeting moiety, so the conjugate comprises a structure of Formula (V) with any two of the targeting moieties removed. In some instances, the conjugate described herein only comprises two asialoglycoprotein receptor targeting moieties, so the conjugate described herein comprises a structure of Formula (V) with any one of the targeting moieties removed.

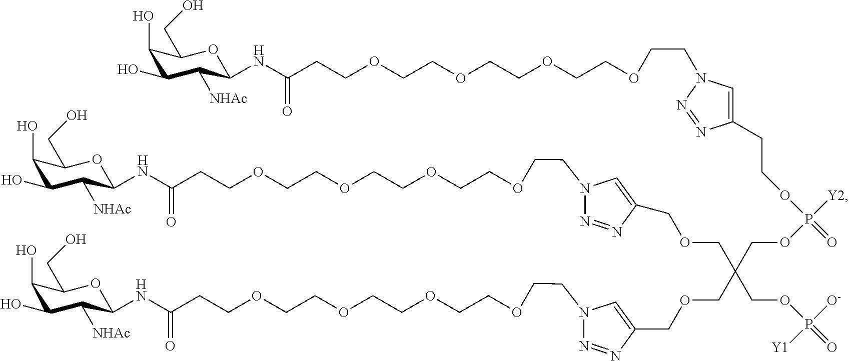

wherein one of Y1 and Y2 is nucleotide, or wherein both Y1 and Y2 are nucleotides and Y1 and Y2 are consecutive or neighboring nucleotides from the polynucleic acid molecule described herein.

In some aspects, the linker and the targeting moieties described herein are conjugated to 3′ end of the sense strand (e.g., as shown in Formula (V′, V″″, V′″″, V″″″)). In some aspects, the linker and the targeting moieties described herein are conjugated to 5′ end of the sense strand (e.g., as shown in Formula (V″) or (V′″)). In some aspects, the linker and the targeting moieties described herein are conjugated to 3′ end of the antisense strand (e.g., as shown in Formula (V′, V″″, V′″″, V″″″)). In some aspects, the linker and the targeting moieties described herein are conjugated to 5′ end of the antisense strand (e.g., as shown in Formula (V″) or (V′″)).

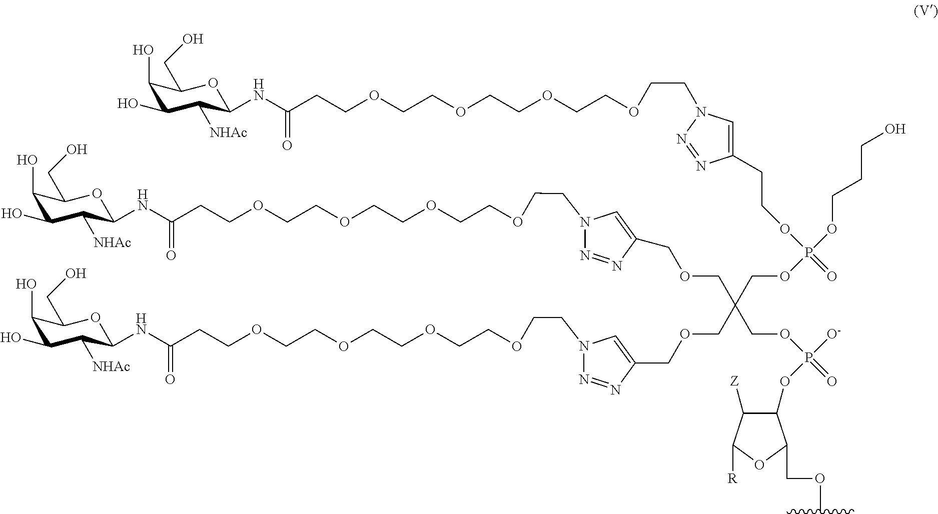

wherein Z in formula (V′) corresponds to one of the sugar modifications described herein (e.g., —H, —OH, —O-Methyl, —F, or —O-methoxyethyl) and R in formula (V′) is adenine, uracil, guanine, cytosine, thymine, abasic, or others.

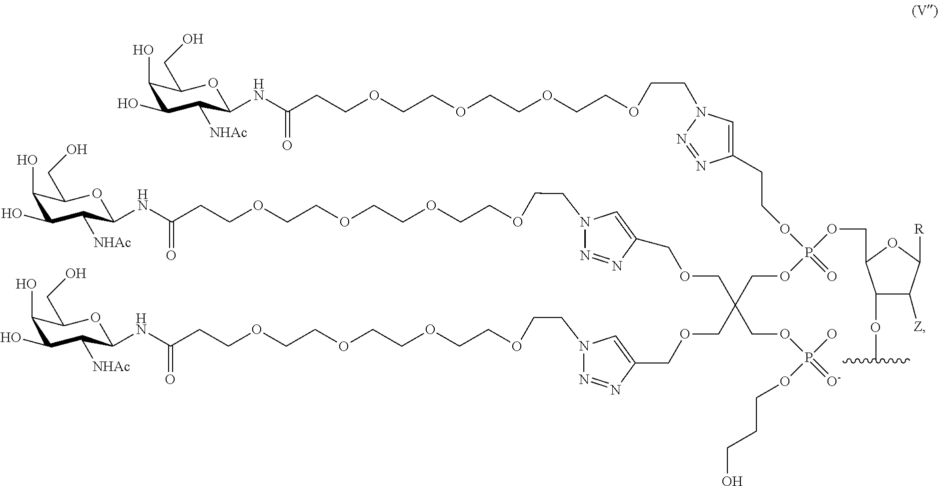

wherein Z in formula (V″) is a moiety that corresponds to one of the sugar modifications described herein (e.g., —H, —OH, —O-Methyl, —F, or —O-methoxyethyl) and R in formula (V″) is adenine, uracil, guanine, cytosine, thymine, abasic, or others.

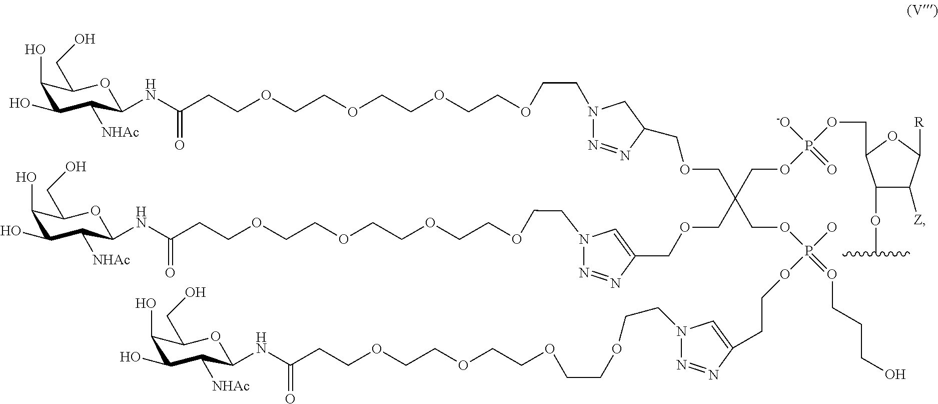

wherein Z in formula (V′″) is a moiety that corresponds to one of the sugar modifications described herein (e.g., —H, —OH, —O-Methyl, —F, or —O-methoxyethyl) and R in formula (V′″) is adenine, uracil, guanine, cytosine, thymine, abasic, or others.

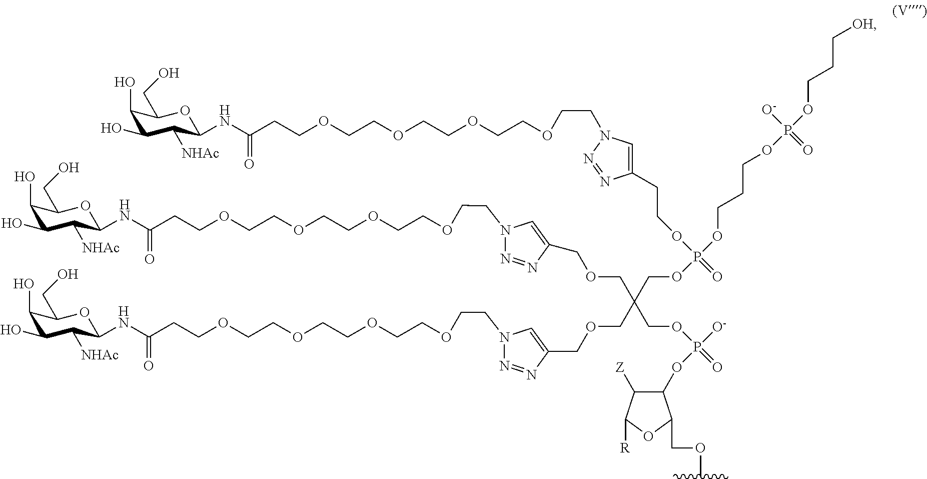

wherein Z in formula (V″″) is a moiety that corresponds to one of the sugar modifications described herein (e g., —H, —OH, —O-Methyl, —F, or —O-methoxyethyl) and R in formula (V″″) is adenine, uracil, guanine, cytosine, thymine, abasic, or others.

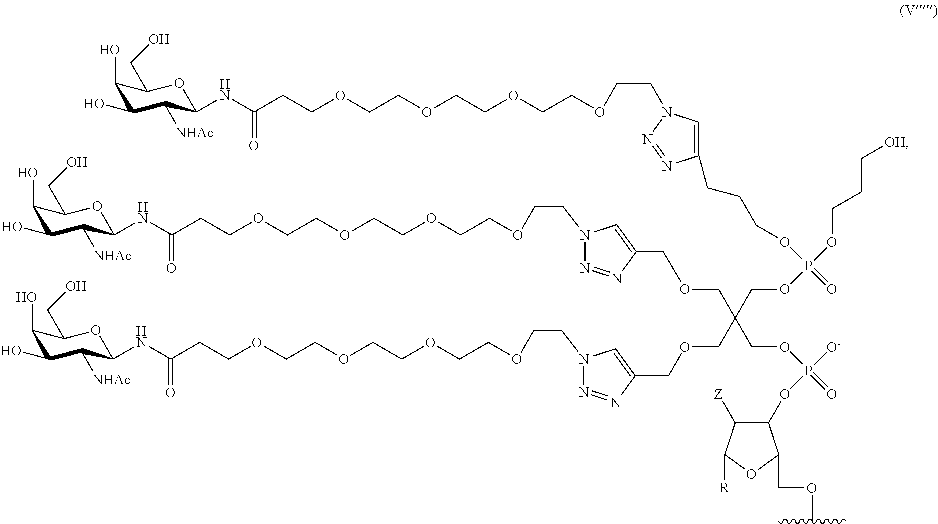

wherein Z in formula (V′″″) is a moiety that corresponds to one of the sugar modifications described herein (e.g., —H, —OH, —O-Methyl, —F, or —O-methoxyethyl) and R in formula (V′″″) is adenine, uracil, guanine, cytosine, thymine, abasic, or others.

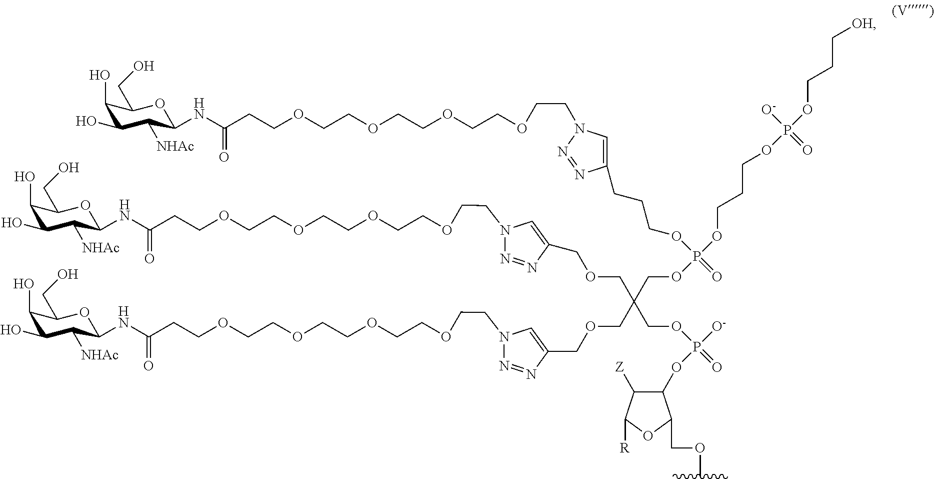

wherein Z in formula (V″″″) is a moiety that corresponds to one of the sugar modifications described herein (e.g., —H, —OH, —O-Methyl, —F, or —O-methoxyethyl) and R in formula (V″″″) is adenine, uracil, guanine, cytosine, thymine, abasic, or others.

In some instances, the 3′ end of passenger/sense strand from Table 1, Table 3, Table 5, or Table 12is conjugated with X2-GalNAc (see Formula (V), (V′) (V″″), (V′″″), (V″″″)). In some instances, the 5′ end of passenger/sense strand from Table 1, Table 3, Table 5, or Table 12 is conjugated with X2-GalNAc (see Formula (V), (V″), or or (V′″)). In some instances, a nucleic acid within passenger/sense strand (not at the 5′ or 3′ end) from Table 1, Table 3, Table 5, or Table 12 is conjugated with X2-GalNAc (see Formula (V)). In some instances, the 3′ end of guide/antisense strand from Table 1, Table 3, Table 5, or Table 12 is conjugated with X2-GalNAc (see Formula (V), (V′) (V″″), (V′″″), (V″″″)). In some instances, the 5′ end of guide/antisense strand from Table 1, Table 3, Table 5, or Table 12 is conjugated with X2-GalNAc (see Formula (V), (V′), or or (V′″)). In some instances, a nucleic acid within guide/antisense strand (not at the 5′ or 3′ end) from Table 1, Table 3, Table 5, or Table 12 is conjugated with X2-GalNAc (see Formula (V)).

In some instances, one or more endosomal escape moieties (e.g., from 1 to 6 or from 1 to 3) can be attached to a polynucleotide construct or a hybridized polynucleotide construct disclosed herein as an auxiliary moiety. Exemplary endosomal escape moieties include chemotherapeutics (e.g., quinolones such as chloroquine); fusogenic lipids (e.g., dioleoylphosphatidyl-ethanolamine (DOPE)); and polymers such as polyethylenimine (PEI); poly(beta-amino ester)s; polypeptides, such as polyarginines (e.g., octaarginine) and polylysines (e.g., octalysine); proton sponges, viral capsids, and peptide transduction domains as described herein. For example, fusogenic peptides can be derived from the M2 protein of influenza A viruses; peptide analogs of the influenza virus hemagglutinin; the HEF protein of the influenza C virus; the transmembrane glycoprotein of filoviruses; the transmembrane glycoprotein of the rabies virus: the transmembrane glycoprotein (G) of the vesicular stomatitis virus; the fusion protein of the Sendai virus; the transmembrane glycoprotein of the Semliki forest virus; the fusion protein of the human respiratory syncytial virus (RSV); the fusion protein of the measles virus; the fusion protein of the Newcastle disease virus; the fusion protein of the visna virus; the fusion protein of murine leukemia virus; the fusion protein of the HTL virus; and the fusion protein of the simian immunodeficiency virus (SIV). Other moieties that can be employed to facilitate endosomal escape are described in Dominska et al., Journal of Cell Science, 123(8):1183-1189, 2010. Specific examples of endosomal escape moieties including moieties suitable for conjugation to the hybridized polynucleotide constructs disclosed herein are provided, e.g., in WO 2015/188197; the disclosure of these endosomal escape moieties is incorporated by reference herein.

One or more endosomal escape moieties (e.g., from 1 to 6 or from 1 to 3) can be attached to a MOIETY or X2 in formula (V′, V″, V′″, V″″, V′″″, V″″″) through -LinkA-, as described herein.

One or more cell penetrating peptides (CPP) (e.g., from 1 to 6 or from 1 to 3) can be attached to a polynucleotide construct or a hybridized polynucleotide construct disclosed herein as an auxiliary moiety. The CPP can be linked to the hybridized polynucleotide bioreversibly through a disulfide linkage, as disclosed herein. Thus, upon delivery to a cell, the CPP can be cleaved intracellularly, e.g., by an intracellular enzyme (e.g., protein disulfide isomerase, thioredoxin, or a thioesterase) and thereby release the polynucleotide.

CPPs are known in the art (e g., TAT or Arg8) (Snyder and Dowdy, 2005, Expert Opin. Drug Deliv. 2, 43-51). Specific examples of CPPs including moieties suitable for conjugation to the hybridized polynucleotide constructs disclosed herein are provided, e.g., in WO 2015/188197; the disclosure of these CPPs is incorporated by reference herein.

CPPs are positively charged peptides that are capable of facilitating the delivery of biological cargo to a cell. It is believed that the cationic charge of the CPPs is essential for their function. Moreover, the transduction of these proteins does not appear to be affected by cell type, and these proteins can efficiently transduce nearly all cells in culture with no apparent toxicity (Nagahara et al., Nat. Med. 4:1449-52, 1998). In addition to full-length proteins, CPPs have also been used successfully to induce the intracellular uptake of DNA (Abu-Amer, supra), antisense polynucleotides (Astriab-Fisher et al., Pharm. Res, 19:744-54, 2002), small molecules (Polyakov et al., Bioconjug. Chem. 11:762-71, 2000) and even inorganic 40 nm iron particles (Dodd et al., J. Immunol. Methods 256:89-105, 2001; Wunderbaldinger et al., Bioconjug. Chem. 13:264-8, 2002; Lewin et al., Nat. Biotechnol. 18:410-4, 2000; Josephson et al., Bioconjug. Chem. 10:186-91, 1999) suggesting that there is considerable flexibility in particle size in this process.

In one case, a CPP useful in the methods and compositions as described herein includes a peptide featuring substantial alpha-helicity. It has been discovered that transfection is optimized when the CPP exhibits significant alpha-helicity. In another case, the CPP includes a sequence containing basic amino acid residues that are substantially aligned along at least one face of the peptide. A CPP described herein may be a naturally occurring peptide or a synthetic peptide.

One or more cell penetrating peptides (e.g., from 1 to 6 or from 1 to 3) can be attached to a MOIETY or X2 in formula (V′, V″, V′″, V″″, V′″″, V″″″) through -LinkA-, as described herein.

The polynucleotide constructs and the hybridized polynucleotide constructs disclosed herein can also include covalently attached neutral polymer-based auxiliary moieties. Neutral polymers include poly(C1-6 alkylene oxide), e.g., poly(ethylene glycol) and poly(propylene glycol) and copolymers thereof, e.g., di- and triblock copolymers. Other examples of polymers include esterified poly(acrylic acid), esterified poly(glutamic acid), esterified poly(aspartic acid), poly(vinyl alcohol), poly(ethylene-co-vinyl alcohol), poly(N-vinyl pyrrolidone), poly(ethyloxazoline), poly(alkylacrylates), poly(acrylamide), poly(N-alkylacrylamides), poly(N-acryloylmorpholine), poly(lactic acid), poly(glycolic acid), poly(dioxanone), poly(caprolactone), styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyurethane, N-isopropylacrylamide polymers, and poly(N,N-dialkylacrylamides). Exemplary polymer auxiliary moieties may have molecular weights of less than 100, 300, 500, 1000, or 5000 Da (e.g., greater than 100 Da). Other polymers are known in the art.

One or more polymers (e.g., from 1 to 6 or from 1 to 3) can be attached to a MOIETY or X2 in formula (V′, V″, V′″, V″″, V′″″, V″″″) through -LinkA-, as described herein.

### Conjugation Linkers

In some aspects, the polynucleic acid molecules described herein comprises a sense or antisense strand bonded to at least one group of formula (I)

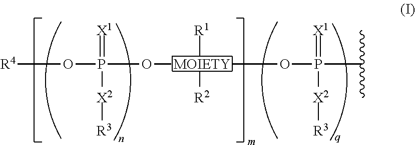

or a salt thereof, or a stereoisomer thereof,
where
each X¹ is independently O or S;
each X² is independently O, S, NH, or a bond;
MOIETY is optionally substituted C₂₋₁₀ alkane-tetrayl or a group -M¹-M²-M³-, wherein each M¹ and each M³ is independently absent or optionally substituted C₁₋₆ alkylene, and M² is optionally substituted C₃₋₉ heterocycle-tetrayl, optionally substituted C₆₋₁₀ arene-tetrayl, or optionally substituted C₃₋₈ cycloalkane-tetrayl;
each R¹ and each R² is independently H, optionally substituted C₁₋₁₆ alkyl, optionally substituted C₂₋₁₆ heteroalkyl, a conjugation moiety, or -LinkA(-T)ₚ, provided that at least one R¹ or at least one R² is a conjugation moiety or -LinkA(-T)ₚ;
each R³ is independently H, optionally substituted C₁₋₁₆ alkyl, optionally substituted C₂₋₁₆ heteroalkyl, optionally substituted C₂₋₁₆ alkenyl, optionally substituted C₂₋₁₆ alkynyl, optionally substituted (C₁₋₉ heterocyclyl)-C₁₋₆-alkyl, optionally substituted (C₆₋₁₀ aryl)-C₁₋₆-alkyl, optionally substituted (C₃₋₈ cycloalkyl)-C₁₋₆-alkyl, a conjugation moiety, or -LinkA(-T)ₚ;
R⁴ is H, optionally substituted C₁₋₆ alkyl, -LinkA(-T)ₚ, or -Sol;
each LinkA is independently a multivalent linker (e.g., including —C(O)—N(H)— (e.g., at least one multivalent linker including —C(O)—N(H)— bonded to T));
each T is independently an auxiliary moiety;
Sol is solid support;
m is an integer from 1 to 6;
each n is independently 0 or 1;
each p is independently an integer from 1 to 6; and
q is an integer from 0 to 3.

The at least one group of formula (I) may be bonded to a 5′-terminus, 3′-terminus, internucleoside phosphate, internucleoside phosphorothioate, or internucleoside phosphorodithioate of the polynucleotide. When the at least one group of formula (I) is bonded to the internucleoside phosphate, internucleoside phosphorothioate, or internucleoside phosphorodithioate, q is 0. The polynucleotide construct contains no more than one Sol.

Group -LinkA- can include from 0 to 3 multivalent monomers (e.g., optionally substituted C1-6 alkane-triyl, optionally substituted C1-6 alkane-tetrayl, or trivalent nitrogen atom) and one or more divalent monomers (e.g., from 1 to 40), where each divalent monomer is independently optionally substituted C1-6 alkylene; optionally substituted C2-6 alkenylene; optionally substituted C2-6 alkynylene; optionally substituted C3-8 cycloalkylene; optionally substituted C3-8 cycloalkenylene; optionally substituted C6-14 arylene; optionally substituted C1-9 heteroarylene having 1 to 4 heteroatoms selected from N, O, and S; optionally substituted C1-9 heterocyclylene having 1 to 4 heteroatoms selected from N, O, and S; imino; optionally substituted N; O; or S(O)m, wherein m is 0, 1, or 2. In some aspects, each monomer is independently optionally substituted C1-6 alkylene; optionally substituted C3-8 cycloalkylene; optionally substituted C3-8 cycloalkenylene; optionally substituted C6-14 arylene; optionally substituted C1-9 heteroarylene having 1 to 4 heteroatoms selected from N, O, and S; optionally substituted C1-9 heterocyclylene having 1 to 4 heteroatoms selected from N, O, and S; imino; optionally substituted N; O; or S(O)m, where m is 0, 1, or 2 (e.g., n is 2). In certain aspects, each monomer is independently optionally substituted C1-6 alkylene; optionally substituted C3-8 cycloalkylene; optionally substituted C3-8 cycloalkenylene; optionally substituted C6-14 arylene; optionally substituted C1-9 heteroarylene having 1 to 4 heteroatoms selected from N, O, and S; optionally substituted C1-9 heterocyclylene having 1 to 4 heteroatoms selected from N, O, and S; optionally substituted N; O; or S(O)m, where m is 0, 1, or 2 (e.g., m is 2). The non-bioreversible linker connecting the auxiliary moiety to the conjugating moiety or to the reaction product thereof can include from 2 to 500 (e.g., from 2 to 300 or from 2 to 200) of such monomers. Group -LinkA- may include a poly(alkylene oxide) (e.g., polyethylene oxide, polypropylene oxide, poly(trimethylene oxide), polybutylene oxide, poly(tetramethylene oxide), and diblock or triblock co-polymers thereof). In some aspects, the non-bioreversible linker includes polyethylene oxide (e.g., poly(ethylene oxide) having a molecular weight of less than 1 kDa).

Group -LinkA(-T)ₚ in formula (I) may be prepared by a process described in the sections below. In some instances, -LinkA(-T)ₚ is of formula (II):

-Q¹-Q²([-Q³-Q⁴-Q⁵]ₛ-Q⁶-T)ₚ, (II)
where
each s is independently an integer from 0 to 20 (e.g., from 0 to 10), where the repeating units are the same or different;
Q¹ is a conjugation linker (e.g., [-Q³-Q⁴-Q⁵]ₛ-Q^{C} where Q^{C} is optionally substituted C₂₋₁₂ heteroalkylene (e.g., a heteroalkylene containing —C(O)—N(H)—, —N(H)—C(O)—, —S(O)₂—N(H)—, or —N(H)—S(O)₂—), optionally substituted C₁₋₁₂ thioheterocyclylene

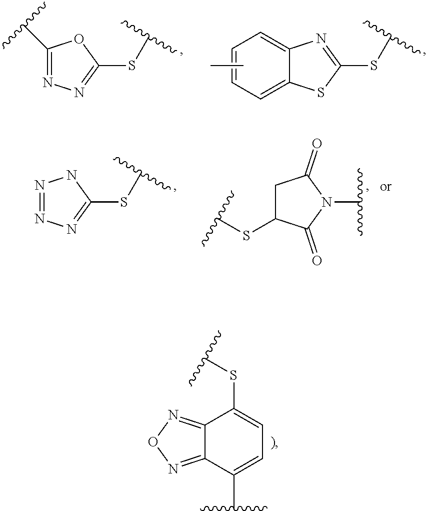

optionally substituted C₁₋₁₂ heterocyclylene (e.g., 1,2,3-triazole-1,4-diyl or

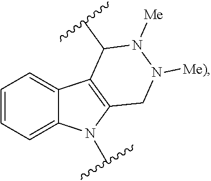

cyclobut-3-ene-1,2-dione-3,4-diyl, or pyrid-2-yl hydrazone);
Q² is a linear group (e.g., [-Q³-Q⁴-Q⁵]ₛ-), if p is 1, or a branched group (e.g., [-Q³-Q⁴-Q⁵]ₛ-Q⁷([-Q³-Q⁴-Q⁵]ₛ-(Q⁷)ₚ₁)ₚ₂, where p1 is 0 or 1, p2 is 0, 1, 2, or 3), if p is an integer from 2 to 6;
each Q³ and each Q⁶ is independently absent, —CO—, —NH—, —O—, —S—, —SO₂—, —OC(O)—, —COO—, —NHC(O)—, —C(O)NH—, —CH₂—, —CH₂NH—, —NHCH₂—, —CH₂O—, or —OCH₂—;
each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₆₋₁₀ arylene, optionally substituted C₁₋₉ heteroarylene, or optionally substituted C₉, heterocyclylene;
each Q⁵ is independently absent, —CO—, —NH—, —O—, —S—, —SO₂—, —CH₂—, —C(O)O—, —OC(O)—, —C(O)NH—, —NH—C(O)—, —NH—CH(R^{a})—C(O)—, or —C(O)—CH(R^{a})—NH—,
each Q⁷ is independently optionally substituted C₁₋₆ alkane-triyl, optionally substituted C₁₋₆ alkane-tetrayl, optionally substituted C₂₋₆ heteroalkane-triyl, or optionally substituted C₂₋₆ heteroalkane-tetrayl; and
each R^{a} is independently H or an amino acid side chain;
provided that at least one of Q³, Q⁴, and Q⁵ is present.

In some aspects, each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, or optionally substituted C₁₋₉ heterocyclylene. In certain aspects, s is 1, 2, 3, 4, 5, 6, 7, 8, 9, 10, 11, 12, 13, 14, 15, 16, 17, 18, 19, or 20.

Thus, in formula (II), LinkA may include a single branching point, if each p1 is 0, or multiple branching points, if at least one p1 is 1.

In formula (II), Q¹ may be —O-Q^{L}-Q^{C}-, where Q^{L} is optionally substituted C₂₋₁₂ heteroalkylene, optionally substituted C₁₋₁₂ alkylene, or -(optionally substituted C₁₋₆ alkylene)-(optionally substituted C₆₋₁₀ arylene)-. In some aspects, Q^{L} is optionally substituted C₂₋₁₂ heteroalkylene or optionally substituted C₁₋₁₂ alkylene. In formula (II), Q^{C} may be:

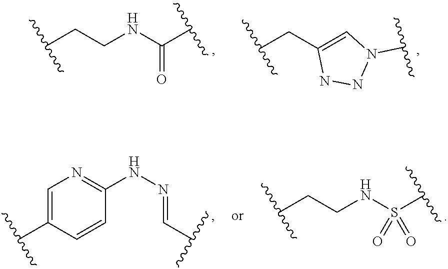 [SMILES: *CCNC(*)=O ; *CC1=CN(*)NN1 ; */C=N/Nc1ccc(*)cn1 ; *CCNS(*)(=O)=O]

In formula (II), Q² may be a linear group of formula [-Q³-Q⁴-Q⁵]ₛ-, where Q³, Q⁴, and Q⁵ are as defined for formula (II). Alternatively, Q² may be a branched group [-Q³-Q⁴-Q⁵]ₛ-Q⁷([-Q³-Q⁴-Q⁵]ₛ-(Q⁷)ₚ₁)ₚ₂, where each Q⁷ is independently optionally substituted C₁₋₆ alkane-triyl, optionally substituted C₁₋₆ alkane-tetrayl, optionally substituted C₂₋₆ heteroalkane-triyl, or optionally substituted C₂₋₆ heteroalkane-tetrayl;
where
p1 is 0 or 1;
p2 is 0, 1, 2, or 3;
where,
   when p1 is 0, LinkA is a trivalent or tetravalent linker, and,
   when p1 is 1, LinkA is a tetravalent, pentavalent, or hexavalent linker.

In certain aspects, p1 is 0.

In some aspects, Q⁷ is:

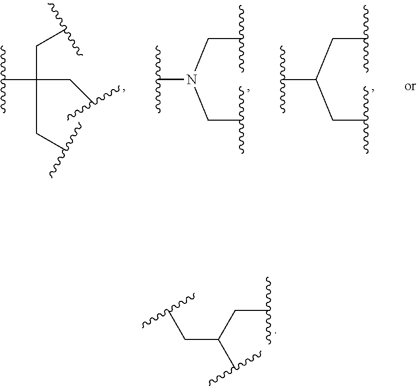 [SMILES: *CC(*)(C*)C* ; *CN(*)C* ; *CC(*)C* ; *CC(*)C*]

Compounds that may be used in the preparation of group -LinkA(-T)p in formula (I) are described herein as well as in WO 2015/188197. Non-limiting examples of -LinkA include:

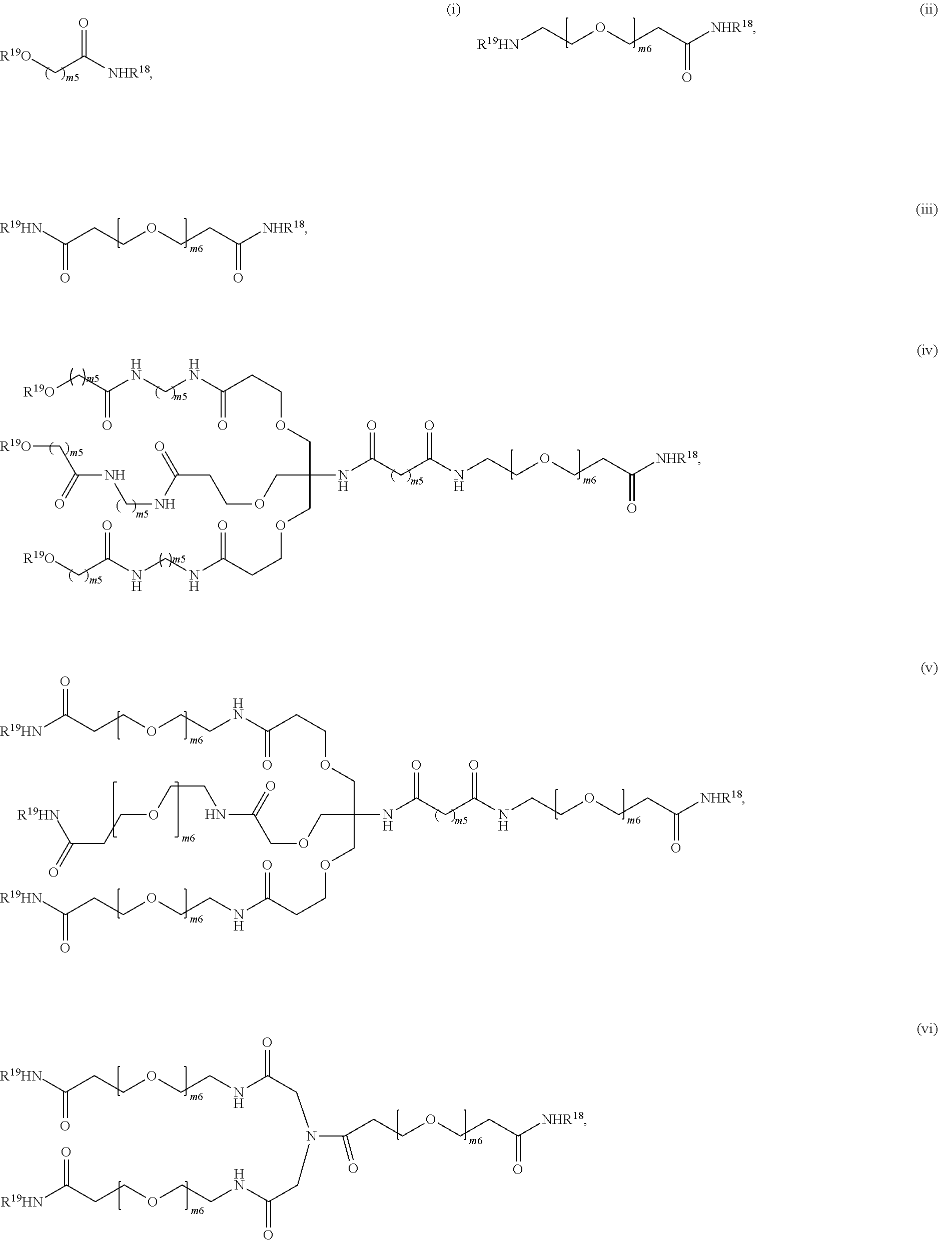

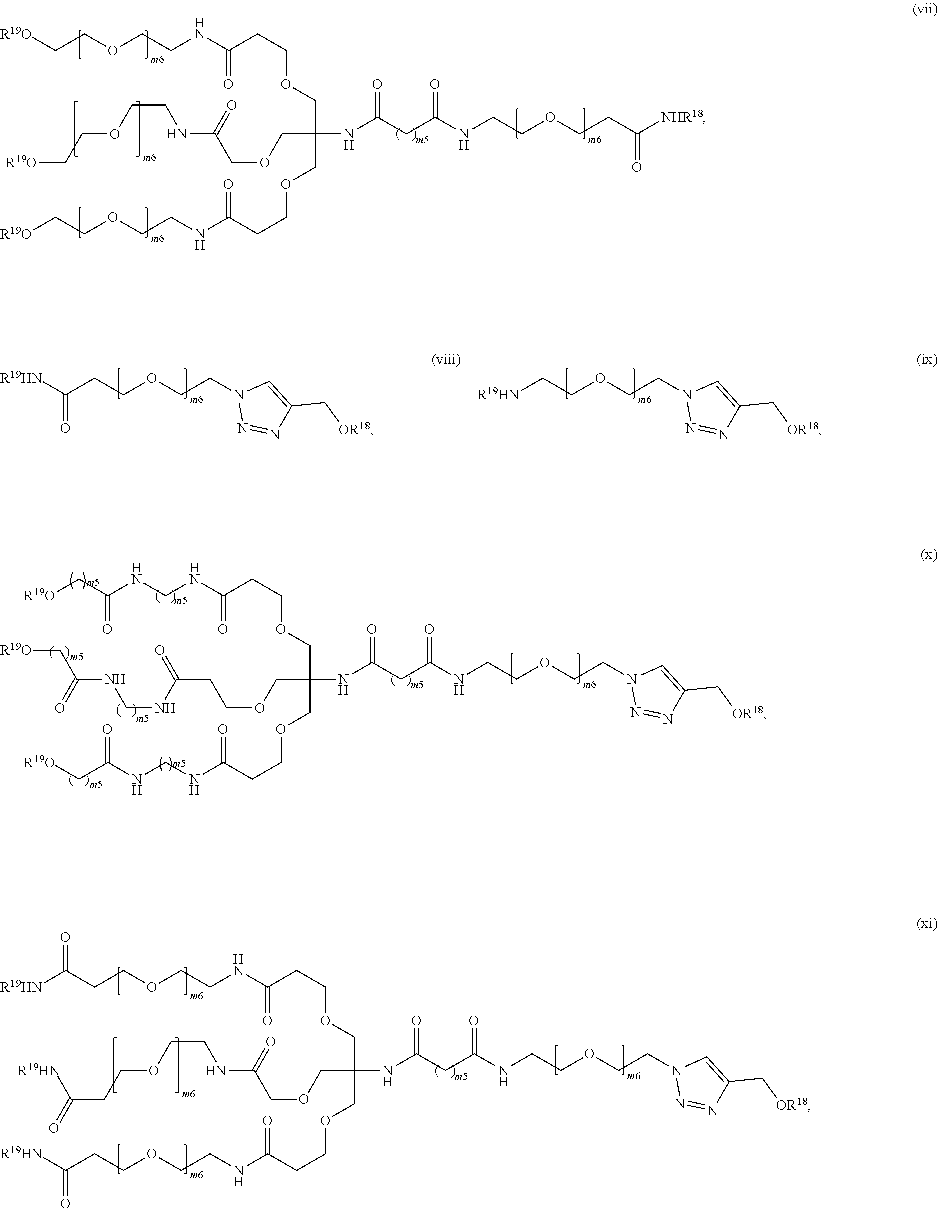

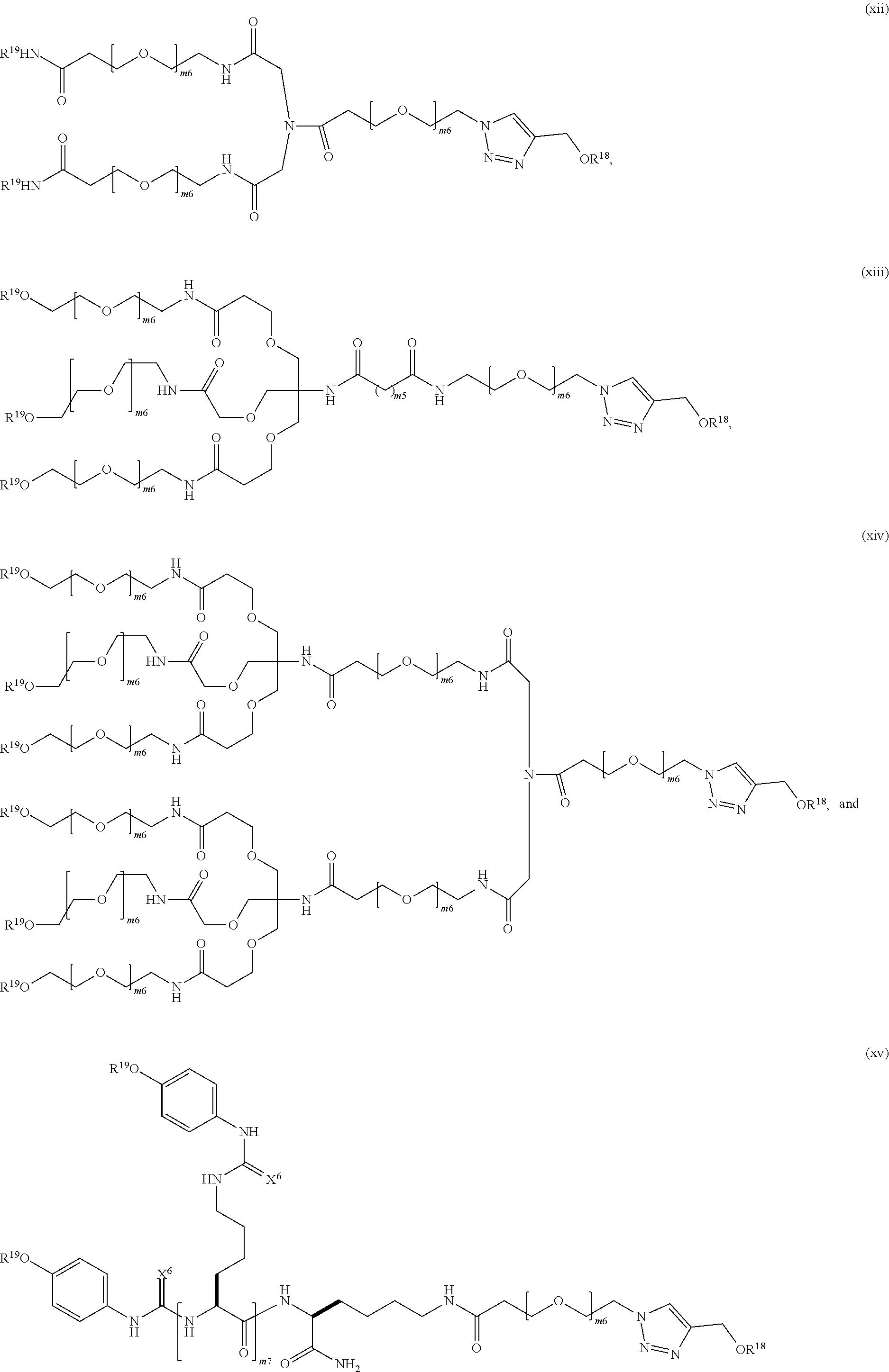

where
R¹⁸ is a bond to MOIETY,
each R¹⁹ is independently a bond to auxiliary moiety,
each m5 is independently an integer from 1 to 20,
each m6 is independently an integer from 1 to 10,
m7 is an integer from 1 to 6, and
each X⁶ is independently O or S.

In formula (II), when the conjugation linker is of formula [-Q³-Q⁴-Q⁵]ₛ-Q^{C}-, -Q²([-Q³-Q⁴-Q⁵]ₛ-Q⁶-T)_{P} may be:

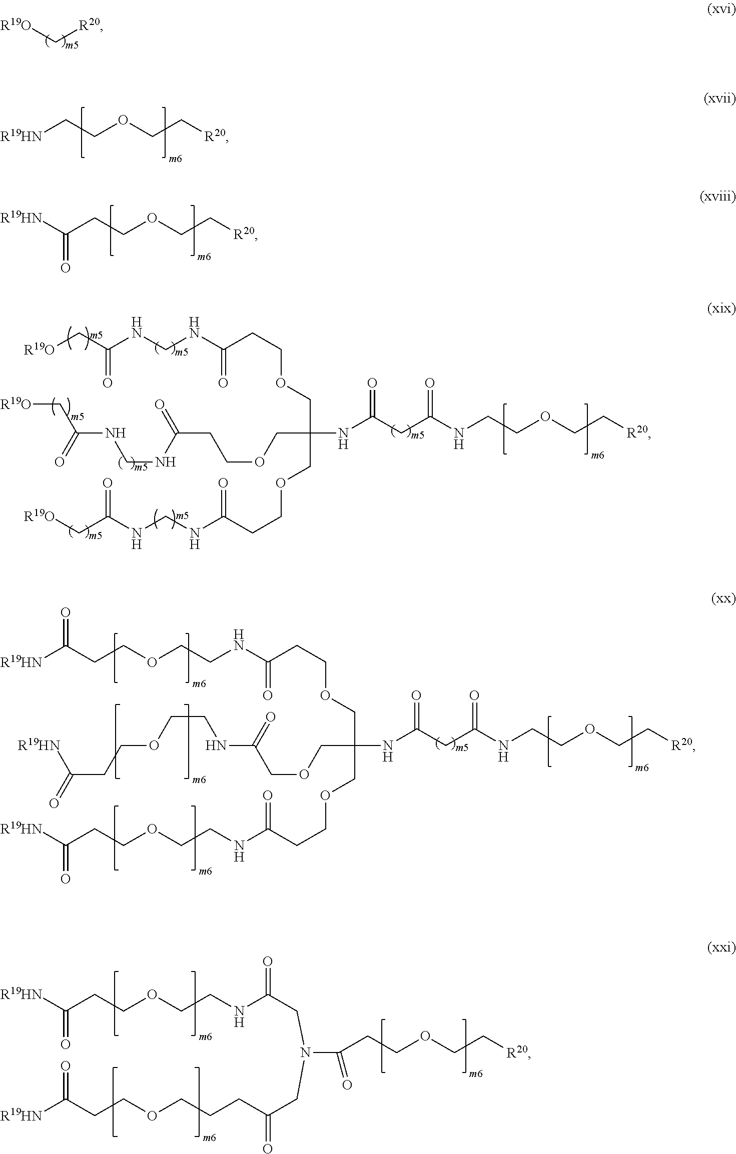

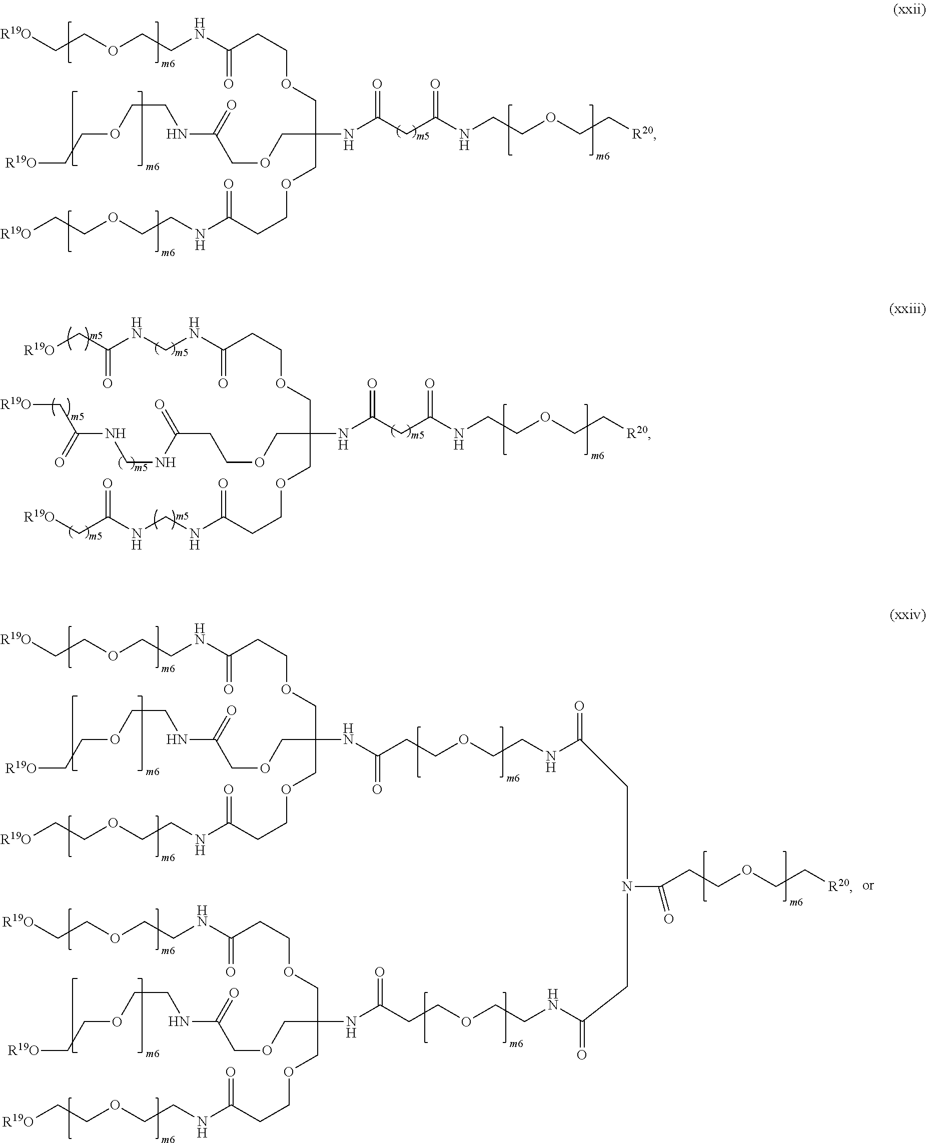

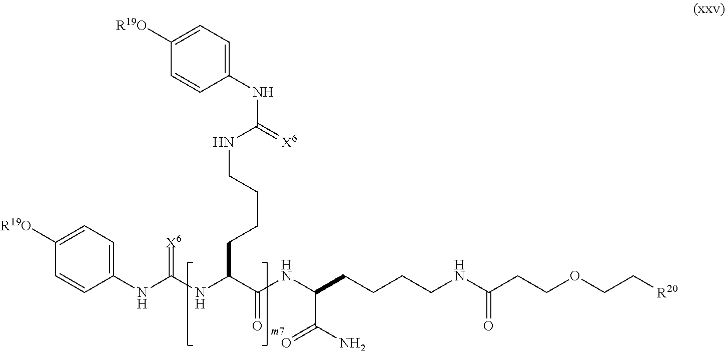

where
R²⁰ is a bond to Q^{C} in Q¹,
each R¹⁹ is independently a bond to an auxiliary moiety,
each m5 is independently an integer from 1 to 20,
each m6 is independently an integer from 1 to 10,
m7 is an integer from 1 to 6, and
each X⁶ is independently O or S.

In some aspects, the linker described herein is cleavable. In some aspects, the linker described herein is non-cleavable.

In some aspects, the polynucleic acid molecule described herein comprises a sense or antisense strand bonded to at least one group of formula (IV),

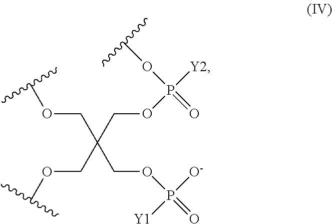

wherein at least one of Y1 or Y2 is a nucleotide from the polynucleic acid molecule.

In some instances, the Y1 is the last nucleotide on the 3′-terminus or the first nucleotide on the 5′-terminus of one of the strands of the polynucleic acid molecule. In some instances, the Y1 is the last nucleotide on the 3′-terminus or the first nucleotide on the 5′-terminus of the sense strand of the polynucleic acid molecule. In some instances, the Y1 is the last nucleotide on the 3′-terminus or the first nucleotide on the 5′-terminus of the sense strand of the polynucleic acid molecule, and the Y2 is a 3-hydroxy-propoxy group. In some instances, the Y2 is the first nucleotide on the 5′-terminus or the last nucleotide on the 3′-terminus of one of the strands of the polynucleic acid molecule. In some instances, the Y2 is the first nucleotide on the 5′-terminus or the last nucleotide on the 3′-terminus of the sense strand of the polynucleic acid molecule. In some instances, the Y2 is the first nucleotide on the 5′-terminus or the last nucleotide on the 3′-terminus of the sense strand of the polynucleic acid molecule, and the Y1 is a 3-hydroxy-propoxy group. In other instances, the Y1 and Y2 are two consecutive nucleotides in one of the strands of the polynucleic acid molecule.

In some aspects, the targeting moiety described herein is conjugated to 3′ end of the sense strand (e.g., formula (IV′) or (IV″″)). In some aspects, the targeting moiety described herein is conjugated to 5′ end of the sense strand (e.g., formula (IV″) or (IV′″)). In some aspects, the targeting moiety described herein is conjugated to 3′ end of the antisense strand (e.g., formula (IV′) or (IV″″)). In some aspects, the targeting moiety described herein is conjugated to 5′ end of the antisense strand (e.g., formula (IV″) or (IV′″)).

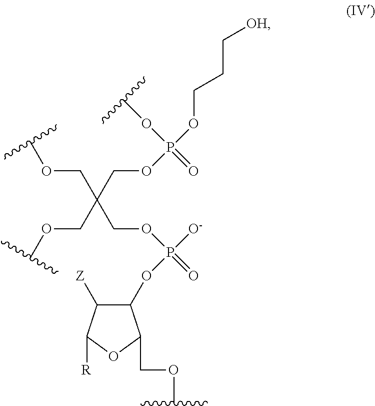

wherein Z in formula (IV′) is a moiety that corresponds to one of the sugar modifications described herein (e.g., —H, —OH, —O-Methyl, —F, or —O-methoxyethyl) and R in formula (IV′) is adenine, uracil, guanine, cytosine, thymine, abasic, or others.

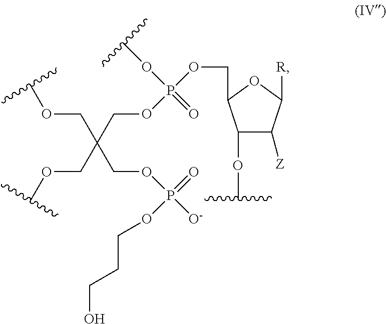

wherein Z in formula (IV″) is a moiety that corresponds to one of the sugar modifications described herein (e.g., —H, —OH, —O-Methyl, —F, or —O-methoxyethyl) and R in formula (IV″) is adenine, uracil, guanine, cytosine, thymine, abasic, or others.

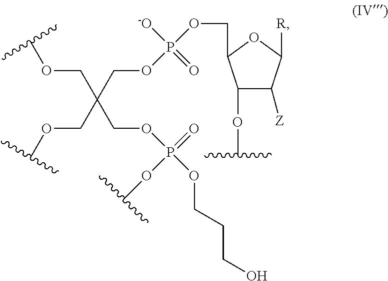

wherein Z in formula (IV′″) is a moiety that corresponds to one of the sugar modifications described herein (e.g., —H, —OH, —O-Methyl, —F, or —O-methoxyethyl) and R in formula (IV′″) is adenine, uracil, guanine, cytosine, thymine, abasic, or others.

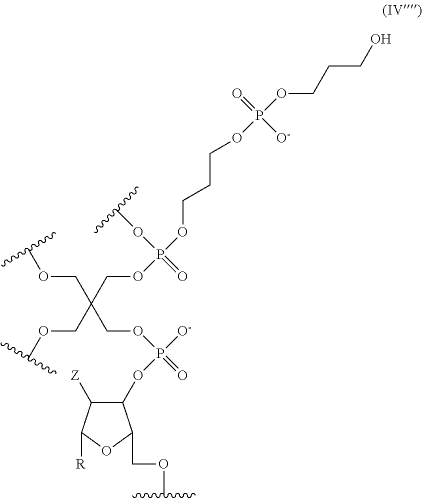

wherein Z in formula (IV″″) is a moiety that corresponds to one of the sugar modifications described herein (e.g., —H, —OH, —O-Methyl, —F, or —O-methoxyethyl) and R in formula (IV″″) is adenine, uracil, guanine, cytosine, thymine, abasic, or others.

### Pharmaceutical Compositions

Delivery of the polynucleotide molecules described herein can be achieved by contacting a cell with the polynucleotide molecules using a variety of methods. In particular aspects, the polynucleotide molecule described herein is formulated with various excipients, vehicles, and carriers, as described more fully elsewhere herein.

A pharmaceutical composition described herein can be prepared to include a hybridized polynucleotide construct disclosed herein, into a form suitable for administration to a subject using carriers, excipients, and vehicles. Frequently used excipients include magnesium carbonate, titanium dioxide, lactose, mannitol and other sugars, tale, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable vehicles include aqueous solutions, non-toxic excipients, including salts, preservatives, buffers and the like, as described, for instance, in Remington: The Science and Practice of Pharmacy, 21 st Ed., Gennaro, Ed., Lippencott Williams & Wilkins (2005), and The United States Pharmacopeia: The National Formulary (USP 36 NF31), published in 2013. The pH and exact concentration of the various components of the pharmaceutical composition are adjusted according to routine skills in the art. See Goodman and Gilman's, The Pharmacological Basis for Therapeutics.

The pharmaceutical compositions described herein may be administered locally or systemically. The therapeutically effective amounts will vary according to factors, such as the degree of infection in a subject, the age, sex, and weight of the individual. Dosage regimes can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g., subcutaneous, intravenous, intraorbital, and the like), oral administration, ophthalmic application, inhalation, topical application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethyleneglycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically the composition will be stable under the conditions of manufacture and storage and preserved against the contaminating action of microorganisms, such as bacteria and fungi. The vehicle can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

It is especially advantageous to formulate parenteral compositions in dosage unit form for ease of administration and uniformity of dosage. Dosage unit form as used herein, refers to physically discrete units suited as unitary dosages for the subject to be treated; each unit containing a predetermined quantity of pharmaceutical composition is calculated to produce the desired therapeutic effect in association with the required pharmaceutical vehicle. The specification for the dosage unit forms is related to the characteristics of the pharmaceutical composition and the particular therapeutic effect to be achieve. The principal pharmaceutical composition is compounded for convenient and effective administration in effective amounts with a suitable pharmaceutically acceptable vehicle in an acceptable dosage unit. In the case of compositions containing supplementary active ingredients, the dosages are determined by reference to the usual dose and manner of administration of the ingredients.

The pharmaceutical composition can be orally administered, for example, in a carrier, e.g., in an enteric-coated unit dosage form. The pharmaceutical composition and other ingredients can also be enclosed in a hard or soft-shell gelatin capsule or compressed into tablets. For oral therapeutic administration, the pharmaceutical composition can be incorporated with excipients and used in the form of ingestible tablets, troches, capsules, pills, wafers, and the like. Such compositions and preparations should contain at least 1% by weight of active compound. The percentage of the compositions and preparations can, of course, be varied and can conveniently be between about 5% to about 80% of the weight of the unit. The tablets, troches, pills, capsules, and the like can also contain the following: a binder, such as gum tragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Any material used in preparing any dosage unit form should be of pharmaceutically acceptable purity and substantially non-toxic in the amounts employed. In addition, the pharmaceutical composition can be incorporated into sustained-release preparations and formulations.

The pharmaceutical composition described herein may comprise one or more permeation enhancer that facilitates bioavailability of the polynucleotide molecule described herein. WO 2000/67798, Muranishi, 1990, Crit. Rev. Ther. Drug Carrier Systems, 7, 1, Lee et al., 1991, Crit. Rev. Ther. Drug Carrier Systems, 8, 91 are herein incorporated by reference in its entirety. In some aspects, the permeation enhancer is intestinal. In some aspects, the permeation enhancer is transdermal. In some aspects, the permeation enhancer is to facilitate crossing the brain-blood barrier. In some aspects, the permeation enhancer improves the permeability in the oral, nasal, buccal, pulmonary, vaginal, or corneal delivery model. In some aspects, the permeation enhancer is a fatty acid or a derivative thereof. In some aspects, the permeation enhancer is a surfactant or a derivative thereof. In some aspects, the permeation enhancer is a bile salt or a derivative thereof. In some aspects, the permeation enhancer is a chelating agent or a derivative thereof. In some aspects, the permeation enhancer is a non-chelating non-surfactant or a derivative thereof. In some aspects, the permeation enhancer is an ester or a derivative thereof. In some aspects, the permeation enhancer is an ether or a derivative thereof. In some aspects, the permeation enhancer is arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof. In one specific aspect, the permeation enhancer is sodium caprate (C10). In some aspects, the permeation enhancer is chenodeoxycholic acid (CDCA), ursodeoxychenodeoxycholic acid (UDCA), cholic acid, dehydrocholic acid, deoxycholic acid, glucholic acid, glycholic acid, glycodeoxycholic acid, taurocholic acid taurodeoxycholic acid, sodium tauro-24,25-dihydro-fusidate or sodium glycodihydrofusidate. In some aspects, the permeation enhancer is polyoxyethylene-9-lauryl ether, or polyoxyethylene-20-cetyl ether.

For the polynucleotide molecule described herein, suitable pharmaceutically acceptable salts include (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, and the like; and (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like.

While the hybridized polynucleotide constructs described herein may not require the use of excipients for delivery to the target cell, the use of excipients may be advantageous in some aspects. Thus, for delivery to the target cell, the hybridized polynucleotide molecule described herein can non-covalently bind an excipient to form a complex. The excipient can be used to alter biodistribution after delivery, to enhance uptake, to increase half-life or stability of the strands in the hybridized polynucleotide constructs (e.g., improve nuclease resistance), and/or to increase targeting to a particular cell or tissue type.

Exemplary excipients include a condensing agent (e.g., an agent capable of attracting or binding a nucleic acid through ionic or electrostatic interactions); a fusogenic agent (e.g., an agent capable of fusing and/or being transported through a cell membrane); a protein to target a particular cell or tissue type (e.g., thyrotropin, melanotropin, lectin, glycoprotein, surfactant protein A, or any other protein); a lipid; a lipopolysaccharide; a lipid micelle or a liposome (e.g., formed from phospholipids, such as phosphotidylcholine, fatty acids, glycolipids, ceramides, glycerides, cholesterols, or any combination thereof); a nanoparticle (e.g., silica, lipid, carbohydrate, or other pharmaceutically-acceptable polymer nanoparticle); a polyplex formed from cationic polymers and an anionic agent (e.g., a CRO), where exemplary cationic polymers include polyamines (e.g., polylysine, polyarginine, polyamidoamine, and polyethylene imine); cholesterol; a dendrimer (e.g. a polyamidoamine (PAMAM) dendrimer); a serum protein (e.g., human serum albumin (HSA) or low-density lipoprotein (LDL); a carbohydrate (e.g., dextran, pullulan, chitin, chitosan, inulin, cyclodextrin, or hyaluronic acid); a lipid; a synthetic polymer, (e.g., polylysine (PLL), polyethylenimine, poly-L-aspartic acid, poly-L-glutamic acid, styrene-maleic acid anhydride copolymer, poly(L-lactide-co-glycolic) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacrylic acid), N-isopropylacrylamide polymer, pseudopeptide-polyamine, peptidomimetic polyamine, or polyamine); a cationic moiety (e.g., cationic lipid, cationic porphyrin, quaternary salt of a polyamine, or alpha helical peptide); a multivalent sugar (e.g., multivalent lactose, multivalent galactose, N-acetyl-galactosamine, N-acetyl-glucosamine, multivalent mannose, or multivalent fucose); a vitamin (e.g., vitamin A, vitamin E, vitamin K, vitamin B, folic acid, vitamin B12, riboflavin, biotin, or pyridoxal); a cofactor; or a drug to disrupt cellular cytoskeleton to increase uptake (e.g., taxol, vincristine, vinblastine, cytochalasin, nocodazole, japlakinolide, latrunculin A, phalloidin, swinholide A, indanocine, or myoservin).

Other therapeutic agents as described herein may be included in a pharmaceutical composition described herein in combination with a polynucleotide molecule described herein.

### Methods of Treatment

In some aspects, described herein is a method of modulating expression of APOC3 gene in a subject, comprising: administering to the subject a polynucleic acid molecule described herein, a polynucleic acid molecule conjugate described herein, or a pharmaceutical composition described herein, thereby modulating the expression of APOC3 gene in the subject.

In some aspects, the method described herein reduces expression of APOC3 gene in a subject by about or at least 10% compared to a negative control. In some aspects, the method described herein reduces expression of APOC3 gene in a subject by about or at least 20% compared to a negative control. In some aspects, the method described herein reduces expression of APOC3 gene in a subject by about or at least 30% compared to a negative control. In some aspects, the method described herein reduces expression of APOC3 gene in a subject by about or at least 40% compared to a negative control. In some aspects, the method described herein reduces expression of APOC3 gene in a subject by about or at least 50% compared to a negative control. In some aspects, the method described herein reduces expression of APOC3 gene in a subject by about or at least 60% compared to a negative control. In some aspects, the method described herein reduces expression of APOC3 gene in a subject by about or at least 70% compared to a negative control. In some aspects, the method described herein reduces expression of APOC3 gene in a subject by about or at least 80% compared to a negative control. In some aspects, the method described herein reduces expression of APOC3 gene in a subject by about or at least 90% compared to a negative control. In some aspects, the method described herein reduces expression of APOC3 gene in a subject by about 100% compared to a negative control.

In some aspects, the method described herein achieves an IC50 value of about 5 nM. In some aspects, the method described herein achieves an IC50 value of about 10 nM. In some aspects, the method described herein achieves an IC50 value of about 15 nM. In some aspects, the method described herein achieves an IC50 value of about 20 nM. In some aspects, the method described herein achieves an IC50 value of about 25 nM. In some aspects, the method described herein achieves an IC50 value of about 30 nM. In some aspects, the method described herein achieves an IC50 value of about 35 nM. In some aspects, the method described herein achieves an IC50 value of about 40 nM, In some aspects, the method described herein achieves an IC50 value of about 45 nM. In some aspects, the method described herein achieves an IC50 value of about 50 nM. In some aspects, the method described herein achieves an IC50 value of about 55 nM. In some aspects, the method described herein achieves an IC50 value of about 60 nM. In some aspects, the method described herein achieves an IC50 value of about 65 nM. In some aspects, the method described herein achieves an IC50 value of about 70 nM. In some aspects, the method described herein achieves an IC50 value of about 75 nM. In some aspects, the method described herein achieves an IC50 value of about 80 nM. In some aspects, the method described herein achieves an IC50 value of about 85 nM. In some aspects, the method described herein achieves an IC50 value of about 90 nM. In some aspects, the method described herein achieves an IC50 value of about 95 nM. In some aspects, the method described herein achieves an IC50 value of about 100 nM.

In some aspects, the method described herein achieves an IC50 value of about 1 μM. In some aspects, the method described herein achieves an IC50 value of about 1.1 μM. In some aspects, the method described herein achieves an IC50 value of about 1.2 μM, In some aspects, the method described herein achieves an IC50 value of about 1.3 μM. In some aspects, the method described herein achieves an IC50 value of about 1.4 μM. In some aspects, the method described herein achieves an IC50 value of about 1.5 μM. In some aspects, the method described herein achieves an IC50 value of about 2 μM. In some aspects, the method described herein achieves an IC50 value of about 4 μM. In some aspects, the method described herein achieves an IC50 value of about 6 μM. In some aspects, the method described herein achieves an IC50 value of about 8 μM. In some aspects, the method described herein achieves an IC50 value of about 10 μM. In some aspects, the method described herein achieves an IC50 value of about 12 μM. In some aspects, the method described herein achieves an IC50 value of about 13 μM. In some aspects, the method described herein achieves an IC50 value of about 14 μM. In some aspects, the method described herein achieves an IC50 value of about 15 μM. In some aspects, the method described herein achieves an IC50 value of about 30 μM. In some aspects, the method described herein achieves an IC50 value of about 35 μM. In some aspects, the method described herein achieves an IC50 value of about 40 μM. In some aspects, the method described herein achieves an IC50 value of about 50 μM. In some aspects, the method described herein achieves an IC50 value of about 60 μM. In some aspects, the method described herein achieves an IC50 value of about 80 μM. In some aspects, the method described herein achieves an IC50 value of about 100 μM. In some aspects, the method described herein achieves an IC50 value of about 120 μM In some aspects, the method described herein achieves an IC50 value of about 160 μM.

In some aspects, described herein is a method of modulating triglyceride in a subject in need thereof, comprising administering to the subject a polynucleic acid molecule described herein, a polynucleic acid molecule conjugate described herein, or a pharmaceutical composition described herein, wherein the polynucleic acid molecule described herein, the polynucleic acid molecule conjugate described herein, or the pharmaceutical composition described herein reduces the expression of APOC3 gene in the subject.

In some aspects, the method described herein reduces triglyceride level in a subject by about or at least 10% compared to a negative control. In some aspects, the method described herein reduces triglyceride level in a subject by about or at least 20% compared to a negative control. In some aspects, the method described herein reduces triglyceride level in a subject by about or at least 30% compared to a negative control. In some aspects, the method described herein reduces triglyceride level in a subject by about or at least 40% compared to a negative control. In some aspects, the method described herein reduces triglyceride level in a subject by about or at least 50% compared to a negative control. In some aspects, the method described herein reduces triglyceride level in a subject by about or at least 60% compared to a negative control. In some aspects, the method described herein reduces triglyceride level in a subject by about or at least 70% compared to a negative control. In some aspects, the method described herein reduces triglyceride level in a subject by about or at least 80% compared to a negative control. In some aspects, the method described herein reduces triglyceride level in a subject by about or at least 90% compared to a negative control. In some aspects, the method described herein reduces triglyceride level in a subject by about 100% compared to a negative control.

In some aspects, the subject receiving the method described herein suffers from severe hypertriglyceridemia. In some specific case, the subject receiving the method described herein suffers from familial chylomicron syndrome (FCS). In other aspects, the subject receiving the method described herein suffers from high fasting triglycerides on restricted low-fat diet. In other aspects, the subject receiving the method described herein suffers from overweight or obese. In other aspects, the subject receiving the method described herein suffers from type 2 diabetes. In other aspects, the subject receiving the method described herein suffers from dyslipidaemia. In other aspects, the subject receiving the method described herein suffers a cardiovascular disease. In other aspects, the subject receiving the method described herein suffers from atherosclerosis. In other aspects, the subject receiving the method described herein suffers from stroke. In other aspects, the subject receiving the method described herein suffers from acute pancreatitis. In other aspects, the subject receiving the method described herein suffers from atherosclerotic cardiovascular disease. In other aspects, the subject receiving the method described herein suffers from atrial fibrillation. In other aspects, the subject receiving the method described herein suffers from myocardial infarction. In other aspects, the subject receiving the method described herein suffers from calcific aortic valve stenosis. In other aspects, the subject receiving the method described herein suffers from cardiac arrest. In other aspects, the subject receiving the method described herein suffers from peripheral arterial disease.

### EXEMPLARY EMBODIMENTS

Embodiment 1. A polynucleic acid molecule for modulating expression of apolipoprotein C3 (APOC3) gene, wherein the polynucleic acid molecule comprises a nucleic acid sequence that is at least 80%, at least 85%, at least 90% identical to a nucleic acid sequence in Table 1, Table 3, Table 5, or Table 12.

Embodiment 2. The polynucleic acid molecule of embodiment 1, wherein the polynucleic acid molecule comprises a nucleic acid sequence in Table 1, Table 3, Table 5, or Table 12.

Embodiment 3. The polynucleic acid molecule of any one of embodiments 1-2, wherein the polynucleic acid molecule is a single-stranded nucleic acid molecule.

Embodiment 4. The polynucleic acid molecule of embodiment 3, wherein the single-stranded nucleic acid molecule comprises at least 14, 15, 16, 17, 18 consecutive nucleotides that are complementary to a nucleic acid sequence selected from SEQ ID NOs: 217-324, 481-504, 541-546, and 575-585, with no more than 1, 2, 3, 4 mismatches.

Embodiment 5. The polynucleic acid molecule of embodiment 3, wherein the single-stranded nucleic acid molecule comprises a sequence that is at least 80%, at least 85%, at least 90%, at least 95% complementary to a nucleic acid sequence selected from SEQ ID NOs: 217-324, 481-504, 541-546, and 575-585.

Embodiment 6. The polynucleic acid molecule of embodiment 3, wherein the single-stranded nucleic acid molecule comprises a sequence that is at least 80%, at least 85%, at least 90%, at least 95% identical to a nucleic acid sequence selected from SEQ ID NOs: 1-108, 433-456, 529-534, and 553-563.

Embodiment 7. The polynucleic acid molecule of embodiment 3, wherein the single-stranded nucleic acid molecule comprises at least 14, 15, 16, 17, 18 consecutive nucleotides that are identical to a nucleic acid sequence selected from SEQ ID NOs: 1-108, 433-456, 529-534, and 553-563.

Embodiment 8. The polynucleic acid molecule of embodiment 1, wherein the polynucleic acid molecule is a double-stranded nucleic acid molecule comprising a sense strand and an antisense strand.

Embodiment 9. The polynucleic acid molecule of embodiment 8, wherein the sense strand comprises a nucleic acid sequence that is at least 80%, at least 85%, at least 90%, at least 95% identical to a nucleic acid sequence selected from SEQ ID NOs: 217-324, 481-504, 541-546, and 575-585.

Embodiment 10. The polynucleic acid molecule of embodiment 8, wherein the sense strand comprises a nucleic acid sequence that is at least 90%, at least 95% identical to a nucleic acid sequence selected from SEQ ID NOs: 541-546 and 575-585.

Embodiment 11. The polynucleic acid molecule of any one of embodiments 8-10, wherein the antisense strand comprises a nucleic acid sequence that is at least 80%, at least 85%, at least 90%, at least 95% identical to a nucleic acid sequence selected from SEQ ID NOs: 1-108, 433-456, 529-534, and 553-563.

Embodiment 12. The polynucleic acid molecule of embodiment 11, wherein the antisense strand comprises a nucleic acid sequence that is at least 90%, at least 95% identical to a nucleic acid sequence selected from SEQ ID NOs: 529-534 and 553-563.

Embodiment 13. The polynucleic acid molecule of any one of embodiments 8-12, wherein the sense strand comprises a nucleic acid sequence comprising at least 14, 15, 16, 17, 18, 19, or 20 consecutive sequences of a nucleic acid sequence selected from SEQ ID NOs: 217-324, 481-504, 541-546, and 575-585 with no more than 1, 2, 3, or 4 mismatches.

Embodiment 14. The polynucleic acid molecule of any one of embodiments 8-13, wherein the antisense strand comprises a nucleic acid sequence comprising at least 14, 15, 16, 17, 18, 19, 20, 21, or 22 consecutive sequences of a nucleic acid sequence selected from SEQ ID NOs: 1-108, 433-456, 529-534, and 553-563 with no more than 1, 2, 3, or 4 mismatches.

Embodiment 15. The polynucleic acid molecule of any one of embodiments 8-14, wherein the sense strand comprises a nucleic acid sequence of SEQ ID NOs: 217-324, 481-504, 541-546, and 575-585 and the antisense strand comprises a nucleic acid sequence selected from SEQ ID NOs: 1-108, 433-456, 529-534, and 553-563.

Embodiment 16. The polynucleic acid molecule of embodiment 15, wherein the sense strand comprises a nucleic acid sequence selected from SEQ ID NOs: 541-546 and 575-585 and the antisense strand comprises a nucleic acid sequence selected from a nucleic acid sequence of SEQ ID NOs: 529-534 and 553-563.

Embodiment 17. The polynucleic acid molecule of any one of embodiments 1-16, wherein the polynucleic acid molecule comprises (1) a 2′-fluoro modified nucleotides; (2) a 2′-O-methyl modified nucleotides; (3) 2′-deoxy modified nucleotides, or (4) a modified internucleotide linkage.

Embodiment 18. The polynucleic acid molecule of any one of embodiments 1-17, wherein the polynucleic acid molecule comprise at least two consecutive modified internucleotide linkages at the 5′ end.

Embodiment 19. The polynucleic acid molecule of any one of embodiments 8-18, wherein the antisense strand comprises at least two internucleotide linkages among 3 internucleotide linkages at the 3′end substituted with modified internucleotide linkages.

Embodiment 20. The polynucleic acid molecule of any one of embodiments 8-19, wherein the antisense strand comprises 5′-nNfnnnNfnNfNfnnnnNfnNfnnnnnnn-3′, 5′-nNfnnnNfnnnnnnnNfnNfnnnnnnn-3′, 5′-nNfnnnnNfnnnnNfnNfnnnnnnnnn-3′, 5′-nNfnnnnNfnnnnNfnNfnNfnnnnnnn-3′, or 5′-nNfnnnnnnnnnNfnNfnNfnnnnnnn-3′, wherein “Nf” stands for a 2′-fluoro modified nucleotide, and wherein “n” stands for a 2′-O-methyl modified nucleotide.

Embodiment 21. The polynucleic acid molecule of any one of embodiments 8-20, wherein the sense strand comprises 5′-nnnnnnNfnNfnNfnnnnnnnnnn-3′, 5′-nnnnnnNfnNfNfNfnnnnnnnnnn-3′, 5′-nnnnnnnnNfNfNfnnnnnnnnnn-3′, or 5′-nnnnnnNfnNfnNfnnnnnnnnnn-invdN-invdN-3′ wherein “Nf” stands for a 2′-fluoro modified nucleotide, wherein “n” stands for a 2′-O-methyl modified nucleotide, and wherein “invdN” stands for an inverted deoxy-nucleotide.

Embodiment 22. The polynucleic acid molecule of any one of embodiments 8-21, wherein the sense strand comprises 5′-NfnNfnNfnNfnNfNfNfnNfnNfnNfnNfnNf-3′, wherein the antisense strand comprises 5′-nNfnNfnNfnNfnNfnnnNfnNfnNfnNfnnn-3′, wherein “Nf” stands for a 2′-fluoro modified nucleotide, and wherein “n” stands for a 2′-O-methyl modified nucleotide.

Embodiment 23. The polynucleic acid molecule of any one of embodiments 8-21, wherein the sense strand comprises 5′-nnnnnnNfnNfNfNfnnnnnnnnnn-3′, wherein the antisense strand comprises 5′-nNfnnnNfnNfNfnnnnNfNnNfnnnnnnn-3′, wherein “NF” stands for a 2′-fluoro modified nucleotide, and wherein “n” stands for a 2′-O-methyl modified nucleotide.

Embodiment 24. The polynucleic acid molecule of any one of embodiments 8-21, wherein the sense strand comprises 5′-nnnnnnnnNfnNfnnnnnnnnnn-3′, wherein the antisense strand comprises 5′-nNfnnnnnnnnnNfnNfnnnnnnnnn-3′, wherein “Nf” stands for a 2′-fluoro modified nucleotide, and wherein “n” stands for a 2′-O-methyl modified nucleotide.

Embodiment 25. The polynucleic acid molecule of any one of embodiments 8-21, wherein the sense strand comprises 5′-nnnnnnNfnNfnNfnnnnnnnnnn-3′, wherein the antisense strand comprises 5′-nNfnnnnnnnnnNfnNfnNfnnnnnnn-3′, wherein “Nf” stands for a 2′-fluoro modified nucleotide, and wherein “n” stands for a 2′-O-methyl modified nucleotide.

Embodiment 26. The polynucleic acid molecule of any one of embodiments 8-21, wherein the sense strand comprises 5′-nnnnnnNfnNfnNfnnnnnnnnnn-3′, wherein the antisense strand comprises 5′-nNfnnnnNfnnnnNfnNfnNfnnnnnnn-3′, wherein “Nf” stands for a 2′-fluoro modified nucleotide, and wherein “n” stands for a 2′-O-methyl modified nucleotide.

Embodiment 27. The polynucleic acid molecule of any one of embodiments 17-26, wherein the modified internucleotide linkage is a phosphorothioate internucleotide linkage.

Embodiment 28. The polynucleic acid molecule of any embodiment 27, wherein the modified internucleotide linkage comprises a stereochemically enriched phosphorothioate internucleotide linkage.

Embodiment 29. The polynucleic acid molecule of any one of embodiments 17-28, wherein the modified internucleotide linkage is an SP chiral internucleotide phosphorothioate linkage.

Embodiment 30. The polynucleic acid molecule of any one of embodiments 17-29, wherein the polynucleic acid comprises a plurality of modified internucleotide linkages, and at least 1, 2, 3, or 4 of the plurality of modified internucleotide linkages are stereochemically enriched phosphorothioate internucleotide linkages.

Embodiment 31. The polynucleic acid molecule of embodiment 30, wherein the stereochemically enriched phosphorothioate internucleotide linkages comprise both R- and S-isomers.

Embodiment 32. The polynucleic acid molecule of one of embodiments 30-31, wherein the stereochemically enriched phosphorothioate is disposed between two consecutive nucleosides that are two of six 5′ or 3′-terminal nucleosides of the sense strand or the antisense strand.

Embodiment 33. The polynucleic acid molecule of any one of embodiments 1-32, wherein the polynucleic acid molecule comprises a hypoxanthine nucleobase-containing nucleoside substitution.

Embodiment 34. The polynucleic acid molecule of embodiment 33, wherein the hypoxanthine nucleobase-containing nucleoside substitution is an inosine substitution.

Embodiment 35. The polynucleic acid molecule of embodiment 34, wherein the inosine substitution is within a seed region of the antisense strand.

Embodiment 36. The polynucleic acid molecule of embodiment 34, wherein the inosine substitution is within 7 nucleotides from the 5′ end of the antisense strand.

Embodiment 37. The polynucleic acid molecule of any one of embodiments 1-36, wherein the polynucleic acid molecule comprises an abasic substitution.

Embodiment 38. The polynucleic acid molecule of embodiment 37, wherein the abasic substitution is at the 5th or 7th nucleotide from the 5′ end.

Embodiment 39. The polynucleic acid molecule of one of embodiments 17-38, wherein the cytotoxicity of the polynucleic acid molecule is decreased compared to unmodified polynucleic acid.

Embodiment 40. The polynucleic acid molecule of any one of embodiments 17-39, wherein the sense strand comprises a nucleic acid sequence that is at least 80%, at least 85%, at least 90%, at least 95% identical to a nucleic acid sequence selected from SEQ ID NOs: 325-432, 505-528, 547-552, and 586-596.

Embodiment 41. The polynucleic acid molecule of any one of embodiments 17-39, wherein the antisense strand comprises a nucleic acid sequence that is at least 80%, at least 85%, at least 90%, at least 95% identical to a nucleic acid sequence selected from SEQ ID NOs: 109-216, 457-480, 535-540, and 564-574.

Embodiment 42. The polynucleic acid molecule of any one of embodiments 17-41, wherein the sense strand comprises a nucleic acid sequence selected from SEQ ID NOs: 325-432, 505-528, 547-552, and 586-596, and the antisense strand comprises a nucleic acid sequence selected from SEQ ID NOs: 109-216, 457-480, 535-540, and 564-574.

Embodiment 43. The polynucleic acid molecule of any one of embodiments 1-42, wherein the polynucleic acid molecule is 19-25 or 21-23 nucleotides in length.

Embodiment 44. A polynucleic acid molecule for modulating expression of apolipoprotein C3 (APOC3) gene, wherein polynucleic acid molecule comprises:
(a) an antisense strand comprising the nucleotide sequence of UUUCAGGGAACUGAAGCCAUCGG (SEQ ID NO:529) and a sense strand comprising the nucleotide sequence of GAUGGCUUCAGUUCCCUGAAA (SEQ ID NO:541);
(b) an antisense strand comprising the nucleotide sequence of UGAAUACUGUCCCUUUUAAGCAA (SEQ ID NO:530) and a sense strand comprising the nucleotide sequence of GCUUAAAAGGGACAGUAUUCA (SEQ ID NO:542);
(c) an antisense strand comprising the nucleotide sequence of UAGAAUACUGUCCCUUUUAAGCA (SEQ ID NO:531) and a sense strand comp rising the nucleotide sequence of CUUAAAAGGGACAGUAUUCUA (SEQ ID NO:543);
(d) an antisense strand comprising the nucleotide sequence of UUGAGAAUACUGUCCCUUUUAAG (SEQ ID NO:532) and a sense strand comprising the nucleotide sequence of UAAAAGGGACAGUAUUCUCAA (SEQ ID NO:544);
(e) an antisense strand comprising the nucleotide sequence of UCUGAGAAUACUGUCCCUUUUAA (SEQ ID NO:533) and a sense strand comprising the nucleotide sequence of AAAAGGGACAGUAUUCUCAGA (SEQ ID NO:545);
(f) an antisense strand comprising the nucleotide sequence of UCACUGAGAAUACUGUCCCUUUU (SEQ ID NO:534) and a sense strand comprising the nucleotide sequence of AAGGGACAGUAUUCUCAGUGA (SEQ ID NO:546);
(g) an antisense strand comprising the nucleotide sequence of UCUGAGAAUACUGUCCCCUUUUAA (SEQ ID NO: 554) and a sense strand comprising the nucleotide sequence of AAAAGGGACAGUAUUCUCAGA (SEQ ID NO: 576);
(h) an antisense strand comprising the nucleotide sequence of UCUGAGAAUACUGUCCCUUUCAA (SEQ ID NO: 555) and a sense strand comprising the nucleotide sequence of GAAAGGGACAGUAUUCUCAGA (SEQ ID NO: 577);
(i) an antisense strand comprising the nucleotide sequence of UCUGAGAAUACUGUCCCUUUGAA (SEQ ID NO: 556) and a sense strand comprising the nucleotide sequence of CAAAGGGACAGUAUUCUCAGA (SEQ ID NO: 578);
(j) an antisense strand comprising the nucleotide sequence of UCUGAGAAUACUGUCCCUUGCAA (SEQ ID NO: 557) and a sense strand comprising the nucleotide sequence of GCAAGGGACAGUAUUCUCAGA (SEQ ID NO: 579);
(k) an antisense strand comprising the nucleotide sequence of UCUGAGAAUACUGUCCCUUCGAA (SEQ ID NO: 558) and a sense strand comprising the nucleotide sequence of CGAAGGGACAGUAUUCUCAGA (SEQ ID NO: 580);
(l) an antisense strand comprising the nucleotide sequence of UCUGAGAAUACUGUCCCUUUUAA (SEQ ID NO: 559) and a sense strand comprising the nucleotide sequence of AAAAGGGACAGUAUUCUCAGA (SEQ ID NO: 581);
(m) an antisense strand comprising the nucleotide sequence of UCUGAGAAUACUGUCCCUUUGAA (SEQ ID NO: 560) and a sense strand comprising the nucleotide sequence of CAAAGGGACAGUAUUCUCAGA (SEQ ID NO: 582);
(n) an antisense strand comprising the nucleotide sequence of UCUGAGAAUACUGUCCCUUUAA (SEQ ID NO: 561) and a sense strand comprising the nucleotide sequence of AAAAGGGACAGUAUUCUCAGA (SEQ ID NO: 583);
(o) an antisense strand comprising the nucleotide sequence of UCUGAGAAUACUGUCCCUUUUAA (SEQ ID NO: 562) and a sense strand comprising the nucleotide sequence of AAAAGGGACAGUAUUCUCAGA (SEQ ID NO: 584); or
(p) an antisense strand comprising the nucleotide sequence of UCUGAGAAUACUGUCCCUUUUAA (SEQ ID NO: 563) and a sense strand comprising the nucleotide sequence of AAAAGGGACAGUAUUCUCAGA (SEQ ID NO: 585).

Embodiment 45. A polynucleic acid molecule for modulating expression of apolipoprotein C3 (APOC3) gene, wherein polynucleic acid molecule comprises:
(a) an antisense strand comprising the nucleotide sequence of usUfsucagGfgaacUfgAfaGfccaucsgsg (SEQ ID NO:535) and a sense strand comprising the nucleotide sequence of gsasuggcUfuCfaGfuucccugaaa (SEQ ID NO:547),
(b) an antisense strand comprising the nucleotide sequence of usGfsaauaCfugucCfcUfuUfuaagcsasa (SEQ ID NO:536) and a sense strand comprising the nucleotide sequence of gscsuuaaAfaGfgGfacaguauuca (SEQ ID NO:548);
(c) an antisense strand comprising the nucleotide sequence of usAfsgaauAfcuguCfcCfuUfuuaagscsa (SEQ ID NO:537) and a sense strand comprising the nucleotide sequence of csusuaaaAfgGfgAfcaguauucua (SEQ ID NO:549);
(d) an antisense strand comprising the nucleotide sequence of usUfsgagaAfuacuGfuCfcCfuuuuasasg (SEQ ID NO:538) and a sense strand comprising the nucleotide sequence of usasaaagGfgAfcAfguauucucaa (SEQ ID NO:550);
(e) an antisense strand comprising the nucleotide sequence of usCfsugagAfauacUfgUfcCfcuuuusasa (SEQ ID NO:539) and a sense strand comprising the nucleotide sequence of asasaaggGfaCfaGfuauucucaga (SEQ ID NO:551);
(f) an antisense strand comprising the nucleotide sequence of usCfsacugAfgaauAfcUfgUfcccuususu (SEQ ID NO:540) and a sense strand comprising the nucleotide sequence of asasgggaCfaGfuAfuucucaguga (SEQ ID NO:552);
(g) an antisense strand comprising the nucleotide sequence of vpusCfsugagAfauacUfgUfcCfcuuuusasa (SEQ ID NO: 565) and a sense strand comprising the nucleotide sequence of asasaaggGfaCfaGfuauucucaga (SEQ ID NO: 587);
(h) an antisense strand comprising the nucleotide sequence of usCfsugagAfauacUfgUfcCfcuuucsasa (SEQ ID NO: 566) and a sense strand comprising the nucleotide sequence of gsasaaggGfaCfaGfuauucucaga (SEQ ID NO: 588);
(i) an antisense strand comprising the nucleotide sequence of usCfsugagAfauacUfgUfcCfcuuugsasa (SEQ ID NO: 567) and a sense strand comprising the nucleotide sequence of csasaaggGfaCfaGfuauucucaga (SEQ ID NO: 589);
(j) an antisense strand comprising the nucleotide sequence of usCfsugagAfauacUfgUfcCfcuugcsasa (SEQ ID NO: 568) and a sense strand comprising the nucleotide sequence of gscsaaggGfaCfaGfuauucucaga (SEQ ID NO: 590);
(k) an antisense strand comprising the nucleotide sequence of usCfsugagAfauacUfgUfcCfcuucgsasa (SEQ ID NO: 569) and a sense strand comprising the nucleotide sequence of csgsaaggGfaCfaGfuauucucaga (SEQ ID NO: 591);
(l) an antisense strand comprising the nucleotide sequence of usCfsugagAfauacUfgUfcCfcuuuusasa (SEQ ID NO: 570) and a sense strand comprising the nucleotide sequence of (invAb)asasaaggGfaCfaGfuauucucaga (SEQ ID NO: 592);
(m) an antisense strand comprising the nucleotide sequence of usCfsugagAfauacUfgUfcCfcuuugsasa (SEQ ID NO: 571) and a sense strand comprising the nucleotide sequence of (invAb)csasaaggGfaCfaGfuauucucaga (SEQ ID NO: 593);
(n) an antisense strand comprising the nucleotide sequence of usCfsugdAgdAauacUfgUfcCfcuuuusasa (SEQ ID NO: 572) and a sense strand comprising the nucleotide sequence of asasaaggGfaCfaGfuauucucaga (SEQ ID NO: 594);
(o) an antisense strand comprising the nucleotide sequence of usCfsugagAfauacUfgUfscCfcuuuusasa (SEQ ID NO: 573) and a sense strand comprising the nucleotide sequence of asasaaggGfaCfaGfuauucucaga (SEQ ID NO: 595); or
(p) an antisense strand comprising the nucleotide sequence of usCfsugagAfauacUfgUfcCfcsuuuusasa (SEQ ID NO: 574) and a sense strand comprising the nucleotide sequence of asasaaggGfaCfaGfuauucucaga (SEQ ID NO: 596),
wherein “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “UF” refers to 2′-fluorouridine-3′-phosphate; “T” refers to 5-methyluridine-3′-phosphate; “t” refers to 2′-O-methyl-5-methyluridine-3′-phosphate; “If” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide.

Embodiment 46. A polynucleic acid molecule conjugate for modulating expression of apolipoprotein C3 (APOC3) gene, wherein the polynucleic acid molecule conjugate comprises a polynucleic acid molecule of any one of embodiments 1-45 and an asialoglycoprotein receptor targeting moiety.

Embodiment 47. The polynucleic acid molecule conjugate of embodiment 46, wherein the polynucleic acid molecule and the asialoglycoprotein receptor targeting moiety is coupled via a linker.

Embodiment 48. The polynucleic acid molecule conjugate of embodiment 47, wherein the linker comprises formula (IV) below,

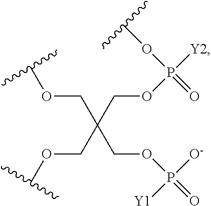

wherein at least one of Y1 and Y2 is a nucleotide in the polynucleic acid molecule.

Embodiment 49. The polynucleic acid molecule conjugate of embodiment 48, wherein the Y1 is the last nucleotide on the 3′-terminus of the sense strand of the polynucleic acid molecule.

Embodiment 50. The polynucleic acid molecule conjugate of embodiment 48, wherein the Y1 and Y2 are two consecutive nucleotides in the polynucleic acid molecule.

Embodiment 51. The polynucleic acid molecule conjugate of any one of embodiments 46-50, wherein the asialoglycoprotein receptor targeting moiety comprises N-Acetylgalactosamine (GalNAc) or galactose.

Embodiment 52. The polynucleic acid molecule conjugate of any one of embodiments 47-51, wherein the linker and the asialoglycoprotein receptor targeting moiety with the last nucleotide on the 3′-terminus of the sense strand of the polynucleic acid molecule are shown in Formula (V′):

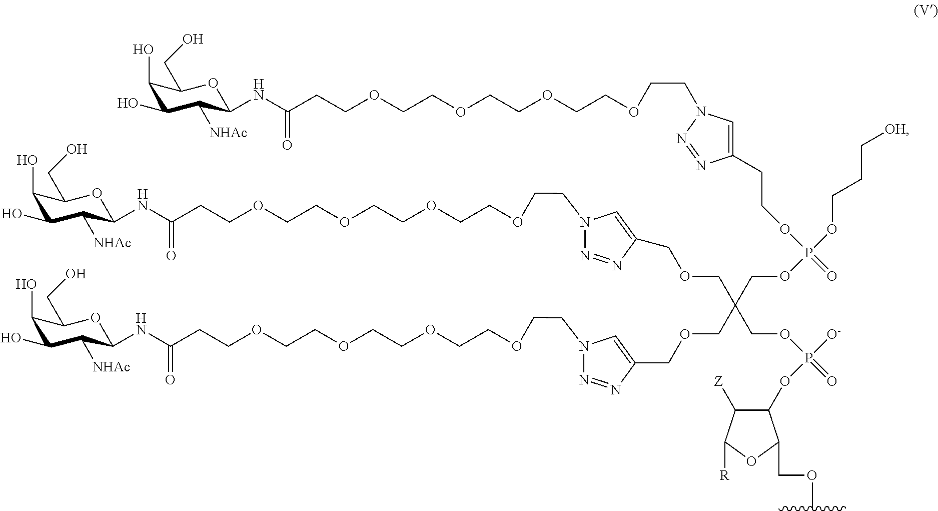

wherein Z in formula (V′) is —H, —OH, —O-Methyl, —F, or —O-methoxyethyl, and R in formula (V′) is adenine, uracil, guanine, cytosine, thymine, abasic, or others.

Embodiment 53. The polynucleic acid molecule conjugate of any one of embodiments 47-51, wherein the linker and the asialoglycoprotein receptor targeting moiety with the last nucleotide on the 3′-terminus of the sense strand of the polynucleic acid molecule are shown in Formula (V″″):

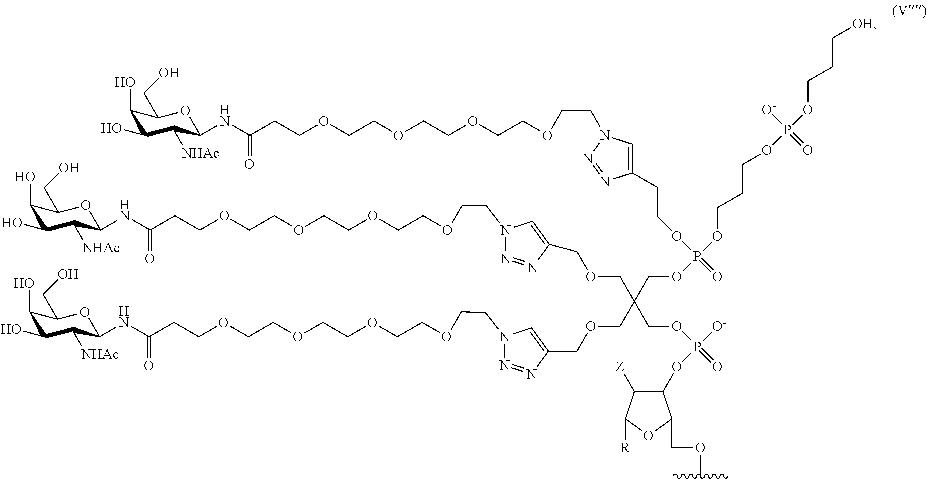

wherein Z in formula (V″″) is a moiety that corresponds to one of the sugar modifications described herein (e g., —H, —OH, —O-Methyl, —F, or —O-methoxyethyl) and R in formula (V″″) is adenine, uracil, guanine, cytosine, thymine, abasic, or others.

Embodiment 54. The polynucleic acid molecule conjugate of any one of embodiments 47-51, wherein the linker and the asialoglycoprotein receptor targeting moiety with the last nucleotide on the 3′-terminus of the sense strand of the polynucleic acid molecule are shown in Formula

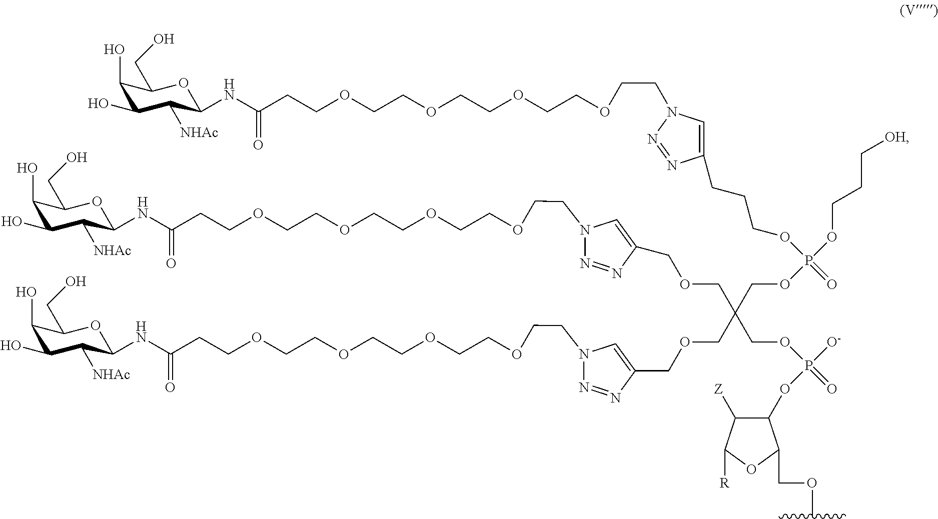

(V′″″), wherein Z in formula (V′″″) is a moiety that corresponds to one of the sugar modifications described herein (e.g., —H, —OH, —O-Methyl, —F, or —O-methoxyethyl) and R in formula (V′″″) is adenine, uracil, guanine, cytosine, thymine, abasic, or others.

Embodiment 55. The polynucleic acid molecule conjugate of any one of embodiments 47-51, wherein the linker and the asialoglycoprotein receptor targeting moiety with the last nucleotide on the 3′-terminus of the sense strand of the polynucleic acid molecule are shown in Formula (V″″″)

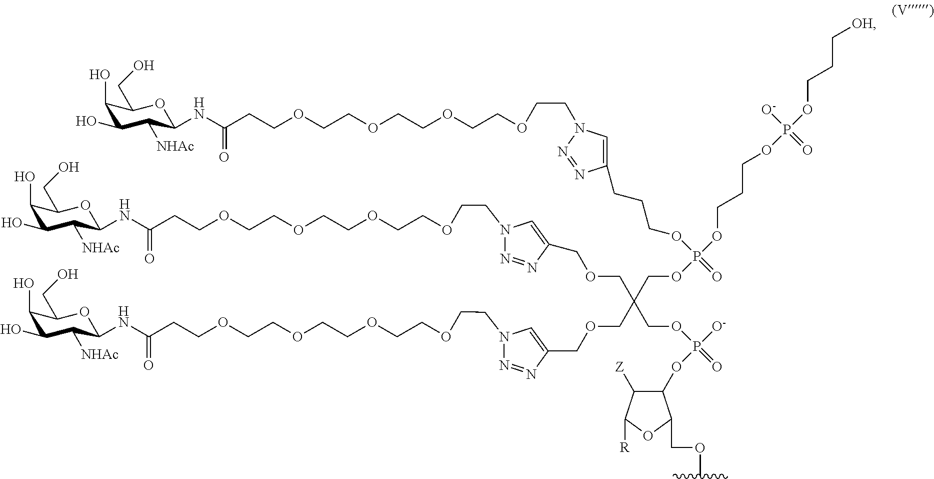

wherein Z in formula (V″″″) is a moiety that corresponds to one of the sugar modifications described herein (e.g., —H, —OH, —O-Methyl, —F, or —O-methoxyethyl) and R in formula (V″″″) is adenine, uracil, guanine, cytosine, thymine, abasic, or others.

Embodiment 56. A pharmaceutical composition comprising a polynucleic acid molecule of any one of embodiments 1-45 or a polynucleic acid molecule conjugate of any one of embodiments 46-55, and a pharmaceutically acceptable excipient.

Embodiment 57. The pharmaceutical composition of embodiment 56, wherein the pharmaceutical composition is formulated as a nanoparticle formulation.

Embodiment 58. The pharmaceutical composition of embodiment 56 or embodiment 57, wherein the pharmaceutical composition is formulated for parenteral, oral, intranasal, buccal, rectal, transdermal, intravenous, subcutaneous, or intrathecal administration.

Embodiment 59. A method of modulating expression of apolipoprotein C3 (APOC3) gene in a subject, comprising: administering to the subject a polynucleic acid molecule of any one of embodiments 1-45 or a polynucleic acid molecule conjugate of any one of embodiments 46-55, or a pharmaceutical composition of any one of embodiments 56-58, thereby modulating the expression of APOC3 gene in the subject.

Embodiment 60. A method of modulating triglyceride in a subject in need thereof, comprising: administering to the subject a polynucleic acid molecule of any one of embodiments 1-45 or a polynucleic acid molecule conjugate of any one of embodiments 46-55, or a pharmaceutical composition of any one of embodiments 56-58, thereby modulating triglyceride in the subject.

Embodiment 61. The method of embodiment 59 or 60, wherein the subject in need thereof suffers from cardiovascular disease or hypertriglyceridemia.

### EXAMPLES

These examples are provided for illustrative purposes only and not to limit the scope of the claims provided herein. For all of the sequences presented herein, oligonucleotide structure representation reads from left to right (5′ to 3′). Monomer codes present in the oligonucleotide code are linked by 5′-3′ phosphodiester bonds unless specified (succeeded by 3′ internucleotide linkage reading left to right). Abbreviations of nucleotide monomers used in oligonucleotide structure representation are as follows. “A” stands for Adenosine-3′-phosphate; “a” stands for 2′-O-methyladenosine-3′-phosphate; “Af” stands for 2′-fluoroadenosine-3′-phosphate; “dA” stand s for 2′-deoxyadenosine-3′-phosphate; “C” stands for Cytidine-3′-phosphate; “c” stands for 2′-O-methylcytidine-3-phosphate; “Cf” stands for 2′-fluorocytidine-3′-phosphate; “dC” stands for 2′-deoxycytidine-3′-phosphate; “G” stands for Guanosine-3′-phosphate; “g” stands for 2′-O-methylguanosine-3′-phosphate; “Gf” stands for 2′-fluoroguanosine-3′-phosphate; “dG” stands for 2′-deoxyguanosine-3′-phosphate; “U” stands for Uridine-3′-phosphate; “u” stands for 2′-O-methyluridine-3′-phosphate; “Uf” stands for 2′-fluorouridine-3′-phosphate; “dU” stands for 2′-deoxyuridine-3′-phosphate; “T” stands for 5-methyluridine-3′-phosphate; “t” stands for 2′-O-methyl-5-methyluridine-3′-phosphate; “Tf” stands for 2′-fluoro-5-methyluridine-3′-phosphate; “dT” stands for thymidine-3′-phosphate; and “s” stands for 3′-phosphorothioate.

### Example 1—In Vitro Efficacy of siRNAs Targeting APOC3

A panel of siRNAs were generated (shown in Table 1), and the 3′ end of each passenger/sense strand was conjugated with a triantennary GalNAc moiety (GalNAc-L96). The siRNA-GalNAc conjugates were evaluated in vitro in primary human hepatocytes.

Cryopreserved primary human hepatocytes (PHHs) were thawed and plated on collagen-coated 96-well plates at a density of 9×10⁴ cells per well. Hepatocytes were treated by incubating with the siRNAs shown in Table 1 with the 3′ end of each passenger/sense strand conjugated with a triantennary GalNAc moiety in the absence of transfection reagents (free uptake) for 48 hours. Cells were treated with the siRNAs at a concentrations of 10 μM or 0.5 μM. Untreated PHHs were used as a negative control. An siRNA targeting an unrelated gene (Ahsa1) was also used as a negative control. At the end of the incubation period, the cells were lysed, and the relative expression of the target gene was measured by branched DNA (bDNA) assay and normalized to a house-keeping gene (e.g., human GapDH) using standard protocols. The in-vitro potency of the siRNAs are listed in Table 2.

### Example 2—Drug Response Curves for Selected APOC3 siRNAs

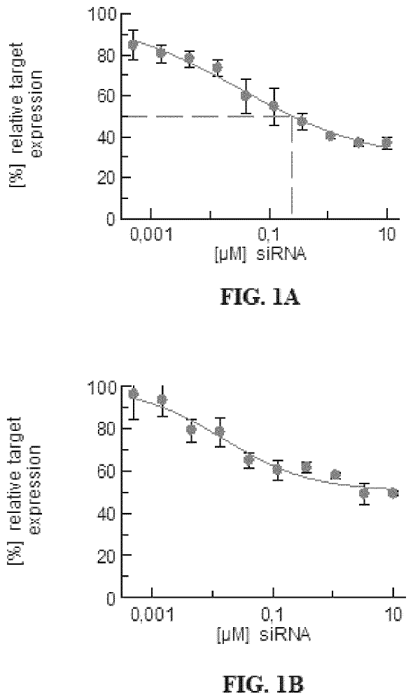

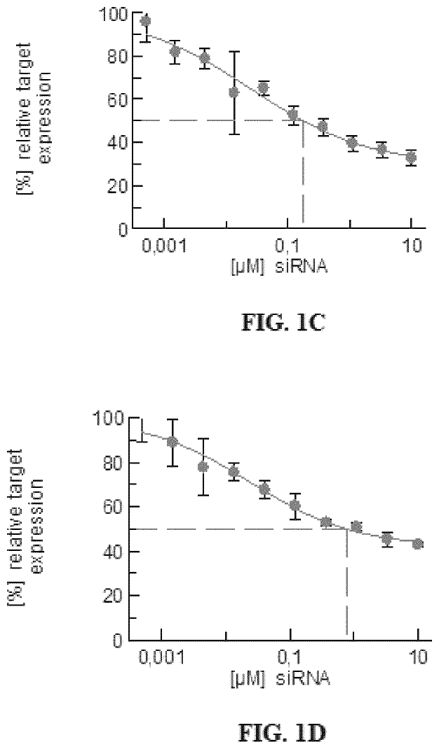

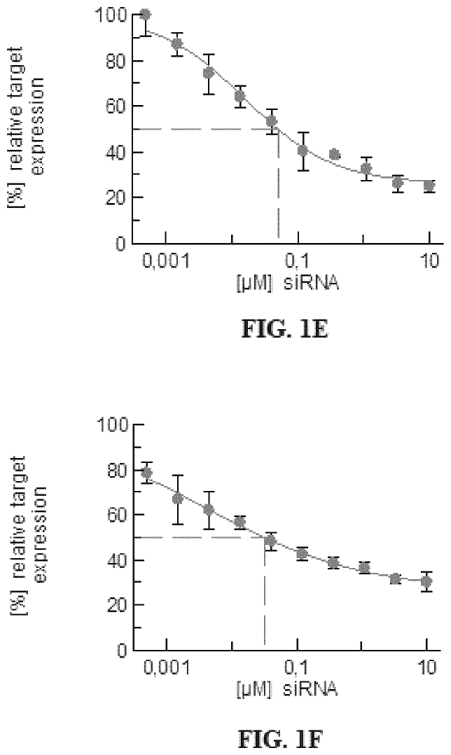

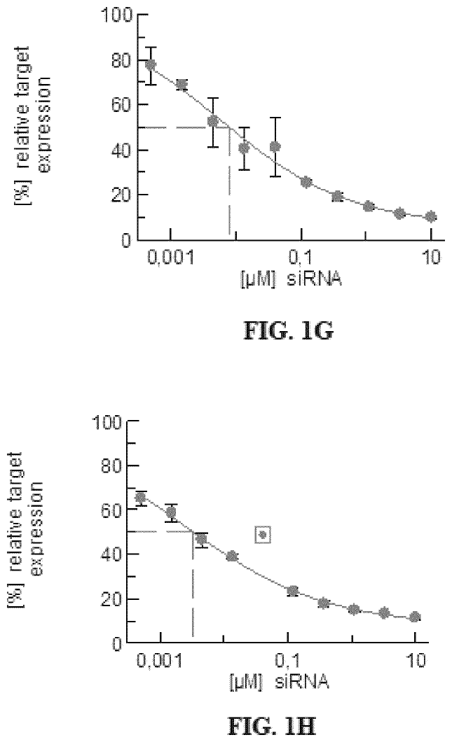

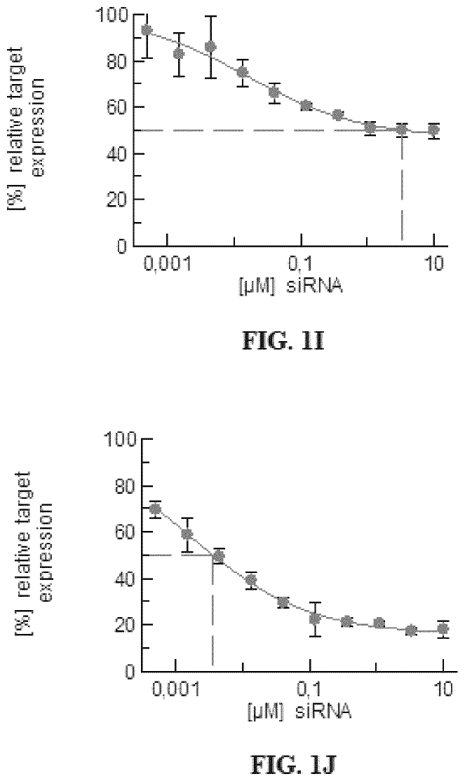

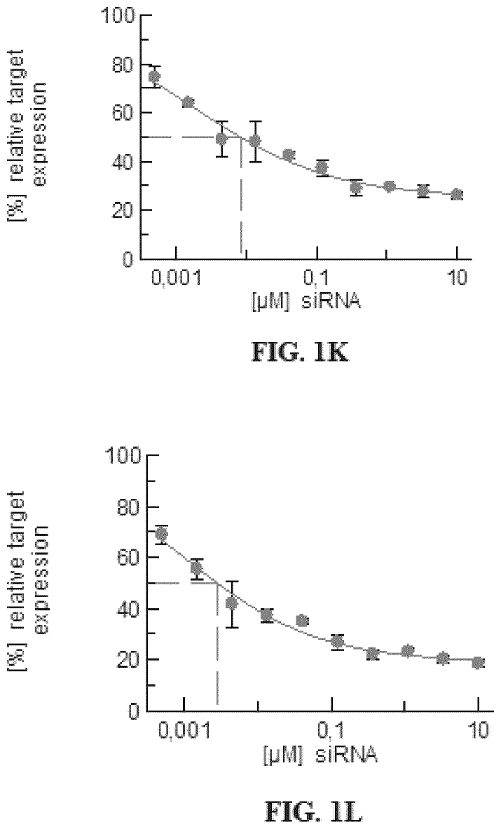

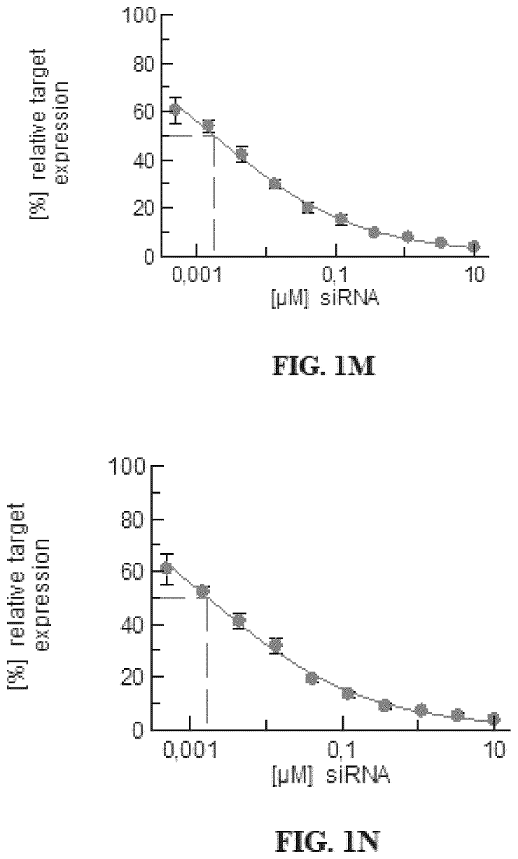

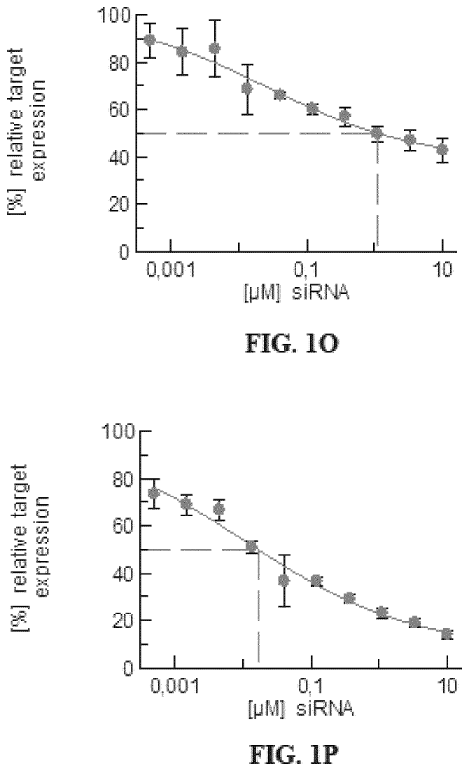

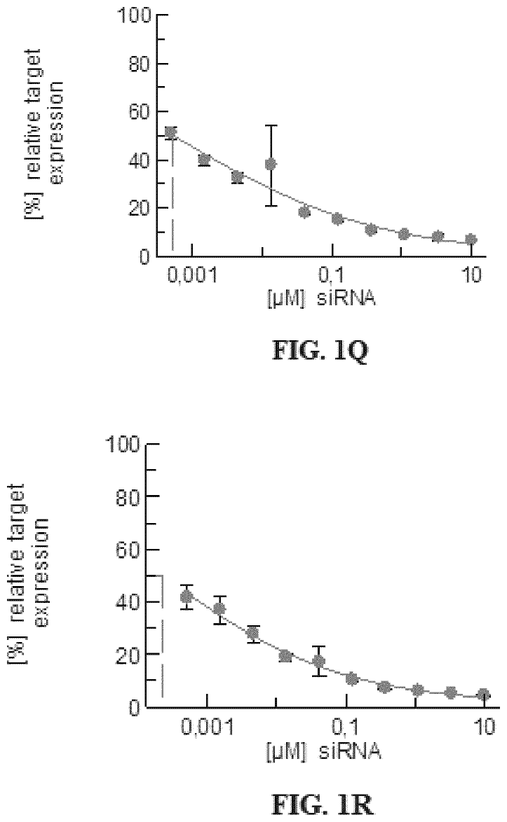

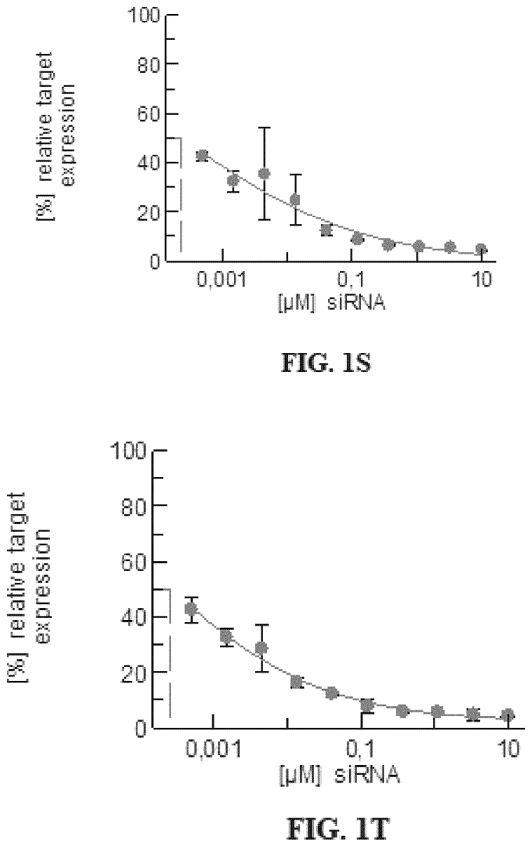

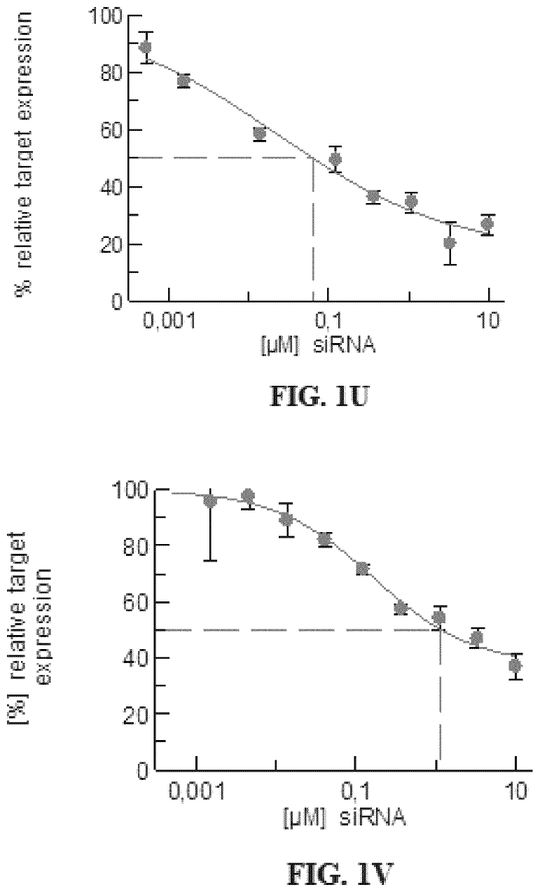

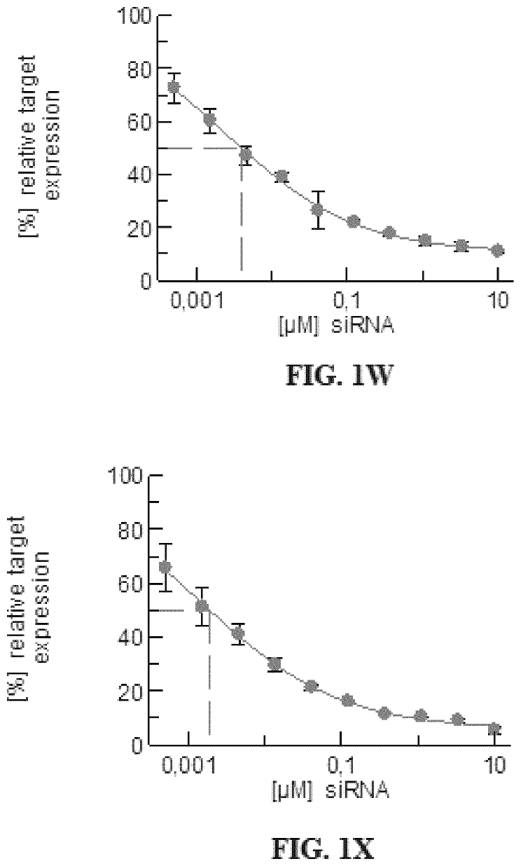

A selected group of siRNAs targeting APOC3 shown in Table 3 were used, and the 3′ end of each passenger/sense strand was conjugated with a triantennary GalNAc moiety (GalNAc-L96). The siRNA-GalNAc conjugates were evaluated at 10 different doses in primary human hepatocytes, using a similar experimental setup in Example 1. The corresponding drug response curves are depicted in FIG. 1A to FIG. 1X, and the IC₅₀ and IC₈₀ are specified in Table 4.

### Example 3—Testing APOC3 siRNAs in Transgenic Mice

SRS-000231, SRS-000228, SRS-000229, or vehicle (1×PBS) were administered to APOC3 transgenic mice (The Jackson Laboratory. B6;CBA-Tg(APOC3)3707/Bres/J, strain #006907) by subcutaneous injection (10 mL/kg). SRS-000231 and SRS-000228 treated groups were administered single doses at 10 mg/kg, 3 mg/kg, 1 mg/kg, or 0.3 mg/kg, and SRS-000229 treated groups were administered single doses at 10 mg/kg, 3 mg/kg, or 1 mg/kg. The 3′ end of the passenger strand of each si RNA was conjugated with a triantennary GalNAc moiety (X2-GalNAc as in Formula (V′)). Plasma from all groups was collected on Days −4, 1, 7, 14, 21, 28, and 35. On day 35, liver tissue was collected for mRNA expression analysis.

Plasma total cholesterol levels, triglyceride levels, HDL-c levels, and LDL-c levels were measured. For each measurement and timepoint, the percentage relative to Day −4 values for each individual animal were determined, and the results were averaged. Group mean results along with standard deviations are listed in Table 6, Table 7, Table 8, and Table 9.

Plasma hAPOC3 protein levels on Days 1, 7, 14, 21, 28, and 35 were measured using an ELISA assay (Abcam, catalog #: ab154131). Due to insufficient residual sample for some animals and timepoints, results for each individual animal and timepoint were calculated as the percentage of circulating hAPOC3 protein relative to it's group mean Day 1 value. The mean results along with standard deviation are listed in Table 10.

Total RNA from the day 35 liver samples was extracted, and levels of APOC3 mRNA were measured by RT-qPCR. Values of the SRS-000231, SRS-000228, and SRS-000229 treated animals were normalized to the PBS treated group APOC3 mRNA measurements. The mean results of percent remaining APOC3 mRNA for each group along with standard deviation are listed in Table 11.

SRS-000231, SRS-000228, and SRS-000229 each reduced plasma APOC3 (up to 88.5% in SRS-000228 treated group, for example) dose dependently across all timepoints. In addition, reductions in liver APOC3 mRNA (up to 96.7% in SRS-000231 treated group, for example) were observed at Day 35, relative to the control group. SRS-000231, SRS-000228, and SRS-000229 each reduced total cholesterol, triglycerides, and LDL-cholesterol throughout the study.

### Example 4—Testing APOC3 siRNAs in Non-Human Primates

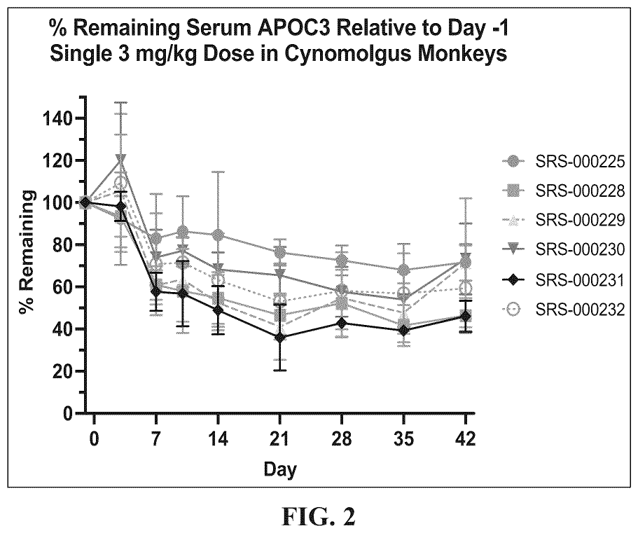

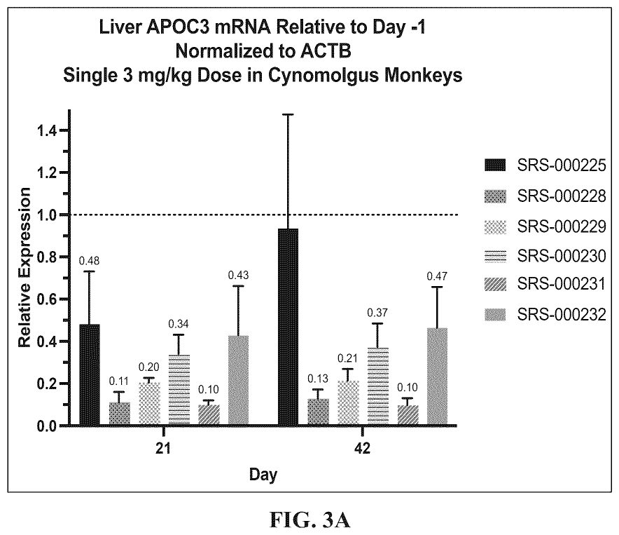

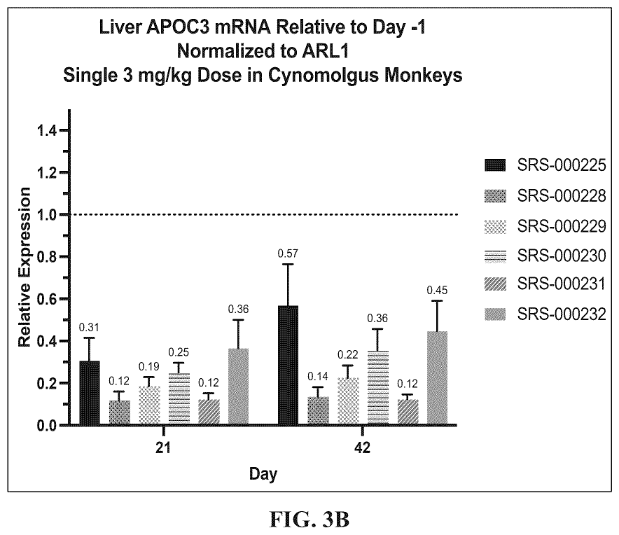

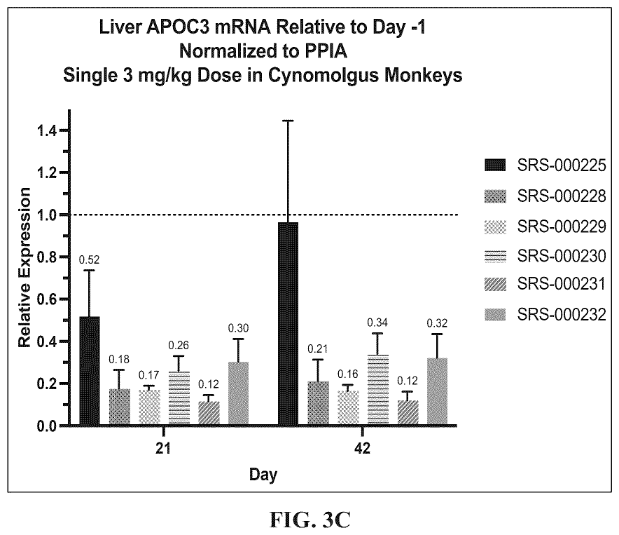

Sequences of siRNAs used for the non-human primate study are specified in Table 5 with the 3′ end of each passenger/sense strand conjugated with a GalNAc via X2 (see Formula (V′)). Male cynomolgus monkeys (n=4 per treatment group/siRNA) were administered a single 3 mg/kg subcutaneous injection of APOC3 siRNA constructs SRS-000225 and SRS-000228 through SRS-000232 (as shown in Table 5). Blood samples were collected pre-dose (Day −1), and on days 3, 7, 10, 14, 21, 28, 35, and 42. APOC3 circulating protein levels in all serum samples were analyzed using an APOC3 immunoturbidimetric assay (Beijing Leadman Biochemistry, WGAB7031). Results were expressed as a percentage of circulating APOC3 protein remaining relative to the pre-dose timepoint, and are depicted in FIG. 2. To assess the depth of APOC3 mRNA silencing in liver-tissue, ultrasound guided liver biopsies were collected pre-dose (day −1) and on days 21 and 42. At each timepoint, RT-qPCR was performed on all samples to measure APOC3 mRNA, normalizing to reference genes ACTB, PPIA, and ARL1. APOC3 mRNA levels relative to the pre-dose timepoint are depicted in FIGS. 3A-3C.

Each siRNA reduced liver APOC3 mRNA relative to the pre-dose timepoint. For example, SRS-000231 reduced liver APOC3 mRNA by 90% on day 21 and day 42 when normalized to ACTB reference gene, and SRS-000228 reduced liver APOC3 mRNA by 89% on day 21 and 87% on day 42 when normalized to ACTB reference gene. Similar reductions were observed when normalized to ARL1 and PPIA.

Further, each siRNA reduced serum APOC3 relative to the pre-dose timepoint. For example, SRS-000231 reduced serum APOC3 by 64% on day 21 and 54% on day 42.

### Example 5—Testing APOC3 siRNAs in Transgenic Mice

Sequences of siRNAs listed in Table 12, or vehicle (1×PBS) were administered to APOC3 transgenic mice (The Jackson Laboratory, B6;CBA-Tg(APOC3)3707/Bres/J, strain #006907) at a single 0.5 mg/kg dose by subcutaneous injection (10 mL/kg). The 3′ end of the passenger strand of each siRNA was conjugated with a triantennary GalNAc moiety (X2-GalNAc as in Formula (V′)). Plasma from all groups was collected on Days −4, 1, 7, 14, 21, 28, 35, 42, 49, and 56. On day 56, liver tissue was collected for mRNA expression analysis.

Plasma total cholesterol levels, triglyceride levels, HDL-c levels, and LDL-c levels were measured. For each measurement and timepoint, the percentage relative to Day 1 values for each individual animal were determined, and the results were averaged. Group mean results along with standard deviations are listed in Table 13, Table 14, Table 15, and Table 16.

Plasma hAPOC3 protein levels on Days 1, 7, 14, 21, 28, 35, 49, and 56 were measured using an ELISA assay (Abcam, catalog #: ab 154131). Due to insufficient residual sample for some animals and timepoints, results for each individual animal and timepoint were calculated as the percentage of circulating hAPOC3 protein relative to it's group mean Day 1 value. The mean results along with standard deviation are listed in Table 17.

Total RNA from the day 56 liver samples was extracted, and levels of APOC3 mRNA were measured by RT-qPCR. Values of the siRNA treated animals were normalized to the PBS treated group APOC3 mRNA measurements. The mean results of APOC3 mRNA expression relative to the vehicle control group along with standard deviation are listed in Table 18.

Following a single 0.5 mg/kg dose, all siRNAs reduced plasma APOC3 levels significantly. For example, the range of plasma APOC3 reduction following treatment of SRS-000231 was 69.6% to 89% from day 7 through day 56. Total cholesterol, triglycerides, and LDL-cholesterol were also reduced following treatment of each siRNA. For example, by Day 7, SRS-000231 reduced total cholesterol, triglycerides, and LDL-cholesterol by 70.1%, 90%, and 95.2%, respectively, and levels remained low through day 56. In addition, liver APOC3 mRNA remained significantly reduced at day 56 (end of study). For example, SRS-000231 reduced liver APOC3 mRNA by 74%. Exemplary siRNAs SRS-001860, SRS-001861, SRS-001862, and SRS-001863 reduced liver APOC3 mRNA by 93%, 95%, 97%, and 94%, respectively.

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

TABLE 1
Sequence Information for siRNAs Evaluated in vitro

| | Tar- | | Guide/ | | Guide/ | | Passenger/ | | Passenger/ |
|---|---|---|---|---|---|---|---|---|---|
| | get | SEQ | Antisense | SEQ | Antisense | SEQ | Sense | SEQ | Sense Base |
| Duplex | Posi- | ID | Structure Code | ID | Base Sequence | ID | Structure | ID | Sequence |
| ID | tion | NO. | (5′-3′) | NO. | (5′-3′) | NO. | Code (5′-3′) | NO. | (5′-3′) |
| SRS- | 31 | 109 | usCfsauggCfaccu | 1 | UCAUGGCACCUCUGU | 325 | cscsaggaAfcAfgA | 217 | CCAGGAACAGAGGUG |
| 000662 | | | CfuGfuUfccuggsa | | UCCUGGAG | | fggugccauga | | CCAUGA |
| | | | sg | | | | | | |
| SRS- | 43 | 110 | usUfsacccGfgggc | 2 | UUACCCGGGGCUGCA | 326 | gsusgccaUfgCfaG | 218 | GUGCCAUGCAGCCCC |
| 000663 | | | UfgCfaUfggcacsc | | UGGCACCU | | fccccggguaa | | GGGUAA |
| | | | su | | | | | | |
| SRS- | 44 | 111 | usGfsuaccCfgggg | 3 | UGUACCCGGGGCUGC | 327 | usgsccauGfcAfgC | 219 | UGCCAUGCAGCCCCG |
| 000664 | | | CfaGfcAfuggcasc | | AUGGCACC | | fcccggguaca | | GGUACA |
| | | | sc | | | | | | |
| SRS- | 45 | 112 | usAfsguacCfcggg | 4 | UAGUACCCGGGGCUG | 328 | gscscaugCfaGfcC | 220 | GCCAUGCAGCCCCGG |
| 000665 | | | GfcUfgCfauggcsa | | CAUGGCAC | | fccggguacua | | GUACUA |
| | | | sc | | | | | | |
| SRS- | 49 | 113 | usAfsaggaGfuacc | 5 | UAAGGAGUACCCGGG | 329 | usgscagcCfcCfgG | 221 | UGCAGCCCCGGGUAC |
| 000666 | | | CfgGfgGfaugcasu | | GCUGCAUG | | fguacuccuua | | UCCUUA |
| | | | sg | | | | | | |
| SRS- | 51 | 114 | usAfscaagGfagua | 6 | UACAAGGAGUACCCG | 330 | csasgcccCfgGfgU | 222 | CAGCCCCGGGUACUC |
| 000667 | | | CfcCfgGfggcugsc | | GGGCUGCA | | facuccuugua | | CUUGUA |
| | | | sa | | | | | | |
| SRS- | 52 | 115 | usAfsacaaGfgagu | 7 | UAACAAGGAGUACCC | 331 | asgsccccGfgGfuA | 223 | AGCCCCGGGUACUCC |
| 000668 | | | AfcCfcGfgggcusg | | GGGGCUGC | | fcuccuuguua | | UUGUUA |
| | | | sc | | | | | | |
| SRS- | 54 | 116 | usAfscaacAfagga | 8 | UACAACAAGGAGUAC | 332 | cscsccggGfuAfcU | 224 | CCCCGGGUACUCCUU |
| 000669 | | | GfuAfcCfcggggsc | | CCGGGGCU | | fccuuguugua | | GUUGUA |
| | | | su | | | | | | |
| SRS- | 55 | 117 | usAfsacaaCfaagg | 9 | UAACAACAAGGAGUA | 333 | cscscgggUfaCfuC | 225 | CCCGGGUACUCCUUG |
| 000670 | | | AfgUfaCfccgggsg | | CCCGGGGC | | fcuuguuguua | | UUGUUA |
| | | | sc | | | | | | |
| SRS- | 57 | 118 | usGfscaacAfacaa | 10 | UGCAACAACAAGGAG | 334 | csgsgguaCfuCfcU | 226 | CGGGUACUCCUUGUU |
| 000671 | | | GfgAfgUfaccggsg | | UACCCGGG | | fuguuguugca | | GUUGCA |
| | | | sg | | | | | | |
| SRS- | 58 | 119 | usGfsgcaaCfaaca | 11 | UGGCAACAACAAGGA | 335 | gsgsguacUfcCfuU | 227 | GGGUACUCCUUGUUG |
| 000672 | | | AfgGfaGfuacccsg | | GUACCCGG | | fguuguugcca | | UUGCCA |
| | | | sg | | | | | | |
| SRS- | 59 | 120 | usGfsggcaAfcaac | 12 | UGGGCAACAACAAGG | 336 | gsgsuacuCfcUfuG | 228 | GGUACUCCUUGUUGU |
| 000673 | | | AfaGfgAfguaccsc | | AGUACCCG | | fuuguugccca | | UGCCCA |
| | | | sg | | | | | | |
| SRS- | 60 | 121 | usAfsgggcAfacaa | 13 | UAGGGCAACAACAAG | 337 | gsusacucCfuUfgU | 229 | GUACUCCUUGUUGUU |
| 000674 | | | CfaAfgGfaguacsc | | GAGUACCC | | fuguugcccua | | GCCCUA |
| | | | sc | | | | | | |
| SRS- | 64 | 122 | usCfsaggaGfggca | 14 | UCAGGAGGGCAACAA | 338 | uscscuugUfuGfuU | 230 | UCCUUGUUGUUGCCC |
| 000675 | | | AfcAfaCfaaggasg | | CAAGGAGU | | fgcccuccuga | | UCCUGA |
| | | | su | | | | | | |
| SRS- | 66 | 123 | usGfsccagGfaggg | 15 | UGCCAGGAGGGCAAC | 339 | csusuguuGfuUfgC | 231 | CUUGUUGUUGCCCUC |
| 000676 | | | CfaAfcAfacaagsg | | AACAAGGA | | fccuccuggca | | CUGGCA |
| | | | sa | | | | | | |
| SRS- | 88 | 124 | usAfsgcucGfggca | 16 | UAGCUCGGGCAGAGG | 340 | uscscuggCfcUfcU | 232 | UCCUGGCCUCUGCCC |
| 000677 | | | GfaGfgCfcaggasg | | CCAGGAGC | | fgcccgagcua | | GAGCUA |
| | | | sc | | | | | | |
| SRS- | 90 | 125 | usGfsaagcUfcggg | 17 | UGAAGCUCGGGCAGA | 341 | csusggccUfcUfgC | 233 | CUGGCCUCUGCCCGA |
| 000678 | | | CfaGfaGfgccagsg | | GGCCAGGA | | fccgagcuuca | | GCUUCA |
| | | | sa | | | | | | |
| SRS- | 92 | 126 | usCfsugaaGfcucg | 18 | UCUGAAGCUCGGGCA | 342 | gsgsccucUfgCfcC | 234 | GGCCUCUGCCCGAGC |
| 000679 | | | GfgCfaGfaggccsa | | GAGGCCAG | | fgagcuucaga | | UUCAGA |
| | | | sg | | | | | | |
| SRS- | 93 | 127 | usUfscugaAfgcuc | 19 | UUCUGAAGCUCGGGC | 343 | 2scscucuGfcCfcG | 235 | GCCUCUGCCCGAGCU |
| 000680 | | | GfgGfcAfgaggcsc | | AGAGGCCA | | fagcuucagaa | | UCAGAA |
| | | | sa | | | | | | |
| SRS- | 95 | 128 | usCfscucuGfaagc | 20 | UCCUCUGAAGCUCGG | 344 | csuscugcCfcGfaG | 236 | CUCUGCCCGAGCUUC |
| 000681 | | | UfcGfcGfcagagsg | | GCAGAGGC | | fcuucagaggs | | AGAGGA |
| | | | sc | | | | | | |
| SRS- | 97 | 129 | usGfsgccuCfugaa | 21 | UGGCCUCUGAAGCUC | 345 | csusgcccGfaGfcU | 237 | CUGCCCGAGCUUCAG |
| 000682 | | | GfcUfcGfggcagsa | | GGGCAGAG | | fucagaggcca | | AGGCCA |
| | | | sg | | | | | | |
| SRS- | 98 | 130 | usCfsggccUfcuga | 22 | UCGGCCUCUGAAGCU | 346 | usgscccgAfgCfuU | 238 | UGCCCGAGCUUCAGA |
| 000683 | | | AfgCfuCfgggcasg | | CGGGCAGA | | fcagaggccga | | GGCCGA |
| | | | sa | | | | | | |
| SRS- | 99 | 131 | usUfscggcCfucug | 23 | UUCGGCCUCUGAAGC | 347 | gscsccgaGfcUfuC | 239 | GCCCGAGCUUCAGAG |
| 000684 | | | AfaGfcUfcgggcsa | | UCGGGCAG | | fagaggccgaa | | GCCGAA |
| | | | sg | | | | | | |
| SRS- | 100 | 132 | usCfsucggCfcucu | 24 | UCUCGGCCUCUGAAG | 348 | cscscgagCfuUfcA | 240 | CCCGAGCUUCAGAGG |
| 000685 | | | GfaAfgCfucgggsc | | CUCGGGCA | | fgaggccgaga | | CCGAGA |
| | | | sa | | | | | | |
| SRS- | 103 | 133 | usAfsuccuCfggcc | 25 | UAUCCUCGGCCUCUG | 349 | csasgcuuCfaGfaG | 241 | GAGCUUCAGAGGCCG |
| 000686 | | | UfcUfgAfagcucsg | | AAGCUCGG | | fgccgaggaua | | AGGAUA |
| | | | sg | | | | | | |
| SRS- | 112 | 134 | usAfsagggAfggca | 26 | UAAGGGAGGCAUCCU | 350 | asgsgccgAfgGfaU | 242 | AGGCCGAGGAUGCCU |
| 000687 | | | UfcCfuCfgcccusc | | CGGCCUCU | | fgccucccuua | | CCCUUA |
| | | | su | | | | | | |
| SRS- | 113 | 135 | usGfsaaggGfaggc | 27 | UGAAGGGAGGCAUCC | 351 | gsgsccgaGfgAfuG | 243 | GGCCGAGGAUGCCUC |
| 000688 | | | AfuCfcUfcggccsu | | UCGGCCUC | | fccucccuuca | | CCUUCA |
| | | | sC | | | | | | |
| SRS- | 114 | 136 | usAfsgaagGfgagg | 28 | UAGAAGGGAGGCAUC | 352 | gscsccagGfaUfgC | 244 | GCCGAGGAUGCCUCC |
| 000689 | | | CfaUfcCfuccgcsc | | CUCGGCCU | | fcucccuucua | | CUUCUA |
| | | | su | | | | | | |
| SRS- | 115 | 137 | usGfsagaaGfggag | 29 | UGAGAAGGGAGGCAU | 353 | cscsgaggAfuGfcC | 245 | CCGAGGAUGCCUCCC |
| 000690 | | | GfcAfuCfcucggsc | | CCUCGGCC | | fucccuucuca | | UUCUCA |
| | | | sc | | | | | | |
| SRS- | 151 | 138 | usCfsuugcUfcgcg | 30 | UCUUGGUGGCGUGCU | 354 | ascsaugaAfgCfaC | 246 | ACAUGAAGCACGCCA |
| 000691 | | | UfgCfaUfcaugusa | | UCAUGUAA | | fcccaccaaga | | CCAAGA |
| | | | sa | | | | | | |
| SRS- | 154 | 139 | usGfsgucuUfggug | 31 | UGGUCUUGGUGGCGU | 355 | usgsaagcAfcGfcC | 247 | UGAAGCACGCCACCA |
| 000692 | | | GfcGfuGfcuucasu | | GCUUCAUG | | faccaagacca | | AGACCA |
| | | | sg | | | | | | |
| SRS- | 156 | 140 | usGfscgguCfuugg | 32 | UGCGGUCUUGGUGGC | 356 | asasgcacGfcCfaC | 248 | AAGCACGCCACCAAG |
| 000693 | | | UfgGfcGfugcuusc | | GUGCUUCA | | fcaagaccgca | | ACCGCA |
| | | | sa | | | | | | |
| SRS- | 157 | 141 | usGfsgcggUfcuug | 33 | UGGCGGUCUUGGUGG | 357 | asgscacgCfcAfcC | 249 | AGCACGCCACCAAGA |
| 000694 | | | GfuGfgCfgugcusu | | CGUGCUUC | | faagaccgcca | | CCGCCA |
| | | | sc | | | | | | |
| SRS- | 162 | 142 | usUfsccuuGfgggg | 34 | UUCCUUGGCGGUCUU | 358 | gscscaccAfaGfaC | 250 | GCCACCAAGACCGCC |
| 000695 | | | UfcUfuGfguggcsg | | GGUGGCGU | | fcgccaaggaa | | AAGGAA |
| | | | su | | | | | | |
| SRS- | 164 | 143 | usCfsauccUfuggc | 35 | UCAUCCUUGGCGGUC | 359 | csasccaaGfaCfcG | 251 | CACCAAGACCGCCAA |
| 000696 | | | GfgUfcUfuggugsg | | UUGGUGGC | | fccaaggauga | | GGAUGA |
| | | | sc | | | | | | |
| SRS- | 166 | 144 | usUfsgcauCfcuug | 36 | UUGCAUCCUUGGCGG | 360 | cscsaagaCfcGfcC | 252 | CCAAGACCGCCAAGG |
| 000697 | | | GfcGfgUfcuuggsu | | UCUUGGUG | | faaggaugcaa | | AUGCAA |
| | | | sg | | | | | | |
| SRS- | 167 | 145 | usGfsugcaUfccuu | 37 | UGUGCAUCCUUGGCG | 361 | csasagacCfgCfcA | 253 | CAAGACCGCCAAGGA |
| 000698 | | | GfgCfgGfucuucsg | | GUCUUGGU | | faggaugcaca | | UGCACA |
| | | | su | | | | | | |
| SRS- | 176 | 146 | usCfsgcugCfucag | 38 | UCGCUGCUCAGUGCA | 362 | csasaggaUfgCfaC | 254 | CAAGGAUGCACUGAG |
| 000699 | | | UfgCfaUfccuugsg | | UCCUUGGC | | fugagcagcga | | CAGCGA |
| | | | sc | | | | | | |
| SRS- | 177 | 147 | usAfscgcuGfcuca | 39 | UACGCUGCUCAGUGC | 363 | asasggauGfcAfcU | 255 | AAGGAUGCACUGAGC |
| 000700 | | | GfuGfcAfuccuusg | | AUCCUUGG | | fgagcagggua | | AGCGUA |
| | | | sg | | | | | | |
| SRS- | 178 | 148 | usCfsacgcUfgcuc | 40 | UCACGCUGCUCAGUG | 364 | asgsgaugCfaCfaG | 256 | AGGAUGCACUGAGCA |
| 000701 | | | AfgUfgCfauccusu | | CAUCCUUG | | fagcagcguga | | GCGUGA |
| | | | sg | | | | | | |
| SRS- | 180 | 149 | usUfsgcacGfcugc | 41 | UUGCACGCUGCUCAG | 365 | gsasugcaCfuGfaG | 257 | GAUGCACUGAGCAGC |
| 000702 | | | UfcAfgUfgcaucsc | | UGCAUCCU | | fcagcgugcaa | | GUGCAA |
| | | | su | | | | | | |
| SRS- | 189 | 150 | usUfsgggaCfuccu | 42 | UUGGGACUCCUGCAC | 366 | asgscagcGfuGfcA | 258 | AGCAGCGUGCAGGAG |
| 000703 | | | GfcAfcGfcugcusc | | GCUGCUCA | | fggagucccaa | | UCCCAA |
| | | | sa | | | | | | |
| SRS- | 222 | 151 | usUfscgguCfaccc | 43 | UUCGGUCACCCAGCC | 367 | gscscaggGfgCfuG | 259 | GCCAGGGGCUGGGUG |
| 000704 | | | AfgCfcCfcugccsc | | CCUGGCCU | | fggugaccgaa | | ACCGAA |
| | | | su | | | | | | |
| SRS- | 223 | 152 | usAfsucggUfcacc | 44 | UAUCGGUCACCCAGC | 368 | cscsagggGfcUfgG | 260 | CCAGGGGCUGGGUGA |
| 000705 | | | CfaGfcCfccugcsc | | CCCUGGCC | | fgugaccgaua | | CCGAUA |
| | | | sc | | | | | | |
| SRS- | 227 | 153 | usAfsgccaffcggu | 45 | UAGCCAUCGGUCACC | 369 | wscsgcugGfcUfgA | 261 | GGGCUGGGUGACCGA |
| 000706 | | | CfaCfcCfagcccsc | | CAGCCCCU | | fccgauggcua | | UGGCUA |
| | | | su | | | | | | |
| SRS- | 229 | 154 | usGfsaagcCfaucg | 46 | UGAAGCCAUCGGUCA | 370 | cscsuggcUfcAfcC | 262 | GCUGGGUGACCGAUG |
| 000707 | | | GfuCfaCfccagcsc | | CCCAGCCC | | fcauggcuuca | | GCUUCA |
| | | | sc | | | | | | |
| SRS- | 231 | 155 | usCfsugaaGfccau | 47 | UCUGAAGCCAUCGGU | 371 | uscsgcugAfcCfgA | 263 | UGGGUGACCGAUGGC |
| 000708 | | | CfgGfuCfacccasg | | CACCCAGC | | fuggcuucaga | | UUCAGA |
| | | | sc | | | | | | |
| SRS- | 232 | 156 | usAfscugaAfgcca | 48 | UACUGAAGCCAUCGG | 372 | gsgsgugaCfcGfaU | 264 | GGGUGACCGAUGGCU |
| 000709 | | | UfcGfgUfcacccsa | | UCACCCAG | | fggcuucagua | | UCAGUA |
| | | | sg | | | | | | |
| SRS- | 233 | 157 | usAfsacugAfagcc | 49 | UAACUGAAGCCAUCG | 373 | gsgsugacCfgAfuG | 265 | GGUGACCGAUGGCUU |
| 000710 | | | AfuCfgGfucaccsc | | GUCACCCA | | fgcuucaguua | | CAGUUA |
| | | | sa | | | | | | |
| SRS- | 234 | 158 | usGfsaacuGfaagc | 50 | UGAACUGAAGCCAUC | 374 | gsusgaccGfaUfgG | 266 | GUGACCGAUGGCUUC |
| 000711 | | | CfaUfcGfgucacsc | | GGUCACCC | | fcuucaguuca | | AGUUCA |
| | | | sc | | | | | | |
| SRS- | 235 | 159 | usGfsgaacUfgaag | 51 | UGGAACUGAAGCCAU | 375 | usgsaccgAfuGfgC | 267 | UGACCGAUGGCUUCA |
| 000712 | | | CfcAfuCfggucasc | | CGGUCACC | | fuucaguucca | | GUUCCA |
| | | | sc | | | | | | |
| SRS- | 240 | 160 | usUfsucagGfgaac | 52 | UUUCAGGGAACUGAA | 376 | gsasuggcffuCfaG | 268 | GAUGGCUUCAGUUCC |
| 000713 | | | UfgAfaGfccaucsg | | GCCAUCGG | | fuucccugaaa | | CUGAAA |
| | | | sg | | | | | | |
| SRS- | 248 | 161 | usAfsguagUfcuuu | 53 | UAGUAGUCUUUCAGG | 377 | csasguucCfcUfgA | 269 | CAGUUCCCUGAAAGA |
| 000714 | | | CfaGfgGfaacugsa | | GAACUGAA | | faagacuacua | | CUACUA |
| | | | sa | | | | | | |
| SRS- | 257 | 162 | usCfsggugCfucca | 54 | UCGGUGCUCCAGUAG | 378 | gsasaagaCfuAfcU | 270 | GAAAGACUACUGGAG |
| 000715 | | | GfuAfgUfcuuucsa | | UCUUUCAG | | fggagcaccga | | CACCGA |
| | | | sg | | | | | | |
| SRS- | 259 | 163 | usAfsacggUfgcuc | 55 | UAACGGUGCUCCAGU | 379 | asasgacuAfcUfgG | 271 | AAGACUACUGGAGCA |
| 000716 | | | CfaGfuAfgucuusu | | AGUCUUUC | | fagcaccguua | | CCGUUA |
| | | | sc | | | | | | |
| SRS- | 260 | 164 | usUfsaacgGfugcu | 56 | UUAACGGUGCUCCAG | 380 | asgsacuaCfuGfgA | 272 | AGACUACUGGAGCAC |
| 000717 | | | CfcAfgUfagucusu | | UAGUCUUU | | fgcacccuuaa | | CGUUAA |
| | | | su | | | | | | |
| SRS- | 261 | 165 | usUfsuaacGfgugc | 57 | UUUAACGGUGCUCCA | 381 | gsascuacUfgGfaG | 273 | GACUACUGGAGCACC |
| 000718 | | | UfcCfaGfuagucsu | | GUAGUCUU | | fcaccguuaaa | | GUUAAA |
| | | | su | | | | | | |
| SRS- | 262 | 166 | usCfsuuaaCfggug | 58 | UCUUAACGGUGCUCC | 382 | ascsuacuGfgAfgC | 274 | ACUACUGGAGCACCG |
| 000719 | | | CfaCfcffguagusc | | AGUAGUCU | | faccguuaaga | | UUAAGA |
| | | | su | | | | | | |
| SRS- | 263 | 167 | usCfscuuaAfcggu | 59 | UCCUUAACGGUGCUC | 383 | csusacugGfaGfcA | 275 | CUACUGGAGCACCGU |
| 000720 | | | GfcUfcCfaguagsu | | CAGUAGUC | | fccguuaagga | | UAAGGA |
| | | | sc | | | | | | |
| SRS- | 264 | 168 | usUfsccuuAfacgg | 60 | UUCCUUAACGGUGCU | 384 | usascuggAfgCfaC | 276 | UACUGGAGCACCGUU |
| 000721 | | | UfgCfuCfcaguasg | | CCAGUAGU | | fcguuaaggaa | | AAGGAA |
| | | | su | | | | | | |
| SRS- | 265 | 169 | usGfsuccuUfaacg | 61 | UGUCCUUAACGGUGC | 385 | ascsuggaGfcAfcC | 277 | ACUGGAGCACCGUUA |
| 000722 | | | GfuGfcUfccagusa | | UCCAGUAG | | fguuaaggaca | | AGGACA |
| | | | sg | | | | | | |
| SRS- | 266 | 170 | usUfsguccUfuaac | 62 | UUGUCCUUAACGGUG | 386 | csusgcagCfaCfcG | 278 | CUGGAGCACCGUUAA |
| 000723 | | | GfgUfgCfuccagsu | | CUCCAGUA | | fuuaaggacaa | | GGACAA |
| | | | sa | | | | | | |
| SRS- | 267 | 171 | usUfsugucCfuuaa | 63 | UUUGUCCUUAACGGU | 387 | usgsgagcAfcCfcU | 279 | UGGAGCACCGUUAAG |
| 000724 | | | CfgGfuGfcuccasg | | GCUCCAGU | | fuaaggacaaa | | GACAAA |
| | | | su | | | | | | |
| SRS- | 268 | 172 | usCfsuuguCfcuua | 64 | UCUUGUCCUUAACGG | 388 | gsgsagcaCfcGfuU | 280 | GGAGCACCGUUAAGG |
| 000725 | | | AfcGfgUfgcuccsa | | UGCUCCAG | | faaggacaaga | | ACAAGA |
| | | | sg | | | | | | |
| SRS- | 269 | 173 | usAfscuugUfccuu | 65 | UACUUGUCCUUAACG | 389 | gsasgcacCfgUfuA | 281 | GAGCACCGUUAAGGA |
| 000726 | | | AfaCfgGfugcucsc | | GUGCUCCA | | faggacaagua | | CAAGUA |
| | | | sa | | | | | | |
| SRS- | 271 | 174 | usGfsaacuUfgucc | 66 | UGAACUUGUCCUUAA | 390 | gscsaccgUfuAfaG | 282 | GCACCGUUAAGGACA |
| 000727 | | | UfuAfaCfggugcsu | | CGGUGCUC | | fgacaaguuca | | AGUUCA |
| | | | sc | | | | | | |
| SRS- | 272 | 175 | usAfsgaacUfuguc | 67 | UAGAACUUGUCCUUA | 391 | csasccguUfaAfgG | 283 | CACCGUUAAGGACAA |
| 000728 | | | CfuUfaAfcggugsc | | ACGGUGCU | | facaaguucua | | GUUCUA |
| | | | su | | | | | | |
| SRS- | 274 | 176 | usAfsgagaAfcuug | 68 | UAGAGAACUUGUCCU | 392 | cscsguuaAfgGfaC | 284 | CCGUUAAGGACAAGU |
| 000729 | | | UfcCfuUfaacggsu | | UAACGGUG | | faaguucucua | | UCUCUA |
| | | | sg | | | | | | |
| SRS- | 275 | 177 | usCfsagagAfacuu | 69 | UCAGAGAACUUGUCC | 393 | csgsuuaaGfgAfcA | 285 | CGUUAAGGACAAGUU |
| 000730 | | | GfuCfcUfuaacgsg | | UUAACGGU | | faguucucuga | | CUCUGA |
| | | | su | | | | | | |
| SRS- | 279 | 178 | usAfsacucAfgaga | 70 | UAACUCAGAGAACUU | 394 | asasggacAfaGfuU | 286 | AAGGACAAGUUCUCU |
| 000731 | | | AfcUfuGfuccuusa | | GUCCUUAA | | fcucugaguua | | GAGUUA |
| | | | sa | | | | | | |
| SRS- | 294 | 179 | usGfsggucCfsaau | 71 | UGGGUCCAAAUCCCA | 395 | gsasguucUfgGfgA | 287 | GAGUUCUGGGAUUUG |
| 000732 | | | CfcCfaGfaacucsa | | GAACUCAG | | fuuuggaccca | | GACCCA |
| | | | sg | | | | | | |
| SRS- | 312 | 180 | usGfsaaguUfgguc | 72 | UGAAGUUGGUCUGAC | 396 | cscsugagGfuCfaG | 288 | CCUGAGGUCAGACCA |
| 000733 | | | UfgAfcCfucaggsg | | CUCAGGGU | | faccaacuuca | | ACUUCA |
| | | | su | | | | | | |
| SRS- | 318 | 181 | usAfscggcUfgaag | 73 | UACGGCUGAAGUUGG | 397 | csuscagaCfcAfaC | 289 | GUCAGACCAACUUCA |
| 000734 | | | UfuGfgUfcugacsc | | UCUGACCU | | fuucagccgua | | GCCGUA |
| | | | su | | | | | | |
| SRS- | 320 | 182 | usCfscacgGfcuga | 74 | UCCACGGCUGAAGUU | 398 | csasgaccAfaCfuU | 290 | CAGACCAACUUCAGC |
| 000735 | | | AfgUfuGfgucugsa | | GGUCUGAC | | fcagccgugga | | CGUGGA |
| | | | sc | | | | | | |
| SRS- | 339 | 183 | usGfsuauuGfaggu | 75 | UGUAUUGAGGUCUCA | 399 | gscsugccUfgAfgA | 291 | GCUGCCUGAGACCUC |
| 000736 | | | CfuCfaGfgcagcsc | | GGCAGCCA | | fccucaauaca | | AAUACA |
| | | | sa | | | | | | |
| SRS- | 340 | 184 | usGfsguauUfgagg | 76 | UGGUAUUGAGGUCUC | 400 | csusgccuGfaGfaC | 292 | CUGCCUGAGACCUCA |
| 000737 | | | UfcUfcAfggcagsc | | AGGCAGCC | | fcucaauacca | | AUACCA |
| | | | sc | | | | | | |
| SRS- | 342 | 185 | usGfsggguAfauga | 77 | UGGGGUAUUGAGGUC | 401 | gscscugaGfaCfcU | 293 | GCCUGAGACCUCAAU |
| 000738 | | | GfgUfcUfcaggcsa | | UCAGGCAG | | fcaauacccca | | ACCCCA |
| | | | sg | | | | | | |
| SRS- | 343 | 186 | usUfsggggUfauug | 78 | UUGGGGUAUUGAGGU | 402 | cscsugagAfcCfuC | 294 | CCUGAGACCUCAAUA |
| 000739 | | | AfgGfuCfucagcsc | | CUCAGGCA | | faauaccccaa | | CCCCAA |
| | | | sa | | | | | | |
| SRS- | 346 | 187 | usAfscuugGfggua | 79 | UACUUGGGGUAUUGA | 403 | gsasgaccUfcAfaU | 295 | GAGACCUCAAUACCC |
| 000740 | | | UfaGfaGfgucucsa | | GGUCUCAG | | faccccaagua | | CAAGUA |
| | | | sg | | | | | | |
| SRS- | 347 | 188 | usGfsacuuGfcgcu | 80 | UGACUUGGGGUAUUG | 404 | asgsaccuCfaAfuA | 296 | AGACCUCAAUACCCC |
| 000741 | | | AfaUfgAfcgucusc | | AGGUCUCA | | fccccaaguca | | AAGUCA |
| | | | sa | | | | | | |
| SRS- | 348 | 189 | usGfsgacuUfgggg | 81 | UGGACUUGGGGUAUU | 405 | gsasccucAfaUfaC | 297 | GACCUCAAUACCCCA |
| 000742 | | | UfaUfuGfaggucsu | | GAGGUCUC | | fcccaagucca | | AGUCCA |
| | | | sc | | | | | | |
| SRS- | 389 | 190 | usGfsauugCfagga | 82 | UGAUUGCAGGACCCA | 406 | gscsuccuUfgGfgU | 298 | GCUCCUUGGGUCCUG |
| 000743 | | | CfcCfaAfcgagcsu | | AGGAGCUC | | fccugcaauca | | CAAUCA |
| | | | sc | | | | | | |
| SRS- | 391 | 191 | usGfsagauUfgcag | 83 | UGAGAUUGCAGGACC | 407 | uscscuugGfgUfcC | 299 | UCCUUGGGUCCUGCA |
| 000744 | | | GfaCfcCfaaggasg | | CAAGGAGC | | fugcaaucuca | | AUCUCA |
| | | | sc | | | | | | |
| SRS- | 392 | 192 | usGfsgagaUfugca | 84 | UGGAGAUUGCAGGAC | 408 | cscsuuggGfuCfcU | 300 | CCUUGGGUCCUGCAA |
| 000745 | | | GfgAfcCfcaagcsa | | CCAAGGAG | | fccaaucucca | | UCUCCA |
| | | | sg | | | | | | |
| SRS- | 393 | 193 | usUfsggagAfuugc | 85 | UUGGAGAUUGCAGGA | 409 | csusucggUfcCfuG | 301 | CUUGGGUCCUGCAAU |
| 000746 | | | AfgGfaCfccaagsg | | CCCAAGGA | | fcaaucuccaa | | CUCCAA |
| | | | sa | | | | | | |
| SRS- | 394 | 194 | usCfsugcaGfauug | 86 | UCUGGAGAUUGCAGG | 410 | ususggcuCfcUfgC | 302 | UUGGGUCCUGCAAUC |
| 000747 | | | CfaGfgAfcccaasg | | ACCCAAGG | | faaucuccaga | | UCCAGA |
| | | | sg | | | | | | |
| SRS- | 395 | 195 | usCfscuggAfgauu | 87 | UCCUGGAGAUUGCAG | 411 | uscsggucCfuGfcA | 303 | UGGGUCCUGCAAUCU |
| 000748 | | | GfcAfgGfacccasa | | GACCCAAG | | faucuccagga | | CCAGGA |
| | | | sg | | | | | | |
| SRS- | 396 | 196 | usCfsccugGfagau | 88 | UCCCUGGAGAUUGCA | 412 | gsgsguccUfgCfaA | 304 | GGGUCCUGCAAUCUC |
| 000749 | | | UfgCfaGfgacccsa | | GGACCCAA | | fucuccaggga | | CAGGGA |
| | | | sa | | | | | | |
| SRS- | 397 | 197 | usGfscccuGfgaga | 89 | UGCCCUGGAGAUUGC | 413 | csgsuccuGfcAfaU | 305 | GGUCCUGCAAUCUCC |
| 000750 | | | UfuGfcAfggaccsc | | AGGACCCA | | fcuccaggcca | | AGGGCA |
| | | | sa | | | | | | |
| SRS- | 422 | 198 | usCfsccuuUfuaag | 90 | UCCCUUUUAAGCAAC | 414 | csusguagGfuUfcC | 306 | CUGUAGGUUGCUUAA |
| 000751 | | | CfaAfcCfuacagsg | | CUACAGGG | | fuuaaaaggga | | AAGGGA |
| | | | sg | | | | | | |
| SRS- | 424 | 199 | usGfsucccUfuuua | 91 | UGUCCCUUUUAAGCA | 415 | gsusagguUfgCfuU | 307 | GUAGGUUGCUUAAAA |
| 000752 | | | AfgCfaAfccuacsa | | ACCUACAG | | faaaagggaca | | GGGACA |
| | | | sg | | | | | | |
| SRS- | 431 | 200 | usGfsaauaCfuguc | 92 | UGAAUACUGUCCCUU | 416 | gscsuuaaAfaGfgG | 308 | GCUUAAAAGGGACAG |
| 000753 | | | CfcUfuUfuaagcsa | | UUAAGCAA | | facaguauuca | | UAUUCA |
| | | | sa | | | | | | |
| SRS- | 433 | 201 | usGfsagaaUfacug | 93 | UGAGAAUACUGUCCC | 417 | ususaaaaGfgGfaC | 309 | UUAAAAGGGACAGUA |
| 000754 | | | UfcCfcUfuuuaasg | | UUUUAAGC | | faguauucuca | | UUCUCA |
| | | | sc | | | | | | |
| SRS- | 434 | 202 | usUfsgagaAfuacu | 94 | UUGAGAAUACUGUCC | 418 | usasaaagGfgAfcA | 310 | UAAAAGGGACAGUAU |
| 00075S | | | GfaCfcCfuuuuasa | | CUUUUAAG | | fguauucucaa | | UCUCAA |
| | | | sg | | | | | | |
| SRS- | 435 | 203 | usCfsugagAfauac | 95 | UCUGAGAAUACUGUC | 419 | asasaagggfaCfaG | 311 | AAAAGGGACAGUAUU |
| 000756 | | | UfgUfcCfcuuuusa | | CCUUUUAA | | fuauucucaga | | CUCAGA |
| | | | sa | | | | | | |
| SRS- | 436 | 204 | usAfscugaGfaaua | 96 | UACUGAGAAUACUGU | 420 | asasagggAfcAfgU | 312 | AAAGGGACAGUAUUC |
| 000757 | | | CfuGfuCfccuuusu | | CCCUUUUA | | fauucucagua | | UCAGUA |
| | | | sa | | | | | | |
| SRS- | 437 | 205 | usCfsacugAfgaau | 97 | UCACUGAGAAUACUG | 421 | asasgggaCfaGfuA | 313 | AAGGGACAGUAUUCU |
| 000758 | | | AfcUfgUfcccuusu | | UCCCUUUU | | fuucucaguga | | CAGUGA |
| | | | su | | | | | | |
| SRS- | 438 | 206 | usGfscacuGfagaa | 98 | UGCACUGAGAAUACU | 422 | asgsggacAfgUfaU | 314 | AGGGACAGUAUUCUC |
| 000759 | | | UfaCfuGfucccusu | | GUCCCUUU | | fucucagugca | | AGUGCA |
| | | | su | | | | | | |
| SRS- | 441 | 207 | usAfsgagcAfcuga | 99 | UAGAGCACUGAGAAU | 423 | csascaguAfuUfcU | 315 | GACAGUAUUCUCAGU |
| 000760 | | | GfaAfaAfcugucsc | | ACUGUCCC | | fcagugcucha | | GCUCUA |
| | | | sc | | | | | | |
| SRS- | 442 | 208 | usGfsagagCfacug | 100 | UGAGAGCACUGAGAA | 424 | ascsaguaUfuCfuC | 316 | ACAGUAUUCUCAGUG |
| 000761 | | | AfgAfaUfacugusc | | UACUGUCC | | fagugcucuca | | CUCUCA |
| | | | sc | | | | | | |
| SRS- | 446 | 209 | usGfsuaggAfgagc | 101 | UGUAGGAGAGCACUG | 425 | usasuucuCfaGfuG | 317 | UAUUCUCAGUGCUCU |
| 000762 | | | AfcUfgAfgaauasc | | AGAAUACU | | fcucuccuaca | | CCUACA |
| | | | su | | | | | | |
| SRS- | 457 | 210 | usCfsaugaGfgugg | 102 | UCAUGAGGUGGGGUA | 426 | csuscuccUfaCfcC | 318 | CUCUCCUACCCCACC |
| 000763 | | | GfgUfaGfgagagsc | | GGAGAGCA | | fcaccucauga | | UCAUGA |
| | | | sa | | | | | | |
| SRS- | 460 | 211 | usAfsggcaUfgagg | 103 | UAGGCAUGAGGUGGG | 427 | uscscuacCfcCfaC | 319 | UCCUACCCCACCUCA |
| 000764 | | | UfgGfgGfuaggasg | | GUAGGAGA | | fcucaugccua | | UGCCUA |
| | | | sa | | | | | | |
| SRS- | 493 | 212 | usUfsuauuGfggag | 104 | UUUAUUGGGAGGCCA | 428 | cscsaugcUfgGfcC | 320 | GCAUGCUGGCCUCCC |
| 000765 | | | GfcCfaGfcaugcsc | | GCAUGCCU | | facccaguaaa | | AAUAAA |
| | | | su | | | | | | |
| SRS- | 494 | 213 | usUfsuuauUfggga | 105 | UUUUAUUGGGAGGCC | 429 | csasugcuGfgCfcU | 321 | CAUGCUGGCCUCCCA |
| 000766 | | | GfgCfcAfgcaugsc | | AGCAUGCC | | fcccaauaaaa | | AUAAAA |
| | | | sc | | | | | | |
| SRS- | 496 | 214 | usGfscuuuAfuugg | 106 | UGCUUUAUUGGGAGG | 430 | usgscuggCfcUfcC | 322 | UGCUGGCCUCCCAAU |
| 000767 | | | GfaGfgCfcagcasu | | CCAGCAUG | | fcaguaaagca | | AAAGCA |
| | | | sg | | | | | | |
| SRS- | 498 | 215 | usCfsagcuUfuauu | 107 | UCAGCUUUAUUGGGA | 431 | csusggccUfcCfcA | 323 | CUGGCCUCCCAAUAA |
| 000768 | | | GfgGfaGfgccagsc | | GGCCAGCA | | fauaaagcuga | | AGCUGA |
| | | | sa | | | | | | |
| SRS- | 499 | 216 | usCfscagcUfuuau | 108 | UCCAGCUUUAUUGGG | 432 | usgsgccuCfcCfaA | 324 | UGGCCUCCCAAUAAA |
| 000769 | | | UfgGfgAfggccasg | | AGGCCAGC | | fuaaagcugga | | GCUGGA |
| | | | sc | | | | | | |

Note:
the target position is relative to human transcript NM_000040.3; the corresponding sequence of a certain SEQ ID NO. is located on its right column of the same row. “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate.
Note that, in some instances, the sense strand sequence is coupled to a targeting moiety (e.g., GalNAc or galactose (e.g., L96)) at the 3′end of the sense strand.

TABLE 2
In vitro Efficacy of candidate siRNAs

| | | | | 10 μM | | 0.5 μM | |
|---|---|---|---|---|---|---|---|
| | | | Tran- | screen | | screen | |
| | | | script | MV | | MV2 | |
| | | | Start | [1% | | [% | 0.5 |
| | | | Site in | residual | 10 μM | residual | μM |
| | | Passenger | NM_ | target | screen | target | screen |
| SRS ID | Guide Sequence | Sequence | 000040.3 | mRNA] | SD | mRNA] | SD2 |
| SRS- | usCfsauggCfaccu | cscsaggaAfcAfgA | 31 | 99 | 13 | 82 | 18 |
| 000662 | CfuGfuUfccuggsa | fggugccauga | | | | | |
| | sg | | | | | | |
| SRS- | usUfsacccGfgggc | gsusgccaUfgCfaG | 43 | 92 | 5 | 86 | 4 |
| 000663 | UfgCfaUfggcacsc | fccccggguaa | | | | | |
| | su | | | | | | |
| SRS- | usGfsuaccCfgggg | usgsccauGfcAfgC | 44 | 97 | 10 | 84 | 3 |
| 000664 | CfuGfcAfuggcasc | fcccggguaca | | | | | |
| | sc | | | | | | |
| SRS- | usAfsguacCfcggg | gscscaugCfaGfcC | 45 | 56 | 19 | 71 | 3 |
| 000665 | GfcUfgCfauggcsa | fccggguacua | | | | | |
| | sc | | | | | | |
| SRS- | usAfsaggaGfuacc | usgscagcCfcCfgG | 49 | 91 | 6 | 92 | 7 |
| 000666 | CfgGfgGfcugcasu | fguacuccuua | | | | | |
| | sg | | | | | | |
| SRS- | usAfscaagGfagua | csasgcccCfgGfgU | 51 | 55 | 10 | 64 | 2 |
| 000667 | CfcCfgGfggcugsc | facuccuugua | | | | | |
| | sa | | | | | | |
| SRS- | usAfsacaaGfgagu | asgsccccGfgGfuA | 52 | 76 | 3 | 83 | 6 |
| 000668 | AfcCfcGfgggcusg | fcuccuuguua | | | | | |
| | sc | | | | | | |
| SRS- | usAfscaacAfagga | cscsccggGfuAfcU | 54 | 45 | 3 | 63 | 2 |
| 000669 | GfuAfcCfcggggsc | fccuuguugua | | | | | |
| | su | | | | | | |
| SRS- | usAfsacaaCfaagg | cscscgggUfaCfuC | 55 | 37 | 3 | 58 | 2 |
| 000670 | AfgUfaCfccgggsg | fcuuguuguua | | | | | |
| | sc | | | | | | |
| SRS- | usGfscaacAfacaa | csgsgguaCfuCfcU | 57 | 15 | 1 | 28 | 1 |
| 000671 | GfgAfgUfacccgsg | fuguuguugca | | | | | |
| | sg | | | | | | |
| SRS- | usGfsgcaaCfaaca | gsgsguacUfcCfuU | 58 | 42 | 1 | 63 | 3 |
| 000672 | AfgGfaGfuacccsg | fguuguugcca | | | | | |
| | sg | | | | | | |
| SRS- | usGfsggcaAfcaac | gsgsuacuCfcUfuG | 59 | 47 | 0 | 64 | 6 |
| 000673 | AfaGfgAfguaccsc | fuuguugccca | | | | | |
| | sg | | | | | | |
| SRS- | usAfsgggcAfacaa | gsusacucCfuUfgU | 60 | 12 | 2 | 23 | 1 |
| 000674 | CfaAfgGfaguacsc | fuguugcccua | | | | | |
| | sc | | | | | | |
| SRS- | usCfsaggaGfggca | uscscuugUfuGfuU | 64 | 37 | 2 | 55 | 2 |
| 000675 | AfcAfaCfaaggasg | fgcccuccuga | | | | | |
| | su | | | | | | |
| SRS- | usGfsccagGfaggg | csusuguuGfuUfgC | 66 | 50 | 7 | 72 | 2 |
| 000676 | CfaAfcAfacaagsg | fccuccuggca | | | | | |
| | sa | | | | | | |
| SRS- | usAfsgcucGfggca | uscscuggCfcUfcU | 88 | 80 | 25 | 101 | 3 |
| 000677 | GfaGfgCfcaggasg | fgcccgagcua | | | | | |
| | sc | | | | | | |
| SRS- | usGfsaagcUfcggg | csusggccUfcUfgC | 90 | 87 | 8 | 98 | 5 |
| 000678 | CfaGfaGfgccagsg | fccgagcuuca | | | | | |
| | sa | | | | | | |
| SRS- | usCfsugaaGfcucg | gsgsccucUfgCfcC | 92 | 88 | 4 | 99 | 7 |
| 000679 | GfgCfaGfaggccsa | fgagcuucaga | | | | | |
| | sg | | | | | | |
| SRS- | usUfscugaAfgcuc | gscscucuGfcCfcG | 93 | 101 | 2 | 95 | 5 |
| 000680 | GfgGfcAfgaggcsc | fagcuucagaa | | | | | |
| | sa | | | | | | |
| SRS- | usCfscucuGfaagc | csuscugcCfcGfaG | 95 | 100 | 15 | 101 | 8 |
| 000681 | UfcGfgGfcagagsg | fcuucagagga | | | | | |
| | sc | | | | | | |
| SRS- | usGfsgccuCfugfa | csusgcccGfaGfcU | 97 | 76 | 18 | 94 | 4 |
| 000682 | GfcUfcGfggcagsa | fucagaggcca | | | | | |
| | sg | | | | | | |
| SRS- | usCfsggccUfcuga | usgscccgAfgCfuU | 98 | 86 | 11 | 90 | 3 |
| 000683 | AfgCfuCfgggcasg | fcagaggccga | | | | | |
| | sa | | | | | | |
| SRS- | usUfscggcCfucug | gscsccgaGfcUfuC | 99 | 95 | 9 | 95 | 2 |
| 000684 | AfaGfcUfcgggcsa | fagaggccgaa | | | | | |
| | sg | | | | | | |
| SRS- | usCfsucggCfcucu | cscscgagCfuUfcA | 100 | 90 | 14 | 87 | 3 |
| 000685 | GfaAfgCfucgggsc | fgaggccgaga | | | | | |
| | sa | | | | | | |
| SRS- | usAfsuccuCfggcc | gsasgcuuCfaGfaG | 103 | 93 | 8 | 91 | 5 |
| 000686 | UfcUfgAfagcucsg | fgccgaggaua | | | | | |
| | sg | | | | | | |
| SRS- | usAfsagggAfggca | asgsgccgAfgGfaU | 112 | 87 | 4 | 97 | 3 |
| 000687 | UfcCfuCfggccusc | fgccucccuua | | | | | |
| | su | | | | | | |
| SRS- | usGfsaaggGfaggc | gsgsccgaGfgAfuG | 113 | 78 | 6 | 100 | 15 |
| 000688 | AfuCfcUfcggccsu | fccucccuuca | | | | | |
| | sc | | | | | | |
| SRS- | usAfsgaagGfgagg | gscscgagGfaUfgC | 114 | 9 | 0 | 22 | 7 |
| 000689 | CfaUfcCfucggcsc | fcucccuucua | | | | | |
| | su | | | | | | |
| SRS- | usGfsagaaGfggag | cscsgaggAfuGfcC | 115 | 16 | 1 | 30 | 2 |
| 000690 | GfcAfuCfcucggsc | fucccuucuca | | | | | |
| | sc | | | | | | |
| SRS- | usCfsuuggUfgggg | ascsaugaAfgCfaC | 151 | 94 | 4 | 98 | 6 |
| 000691 | UfgCfuUfcaugusa | fgccaccaaga | | | | | |
| | sa | | | | | | |
| SRS- | usGfsgucuUfggug | usgsaagcAfcGfcC | 154 | 93 | 5 | 92 | 4 |
| 000692 | GfcGfuGfcuucasu | faccaagacca | | | | | |
| | sg | | | | | | |
| SRS- | usGfscgguCfuugg | asasgcacGfcCfaC | 156 | 93 | 7 | 92 | 3 |
| 000693 | UfgGfcGfugcuusc | fcaagaccgca | | | | | |
| | sa | | | | | | |
| SRS- | usGfsgcggUfcuug | asgscacgCfcAfcC | 157 | 85 | 15 | 96 | 12 |
| 000694 | GfuGfgCfgugcusu | faagaccgcca | | | | | |
| | sc | | | | | | |
| SRS- | usUfsccuuGfgggg | gscscaccAfaGfaC | 162 | 81 | 6 | 99 | 3 |
| 000695 | UfcUfuGfguggcsg | fcgccaaggaa | | | | | |
| | su | | | | | | |
| SRS- | usCfsauccUfuggc | csasccaaGfaCfcG | 164 | 72 | 6 | 93 | 3 |
| 000696 | GfgUfcUfuggugsg | fccaaggauga | | | | | |
| | sc | | | | | | |
| SRS- | usUfsgcauCfcuug | cscsaagaCfcGfcC | 166 | 80 | 13 | 105 | 7 |
| 000697 | GfcGfgUfcuuggsu | faaggaugcaa | | | | | |
| | sg | | | | | | |
| SRS- | usGfsugcaUfccuu | csasagacCfgCfcA | 167 | 73 | 7 | 84 | 10 |
| 000698 | GfgCfgGfucuugsg | faggaugcaca | | | | | |
| | su | | | | | | |
| SRS- | usCfsgcugCfucag | csasaggaUfgCfaC | 176 | 72 | 13 | 89 | 6 |
| 000699 | UfgCfaUfccuugsg | fugagcaggga | | | | | |
| | sc | | | | | | |
| SRS- | usAfscgcuGfcuca | asasggauGfcAfcU | 177 | 77 | 4 | 84 | 10 |
| 000700 | GfuGfcAfuccuusg | fgagcagggua | | | | | |
| | sg | | | | | | |
| SRS- | usCfsacgcUfgcuc | asgsgaugCfaCfuG | 178 | 66 | 12 | 83 | 2 |
| 000701 | AfgUfgCfauccusu | fagcaggguga | | | | | |
| | sg | | | | | | |
| SRS- | usUfsgcacGfcugc | gsasugcaCfuGfaG | 180 | 69 | 4 | 86 | 5 |
| 000702 | UfcAfgUfgcaucsc | fcagggugcaa | | | | | |
| | su | | | | | | |
| SRS- | usUfsgggaCfuccu | asgscagcGfuGfcA | 189 | 65 | 5 | 75 | 5 |
| 000703 | GfcAfcGfcugcusc | fggagucccaa | | | | | |
| | sa | | | | | | |
| SRS- | usUfscgguCfaccc | gscscaggGfgCfuG | 222 | 83 | 5 | 92 | 7 |
| 000704 | AfgCfcCfcuggcsc | fggugaccgaa | | | | | |
| | su | | | | | | |
| SRS- | usAfsucggUfcacc | cscsagggGfcUfgG | 223 | 70 | 1 | 83 | 8 |
| 000705 | CfaGfcCfccuggsc | fgugaccgaua | | | | | |
| | sc | | | | | | |
| SRS- | usAfsgccaUfcggu | gsgsgcugGfgUfgA | 227 | 74 | 2 | 85 | 4 |
| 000706 | CfaCfcCfagcccsc | fccgauggcua | | | | | |
| | su | | | | | | |
| SRS- | usGfsaagcCfaucg | gscsugggUfgAfcC | 229 | 70 | 2 | 95 | 8 |
| 000707 | GfuCfaCfccagcsc | fgauggcuuca | | | | | |
| | sc | | | | | | |
| SRS- | usCfsugaaGfccau | usgsggugAfcCfgA | 231 | 61 | 4 | 83 | 3 |
| 000708 | CfgGfuCfacccasg | fuggcuucaga | | | | | |
| | sc | | | | | | |
| SRS- | usAfscugaAfgcca | gsgsgugaCfcGfaU | 232 | 79 | 18 | 86 | 5 |
| 000709 | UfcGfgUfcacccsa | fggcuucagua | | | | | |
| | sg | | | | | | |
| SRS- | usAfsacugAfagcc | gsgsugacCfgAfuG | 233 | 77 | 18 | 90 | 4 |
| 000710 | AfuCfgGfucaccsc | fgcuucaguua | | | | | |
| | sa | | | | | | |
| SRS- | usGfsaacuGfaagc | gsusgaccGfaUfgG | 234 | 56 | 30 | 79 | 5 |
| 000711 | CfaUfcGfgucacsc | fcuucaguuca | | | | | |
| | sc | | | | | | |
| SRS- | usGfsgaacUfgaag | usgsaccgAfuGfgC | 235 | 53 | 15 | 59 | 3 |
| 000712 | CfcAfuCfggucasc | fuucaguucca | | | | | |
| | sc | | | | | | |
| SRS- | usUfsucagGfgaac | gsasuggcUfuCfaG | 240 | 27 | 19 | 34 | 2 |
| 000713 | UfgAfaGfccaucsg | fuucccugaaa | | | | | |
| | sg | | | | | | |
| SRS- | usAfsguagUfcuuu | csasguucCfcUfgA | 248 | 64 | 6 | 76 | 4 |
| 000714 | CfaGfgGfaacugsa | faagacuacua | | | | | |
| | sa | | | | | | |
| SRS- | usCfsggugCfucca | gsasaagaCfuAfcU | 257 | 83 | 5 | 92 | 3 |
| 000715 | GfuAfgUfcuuucsa | fggagcaccga | | | | | |
| | sg | | | | | | |
| SRS- | usAfsacggUfgcuc | asasgacuAfcUfgG | 259 | 81 | 5 | 83 | 3 |
| 000716 | CfaGfuAfgucuusu | fagcaccguua | | | | | |
| | sc | | | | | | |
| SRS- | usUfsaacgGfugcu | asgsacuaCfuGfgA | 260 | 54 | 7 | 68 | 4 |
| 000717 | CfcAfgUfagucusu | fgcaccguuaa | | | | | |
| | su | | | | | | |
| SRS- | usUfsuaacGfgugc | gsascuacUfgGfaG | 261 | 83 | 17 | 91 | 2 |
| 000718 | UfcCfaGfuagucsu | fcaccguuaaa | | | | | |
| | su | | | | | | |
| SRS- | usCfsuuaaCfggug | ascsuacuGfgAfgC | 262 | 87 | 14 | 98 | 9 |
| 000719 | CfuCfcAfguagusc | faccguuaaga | | | | | |
| | su | | | | | | |
| SRS- | usCfscuuaAfcggu | csusacugGfaGfcA | 263 | 71 | 5 | 95 | 9 |
| 000720 | GfcUfcCfaguagsu | fccguuaagga | | | | | |
| | sc | | | | | | |
| SRS- | usUfsccuuAfacgg | usascuggAfgCfaC | 264 | 95 | 9 | 103 | 7 |
| 000721 | UfgCfuCfcaguasg | fcguuaaggaa | | | | | |
| | su | | | | | | |
| SRS- | usGfsuccuUfaacg | ascsuggaGfcAfcC | 265 | 97 | 3 | 97 | 4 |
| 000722 | GfuGfcUfccagusa | fguuaaggaca | | | | | |
| | sg | | | | | | |
| SRS- | usUfsguccUfuaac | csusggagCfaCfcG | 266 | 32 | 3 | 48 | 3 |
| 000723 | GfgUfgCfuccagsu | fuuaaggacaa | | | | | |
| | sa | | | | | | |
| SRS- | usUfsugucCfuuaa | usgsgagcAfcCfgU | 267 | 52 | 3 | 82 | 16 |
| 000724 | CfgGfuGfcuccasg | fuaaggacaaa | | | | | |
| | su | | | | | | |
| SRS- | usCfsuuguCfcuua | gsgsagcaCfcGfuU | 268 | 104 | 17 | 96 | 5 |
| 000725 | AfcGfgUfgcuccsa | faaggacaaga | | | | | |
| | sg | | | | | | |
| SRS- | usAfscuugUfccuu | gsasgcacCfgUfuA | 269 | 68 | 8 | 81 | 7 |
| 000726 | AfaCfgGfugcucsc | faggacaagua | | | | | |
| | sa | | | | | | |
| SRS- | usGfsaacuUfgucc | gscsaccgUfuAfaG | 271 | 97 | 12 | 89 | 11 |
| 000727 | UfuAfaCfggugcsu | fgacaaguuca | | | | | |
| | sc | | | | | | |
| SRS- | usAfsgaacUfuguc | csasccguUfaAfgG | 272 | 10 | 1 | 24 | 1 |
| 000728 | CfuUfaAfcggugsc | facaaguucua | | | | | |
| | su | | | | | | |
| SRS- | usAfsgagaAfcuug | cscsguuaAfgGfaC | 274 | 42 | 3 | 59 | 1 |
| 000729 | UfcCfuUfaacggsu | faaguucucua | | | | | |
| | sg | | | | | | |
| SRS- | usCfsagagAfacuu | csgsuuaaGfgAfcA | 275 | 24 | 12 | 42 | 16 |
| 000730 | GfuCfcUfuaacgsg | faguucucuga | | | | | |
| | su | | | | | | |
| SRS- | usAfsacucAfgaga | asasggacAfaGfuU | 279 | 10 | 1 | 21 | 9 |
| 000731 | AfcUfuGfuccuusa | fcucugaguua | | | | | |
| | sa | | | | | | |
| SRS- | usGfsggucCfaaau | gsasguucUfgGfgA | 294 | 100 | 4 | 93 | 3 |
| 000732 | CfcCfaGfaacucsa | fuuuggaccca | | | | | |
| | sg | | | | | | |
| SRS- | usGfsaaguUfgguc | cscsugagGfuCfaG | 312 | 48 | 4 | 66 | 8 |
| 000733 | UfgAfcCfucaggsg | faccaacuuca | | | | | |
| | su | | | | | | |
| SRS- | usAfscggcUfgaag | gsuscagaCfcAfaC | 318 | 98 | 5 | 91 | 7 |
| 000734 | UfuGfgUfcugacsc | fuucagccgua | | | | | |
| | su | | | | | | |
| SRS- | usCfscacgGfcuga | csasgaccAfaCfuU | 320 | 99 | 16 | 94 | 9 |
| 000735 | AfgUfuGfgucugsa | fcagccgugga | | | | | |
| | sc | | | | | | |
| SRS- | usGfsuauuGfaggu | gscsugccUfgAfgA | 339 | 105 | 4 | 102 | 34 |
| 000736 | CfuCfaGfgcagcsc | fccucaauaca | | | | | |
| | sa | | | | | | |
| SRS- | usGfsguauUfgagg | csusgccuGfaGfaC | 340 | 90 | 5 | 83 | 6 |
| 000737 | UfcUfcAfggcagsc | fcucaauacca | | | | | |
| | sc | | | | | | |
| SRS- | usGfsggguAfuuga | gscscugaGfaCfcU | 342 | 95 | 0 | 87 | 7 |
| 000738 | GfgUfcUfcaggcsa | fcaauacccca | | | | | |
| | sg | | | | | | |
| SRS- | usUfsggggUfauug | cscsugagAfcCfuC | 343 | 95 | 7 | 101 | 12 |
| 000739 | AfgGfuCfucaggsc | faauaccccaa | | | | | |
| | sa | | | | | | |
| SRS- | usAfscuugGfggua | gsasgaccUfcAfaU | 346 | 94 | 7 | 94 | 6 |
| 000740 | UfuGfaGfgucucsa | faccccaagua | | | | | |
| | sg | | | | | | |
| SRS- | usGfsacuuGfgggu | asgsaccuCfaAfuA | 347 | 89 | 5 | 103 | 4 |
| 000741 | AfuUfgAfggucusc | fccccaaguca | | | | | |
| | sa | | | | | | |
| SRS- | usGfsgacuUfgggg | gsasccucAfaUfaC | 348 | 101 | 12 | 84 | 12 |
| 000742 | UfaUfuGfaggucsu | fcccaagucca | | | | | |
| | sc | | | | | | |
| SRS- | usGfsauugCfagga | gscsuccuUfgGfgU | 389 | 95 | 5 | 85 | 4 |
| 000743 | CfcCfaAfggagcsu | fccugcaauca | | | | | |
| | sc | | | | | | |
| SRS- | usGfsagauUfgcag | uscscuugGfgUfcC | 391 | 78 | 3 | 90 | 9 |
| 000744 | GfaCfcCfaaggasg | fugcaaucuca | | | | | |
| | sc | | | | | | |
| SRS- | usGfsgagaUfugca | cscsuuggGfuCfcU | 392 | 79 | 3 | 81 | 3 |
| 000745 | GfgAfcCfcaaggsa | fgcaaucucca | | | | | |
| | sg | | | | | | |
| SRS- | usUfsggagAfuugc | csusugggUfcCfuG | 393 | 92 | 11 | 95 | 6 |
| 000746 | AfgGfaCfccaagsg | fcaaucuccaa | | | | | |
| | sa | | | | | | |
| SRS- | usCfsuggaGfauug | ususggguCfcUfgC | 394 | 98 | 12 | 100 | 6 |
| 000747 | CfaGfgAfcccaasg | faaucuccaga | | | | | |
| | sg | | | | | | |
| SRS- | usCfscuggAfgauu | usgsggucCfuGfcA | 395 | 88 | 4 | 91 | 7 |
| 000748 | GfcAfgGfacccasa | faucuccagga | | | | | |
| | sg | | | | | | |
| SRS- | usCfsccugGfagau | gsgsguccUfgCfaA | 396 | 91 | 5 | 94 | 3 |
| 000749 | UfgCfaGfgacccsa | fucuccaggga | | | | | |
| | sa | | | | | | |
| SRS- | usGfscccuGfgaga | gsgsuccuGfcAfaU | 397 | 91 | 3 | 95 | 14 |
| 000750 | UfuGfcAfggaccsc | fcuccagggca | | | | | |
| | sa | | | | | | |
| SRS- | usCfsccuuUfuaag | csusguagGfuUfgC | 422 | 65 | 4 | 86 | 6 |
| 000751 | CfaAfcCfuacagsg | fuuaaaaggga | | | | | |
| | sg | | | | | | |
| SRS- | usGfsucccUfuuua | gsusagguUfgCfuU | 424 | 77 | 3 | 86 | 3 |
| 000752 | AfgCfaAfccuacsa | faaaagggaca | | | | | |
| | sg | | | | | | |
| SRS- | usGfsaauaCfuguc | gscsuuaaAfaGfgG | 431 | 9 | 1 | 19 | 4 |
| 000753 | CfcUfuUfuaagcsa | facaguauuca | | | | | |
| | sa | | | | | | |
| SRS- | usGfsagaaUfacug | ususaaaaGfgGfaC | 433 | 52 | 2 | 75 | 2 |
| 000754 | UfcCfcUfuuuaasg | faguauucuca | | | | | |
| | sc | | | | | | |
| SRS- | usUfsgagaAfuacu | usasaaagGfgAfcA | 434 | 9 | 0 | 20 | 1 |
| 000755 | GfuCfcCfuuuuasa | fguauucucaa | | | | | |
| | sg | | | | | | |
| SRS- | usCfsugagAfauac | asasaaggGfaCfaG | 435 | 9 | 1 | 16 | 2 |
| 000756 | UfgUfcCfcuuuusa | fuauucucaga | | | | | |
| | sa | | | | | | |
| SRS- | usAfscugaGfaaua | asasagggAfcAfgU | 436 | 46 | 6 | 65 | 3 |
| 000757 | CfuGfuCfccuuusu | fauucucagua | | | | | |
| | sa | | | | | | |
| SRS- | usCfsacugAfgaau | asasgggaCfaGfuA | 437 | 20 | 11 | 27 | 3 |
| 000758 | AfcUfgUfcccuusu | fuucucaguga | | | | | |
| | su | | | | | | |
| SRS- | usGfscacuGfagaa | asgsggacAfgUfaU | 438 | 54 | 10 | 77 | 2 |
| 000759 | UfaCfuGfucccusu | fucucagugca | | | | | |
| | su | | | | | | |
| SRS- | usAfsgagcAfcuga | gsascaguAfuUfcU | 441 | 24 | 9 | 37 | 1 |
| 000760 | GfaAfuAfcugucsc | fcagugcucua | | | | | |
| | sc | | | | | | |
| SRS- | usGfsagagCfacug | ascsaguaUfuCfuC | 442 | 71 | 8 | 83 | 6 |
| 000761 | AfgAfaUfacugusc | fagugcucuca | | | | | |
| | sc | | | | | | |
| SRS- | usGfsuaggAfgagc | usasuucuCfaGfuG | 446 | 35 | 7 | 32 | 6 |
| 000762 | AfcUfgAfgaauasc | fcucuccuaca | | | | | |
| | su | | | | | | |
| SRS- | usCfsaugaGfgugg | csuscuccUfaCfcC | 457 | 108 | 10 | 81 | 21 |
| 000763 | GfgUfaGfgagagsc | fcaccucauga | | | | | |
| | sa | | | | | | |
| SRS- | usAfsggcaUfgagg | uscscuacCfcCfaC | 460 | 95 | 6 | 83 | 4 |
| 000764 | UfgGfgGfuaggasg | fcucaugccua | | | | | |
| | sa | | | | | | |
| SRS- | usUfsuauuGfggag | gscsaugcUfgGfcC | 493 | 43 | 17 | 55 | 2 |
| 000765 | GfcCfaGfcaugcsc | fucccaauaaa | | | | | |
| | su | | | | | | |
| SRS- | usUfsuuauUfggga | csasugcuGfgCfcU | 494 | 93 | 9 | 82 | 5 |
| 000766 | GfgCfcAfgcaugsc | fcccaauaaaa | | | | | |
| | sc | | | | | | |
| SRS- | usGfscuuuAfuugg | usgscuggCfcUfcC | 496 | 116 | 21 | 93 | 5 |
| 000767 | GfaGfgCfcagcasu | fcaauaaagca | | | | | |
| | sg | | | | | | |
| SRS- | usCfsagcuUfuauu | csusggccUfcCfcA | 498 | 95 | 22 | 115 | 43 |
| 000768 | GfgGfaGfgccagsc | fauaaagcuga | | | | | |
| | sa | | | | | | |
| SRS- | usCfscagcUfuuau | usgsgccuCfcCfaA | 499 | 71 | 9 | 71 | 6 |
| 000769 | UfgGfgAfggccasg | fuaaagcugga | | | | | |
| | sc | | | | | | |

Note:
“A” refers to a denosine-3′-phosphate; “a” refers to 2′-O-methyladenosine-3′-phosphate; “Af” refers to 2′-fluoroadenosine-3′-phosphate; ”dA” refers to 2′-deoxyadenosine-3-phosphate; “C” refers to cy tidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxy guanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate.

TABLE 3
Sequence Information for siRNAs Evaluated for their Drug Response Curves

| | | | Guide/ | | Guide/ | | Passenger/ | | Passenger/ |
|---|---|---|---|---|---|---|---|---|---|
| | | | Antisense | | Antisense | | Sense | | Sense |
| | Target | SEQ | Structure | SEQ | Base | SEQ | Structure | SEQ | Base |
| | Posi- | ID | Code | ID | Sequence | ID | Code | ID | Sequence |
| Duplex ID | tion | NO. | (5′-3′) | NO. | (5′-3′) | NO. | (5′-3′) | NO. | (5′-3′) |
| SRS-000665 | 45 | 457 | usAfsguacC | 433 | UAGUACCCGG | 505 | gscscaugCf | 481 | GCCAUGCAGC |
| | | | fcgggGfcUf | | GGCUGCAUGG | | aGfcCfccgg | | CCCGGGUACU |
| | | | gCfauggcsa | | CAC | | guacua | | A |
| | | | sc | | | | | | |
| SRS-000667 | 51 | 458 | usAfscaagG | 434 | UACAAGGAGU | 506 | csasgcccCf | 482 | CAGCCCCGGG |
| | | | faguaCfcCf | | ACCCGGGGCU | | gGfgUfacuc | | UACUCCUUGU |
| | | | gGfggcugsc | | GCA | | cuugua | | A |
| | | | sa | | | | | | |
| SRS-000712 | 235 | 459 | usGfsgaacU | 435 | UGGAACUGAA | 507 | usgsacccAf | 483 | UGACCGAUGG |
| | | | fcaagCfcAf | | GCCAUCGGUC | | uGfcCfuuca | | CUUCAGUUCC |
| | | | uCfcgucasc | | ACC | | guucca | | A |
| | | | sc | | | | | | |
| SRS-000713 | 240 | 460 | usUfsucagG | 436 | UUUCAGGGAA | 508 | gsasuggcUf | 484 | GAUGGCUUCA |
| | | | fgaacUfgAf | | CUGAAGCCAU | | uCfaGfuucc | | GUUCCCUGAA |
| | | | aGfccaucsg | | CGG | | cugaaa | | A |
| | | | sg | | | | | | |
| SRS-000717 | 260 | 461 | usUfsaacgG | 437 | UUAACGGUGC | 509 | asgsacuaCf | 485 | AGACUACUGG |
| | | | fugcuCfcAf | | UCCAGUAGUC | | uGfgAfgcac | | AGCACCGUUA |
| | | | gUfagucusu | | UUU | | cguuaa | | A |
| | | | su | | | | | | |
| SRS-000723 | 266 | 462 | usUfsguccU | 438 | UUGUCCUUAA | 510 | csusggagCf | 486 | CUGGAGCACC |
| | | | fuaacGfgUf | | CGGUGCUCCA | | aCfcGfuvaa | | GUUAAGGACA |
| | | | gCfuccagsu | | GUA | | ggacaa | | A |
| | | | sa | | | | | | |
| SRS-000753 | 431 | 463 | usGfsaauaC | 439 | UGAAUACUGU | 511 | gscsumaaAf | 487 | GCUUAAAAGG |
| | | | fagucCfcUf | | CCCUUUUAAG | | aGfgGfacag | | GACAGUAUUC |
| | | | uUfuaagcsa | | CAA | | uauuca | | A |
| | | | sa | | | | | | |
| SRS-000754 | 433 | 464 | usGfsagaaU | 440 | UGAGAAUACU | 512 | ususaaaaGf | 488 | UUAAAAGGGA |
| | | | facugUfcCf | | GUCCCUUUUA | | gGfaCfagua | | CAGUAUUCUC |
| | | | cUfuuuaasg | | AGC | | uucuca | | A |
| | | | sc | | | | | | |
| SRS-000755 | 434 | 465 | usUfsgagaA | 441 | UUGAGAAUAC | 513 | usasaaagGf | 489 | UAAAAGGGAC |
| | | | fuscuGfuCf | | UGUCCCUUUU | | gAfcAfguau | | AGUAUUCUCA |
| | | | cCfuuuuasa | | AAG | | ucucaa | | A |
| | | | sg | | | | | | |
| SRS-000756 | 435 | 466 | usCfsugagA | 442 | UCUGAGAAUA | 514 | asasaaggGf | 490 | AAAAGGGACA |
| | | | fauacUfgUf | | CUGUCCCUUU | | aCfaGfuauu | | GUAUUCUCAG |
| | | | cCfcuuuusa | | UAA | | cucaga | | A |
| | | | sa | | | | | | |
| SRS-000757 | 436 | 467 | usAfscugaG | 443 | UACUGAGAAU | 515 | asasagggAf | 491 | AAAGGGACAG |
| | | | faauaCfaGf | | ACUGUCCCUU | | cAfgUfauuc | | UAUUCUCAGU |
| | | | uCfccuuusu | | UUA | | ucagua | | A |
| | | | sa | | | | | | |
| SRS-000758 | 437 | 468 | usCfsacugA | 444 | UCACUGAGAA | 516 | asasgggaCf | 492 | AAGGGACAGU |
| | | | fgaauAfcUf | | UACUGUCCCU | | aGfuAfuucu | | AUUCUCAGUG |
| | | | gUfcccuusu | | UUU | | caguga | | A |
| | | | su | | | | | | |
| SRS-000770 | 45 | 469 | usAfsgudAc | 445 | UAGUACCCGG | 517 | cscscaugCf | 493 | GCCAUGCAGC |
| | | | dCcggggcUf | | GGCUGCAUGG | | aGfcCfccgg | | CCCGGGUACU |
| | | | gCfauggcsa | | CAC | | guacua | | A |
| | | | sc | | | | | | |
| SRS-000771 | 51 | 470 | usAfscadAg | 446 | UACAAGGAGU | 518 | csasgcccCf | 494 | CAGCCCCGGG |
| | | | dGaguaccCf | | ACCCGGGGCU | | gGfgUfacuc | | UACUCCUUGU |
| | | | gGfggcugsc | | GCA | | cuugua | | A |
| | | | sa | | | | | | |
| SRS-000772 | 235 | 471 | usGfsgadAc | 447 | UGGAACTGAA | 519 | usgsaccgAf | 495 | UGACCGAUGG |
| | | | dTgaagccAf | | GCCAUCGGUC | | uGfgCfuuca | | CUUCAGUUCC |
| | | | uCfggucasc | | ACC | | guccca | | A |
| | | | sc | | | | | | |
| SRS-000773 | 240 | 472 | usUfsucdAg | 448 | UUUCAGGGAA | 520 | gsasuggcUf | 496 | GAUGGCUUCA |
| | | | dGgaacugAf | | CUGAAGCCAU | | uCfaGfuucc | | GUUCCCUGAA |
| | | | aGfccaucsg | | CGG | | cugaaa | | A |
| | | | sg | | | | | | |
| SRS-000774 | 260 | 473 | usUfsaadCg | 449 | UUAACGGUGC | 521 | asgsacuaCf | 497 | AGACUACUGG |
| | | | dGugcuccAf | | UCCAGUAGUC | | uGfgAfgcac | | AGCACCGUUA |
| | | | gUfagucusu | | UUU | | cguuaa | | A |
| | | | su | | | | | | |
| SRS-000775 | 266 | 474 | usUfsgudCc | 450 | UUGUCCTUAA | 522 | csusggagCf | 498 | CUGGAGCACC |
| | | | dTuaacggUf | | CGGUGCUCCA | | aCfcGfuuaa | | GUUAAGGACA |
| | | | gCfuccagsu | | GUA | | ggacaa | | A |
| | | | sa | | | | | | |
| SRS-000776 | 431 | 475 | usGfsaadTa | 451 | UGAATACUGU | 523 | gscsuuaaAf | 499 | GCUUAAAAGG |
| | | | dCugucccUf | | CCCUUUUAAG | | aGfgGfacag | | GACAGUAUUC |
| | | | uUfuaagcsa | | CAA | | uauuca | | A |
| | | | sa | | | | | | |
| SRS-000777 | 433 | 476 | usGfsagdAa | 452 | UGAGAATACU | 524 | ususaasaGf | 500 | UUAAAAGGGA |
| | | | dTacugucCf | | GUCCCUUUUA | | gGfaCfagua | | CAGUAUUCUC |
| | | | cUfuuuaasg | | AGC | | uucuca | | A |
| | | | sc | | | | | | |
| SRS-000778 | 434 | 477 | usUfsgadGa | 453 | UUGAGAAUAC | 525 | usasaaagGf | 501 | UAAAAGGGAC |
| | | | dAuacuguCf | | UGUCCCUUUU | | gAfcAfguau | | AGUAUUCUCA |
| | | | cCfuuuuasa | | AAG | | ucucaa | | A |
| | | | sg | | | | | | |
| SRS-000779 | 435 | 478 | usCfsugdAg | 454 | UCUGAGAAUA | 526 | asasaaggGf | 502 | AAAAGGGACA |
| | | | dAauscugUf | | CUGUCCCOUU | | aCfaGfuauu | | GUAUUCUCAG |
| | | | cCfcuuuusa | | UAA | | cucaga | | A |
| | | | sa | | | | | | |
| SRS-000780 | 436 | 479 | usAfscudGa | 455 | UACUGAGAAU | 527 | asasagggAf | 503 | AAAGGGACAG |
| | | | dGaauacuGf | | ACUGUCCCUU | | cAfgUfauuc | | UAUUCUCAGU |
| | | | uCfccuuusu | | UUA | | ucagua | | A |
| | | | sa | | | | | | |
| SRS-000781 | 437 | 480 | usCfsacdTg | 456 | UCACTGAGAA | 528 | asasgggaCf | 504 | AAGGGACAGU |
| | | | dAgaauacUf | | UACUGUCCCU | | aGfuAfuucu | | AUUCUCAGUG |
| | | | gUfcccuusu | | UUU | | caguga | | A |
| | | | su | | | | | | |

Note:
the target position is relative to human transcript NM_000040.3; the corresponding sequence of a certain SEQ ID NO. is located on its right column of the same row. “A” refers to adenosine-3′-phosphate; “a” refers to 2′-O-methyladenosine-3′-phosphate; “Af” refers to 2′-fluoroadenosine-3′-phosphate; “dA” refers to 2′-deoxy adenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxy guanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothicate.

TABLE 4
Summary of Drug Response Curves for Selected APOC3 siRNAs

| | | | IC₅₀ @ | IC₅₀ @ | MV max | SD max |
|---|---|---|---|---|---|---|
| SRS ID | Guide Sequence | Passenger Sequence | [μM] | [μM] | KD | KD |
| SRS-000665 | usAfsguacCfcgggGfcUfgCfauggcsasc | gscscaugCfaGfcCfccggguacua | 0.250 | #N/A | 63.1 | 2.7 |
| SRS-000770 | usAfsgudAcdCcggggcUfgCfauggcsasc | gscscaugCfaGfcCfccggguacua | #N/A | #N/A | 50.8 | 1.1 |
| SRS-000667 | usAfscaagGfaguaCfcCfgGfggcugscsa | csasgcccCfgGfgUfacuccuugua | 0.175 | #N/A | 67.3 | 3.6 |
| SRS-000771 | usAfscadAgdGaguaccCfgGfggcugscsa | csasgcccCfgGfgUfacuccuugua | 0.757 | #N/A | 57.2 | 1.1 |
| SRS-000712 | usGfsgaacUfgaagCfcAfuCfggucascsc | usgsaccgAfuGfgCfuucaguucca | 0.050 | #N/A | 75.1 | 2.5 |
| SRS-000772 | usGfsgadAcdTgaagccAfuCfggucascsc | usgsaccgAfuGfgCfuucaguucca | 0.032 | #N/A | 70.0 | 4.5 |
| SRS-000713 | usUfsucagGfgaacUfgAfaGfccaucsgsg | gsasuggcUfuCfaGfuucccugaaa | 0.008 | 0.328 | 90.1 | 0.8 |
| SRS-000773 | usUfsucdAgdGgaacugAfaGfccaucsgsg | gsasuggcUfuCfaGfuucccugaaa | 0.003 | 0.262 | 88.6 | 1.2 |
| SRS-000717 | usUfsaacgGfugcuCfcAfgUfagucususu | asgsacuaCfuGfgAfgcaccguuaa | 3.301 | #N/A | 50.3 | 3.3 |
| SRS-000774 | usUfsaadCgdGugcuccAfgUfagucususu | asgsacuaCfuGfgAfgcaccguuaa | 0.004 | 0.633 | 82.2 | 3.6 |
| SRS-000723 | usUfsguccUfuaacGfgUfgCfuccagsusa | csusggagCfaCfcGfuuaaggacaa | 0.008 | #N/A | 74.0 | 1.5 |
| SRS-000775 | usUfsgudCcdTuaacggUfgCfuccagsusa | csusggagCfaCfcGfuuaaggacaa | 0.003 | 7.839 | 81.5 | 1.3 |
| SRS-000753 | usGfsaauaCfugucCfcUfuUfuaagcsasa | gscsuuaaAfaGfgGfacaguauuca | 0.002 | 0.052 | 96.4 | 0.4 |
| SRS-000776 | usGfsaadTadCugucccUfuUfuaagcsasa | gscsuuaaAfaGfgGfacaguauuca | 0.002 | 0.048 | 96.4 | 0.3 |
| SRS-000754 | usGfsagaaUfacugUfcCfcUfuuuaasgsc | ususaaaaGfgGfaCfaguauucuca | 1.077 | #N/A | 57.3 | 5.0 |
| SRS-000777 | usGfsagdAadTacugucCfcUfuuuaasgsc | ususaaaaGfgGfaCfaguauucuca | 0.017 | 2.016 | 86.1 | 1.7 |
| SRS-000755 | usUfsgagaAfuacuGfuCfcCfuuuuasasg | usasaaagGfgAfcAfguauucucaa | 0.001 | 0.058 | 93.5 | 0.5 |
| SRS-000778 | usUfsgadGadAuacuguCfcCfuuuuasasg | usasaaagGfgAfcAfguauucucaa | 0.000 | 0.016 | 95.6 | 0.2 |
| SRS-000756 | usCfsugagAfauacUfgUfcCfcuuuusasa | asasaaggGfaCfaGfuauucucaga | 0.000 | 0.018 | 95.5 | 0.4 |
| SRS-000779 | usCfsugdAgdAauacugUfcCfcuuuusasa | asasaaggGfaCfaGfuauucucaga | 0.000 | 0.009 | 95.8 | 0.3 |
| SRS-000757 | usAfscugaGfaauaCfuGfuCfccuuususa | asasagggAfcAfgUfauucucagua | 0.062 | 68.377 | 73.4 | 3.6 |
| SRS-000780 | usAfscudGadGaauacuGfuCfccuuususa | asasagggAfcAfgUfauucucagua | 1.154 | #N/A | 63.1 | 4.4 |
| SRS-000758 | usCfsacugAfgaauAfcUfgUfcccuususu | asasgggaCfaGfuAfuucucaguga | 0.004 | 0.169 | 89.0 | 0.6 |
| SRS-000781 | usCfsacdTgdAgaauacUfgUfcccuususu | asasgggaCfaGfuAfuucucaguga | 0.002 | 0.053 | 94.5 | 1.3 |

Note:
the IC50 values for SRS-000755, SRS-000778, SRS-000756, and SRS-000779 were so low (i.e., highly potent) that they were extrapolated by Xlfit, as they are <0.001 AM (lowest dose of assay) “A” refers to adenosine-3′-phosphate; “a” refers to 2′-O-methyladenosine-3′-phosphate; “ Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxy guanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate.

TABLE 5
Sequence Information for siRNAs Evaluated in Non-Human Primates

| | | | Guide/ | | Guide/ | | Passenger | | Passenger/ |
|---|---|---|---|---|---|---|---|---|---|
| | | | antisense | | antisense | | /Sense | | Sense |
| | | SEQ | Structure | SEQ | Base | SEQ | Structure | SEQ | Base |
| Duplex | Target | ID | Code | ID | Sequence | ID | Code | ID | Sequence |
| ID | Position | NO. | (5′-3′) | NO. | (5′-3′) | NO. | (5′-3′) | NO. | (5′-3′) |
| SRS-000225 | 240 | 535 | usUfsucagG | 529 | UUUCAGGGAA | 547 | gsasuggcUf | 541 | GAUGGCUUCA |
| | | | fgsacUfgAf | | CUGAAGCCAU | | aCfaGfuucc | | GUUCCCUGAA |
| | | | aGfccaucsg | | CGG | | cugaaa | | A |
| | | | sg | | | | | | |
| SRS-000228 | 431 | 536 | usGfsaauaC | 530 | UGAAUACUGU | 548 | gscsuuaaAf | 542 | GCUUAAAAGG |
| | | | fugucCfcUf | | CCCUUUUAAG | | aGfgGfacag | | GACAGUAUUC |
| | | | uUfuaagcsa | | CAA | | uauuca | | A |
| | | | sa | | | | | | |
| SRS-000229 | 432 | 537 | usAfsgaauA | 531 | UAGAAUACUG | 549 | csusuaaaAf | 543 | CUUAAAAGGG |
| | | | fcuguCfcCf | | UCCCOUOUAA | | gGfgAfcagu | | ACAGUAUUCU |
| | | | uUfuuaagsc | | GCA | | auucua | | A |
| | | | sa | | | | | | |
| SRS-000230 | 434 | 538 | usUfsgagaA | 532 | UUGAGAAUAC | 550 | usasaaagGf | 544 | UAAAAGGGAC |
| | | | fuauGfuCfc | | UGUCCIUUUU | | gAfcAfguau | | AGUAUUCUCA |
| | | | Cfcuuuuasa | | AAG | | ucucaa | | A |
| | | | sg | | | | | | |
| SRS-000231 | 435 | 539 | usCfsugagA | 533 | UCUGAGAAUA | 551 | asasaaggGf | 545 | AAAAGGGACA |
| | | | fauacUfgUf | | CUGUCCCUUU | | aCfaGfuauu | | GUAUUCUCAG |
| | | | cCfcuuuusa | | UAA | | cucaga | | A |
| | | | sa | | | | | | |
| SRS-000232 | 437 | 540 | usCfsacugA | 534 | UCACUGAGAA | 552 | asasgggaCf | 546 | AAGGGACAGU |
| | | | fgaauAfcUf | | UACUGUCCCU | | aGfuAfuucu | | AUUCUCAGUG |
| | | | gUfcccuusu | | UUU | | caguga | | A |
| | | | su | | | | | | |

Note:
the target position is relative to human transcript NM_000040.3; the corresponding sequence of a certain SEQ ID NO. is located on its right column of the same row. “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothicate.
Each modified sense sequence is coupled to a targeting moiety (e.g., GalNAc or galactose) at its 3′end via a linker (e.g., Formula V′, V′′′′, V′′′′′, or V′′′′′′).

TABLE 6
Percentage of Total Cholesterol Relative to
Day −4 Following Treatment in Transgenic Mice
Percent Total Cholesterol Relative to Pre-Dose (Day −4)

| SRS ID | | Study Day | | | | | | |
|---|---|---|---|---|---|---|---|---|
| (Dose) | Statistic | −4 | 1 | 7 | 14 | 21 | 28 | 35 |
| PBS | Mean | 100.0 | 1019 | 97.7 | 88.9 | 94.3 | 95.9 | 101.9 |
| | SD | 0.0 | 19.0 | 14.6 | 15.6 | 11.8 | 13.1 | 17.0 |
| | N | 5 | 5 | 5 | 5 | 5 | 5 | 5 |
| SRS-000231 | Mean | 100.0 | 159.5 | 58.6 | 52.3 | 60.6 | 74.2 | 104.5 |
| (0.3 mg/kg) | SD | 0.0 | 55.4 | 19.4 | 15.9 | 19.5 | 20.6 | 37.1 |
| | N | 5 | 5 | 5 | 5 | 5 | 5 | 5 |
| SRS-000231 | Mean | 100.0 | 95.8 | 46.4 | 43.3 | 44.4 | 48.6 | 48.6 |
| (1 mg/kg) | SD | 0.0 | 13.7 | 16.5 | 16.0 | 15.1 | 20.8 | 9.2 |
| | N | 5 | 5 | 5 | 5 | 5 | 5 | 4 |
| SRS-000231 | Mean | 100.0 | 101.7 | 47.2 | 44.7 | 49.4 | 49.6 | 57.1 |
| (3 mg/kg) | SD | 0.0 | 11.9 | 14.1 | 13.2 | 16.2 | 15.7 | 17.3 |
| | N | 5 | 5 | 5 | 5 | 5 | 5 | 5 |
| SRS-000231 | Mean | 100.0 | 131.1 | 50.2 | 54.7 | 53.2 | 61.6 | 63.6 |
| (10 mg/kg) | SD | 0.0 | 81.0 | 6.8 | 13.9 | 7.4 | 19.0 | 15.0 |
| | N | 4 | 4 | 4 | 4 | 4 | 4 | 4 |
| SRS-000228 | Mean | 100.0 | 107.1 | 52.7 | 40.8 | 42.7 | 48.2 | 56.2 |
| (0.3 mg/kg) | SD | 0.0 | 22.8 | 9.6 | 13.3 | 10.4 | 15.6 | 21.2 |
| | N | 5 | 5 | 5 | 5 | 5 | 5 | 5 |
| SRS-000228 | Mean | 100.0 | 129.9 | 38.9 | 36.5 | 38.5 | 41.7 | 43.2 |
| (1 mg/kg) | SD | 0.0 | 28.8 | 10.2 | 14.8 | 13.0 | 11.0 | 19.4 |
| | N | 5 | 5 | 5 | 5 | 5 | 5 | 5 |
| SRS-000228 | Mean | 100.0 | 85.4 | 37.5 | 31.0 | 36.1 | 33.9 | 38.1 |
| (3 mg/kg) | SD | 0.0 | 17.8 | 28.5 | 17.8 | 18.5 | 17.6 | 23.5 |
| | N | 5 | 5 | 5 | 5 | 5 | 5 | 5 |
| SRS-000228 | Mean | 100.0 | 96.2 | 29.4 | 25.7 | 26.7 | 28.3 | 29.7 |
| (10 mg/kg) | SD | 0.0 | 19.3 | 7.3 | 6.2 | 6.3 | 6.3 | 7.1 |
| | N | 5 | 5 | 5 | 5 | 5 | 5 | 5 |
| SRS-000229 | Mean | 100.0 | 106.8 | 38.9 | 42.6 | 50.4 | 60.7 | 73.8 |
| (1 mg/kg) | SD | 0.0 | 18.2 | 13.3 | 17.1 | 193 | 22.1 | 20.8 |
| | N | 5 | 5 | 5 | 5 | 5 | 5 | 5 |
| SRS-000229 | Mean | 100.0 | 124.6 | 37.6 | 37.5 | 40.3 | 45.8 | 58.0 |
| (3 mg/kg) | SD | 0.0 | 32.4 | 10.1 | 11.0 | 9.2 | 10.9 | 14.3 |
| | N | 5 | 5 | 5 | 5 | 5 | 5 | 5 |
| SRS-000229 | Mean | 100.0 | 144.6 | 57.4 | 47.8 | 54.5 | 58.3 | 68.7 |
| (10 mg/kg) | SD | 0.0 | 80.2 | 36.7 | 36.2 | 35.4 | 38.8 | 48.2 |
| | N | 5 | 5 | 5 | 4 | 5 | 5 | 5 |

TABLE 7
Percentage of Triglycerides Relative to
Day −4 Following Treatment in Transgenic Mice
Percent Triglycerides Relative to Pre-Dose (Day −4)

| SRS ID | | Study Day | | | | | | |
|---|---|---|---|---|---|---|---|---|
| (Dose) | Statistic | −4 | 1 | 7 | 14 | 21 | 28 | 35 |
| PBS | Mean | 100.0 | 122.6 | 478.9 | 314.9 | 491.9 | 116.0 | 232.0 |
| | SD | 0.0 | 47.8 | 249.5 | 33.0 | 306.0 | 19.2 | 182.8 |
| | N | 5 | 5 | 5 | 5 | 5 | 5 | 5 |
| SRS-000231 | Mean | 100.0 | 91.3 | 68.8 | 63.6 | 76.0 | 120.7 | 111.1 |
| (0.3 mg/kg) | SD | 0.0 | 35.9 | 33.5 | 29.0 | 24.9 | 24.4 | 77.0 |
| | N | 5 | 5 | 5 | 5 | 5 | 5 | 5 |
| SRS-000231 | Mean | 100.0 | 157.0 | 35.8 | 33.3 | 34.6 | 49.7 | 70.4 |
| (1 mg/kg) | SD | 0.0 | 95.6 | 17.0 | 18.7 | 15.7 | 20.5 | 29.3 |
| | N | 5 | 5 | 5 | 5 | 5 | 5 | 4 |
| SRS-000231 | Mean | 100.0 | 159.1 | 27.5 | 29.5 | 27.5 | 29.3 | 41.3 |
| (3 mg/kg) | SD | 0.0 | 104.3 | 8.3 | 11.5 | 12.4 | 15.0 | 18.9 |
| | N | 5 | 5 | 5 | 5 | 5 | 5 | 5 |
| SRS-000231 | Mean | 1000 | 110.3 | 22.7 | 21.7 | 22.9 | 27.4 | 27.4 |
| (10 mg/kg) | SD | 0.0 | 22.3 | 9.2 | 7.1 | 8.2 | 8.8 | 4.5 |
| | N | 4 | 4 | 4 | 4 | 4 | 4 | 4 |
| SRS-000228 | Mean | 100.0 | 95.8 | 168.4 | 86.1 | 76.7 | 95.0 | 79.6 |
| (0.3 mg/kg) | SD | 0.0 | 23.1 | 121.4 | 12.9 | 27.5 | 59.8 | 21.8 |
| | N | 5 | 5 | 5 | 5 | 5 | 5 | 5 |
| SRS-000228 | Mean | 100.0 | 163.1 | 37.4 | 32.2 | 44.6 | 55.2 | 48.3 |
| (1 mg/kg) | SD | 0.0 | 87.5 | 7.3 | 4.4 | 11.4 | 10.9 | 10.7 |
| | N | 5 | 5 | 5 | 5 | 5 | 5 | 5 |
| SRS-000228 | Mean | 100.0 | 112.1 | 34.1 | 27.1 | 25.4 | 24.3 | 29.8 |
| (3 mg/kg) | SD | 0.0 | 25.1 | 13.1 | 5.0 | 7.9 | 4.7 | 10.1 |
| | N | 5 | 5 | 5 | 5 | 5 | 5 | 5 |
| SRS-000228 | Mean | 100.0 | 94.9 | 27.4 | 26.1 | 20.9 | 31.8 | 27.3 |
| (10 mg/kg) | SD | 0.0 | 20.2 | 7.1 | 6.2 | 6.9 | 9.0 | 9.6 |
| | N | 5 | 5 | 5 | 5 | 5 | 5 | 5 |
| SRS-000229 | Mean | 100.0 | 112.5 | 70.9 | 96.8 | 1009 | 150.7 | 209.8 |
| (1 mg/kg) | SD | 0.0 | 15.7 | 17.4 | 12.0 | 9.0 | 75.4 | 134.6 |
| | N | 5 | 5 | 5 | 5 | 5 | 5 | 5 |
| SRS-000229 | Mean | 100.0 | 94.8 | 31.1 | 32.4 | 43.2 | 76.0 | 71.2 |
| (3 mg/kg) | SD | 0.0 | 17.8 | 19.1 | 10.8 | 27.2 | 56.5 | 25.9 |
| | N | 5 | 5 | 5 | 5 | 5 | 5 | 5 |
| SRS-000229 | Mean | 100.0 | 198.4 | 53.0 | 46.8 | 45.7 | 67.7 | 78.3 |
| (10 mg/kg) | SD | 0.0 | 112.7 | 25.7 | 10.8 | 14.7 | 24.4 | 28.0 |
| | N | 5 | 5 | 5 | 4 | 5 | 5 | 5 |

TABLE 8
Percentage of HDL-Cholesterol Relative to
Day −4 Following Treatment in Transgenic Mice
Percent HDL-Cholesterol Relative to Pre-Dose (Day −4)

| SRS ID | | Study Day | | | | | | |
|---|---|---|---|---|---|---|---|---|
| (Dose) | Statistic | −4 | 1 | 7 | 14 | 21 | 28 | 35 |
| PBS | Mean | 100.0 | 104.1 | 73.1 | 90.8 | 88.7 | 108.9 | 108.9 |
| | SD | 0.0 | 17.9 | 37.8 | 33.7 | 43.5 | 38.7 | 30.9 |
| | N | 5 | 5 | 5 | 5 | 5 | 5 | 5 |
| SRS-000231 | Mean | 100.0 | 124.2 | 143.5 | 158.6 | 149.2 | 116.3 | 159.1 |
| (0.3 mg/kg) | SD | 0.0 | 26.9 | 54.9 | 64.4 | 59.4 | 59.9 | 49.4 |
| | N | 5 | 5 | 5 | 5 | 5 | 5 | 5 |
| SRS-000231 | Mean | 100.0 | 103.1 | 173.8 | 167.1 | 174.0 | 1643 | 177.3 |
| (1 mg/kg) | SD | 0.0 | 34.6 | 57.7 | 51.9 | 60.7 | 48.1 | 43.3 |
| | N | 5 | 5 | 5 | 5 | 5 | 5 | 4 |
| SRS-000231 | Mean | 100.0 | 90.0 | 165.0 | 152.1 | 167.8 | 1672 | 185.6 |
| (3 mg/kg) | SD | 0.0 | 18.8 | 35.8 | 23.5 | 24.1 | 21.1 | 32.2 |
| | N | 5 | 5 | 5 | 5 | 5 | 5 | 5 |
| SRS-000231 | Mean | 100.0 | 1318 | 211.4 | 220.4 | 217.8 | 2359 | 250.7 |
| (10 mg/kg) | SD | 0.0 | 24.7 | 77.3 | 103.1 | 94.7 | 103.4 | 107.1 |
| | N | 4 | 4 | 4 | 4 | 4 | 4 | 4 |
| SRS-000228 | Mean | 100.0 | 92.4 | 86.2 | 102.2 | 124.2 | 106.1 | 99.5 |
| (0.3 mg/kg) | SD | 0.0 | 15.3 | 22.3 | 10.5 | 35.9 | 38.7 | 35.6 |
| | N | 5 | 5 | 5 | 5 | 5 | 5 | 5 |
| SRS-000228 | Mean | 100.0 | 108.2 | 196.7 | 174.7 | 174.2 | 176.0 | 183.3 |
| (1 mg/kg) | SD | 0.0 | 28.3 | 28.1 | 29.9 | 28.0 | 16.3 | 31.3 |
| | N | 5 | 5 | 5 | 5 | 5 | 5 | 5 |
| SRS-000228 | Mean | 100.0 | 83.2 | 122.2 | 108.4 | 128.3 | 119.7 | 132.7 |
| (3 mg/kg) | SD | 0.0 | 18.9 | 29.0 | 13.0 | 16.0 | 14.4 | 22.3 |
| | N | 5 | 5 | 5 | 5 | 5 | 5 | 5 |
| SRS-000228 | Mean | 100.0 | 99.9 | 161.1 | 140.0 | 151.6 | 1499 | 169.0 |
| (10 mg/kg) | SD | 0.0 | 13.4 | 24.5 | 24.1 | 15.0 | 24.0 | 25.7 |
| | N | 5 | 5 | 5 | 5 | 5 | 5 | 5 |
| SRS-000229 | Mean | 100.0 | 94.7 | 1202 | 115.5 | 127.1 | 88.1 | 100.5 |
| (1 mg/kg) | SD | 0.0 | 26.2 | 44.5 | 37.1 | 46.3 | 31.3 | 44.7 |
| | N | 5 | 5 | 5 | 5 | 5 | 5 | 5 |
| SRS-000229 | Mean | 100.0 | 128.7 | 198.0 | 1809 | 1679 | 148.1 | 144.8 |
| (3 mg/kg) | SD | 0.0 | 36.3 | 139.6 | 92.2 | 70.4 | 75.3 | 52.6 |
| | N | 5 | 5 | 5 | 5 | 5 | 5 | 5 |
| SRS-000229 | Mean | 100.0 | 105.7 | 172.0 | 157.7 | 171.6 | 161.5 | 185.2 |
| (10 mg/kg) | SD | 0.0 | 26.6 | 48.9 | 55.1 | 52.3 | 49.7 | 62.5 |
| | N | 5 | 5 | 5 | 4 | 5 | 5 | 5 |

TABLE 9
Percentage of LDL-Cholesterol Relative to
Day −4 Following Treatment in Transgenic Mice
Percent LDL-Cholesterol Relative to Pre-Dose (Day −4)

| SRS ID | | Study Day | | | | | | |
|---|---|---|---|---|---|---|---|---|
| (Dose) | Statistic | −4 | 1 | 7 | 14 | 21 | 28 | 35 |
| PBS | Mean | 100.0 | 1153 | 207.3 | 67.1 | 259.0 | 117.6 | 93.4 |
| | SD | 0.0 | 94.6 | 192.0 | 38.1 | 236.1 | 38.7 | 62.2 |
| | N | 5 | 5 | 5 | 5 | 5 | 5 | 5 |
| SRS-000231 | Mean | 100.0 | 60.2 | 64.8 | 43.4 | 62.2 | 1162 | 130.1 |
| (0.3 mg/kg) | SD | 0.0 | 72.2 | 18.8 | 24.3 | 25.6 | 31.5 | 85.6 |
| | N | 5 | 5 | 5 | 5 | 5 | 5 | 4 |
| SRS-000231 | Mean | 100.0 | 115.7 | 47.7 | 19.5 | 16.9 | 22.0 | 37.4 |
| (1 mg/kg) | SD | 0.0 | 42.0 | 18.7 | 6.6 | 5.2 | 4.6 | 6.5 |
| | N | 5 | 5 | 5 | 5 | 5 | 5 | 4 |
| SRS-000231 | Mean | 100.0 | 157.0 | 64.9 | 32.4 | 21.6 | 20.2 | 27.6 |
| (3 mg/kg) | SD | 0.0 | 125.7 | 18.3 | 27.6 | 6.1 | 6.2 | 6.6 |
| | N | 5 | 5 | 5 | 5 | 5 | 5 | 5 |
| SRS-00023 1 | Mean | 100.0 | 74.9 | 43.2 | 14.8 | 14.4 | 38.4 | 14.4 |
| (10 mg/kg) | SD | 0.0 | 20.1 | 19.7 | 6.4 | 6.6 | 25.8 | 6.6 |
| | N | 4 | 4 | 4 | 4 | 4 | 4 | 4 |
| SRS-000228 | Mean | 100.0 | 65.8 | 84.0 | 37.1 | 37.0 | 49.9 | 55.1 |
| (0.3 mg/kg) | SD | 0.0 | 35.1 | 76.8 | 129 | 33.1 | 36.8 | 40.8 |
| | N | 5 | 5 | 5 | 5 | 5 | 5 | 5 |
| SRS-000228 | Mean | 100.0 | 330.8 | 70.4 | 44.0 | 39.4 | 60.8 | 55.1 |
| (1 mg/kg) | SD | 0.0 | 550.5 | 68.8 | 49.0 | 45.5 | 73.6 | 72.0 |
| | N | 5 | 5 | 5 | 5 | 5 | 5 | 5 |
| SRS-000228 | Mean | 100.0 | 72.4 | 58.2 | 19.4 | 19.6 | 19.4 | 22.4 |
| (3 mg/kg) | SD | 0.0 | 49.3 | 36.6 | 12.2 | 11.9 | 12.2 | 16.2 |
| | N | 5 | 5 | 5 | 5 | 5 | 5 | 5 |
| SRS-000228 | Mean | 100.0 | 155.6 | 72.7 | 24.2 | 44.2 | 26.0 | 29.3 |
| (10 mg/kg) | SD | 0.0 | 100.3 | 48.9 | 16.3 | 59.6 | 18.9 | 22.5 |
| | N | 5 | 5 | 5 | 5 | 5 | 5 | 5 |
| SRS-000229 | Mean | 100.0 | 86.0 | 41.5 | 41.6 | 51.4 | 128.6 | 117.0 |
| (1 mg/kg) | SD | 0.0 | 47.4 | 16.6 | 16.8 | 23.4 | 61.7 | 42.6 |
| | N | 5 | 5 | 5 | 5 | 5 | 5 | 5 |
| SRS-000229 | Mean | 100.0 | 79.0 | 69.0 | 33.1 | 49.4 | 111.0 | 54.0 |
| (3 mg/kg) | SD | 0.0 | 52.8 | 70.1 | 34.1 | 79.4 | 1922 | 38.4 |
| | N | 5 | 5 | 5 | 5 | 5 | 5 | 5 |
| SRS-000229 | Mean | 100.0 | 272.3 | 90.1 | 30.0 | 53.2 | 37.1 | 48.3 |
| (10 mg/kg) | SD | 0.0 | 179.1 | 70.8 | 23.6 | 47.9 | 25.1 | 34.4 |
| | N | 5 | 5 | 5 | 5 | 5 | 5 | 5 |

TABLE 10
Percentage of Plasma APOC3 Relative to
Day 1 Following Treatment in Transgenic Mice
Percent Plasma APOC3 Relative to Day 1

| SRS ID | | Study Day | | | | | |
|---|---|---|---|---|---|---|---|
| (Dose) | Statistic | 1 | 7 | 14 | 21 | 28 | 35 |
| PBS | Mean | 100.0 | 91.0 | 75.4 | 75.6 | 1065 | 96.9 |
| | SD | 3.9 | 10.1 | 6.3 | 5.1 | 8.6 | 112 |
| | N | 1 | 5 | 5 | 2 | 5 | 5 |
| SRS-000231 | Mean | 100.0 | 479 | 42.7 | 43.7 | 84.2 | 913 |
| (0.3 mg/kg) | SD | 10.9 | 20.7 | 8.0 | 7.7 | 12.8 | 14.0 |
| | N | 4 | 5 | 5 | 3 | 4 | 5 |
| SRS-000231 | Mean | 100.0 | 23.8 | 24.9 | 28.5 | 48.2 | 56.6 |
| (1 mg/kg) | SD | 21.7 | 8.8 | 6.8 | 9.2 | 9.0 | 10.9 |
| | N | 2 | 5 | 5 | 5 | 4 | 4 |
| SRS-000231 | Mean | 100.0 | 22.1 | 22.2 | 219 | 47.5 | 43.4 |
| (3 mg/kg) | SD | 27.3 | 3.5 | 2.8 | 2.6 | 7.5 | 8.1 |
| | N | 3 | 5 | 5 | 5 | 5 | 5 |
| SRS-000231 | Mean | 1000 | 17.7 | 22.0 | 232 | 45.6 | 342 |
| (10 mg/kg) | SD | 7.1 | 6.5 | 1.9 | 1.5 | 5.4 | 2.0 |
| | N | 1 | 4 | 4 | 2 | 3 | 4 |
| SRS-000228 | Mean | 100.0 | 62.6 | 41.2 | 49.1 | 57.6 | 65.7 |
| (0.3 mg/kg) | SD | 10.7 | 18.7 | 4.5 | 16.5 | 19.1 | 18.3 |
| | N | 3 | 5 | 5 | 4 | 5 | 5 |
| SRS-000228 | Mean | 100.0 | 24.6 | 169 | 23.8 | 39.3 | 38.6 |
| (1 mg/kg) | SD | 4.8 | 2.4 | 2.8 | 3.8 | 10.0 | 4.6 |
| | N | 2 | 5 | 4 | 4 | 5 | 5 |
| SRS-000228 | Mean | 100.0 | 28.0 | 16.6 | 16.5 | 34.1 | 323 |
| (3 mg/kg) | SD | 14.8 | 3.0 | 2.2 | 2.4 | 8.4 | 8.2 |
| | N | 3 | 5 | 5 | 1 | 5 | 5 |
| SRS-000228 | Mean | 100.0 | 182 | 11.5 | 11.7 | 19.2 | 22.7 |
| (10 mg/kg) | SD | 13.0 | 3.2 | 1.5 | 2.8 | 1.9 | 3.3 |
| | N | 4 | 5 | 5 | 3 | 5 | 5 |
| SRS-000229 | Mean | 100.0 | 42.6 | 39.7 | 43.7 | 79.5 | 87.0 |
| (1 mg/kg) | SD | 9.0 | 9.6 | 5.7 | 4.7 | 7.4 | 11.4 |
| | N | 5 | 5 | 5 | 5 | 5 | 5 |
| SRS-000229 | Mean | 100.0 | 22.8 | 26.5 | 30.0 | 60.8 | 74.0 |
| (3 mg/kg) | SD | 13.6 | 9.3 | 8.2 | 11.7 | 18.7 | 23.2 |
| | N | 2 | 5 | 5 | 5 | 5 | 5 |
| SRS-000229 | Mean | 100.0 | 19.6 | 18.6 | 213 | 35.1 | 43.5 |
| (10 mg/kg) | SD | 12.7 | 5.1 | 3.0 | 1.9 | 7.2 | 6.9 |
| | N | 4 | 5 | 5 | 2 | 5 | 5 |

TABLE 11
Percent Remaining APOC3 mRNA Relative to PBS Control
Following Treatment in Transgenic Mice
% Remaining APOC3 mRNA on Day 35 Relative to PBS

| | | Dose Level | | | |
|---|---|---|---|---|---|
| SRS ID | Statistic | 0.3 mg/kg | 1 mg/kg | 3 mg/kg | 10 mg/kg |
| SRS-000231 | Mean | 67.7 | 45.4 | 8.5 | 3.3 |
| | SD | 19.5 | 27.3 | 1.8 | 1.6 |
| | N | 5 | 4 | 5 | 4 |
| SRS-000228 | Mean | 21.2 | 16.2 | 9.7 | 3.8 |
| | SD | 4.5 | 5.0 | 4.5 | 1.8 |
| | N | 5 | 5 | 5 | 5 |
| SRS-000229 | Mean | — | 22.7 | 19.1 | 10.5 |
| | SD | — | 6.4 | 5.6 | 2.9 |
| | N | — | 5 | 5 | 5 |

TABLE 12
Additional siRNA sequences evaluated in Example 5

| | | Guide/ | | Guide/ | | Passenger/ | | Passenger/ |
|---|---|---|---|---|---|---|---|---|
| | | antisense | | antisense | | sense | | sense |
| | SEQ | Structure | SEQ | Base | SEQ | Structure | SEQ | Base |
| Duplex | ID | Code | ID | Sequence | ID | Code | ID | Sequence |
| ID | NO | (5′-3′) | NO | (5′-3′) | NO | (5′-3′) | NO | (5′-3′) |
| SRS-000231 | 564 | usCfsugagA | 553 | UCUGAGAAUA | 586 | asasaaggGf | 575 | AAAAGGGACA |
| | | fauacUfgUf | | CUGUCCCUUU | | aCfaGfuauu | | GUAUUCUCAG |
| | | cCfcuuuusa | | UAA | | cucaga | | A |
| | | sa | | | | | | |
| SRS-001860 | 565 | vpusCfsuga | 554 | UCUGAGAAUA | 587 | asasaaggGf | 576 | AAAAGGGACA |
| | | gAfauacUfg | | CUGUCCCUUU | | aCfaGfuauu | | GUAUUCUCAG |
| | | UfcCfcuuuu | | UAA | | cucaga | | A |
| | | sasa | | | | | | |
| SRS-001861 | 566 | usCfsugagA | 555 | UCUGAGAAUA | 588 | gsasaaggGf | 577 | GAAAGGGACA |
| | | fauacUfgUf | | CUGUCCCUUU | | aCfaGfuauu | | GUAUUCUCAG |
| | | cCfcuuucsa | | CAA | | cucaga | | A |
| | | sa | | | | | | |
| SRS-001862 | 567 | usCfsugagA | 556 | UCUGAGAAUA | 589 | csasaaggGf | 578 | CAAAGGGACA |
| | | fauacUfgUf | | CUGUCCCUUU | | aCfaGfuauu | | GUAUUCUCAG |
| | | cCfcumugsa | | GAA | | cucaga | | A |
| | | sa | | | | | | |
| SRS-001863 | 568 | usCfsugagA | 557 | UCUGAGAAUA | 590 | gscsaaggGf | 579 | GCAAGGGACA |
| | | fsuacUfgUf | | CUGUCCCUUG | | aCfaGfusuu | | GUAUUCUCAG |
| | | cCfcuugcsa | | CAA | | cucaga | | A |
| | | sa | | | | | | |
| SRS-001864 | 569 | usCfsugagA | 558 | UCUGAGAAUA | 591 | csgsaaggGf | 580 | CGAAGGGACA |
| | | fauacUfgUf | | CUGUCCCUUC | | aCfaGfuauu | | GUAUUCUCAG |
| | | cCfcuucgsa | | GAA | | cucaga | | A |
| | | sa | | | | | | |
| SRS-001865 | 570 | usCfsugagA | 559 | UCUGAGAAUA | 592 | (invAb)asa | 581 | AAAAGGGACA |
| | | fauacUfgUf | | CUGUCCCUUU | | saaggGfaCf | | GUAUUCUCAG |
| | | cCfcuuuusa | | UAA | | aGfuauucuc | | A |
| | | sa | | | | aga | | |
| SRS-001866 | 571 | usCfsugagA | 560 | UCUGAGAAUA | 593 | (invAb)csa | 582 | CAAAGGGACA |
| | | fauacUfgUf | | CUGUCCCUUU | | saaggGfaCf | | GUAUUCUCAG |
| | | cCfcuuugsa | | GAA | | aGfuauucuc | | A |
| | | sa | | | | aga | | |
| SRS-001868 | 572 | usCfsugdAg | 561 | UCUGAGAAUA | 594 | asasaaggGf | 583 | AAAAGGGACA |
| | | dAauacUfgU | | CUGUCCCUUU | | aCfaGfuauu | | GUAUUCUCAG |
| | | fcCfcuuuus | | UAA | | cucaga | | A |
| | | asa | | | | | | |
| SRS-001869 | 573 | usCfsugagA | 562 | UCUGAGAAUA | 595 | asasaaggGf | 584 | AAAAGGGACA |
| | | fauacUfgUf | | CUGUCCCUUU | | aCfaGfusuu | | GUAUUCUCAG |
| | | scCfcuuuus | | UAA | | cucaga | | A |
| | | asa | | | | | | |
| SRS-001870 | 574 | usCfsugagA | 563 | UCUGAGAAUA | 59% | asasaaggGf | 585 | AAAAGGGACA |
| | | fauacUfgUf | | CUGUCCCUUU | | aCfaGfuauu | | GUAUUCUCAG |
| | | cCfcsuuuus | | UAA | | cucaga | | A |
| | | asa | | | | | | |

Note:
the target position of each of the sequences listed in Table 12 is 435 (relative to human transcript NM_000040.3); the corresponding sequence of a certain SEQ ID NO. is located on its right column of the same row. “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide. Each modified sense sequence is coupled to a targeting moiety (e.g., Gal NAc) at its 3′ end via a linker (e.g., Formula V′, V′′′′, V′′′′′, or V′′′′′′).

TABLE 13
Percentage of Total Cholesterol Relative to
Day 1 Following Treatment in Transgenic Mice
Percent Total Cholesterol Relative to Day 1

| | | Study Day | | | | | | | | |
|---|---|---|---|---|---|---|---|---|---|---|
| SRS ID | Statistics | 1 | 7 | 14 | 21 | 28 | 35 | 42 | 49 | 56 |
| 1x PBS | Mean | 100.0 | 1132 | 109.8 | 119.0 | 98.6 | 104.8 | 98.5 | 99.0 | 99.5 |
| | SD | 0.0 | 19.6 | 20.6 | 32.4 | 878 | 27.7 | 24.0 | 199 | 212 |
| | N | 5 | 5 | 5 | 5 | 5 | 5 | 5 | 5 | 5 |
| SRS-000231 | Mean | 100.0 | 29.9 | 40.1 | 34.9 | 612 | 31.3 | 33.2 | 313 | 30.7 |
| | SD | 0.0 | 12.9 | 11.4 | 15.8 | 33.1 | 12.3 | 12.4 | 12.6 | 10.3 |
| | N | 4 | 4 | 3 | 4 | 4 | 4 | 4 | 4 | 4 |
| SRS-001860 | Mean | 100.0 | 46.3 | 64.1 | 64.0 | 55.4 | 53.2 | 53.7 | 43.3 | 41.5 |
| | SD | 0.0 | 20.3 | 34.5 | 36.5 | 28.0 | 27.4 | 24.7 | 18.0 | 16.7 |
| | N | 4 | 4 | 4 | 4 | 4 | 4 | 4 | 4 | 4 |
| SRS-001861 | Mean | 100.0 | 49.7 | 58.0 | 56.9 | 57.2 | 53.6 | 515 | 47.2 | 46.7 |
| | SD | 0.0 | 19.5 | 22.9 | 21.3 | 22.8 | 20.4 | 19.3 | 16.4 | 15.9 |
| | N | 5 | 5 | 5 | 5 | 5 | 5 | 5 | 5 | 5 |
| SRS-001862 | Mean | 100.0 | 56.4 | 60.9 | 64.2 | 61.3 | 58.1 | 54.4 | 51.6 | 48.9 |
| | SD | 0.0 | 29.7 | 26.8 | 32.6 | 29.9 | 28.6 | 26.1 | 24.4 | 23.7 |
| | N | 5 | 5 | 5 | 5 | 5 | 5 | 5 | 5 | 5 |
| SRS-001863 | Mean | 100.0 | 49.1 | 63.2 | 50.6 | 50.7 | 47.1 | 50.2 | 49.4 | 46.6 |
| | SD | 0.0 | 28.4 | 33.1 | 25.7 | 26.8 | 22.7 | 27.1 | 27.8 | 26.1 |
| | N | 5 | 5 | 5 | 5 | 5 | 5 | 5 | 5 | 5 |
| SRS-001864 | Mean | 100.0 | 39.3 | 46.3 | 45.5 | 47.4 | 49.3 | 49.0 | 50.4 | 45.9 |
| | SD | 0.0 | 19.1 | 19.0 | 18.4 | 21.8 | 20.4 | 21.5 | 20.8 | 17.6 |
| | N | 5 | 5 | 5 | 5 | 5 | 5 | 5 | 5 | 5 |
| SRS-001865 | Mean | 100.0 | 50.8 | 57.9 | 55.8 | 57.6 | 52.7 | 55.5 | 49.9 | 63.2 |
| | SD | 0.0 | 17.8 | 18.7 | 20.5 | 20.5 | 16.2 | 18.4 | 17.0 | 36.4 |
| | N | 5 | 5 | 5 | 5 | 5 | 5 | 5 | 5 | 5 |
| SRS-001866 | Mean | 100.0 | 43.6 | 56.6 | 54.7 | 58.1 | 4.2 | 56.0 | 52.3 | 51.9 |
| | SD | 0.0 | 21.3 | 30.4 | 27.8 | 30.1 | 31.2 | 31.4 | 30.1 | 35.5 |
| | N | 5 | 4 | 4 | 4 | 4 | 4 | 4 | 4 | 4 |
| SRS-001868 | Mean | 1000 | 37.2 | 42.8 | 38.0 | 37.5 | 33.0 | 362 | 33.2 | 31.2 |
| | SD | 0.0 | 11.8 | 15.6 | 11.4 | 13.8 | 12.4 | 12.2 | 12.4 | 10.8 |
| | N | 5 | 5 | 5 | 5 | 5 | 5 | 5 | 5 | 5 |
| SRS-001869 | Mean | 1000 | 47.9 | 48.9 | 47.0 | 479 | 44.0 | 46.4 | 42.6 | 40.3 |
| | SD | 0.0 | 21.4 | 25.5 | 22.7 | 26.2 | 22.7 | 24.1 | 20.7 | 169 |
| | N | 4 | 4 | 4 | 4 | 4 | 4 | 4 | 4 | 4 |
| SRS-001870 | Mean | 100.0 | 36.1 | 54.1 | 45.1 | 44.5 | 38.2 | 38.8 | 38.9 | 39.2 |
| | SD | 0.0 | 14.5 | 28.8 | 12.7 | 15.0 | 13.1 | 119 | 12.8 | 12.5 |
| | N | 4 | 4 | 4 | 4 | 3 | 3 | 3 | 3 | 3 |

TABLE 14
Percentage of Triglycerides Relative to
Day 1 Following Treatment in Transgenic Mice
Percent Triglycerides Relative to Day 1

| | | Study Day | | | | | | | | |
|---|---|---|---|---|---|---|---|---|---|---|
| SRS ID | Statistics | 3 | 7 | 14 | 21 | 28 | 35 | 42 | 49 | 56 |
| 1x PBS | Mean | 100.0 | 182.5 | 158.6 | 361.0 | 186.5 | 135.8 | 202.7 | 297.7 | 204.0 |
| | SD | 0.0 | 137.6 | 118.1 | 324.5 | 188.8 | 70.5 | 182.9 | 197.3 | 239.1 |
| | N | 5 | 5 | 5 | 5 | 5 | 5 | 5 | 5 | 5 |
| SRS-000231 | Mean | 100.0 | 10.0 | 15.9 | 12.4 | 43.6 | 9.7 | 14.9 | 13.2 | 18.8 |
| | SD | 0.0 | 4.8 | 6.8 | 8.5 | 42.0 | 5.2 | 5.3 | 6.0 | 7.0 |
| | N | 4 | 4 | 3 | 4 | 4 | 4 | 4 | 4 | 4 |
| SRS-001860 | Mean | 1000 | 12.0 | 20.5 | 12.5 | 11.9 | 10.4 | 16.7 | 9.7 | 15.0 |
| | SD | 0.0 | 5.6 | 17.4 | 6.2 | 5.5 | 5.3 | 10.1 | 6.1 | 8.0 |
| | N | 4 | 4 | 4 | 4 | 4 | 4 | 4 | 4 | 4 |
| SRS-001861 | Mean | 100.0 | 12.9 | 20.8 | 21.9 | 19.8 | 21.2 | 22.0 | 17.6 | 26.8 |
| | SD | 0.0 | 8.3 | 13.8 | 16.5 | 12.9 | 18.2 | 15.7 | 12.2 | 22.4 |
| | N | 5 | 5 | 5 | 5 | 5 | 5 | 5 | 5 | 5 |
| SRS-001862 | Mean | 100.0 | 12.2 | 22.7 | 25.6 | 19.0 | 19.3 | 24.2 | 17.0 | 22.6 |
| | SD | 0.0 | 4.4 | 12.4 | 14.7 | 8.4 | 13.1 | 14.9 | 7.5 | 14.2 |
| | N | 5 | 5 | 5 | 5 | 5 | 5 | 5 | 5 | 5 |
| SRS-001863 | Mean | 100.0 | 14.2 | 20.0 | 25.1 | 19.7 | 19.0 | 24.3 | 26.5 | 25.7 |
| | SD | 0.0 | 17.2 | 26.1 | 38.2 | 22.3 | 26.6 | 28.7 | 37.4 | 31.0 |
| | N | 5 | 5 | 5 | 5 | 5 | 5 | 5 | 5 | 5 |
| SRS-001864 | Mean | 100.0 | 17.7 | 27.7 | 18.0 | 19.9 | 16.6 | 25.9 | 18.7 | 19.7 |
| | SD | 0.0 | 8.6 | 12.3 | 8.0 | 6.2 | 8.2 | 11.3 | 9.1 | 8.8 |
| | N | 5 | 5 | 5 | 5 | 5 | 5 | 5 | 5 | 5 |
| SRS-001865 | Mean | 100.0 | 14.8 | 27.8 | 16.2 | 23.3 | 24.4 | 24.9 | 32.6 | 39.2 |
| | SD | 0.0 | 7.5 | 13.9 | 4.7 | 13.7 | 9.8 | 5.2 | 12.1 | 12.0 |
| | N | 5 | 5 | 5 | 5 | 5 | 5 | 5 | 5 | 5 |
| SRS-001866 | Mean | 100.0 | 13.3 | 20.5 | 21.3 | 18.8 | 14.9 | 20.0 | 17.7 | 23.6 |
| | SD | 0.0 | 5.6 | 8.7 | 9.6 | 6.1 | 5.0 | 8.6 | 8.5 | 18.3 |
| | N | 5 | 4 | 4 | 4 | 4 | 4 | 4 | 4 | 4 |
| SRS-001868 | Mean | 100.0 | 20.5 | 28.7 | 22.5 | 18.9 | 18.3 | 25.8 | 18.9 | 23.7 |
| | SD | 0.0 | 11.6 | 15.5 | 12.0 | 7.7 | 8.0 | 14.9 | 7.6 | 10.3 |
| | N | 5 | 5 | 5 | 5 | 5 | 5 | 5 | 5 | 5 |
| SRS-001869 | Mean | 100.0 | 12.7 | 17.4 | 20.0 | 20.8 | 17.0 | 29.0 | 17.3 | 21.5 |
| | SD | 0.0 | 7.1 | 11.5 | 11.0 | 19.9 | 15.0 | 32.3 | 13.1 | 11.3 |
| | N | 4 | 4 | 4 | 4 | 4 | 4 | 4 | 4 | 4 |
| SRS-001870 | Mean | 100.0 | 15.6 | 24.3 | 19.8 | 21.6 | 10.1 | 20.9 | 14.8 | 19.1 |
| | SD | 0.0 | 10.3 | 16.0 | 13.9 | 20.2 | 8.1 | 21.1 | 13.1 | 15.3 |
| | N | 4 | 4 | 4 | 4 | 3 | 3 | 3 | 3 | 3 |

TABLE 15
Percentage of HDL-Cholesterol Relative to
Day 1 Following Treatment in Transgenic Mice
Percent HDL-Cholesterol Relative to Day 1

| | | Study Day | | | | | | | | |
|---|---|---|---|---|---|---|---|---|---|---|
| SRS ID | Statistics | 1 | 7 | 14 | 21 | 28 | 35 | 42 | 49 | 56 |
| 1x PBS | Mean | 100.0 | 107.6 | 94.9 | 104.6 | 1092 | 102.1 | 91.5 | 77.5 | 87.4 |
| | SD | 0.0 | 23.4 | 18.0 | 28.4 | 42.1 | 28.1 | 23.9 | 22.1 | 31.8 |
| | N | 5 | 5 | 5 | 5 | 5 | 5 | 5 | 5 | 5 |
| SRS-000231 | Mean | 100.0 | 208.2 | 2483 | 2449 | 1952 | 219.7 | 223.8 | 206.4 | 196.7 |
| | SD | 0.0 | 107.1 | 97.8 | 142.6 | 143.6 | 114.7 | 110.9 | 98.1 | 109.8 |
| | N | 4 | 4 | 3 | 4 | 4 | 4 | 4 | 4 | 4 |
| SRS-001860 | Mean | 1000 | 258.7 | 292.7 | 277.1 | 2432 | 243.1 | 260.1 | 227.0 | 213.6 |
| | SD | 0.0 | 190.7 | 232.7 | 184.1 | 163.6 | 1653 | 178.1 | 150.8 | 138.5 |
| | N | 4 | 4 | 4 | 4 | 4 | 4 | 4 | 4 | 4 |
| SRS-001861 | Mean | 100.0 | 202.6 | 210.7 | 215.6 | 209.0 | 1955 | 200.3 | 191.0 | 191.7 |
| | SD | 0.0 | 78.3 | 85.1 | 76.7 | 79.0 | 67.7 | 71.3 | 77.8 | 72.6 |
| | N | 5 | 5 | 5 | 5 | 5 | 5 | 5 | 5 | 5 |
| SRS-001862 | Mean | 100.0 | 178.7 | 178.7 | 185.5 | 1818 | 1695 | 168.7 | 158.6 | 160.7 |
| | SD | 0.0 | 65.9 | 72.1 | 59.7 | 67.3 | 60.9 | 60.6 | 60.0 | 55.8 |
| | N | | 5 | 5 | 5 | 5 | 5 | 5 | 5 | 5 |
| SRS-001863 | Mean | 1000 | 1719 | 1949 | 176.7 | 1743 | 159.6 | 174.1 | 159.1 | 159.6 |
| | SD | 0.0 | 104.8 | 136.8 | 116.7 | 1025 | 96.8 | 100.8 | 93.1 | 89.3 |
| | N | 5 | 5 | 5 | 5 | 5 | 5 | 5 | 5 | 5 |
| SRS-001864 | Mean | 100.0 | 125.8 | 140.7 | 149.1 | 147.7 | 148.4 | 153.8 | 153.6 | 147.3 |
| | SD | 0.0 | 45.1 | 41.5 | 48.1 | 44.6 | 44.5 | 46.2 | 51.2 | 47.2 |
| | N | 5 | 5 | 5 | 5 | 5 | 5 | 5 | 5 | 5 |
| SRS-001865 | Mean | 100.0 | 221.6 | 229.1 | 244.4 | 2453 | 2182 | 228.3 | 195.7 | 209.1 |
| | SD | 0.0 | 74.3 | 79.3 | 84.8 | 83.0 | 68.7 | 74.9 | 71.6 | 106.1 |
| | N | 5 | 5 | 5 | 5 | 5 | 5 | 5 | 5 | 5 |
| SRS-001866 | Mean | 100.0 | 167.5 | 191.0 | 197.7 | 211.6 | 196.7 | 201.4 | 1843 | 163.2 |
| | SD | 0.0 | 62.2 | 69.6 | 64.1 | 74.2 | 65.5 | 67.4 | 63.1 | 59.2 |
| | N | 5 | 4 | 4 | 4 | 4 | 4 | 4 | 4 | 4 |
| SRS-001868 | Mean | 100.0 | 1799 | 1845 | 184.4 | 178.4 | 158.9 | 180.8 | 161.4 | 1579 |
| | SD | 0.0 | 67.8 | 84.0 | 80.5 | 82.8 | 73.2 | 79.2 | 59.9 | 69.4 |
| | N | 5 | 5 | 5 | 5 | 5 | 5 | 5 | 5 | 5 |
| SRS-001869 | Mean | 100.0 | 193.3 | 187.5 | 193.1 | 196.6 | 175.3 | 183.5 | 1718 | 172.2 |
| | SD | 0.0 | 103.6 | 79.6 | 89.2 | 95.0 | 80.0 | 70.2 | 68.4 | 77.9 |
| | N | 4 | 4 | 4 | 4 | 4 | 4 | 4 | 4 | 4 |
| SRS-001870 | Mean | 100.0 | 201.3 | 289.8 | 223.7 | 2293 | 205.8 | 202.6 | 203.1 | 200.4 |
| | SD | 0.0 | 169.8 | 305.8 | 163.1 | 203.6 | 186.7 | 174.1 | 179.8 | 179.0 |
| | N | 4 | 4 | 4 | 4 | 3 | 3 | 3 | 3 | 3 |

TABLE 16
Percentage of LDL-Cholesterol Relative to
Day 1 Following Treatment in Transgenic Mice
Percent LDL-Cholesterol Relative to Day 1

| | | Study Day | | | | | | | | |
|---|---|---|---|---|---|---|---|---|---|---|
| SRS ID | Statistics | 1 | 7 | 14 | 21 | 28 | 35 | 42 | 49 | 56 |
| 1x PBS | Mean | 100.0 | 120.7 | 55.4 | 59.3 | 99.4 | 71.1 | 63.4 | 109.7 | 64.1 |
| | SD | 0.0 | 56.2 | 32.5 | 42.0 | 126.8 | 31.6 | 369 | 127.2 | 64.0 |
| | N | 5 | 5 | 5 | 5 | 5 | 5 | 5 | 5 | 5 |
| SRS-000231 | Mean | 100.0 | 4.8 | 6.0 | 5.2 | 38.4 | 4.7 | 4.9 | 4.8 | 6.6 |
| | SD | 0.0 | 1.9 | 1.2 | 2.4 | 49.3 | 2.0 | 1.8 | 1.9 | 2.4 |
| | N | 4 | 4 | 3 | 4 | 4 | 4 | 4 | 4 | 4 |
| SRS-001860 | Mean | 100.0 | 4.6 | 7.4 | 9.0 | 7.7 | 5.9 | 6.9 | 4.2 | 6.3 |
| | SD | 0.0 | 1.7 | 5.1 | 6.2 | 4.7 | 3.6 | 3.8 | 1.9 | 3.3 |
| | N | 4 | 4 | 4 | 4 | 4 | 4 | 4 | 4 | 4 |
| SRS-001861 | Mean | 100.0 | 12.6 | 14.9 | 15.0 | 23.3 | 13.3 | 12.8 | 12.2 | 3.5 |
| | SD | 0.0 | 12.7 | 139 | 13.1 | 14.0 | 12.7 | 12.2 | 12.6 | 13.0 |
| | N | 5 | 5 | 5 | 5 | 5 | 5 | 5 | 5 | 5 |
| SRS-001862 | Mean | 100.0 | 14.3 | 19.0 | 21.6 | 22.3 | 15.1 | 15.7 | 13.5 | 14.3 |
| | SD | 0.0 | 14.1 | 22.5 | 22.5 | 18.1 | 14.6 | 14.7 | 13.9 | 13.7 |
| | N | 5 | 5 | 5 | 5 | 5 | 5 | 5 | 5 | 5 |
| SRS-001863 | Mean | 100.0 | 17.0 | 17.4 | 229 | 18.8 | 174 | 17.3 | 19.7 | 20.1 |
| | SD | 0.0 | 18.9 | 18.5 | 29.5 | 21.2 | 18.5 | 18.7 | 24.3 | 22.0 |
| | N | 5 | 5 | 5 | 5 | 5 | 5 | 5 | 5 | |
| SRS-001864 | Mean | 100.0 | 9.0 | 9.1 | 9.0 | 9.0 | 9.0 | 9.0 | 9.0 | 9.0 |
| | SD | 0.0 | 5.8 | 5.7 | 5.8 | 5.8 | 5.8 | 5.8 | 5.8 | 5.8 |
| | N | 5 | 5 | 5 | 5 | 5 | 5 | 5 | 5 | 5 |
| SRS-001865 | Mean | 100.0 | 16.0 | 16.5 | 16.0 | 24.0 | 16.2 | 163 | 17.1 | 30.0 |
| | SD | 0.0 | 10.8 | 10.8 | 10.8 | 15.5 | 10.6 | 10.5 | 9.6 | 29.6 |
| | N | 5 | 5 | 5 | 5 | 5 | 5 | 5 | 5 | 5 |
| SRS-001866 | Mean | 100.0 | 12.4 | 12.4 | 12.4 | 23.7 | 22.7 | 12.4 | 12.4 | 13.8 |
| | SD | 0.0 | 7.6 | 7.6 | 7.6 | 26.1 | 26.6 | 7.6 | 7.6 | 8.3 |
| | N | 5 | 4 | 4 | 4 | 4 | 4 | 4 | 4 | 4 |
| SRS-001868 | Mean | 100.0 | 5.3 | 6.3 | 5.4 | 7.1 | 5.3 | 5.6 | 5.2 | 5.4 |
| | SD | 0.0 | 4.0 | 3.5 | 4.0 | 5.1 | 4.0 | 4.0 | 4.0 | 3.9 |
| | N | 5 | 5 | 5 | 5 | 5 | 5 | 5 | 5 | 5 |
| SRS-001869 | Mean | 100.0 | 13.6 | 13.5 | 13.9 | 39.6 | 13.5 | 13.5 | 13.5 | 13.5 |
| | SD | 0.0 | 18.4 | 18.5 | 18.2 | 56.7 | 18.5 | 18.5 | 18.5 | 18.5 |
| | N | 4 | 4 | 4 | 4 | 4 | 4 | 4 | 4 | 4 |
| SRS-001870 | Mean | 100.0 | 7.0 | 9.4 | 7.5 | 9.9 | 6.9 | 6.9 | 6.9 | 7.2 |
| | SD | 0.0 | 5.1 | 5.2 | 4.6 | 7.0 | 6.2 | 6.2 | 6.2 | 6.0 |
| | N | 4 | 4 | 4 | 4 | 3 | 3 | 3 | 3 | 3 |

TABLE 17
Percentage of Plasma APOC3 Relative to
Day 1 Following Treatment in Transgenic Mice
Percent Plasma APOC3 Relative to Day 1

| | | Study Day | | | | | | | |
|---|---|---|---|---|---|---|---|---|---|
| SRS ID | Statistics | 3 | 7 | 14 | 21 | 28 | 35 | 49 | 56 |
| 1x PBS | Mean | 100.0 | 119.8 | 112.2 | 97.5 | 68.6 | 68.5 | 65.1 | 59.5 |
| | SD | 2.8 | 12.6 | 16.7 | 16.1 | 20.0 | 15.6 | 179 | 14.0 |
| | N | 5 | 4 | 4 | 5 | 5 | 4 | 5 | 5 |
| SRS-000231 | Mean | 1000 | 26.5 | 30.4 | 25.6 | 11.4 | 11.0 | 12.7 | 19.5 |
| | SD | 3.0 | 5.2 | 6.2 | 10.9 | 6.9 | 4.0 | 6.7 | 9.8 |
| | N | 5 | 3 | 4 | 3 | 4 | 4 | 4 | 4 |
| SRS-001860 | Mean | 100.0 | 35.2 | 29.8 | 22.6 | 10.7 | 7.2 | 10.7 | 11.6 |
| | SD | 5.3 | 2.9 | 6.0 | 6.7 | 1.5 | 2.3 | 2.5 | 1.7 |
| | N | 4 | 3 | 4 | 3 | 5 | 5 | 4 | 5 |
| SRS-001861 | Mean | 100.0 | 30.2 | 35.2 | 23.1 | 16.5 | 9.0 | 13.9 | 12.5 |
| | SD | 4.4 | 8.3 | 5.5 | 7.5 | 3.9 | 2.9 | 3.4 | 3.8 |
| | N | 5 | 3 | 4 | 3 | 4 | 5 | 5 | 5 |
| SRS-001862 | Mean | 100.0 | 38.8 | 34.1 | 29.4 | 149 | 9.2 | 11.8 | 13.0 |
| | SD | 5.0 | 11.6 | 4.0 | 4.2 | 4.2 | 2.5 | 2.1 | 3.5 |
| | N | 4 | 5 | 4 | 4 | 5 | 5 | 5 | 5 |
| SRS-001863 | Mean | 1000 | 27.6 | 35.5 | 20.6 | 10.7 | 7.9 | 13.3 | 10.0 |
| | SD | 3.3 | 8.7 | 13.8 | 4.8 | 5.0 | 4.5 | 6.8 | 5.0 |
| | N | 5 | 5 | 5 | 5 | 5 | 5 | 4 | 5 |
| SRS-001864 | Mean | 100.0 | 28.9 | 37.6 | 25.7 | 0.8 | 7.6 | 12.8 | 9.7 |
| | SD | 2.7 | 10.6 | 8.2 | 6.8 | 2.5 | 2.0 | 3.2 | 2.5 |
| | N | 5 | 5 | 5 | 5 | 5 | 5 | 5 | 5 |
| SRS-001865 | Mean | 100.0 | 38.3 | 45.3 | 34.2 | 12.7 | 9.6 | 27.1 | 25.0 |
| | SD | 4.0 | 14.1 | 12.6 | 8.5 | 2.6 | 3.3 | 8.8 | 15.4 |
| | N | 5 | 5 | 5 | 5 | 5 | 5 | 5 | 4 |
| SRS-001866 | Mean | 100.0 | 25.2 | 33.3 | 23.8 | 13.1 | 5.2 | 10.3 | 11.0 |
| | SD | 4.2 | 5.1 | 17.9 | 4.4 | 3.8 | 2.5 | 1.9 | 5.5 |
| | N | 5 | 4 | 2 | 4 | 4 | 2 | 4 | 5 |
| SRS-001868 | Mean | 100.0 | 28.6 | 40.0 | 25.9 | 7.2 | 5.6 | 14.4 | 7.4 |
| | SD | 5.7 | 6.7 | 3.7 | 5.3 | 2.1 | 1.9 | 7.2 | 1.7 |
| | N | 5 | 3 | 4 | 2 | 4 | 5 | 5 | 5 |
| SRS-001869 | Mean | 100.0 | 31.6 | 41.8 | 25.5 | 13.9 | 7.0 | 15.6 | 17.8 |
| | SD | 2.9 | 10.4 | 15.3 | 12.1 | 2.9 | 2.0 | 2.2 | 3.2 |
| | N | 4 | 3 | 3 | 4 | 3 | 3 | 2 | 3 |
| SRS-001870 | Mean | 100.0 | 19.9 | 34.6 | 26.0 | 9.5 | 8.3 | 13.2 | 14.5 |
| | SD | 7.0 | 3.9 | 6.2 | 7.5 | 1.4 | 1.7 | 2.3 | 3.5 |
| | N | 4 | 3 | 3 | 4 | 4 | 3 | 3 | 3 |

TABLE 18
APOC3 mRNA Expression Relative to PBS Control
Following Treatment in Transgenic Mice
Relative Expression of Liver APOC3 mRNA

| SRS ID | Statistics | Day 56 Results |
|---|---|---|
| 1x PBS | Mean | 1.01 |
| | SD | 0.11 |
| | N | 3 |
| SRS-000231 | Mean | 0.26 |
| | SD | 0.16 |
| | N | 5 |
| SRS-001860 | Mean | 0.07 |
| | SD | 0.06 |
| | N | 4 |
| SRS-001861 | Mean | 0.05 |
| | SD | 0.04 |
| | N | 5 |
| SRS-001862 | Mean | 0.03 |
| | SD | 0.01 |
| | N | 5 |
| SRS-001863 | Mean | 0.06 |
| | SD | 0.06 |
| | N | 5 |
| SRS-001864 | Mean | 0.13 |
| | SD | 0.11 |
| | N | 5 |
| SRS-001865 | Mean | 0.62 |
| | SD | 0.35 |
| | N | 4 |
| SRS-001866 | Mean | 0.12 |
| | SD | 0.05 |
| | N | 4 |
| SRS-001868 | Mean | 0.14 |
| | SD | 0.06 |
| | N | 5 |
| SRS-001869 | Mean | 0.21 |
| | SD | 0.10 |
| | N | 4 |
| SRS-0018670 | Mean | 0.18 |
| | SD | 0.11 |
| | N | 3 |

What is claimed is:

1. A polynucleic acid molecule for modulating expression of apolipoprotein C3 (APOC3) gene, comprising a sense strand and an antisense strand, wherein the antisense strand comprises a nucleic acid sequence that is at least 80%, at least 85%, at least 90%, at least 95% identical to a nucleic acid sequence selected from SEQ ID NOs: 1-108, 433-456, 529-534, and 553-563.

2. The polynucleic acid molecule of claim 1, wherein the antisense strand comprises a nucleic acid sequence comprising at least 14, 15, 16, 17, 18, 19, 20, 21, or 22 consecutive sequences of a nucleic acid sequence selected from SEQ ID NOs: 1-108, 433-456, 529-534, and 553-563 with no more than 1, 2, 3, or 4 mismatches.

3. The polynucleic acid molecule of claim 1 or 2, wherein the sense strand comprises a nucleic acid sequence that is at least 80%, at least 85%, at least 90%, at least 95% identical to a nucleic acid sequence selected from SEQ ID NOs: 217-324, 481-504, 541-546, and 575-585.

4. The polynucleic acid molecule of any one of claims 1-3, wherein the sense strand comprises a nucleic acid sequence comprising at least 14, 15, 16, 17, 18, 19, or 20 consecutive sequences of a nucleic acid sequence selected from SEQ ID NOs: 217-324, 481-504, 541-546, and 575-585 with no more than 1, 2, 3, or 4 mismatches.

5. The polynucleic acid molecule of any one of claims 1-4, wherein the sense strand comprises a nucleic acid sequence of SEQ ID NOs: 217-324, 481-504, 541-546, and 575-585 and the antisense strand comprises a nucleic acid sequence selected from SEQ ID NOs: 1-108, 433-456, 529-534, and 553-563.

6. The polynucleic acid molecule of any one of claims 1-5, wherein the polynucleic acid molecule comprises (1) a 2′-fluoro modified nucleotides; (2) a 2′-O-methyl modified nucleotides; (3) 2′-deoxy modified nucleotides, or (4) a modified internucleotide linkage.

7. The polynucleic acid molecule of claim 6, wherein the polynucleic acid molecule comprise at least two consecutive modified internucleotide linkages at the 5′ end or at the 3′ end.

8. The polynucleic acid molecule of any one of claims 6-7, wherein the antisense strand comprises 5′-nNfnnnNfnfNffnnnnNfnNfnnnnnnn-3′, 5′-nNfnnnNfnnnnnnnNfnNfnnnnnnn-3′, 5′-nNfnnnnNfnnnnNfnNfnnnnnnnnn-3′, 5′-nNfnnnnNfnnnnNfnNfnNfnnnnnnn-3′, or 5′-nNfnnnnnrnnnnNfnNfnNfnnnnnnn-3′, wherein “Nf” stands for a 2′-fluoro modified nucleotide, and wherein “n” stands for a 2′-O-methyl modified nucleotide.

9. The polynucleic acid molecule of any one of claims 6-8, wherein the sense strand comprises 5′-nnnnnnNfnNfnNfnnnnnnnnnn-3′, 5′-nnnnnnNfnNfNfNfnnnnnnnnn-3′, or 5′-nnnnnnnnNfNfNfnnnnnnnnnn-3′, wherein “Nf” stands for a 2′-fluoro modified nucleotide, and wherein “n” stands for a 2′-O-methyl modified nucleotide.

10. The polynucleic acid molecule of any one of claims 6-9, wherein
i) the sense strand comprises 5′-NfnNfnNfnNfnNfNfNfnNfnNfnNfnNfnNf-3′, the antisense strand comprises 5′-nNfnNfnNfnNfnNfnnnNfnNfnNfnNfnnn-3′;
ii) the sense strand comprises 5′-nnnnnnNfnNfNfNfnnnnnnnnnn-3′, the antisense strand comprises 5′-nNfnnnNfnNfNfnnnnNfnNfnnnnnnn-3′;
iii) the sense strand comprises 5′-nnnnnnnnNfnNfnnnnnnnnnn-3′, wherein the antisense strand comprises 5′-nNfnnnnnnnnnNfnNfnnnnnnnnn-3′;
iv) the sense strand comprises 5′-nnnnnnNfnNfnNfnnnnnnnnnn-3′, the antisense strand comprises 5′-nNfnnnnnnnnnNfnNfnNfnnnnnnn-3′; or
v) the sense strand comprises 5′-nnnnnnNfnNfnNfnnnnnnnnnn-3′, the antisense strand comprises 5′-nNfnnnnNfnnnnNfnNfnNfnnnnnnn-3′,
wherein “Nf” stands for a 2′-fluoro modified nucleotide, and wherein “n” stands for a 2′-O-methyl modified nucleotide.

11. The polynucleic acid molecule of any one of claims 6-10, wherein the modified internucleotide linkage is a phosphorothioate internucleotide linkage.

12. The polynucleic acid molecule of any one of claims 6-11, wherein the modified internucleotide linkage comprises a stereochemically enriched phosphorothioate internucleotide linkage.

13. The polynucleic acid molecule of any one of claims 1-12, wherein the sense strand comprises a nucleic acid sequence that is at least 80%, at least 85%, at least 90%, at least 95% identical to a nucleic acid sequence selected from SEQ ID NOs: 325-432, 505-528, 547-552, and 586-596.

14. The polynucleic acid molecule of any one of claims 1-13, wherein the antisense strand comprises a nucleic acid sequence that is at least 80%, at least 85%, at least 90%, at least 95% identical to a nucleic acid sequence selected from SEQ ID NOs: 109-216, 457-480, 535-540, and 564-574.

15. The polynucleic acid molecule ofany one of claims 1-14, wherein the sense strand comprises a nucleic acid sequence selected from SEQ ID NOs: 325-432, 505-528, 547-552, and 586-596, and the antisense strand comprises a nucleic acid sequence selected from SEQ ID NOs: 109-216, 457-480, 535-540, and 564-574.

16. A polynucleic acid molecule for modulating expression of apolipoprotein C3 (APOC3) gene, wherein polynucleic acid molecule comprises:
(a) an antisense strand comprising the nucleotide sequence of UUUCAGGGAACUGAAGCCAUCGG (SEQ ID NO:529) and a sense strand comprising the nucleotide sequence of GAUGGCUUCAGUUCCCUGAAA (SEQ ID NO:541);
(b) an antisense strand comprising the nucleotide sequence of UGAAUACUGUCCCUUUUAAGCAA (SEQ ID NO:530) and a sense strand comprising the nucleotide sequence of GCUUAAAAGGGACAGUAUUCA (SEQ ID NO:542);
(c) an antisense strand comprising the nucleotide sequence of UAGAAUACUGUCCCUUUUAAGCA (SEQ ID NO:531) and a sense strand comprising the nucleotide sequence of CUUAAAAGGGACAGUAUUCUA (SEQ ID NO:543);
(d) an antisense strand comprising the nucleotide sequence of UUGAGAAUACUGUCCCUUUUAAG (SEQ ID NO:532) and a sense strand comprising the nucleotide sequence of UAAAAGGGACAGUAUUCUCAA (SEQ ID NO:544);
(e) an antisense strand comprising the nucleotide sequence of UCUGAGAAUACUGUCCCUUUAA (SEQ ID NO:533) and a sense strand comprising the nucleotide sequence of AAAAGGGACAGUAUUCUCAGA (SEQ ID NO:545);
(f) an antisense strand comprising the nucleotide sequence of UCACUGAGAAUACUGUCCCUUUU (SEQ ID NO:534) and a sense strand comprising the nucleotide sequence of AAGGGACAGUAUUCUCAGUGA (SEQ ID NO:546);
(g) an antisense strand comprising the nucleotide sequence of UCUGAGAAUACUGUCCCUUUUAA (SEQ ID NO: 554) and a sense strand comprising the nucleotide sequence of AAAAGGGACAGUAUUCUCAGA (SEQ ID NO: 576);
(h) an antisense strand comprising the nucleotide sequence of UCUGAGAAUACUGUCCCUUUCAA (SEQ ID NO: 555) and a sense strand comprising the nucleotide sequence of GAAAGGGACAGUAUUCUCAGA (SEQ ID NO: 577);
(i) an antisense strand comprising the nucleotide sequence of UCUGAGAAUACUGUCCCUUUGAA (SEQ ID NO: 556) and a sense strand comprising the nucleotide sequence of CAAAGGGACAGUAUUCUCAGA (SEQ ID NO: 578);
(j) an antisense strand comprising the nucleotide sequence of UCUGAGAAUACUGUCCCUUGCAA (SEQ ID NO: 557) and a sense strand comprising the nucleotide sequence of GCAAGGGACAGUAUUCUCAGA (SEQ ID NO: 579);
(k) an antisense strand comprising the nucleotide sequence of UCUGAGAAUACUGUCCCUUCGAA (SEQ ID NO: 558) and a sense strand comprising the nucleotide sequence of CGAAGGGACAGUAUUCUCAGA (SEQ ID NO: 580);
(l) an antisense strand comprising the nucleotide sequence of UCUGAGAAUACUGUCCCUUUUAA (SEQ ID NO: 559) and a sense strand comprising the nucleotide sequence of AAAAGGGACAGUAUUCUCAGA (SEQ ID NO: 581);
(m) an antisense strand comprising the nucleotide sequence of UCUGAGAAUACUGUCCCUUUGAA (SEQ ID NO: 560) and a sense strand comprising the nucleotide sequence of CAAAGGGACAGUAUUCUCAGA (SEQ ID NO: 582);
(n) an antisense strand comprising the nucleotide sequence of UCUGAGAAUACUGUCCCUUUUAA (SEQ ID NO: 561) and a sense strand comprising the nucleotide sequence of AAAAGGGACAGUAUUCUCAGA (SEQ ID NO: 583);
(o) an antisense strand comprising the nucleotide sequence of UCUGAGAAUACUGUCCCUUUUAA (SEQ ID NO: 562) and a sense strand comprising the nucleotide sequence of AAAAGGGACAGUAUUCUCAGA (SEQ ID NO: 584); or
(p) an antisense strand comprising the nucleotide sequence of UCUGAGAAUACUGUCCCUUUUAA (SEQ ID NO: 563) and a sense strand comprising the nucleotide sequence of AAAAGGGACAGUAUUCUCAGA (SEQ ID NO: 585).

17. A polynucleic acid molecule for modulating expression of apolipoprotein C3 (APOC3) gene, wherein polynucleic acid molecule comprises:
(a) an antisense strand comprising the nucleotide sequence of usUfsucagGfgaacUfgAfaGfccaucsgsg (SEQ ID NO:535) and a sense strand comprising the nucleotide sequence of gsasuggcUfuCfaGfuucccugaaa (SEQ ID NO:547);
(b) an antisense strand comprising the nucleotide sequence of usGfsaauaCfugucCfcUfuUfuaagcsasa (SEQ ID NO:536) and a sense strand comprising the nucleotide sequence of gscsuuaaAfaGfgGfacaguauuca (SEQ ID NO:548);
(c) an antisense strand comprising the nucleotide sequence of usAfsgaauAfcuguCfcCfuUfuuaagscsa (SEQ ID NO:537) and a sense strand comprising the nucleotide sequence of csusuaaaAfgGfgAfcaguauucua (SEQ ID NO:549);
(d) an antisense strand comprising the nucleotide sequence of usUfsgagaAfuacuCfuCfcCfuuuuasasg (SEQ ID NO:538) and a sense strand comprising the nucleotide sequence of usasaaagGfgAfcAfguauucucaa (SEQ ID NO:550);
(e) an antisense strand comprising the nucleotide sequence of usCfsugagAfauacUfgUfcCfcuuuusasa (SEQ ID NO:539) and a sense strand comprising the nucleotide sequence of asasaaggGfaCfaGfuauucucaga (SEQ ID NO:551);
(f) an antisense strand comprising the nucleotide sequence of usCfsacugAfgaauAfcUfgUfcccuususu (SEQ ID NO:540) and a sense strand comprising the nucleotide sequence of asasgggaCfaGfuAfuucucaguga (SEQ ID NO:552);
(g) an antisense strand comprising the nucleotide sequence of vpusCfsugagAfauacUfgUfcCfcuuuusasa (SEQ ID NC: 565) and a sense strand comprising the nucleotide sequence of asasaaggGfaCfaGfuauucucaga (SEQ ID NO: 587);
(h) an antisense strand comprising the nucleotide sequence of usCfsugagAfauacUfgUfcCfcuuucsasa (SEQ ID NO: 566) and a sense strand comprising the nucleotide sequence of gsasaaggGfaCfaGfuauucucaga (SEQ ID NO: 588);
(i) an antisense strand comprising the nucleotide sequence of usCfsugagAfauacUfgUfcCfcuuugsasa (SEQ ID NO: 567) and a sense strand comprising the nucleotide sequence of csasaaggGfaCfaGfuauucucaga (SEQ ID NO: 589);
(j) an antisense strand comprising the nucleotide sequence of usCfsugagAfauacUfgUfcCfcuugcsasa (SEQ ID NO: 568) and a sense strand comprising the nucleotide sequence of gscsaaggGfaCfaGfuauuccaga (SEQ ID NO: 590);
(k) an antisense strand comprising the nucleotide sequence of usCfsugagAfauacUfgUfcCfuucgsasa (SEQ II) NO: 569) and a sense strand comprising the nucleotide sequence of csgsaaggGfaCfaGfuauucucaga (SEQ ID NO: 591);
(l) an antisense strand comprising the nucleotide sequence of usCfsugagAfauacUfgUfcCfcuuuusasa (SEQ ID NO: 570) and a sense strand comprising the nucleotide sequence of (invAb)asasaaggGfaCfaGfuauucucaga (SEQ ID NO: 592);
(in) an antisense strand comprising the nucleotide sequence of usCfsugagAfauacUfgUfcCfcuuugsasa (SEQ ID NO: 571) and a sense strand comprising the nucleotide sequence of (invAb)csasaaggGfaCfaGfuauucucaga (SEQ II) NO: 593);
(n) an antisense strand comprising the nucleotide sequence of usCfsugdAgdAauacUfgUfcCfcuuuusasa (SEQ ID NO: 572) and a sense strand comprising the nucleotide sequence of asasaaggGfaCfaGfuauucucaga (SEQ ID NO: 594);
(o) an antisense strand comprising the nucleotide sequence of usCfsugagAfauacUfgUfscCfcuuuusasa (SEQ ID NO: 573) and a sense strand comprising the nucleotide sequence of asasaaggGfaCfaCGfuauucucaga (SEQ ID NO: 595); or
(p) an antisense strand comprising the nucleotide sequence of usCfsugagAfauacUfgUfcCfcsuuuusasa (SEQ ID NO: 574) and a sense strand comprising the nucleotide sequence of asasaaggGfaCfaGfuauucucaga (SEQ ID NO: 596),
wherein “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “(invAb)” refers to inverted abasic deoxyribonucleotide, and “vp” refers to 5′-vinylphosphonate modified nucleotide.

18. A polynucleic acid molecule conjugate for modulating expression of apolipoprotein C3 (APOC3) gene, wherein the polynucleic acid molecule conjugate comprises a polynucleic acid molecule of any one of claims 1-17 and an asialoglycoprotein receptor targeting moiety.

19. The polynucleic acid molecule conjugate of claim 18, wherein the asialoglycoprotein receptor targeting moiety comprises N-Acetylgalactosamine (GalNAc) or galactose.

20. The polynucleic acid molecule conjugate of claim 18 or 19, wherein the polynucleic acid molecule and the asialoglycoprotein receptor targeting moiety is coupled via a linker.

21. The polynucleic acid molecule conjugate of claim 20, wherein the linker comprises formula (IV) below, 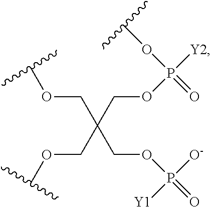
wherein at least one of Y1 and Y2 is a nucleotide in the polynucleic acid molecule.

22. The polynucleic acid molecule conjugate of claim 21, wherein the Y1 is the last nucleotide on the 3′-terminus of the sense strand of the polynucleic acid molecule or the Y1 and Y2 are two consecutive nucleotides in the polynucleic acid molecule.

23. The polynucleic acid molecule conjugate of any one of claims 20-22, wherein the linker and the asialoglycoprotein receptor targeting moiety with the last nucleotide on the 3′-terminus of the sense strand of the polynucleic acid molecule are shown in Formula (V′), (V″″), (V′″″), or (V″″″): 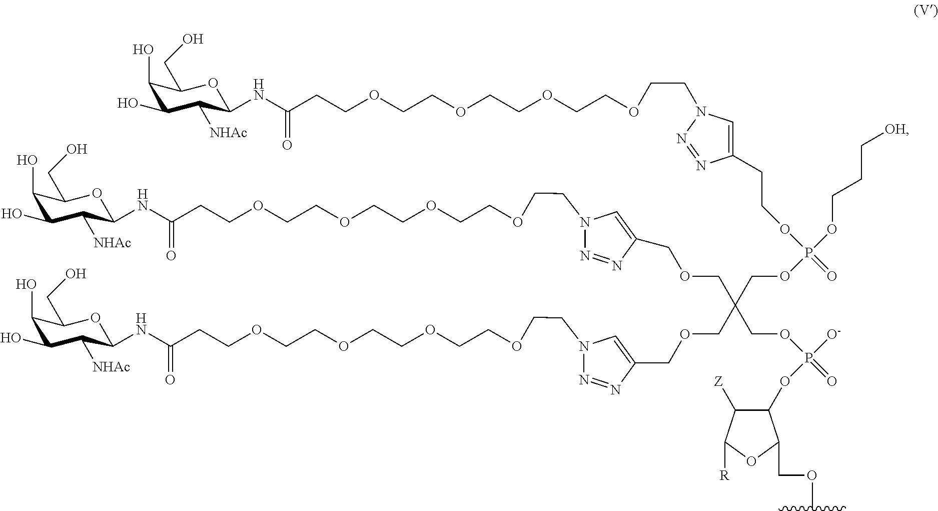
wherein Z in formula (V′) is —H, —OH, —O-Methyl, —F, or —O-methoxyethyl, and R in formula (V′) is adenine, uracil, guanine, cytosine, thymine, abasic, or others; 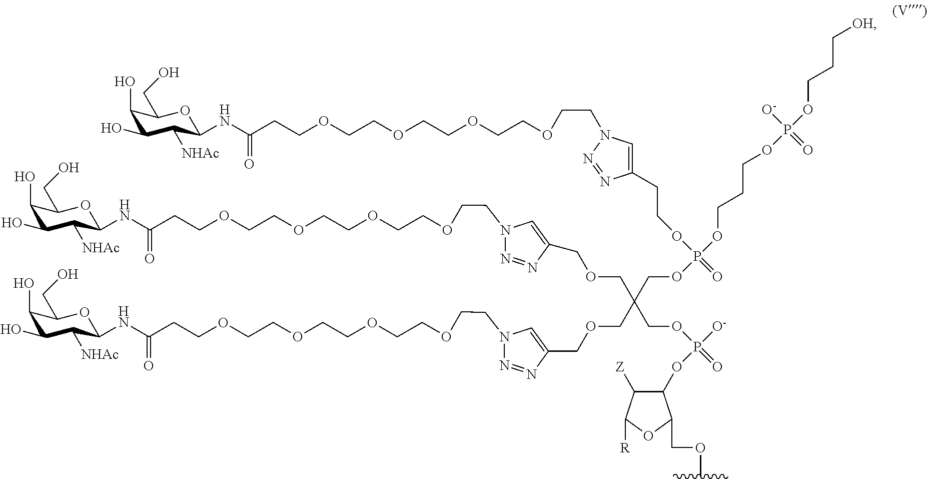
wherein Z in formula (V″″) is a moiety that corresponds to one of the sugar modifications described herein (e.g., —H, —OH, —O-Methyl, —F, or —O-methoxyethyl) and R in formula (V″″) is adenine, uracil, guanine, cytosine, thymine, abasic, or others; 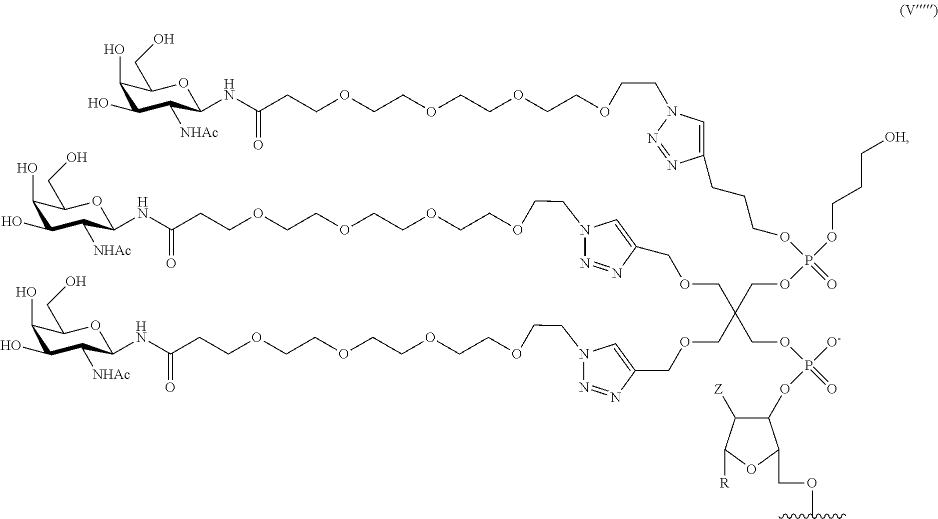
wherein Z in formula (V′″″) is a moiety that corresponds to one of the sugar modifications described herein (e.g., —H, —OH, —O-Methyl, —F, or —O-methoxyethyl) and R in formula (V′″″) is adenine, uracil, guanine, cytosine, thymine, abasic, or others; 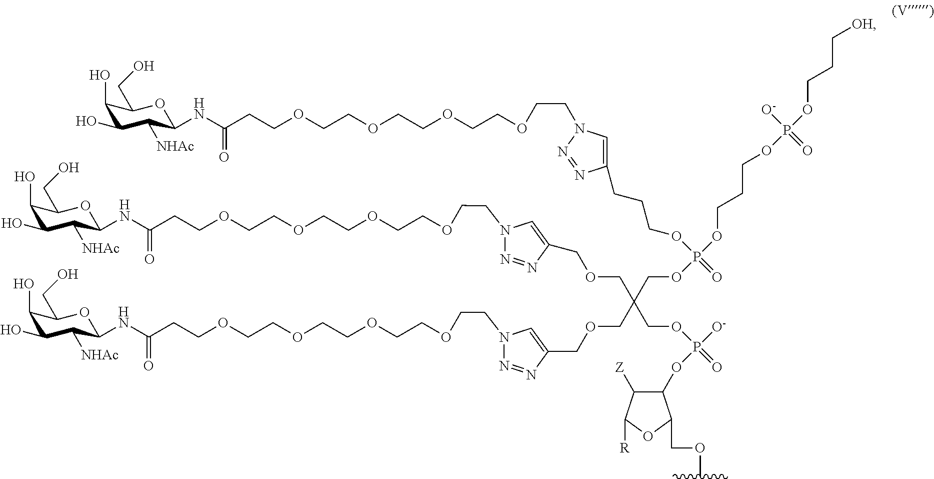
wherein Z in formula (V″″″) is a moiety that corresponds to one of the sugar modifications described herein (e.g., —H, —OH, —O-Methyl, —F, or —O-methoxyethyl) and R in formula (V″″″) is adenine, uracil, guanine, cytosine, thymine, abasic, or others.

24. The polynucleic acid molecule conjugate of claim 23, wherein the linker and the asialoglycoprotein receptor targeting moiety with the last nucleotide on the 3′-terminus of the sense strand of the polynucleic acid molecule are shown in Formula (V′): 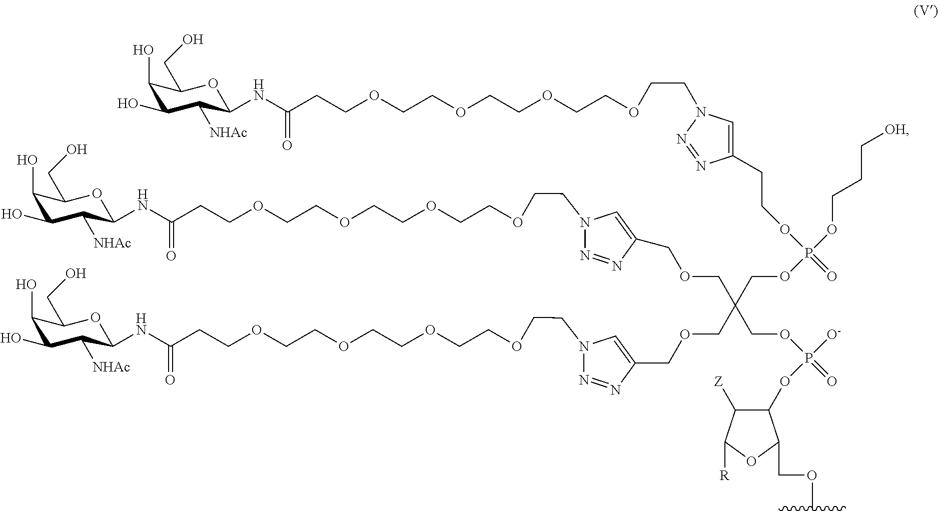
wherein Z in formula (V′) is —H, —OH, —O-Methyl, —F, or —O-methoxyethyl, and R in formula (V′) is adenine, uracil, guanine, cytosine, thymine, abasic, or others.

25. A pharmaceutical composition comprising a polynucleic acid molecule of any one of claims 1-17 or a polynucleic acid molecule conjugate of any one of claims 18-24, and a pharmaceutically acceptable excipient.

26. The pharmaceutical composition of claim 25, wherein the pharmaceutical composition is formulated as a nanoparticle formulation.

27. The pharmaceutical composition of claim 25 or 26, wherein the pharmaceutical composition is formulated for parenteral, oral, intranasal, buccal, rectal, transdermal, intravenous, subcutaneous, or intrathecal administration.

28. A method of modulating expression of apolipoprotein C3 (APOC3) gene in a subject in need thereof, comprising: administering to the subject a polynucleic acid molecule of any one of claims 1-17 or a polynucleic acid molecule conjugate of any one of claims 18-24, or a pharmaceutical composition of any one of claims 25-27, thereby modulating the expression of APOC3 gene in the subject.

29. A method of modulating triglyceride level in a subject in need thereof, comprising: administering to the subject a polynucleic acid molecule of any one of claims 1-17 or a polynucleic acid molecule conjugate of any one of claims 18-24, or a pharmaceutical composition of any one of claims 25-27, thereby modulating triglyceride level in the subject.

30. The method of claim 28 or 29, wherein the subject in need thereof is diagnosed of, suffers from, or having a symptom of cardiovascular disease or hypertriglyceridemia.


SEQUENCE LISTING (598 sequences)

SEQ ID NO: 1 - RNA, 23 nt, synthetic construct
  source 1..23: mol_type = other RNA
tcatggcacctctgttcctggag

SEQ ID NO: 2 - RNA, 23 nt, synthetic construct
  source 1..23: mol_type = other RNA
ttacccggggctgcatggcacct

SEQ ID NO: 3 - RNA, 23 nt, synthetic construct
  source 1..23: mol_type = other RNA
tgtacccggggctgcatggcacc

SEQ ID NO: 4 - RNA, 23 nt, synthetic construct
  source 1..23: mol_type = other RNA
tagtacccggggctgcatggcac

SEQ ID NO: 5 - RNA, 23 nt, synthetic construct
  source 1..23: mol_type = other RNA
taaggagtacccggggctgcatg

SEQ ID NO: 6 - RNA, 23 nt, synthetic construct
  source 1..23: mol_type = other RNA
tacaaggagtacccggggctgca

SEQ ID NO: 7 - RNA, 23 nt, synthetic construct
  source 1..23: mol_type = other RNA
taacaaggagtacccggggctgc

SEQ ID NO: 8 - RNA, 23 nt, synthetic construct
  source 1..23: mol_type = other RNA
tacaacaaggagtacccggggct

SEQ ID NO: 9 - RNA, 23 nt, synthetic construct
  source 1..23: mol_type = other RNA
taacaacaaggagtacccggggc

SEQ ID NO: 10 - RNA, 23 nt, synthetic construct
  source 1..23: mol_type = other RNA
tgcaacaacaaggagtacccggg

SEQ ID NO: 11 - RNA, 23 nt, synthetic construct
  source 1..23: mol_type = other RNA
tggcaacaacaaggagtacccgg

SEQ ID NO: 12 - RNA, 23 nt, synthetic construct
  source 1..23: mol_type = other RNA
tgggcaacaacaaggagtacccg

SEQ ID NO: 13 - RNA, 23 nt, synthetic construct
  source 1..23: mol_type = other RNA
tagggcaacaacaaggagtaccc

SEQ ID NO: 14 - RNA, 23 nt, synthetic construct
  source 1..23: mol_type = other RNA
tcaggagggcaacaacaaggagt

SEQ ID NO: 15 - RNA, 23 nt, synthetic construct
  source 1..23: mol_type = other RNA
tgccaggagggcaacaacaagga

SEQ ID NO: 16 - RNA, 23 nt, synthetic construct
  source 1..23: mol_type = other RNA
tagctcgggcagaggccaggagc

SEQ ID NO: 17 - RNA, 23 nt, synthetic construct
  source 1..23: mol_type = other RNA
tgaagctcgggcagaggccagga

SEQ ID NO: 18 - RNA, 23 nt, synthetic construct
  source 1..23: mol_type = other RNA
tctgaagctcgggcagaggccag

SEQ ID NO: 19 - RNA, 23 nt, synthetic construct
  source 1..23: mol_type = other RNA
ttctgaagctcgggcagaggcca

SEQ ID NO: 20 - RNA, 23 nt, synthetic construct
  source 1..23: mol_type = other RNA
tcctctgaagctcgggcagaggc

SEQ ID NO: 21 - RNA, 23 nt, synthetic construct
  source 1..23: mol_type = other RNA
tggcctctgaagctcgggcagag

SEQ ID NO: 22 - RNA, 23 nt, synthetic construct
  source 1..23: mol_type = other RNA
tcggcctctgaagctcgggcaga

SEQ ID NO: 23 - RNA, 23 nt, synthetic construct
  source 1..23: mol_type = other RNA
ttcggcctctgaagctcgggcag

SEQ ID NO: 24 - RNA, 23 nt, synthetic construct
  source 1..23: mol_type = other RNA
tctcggcctctgaagctcgggca

SEQ ID NO: 25 - RNA, 23 nt, synthetic construct
  source 1..23: mol_type = other RNA
tatcctcggcctctgaagctcgg

SEQ ID NO: 26 - RNA, 23 nt, synthetic construct
  source 1..23: mol_type = other RNA
taagggaggcatcctcggcctct

SEQ ID NO: 27 - RNA, 23 nt, synthetic construct
  source 1..23: mol_type = other RNA
tgaagggaggcatcctcggcctc

SEQ ID NO: 28 - RNA, 23 nt, synthetic construct
  source 1..23: mol_type = other RNA
tagaagggaggcatcctcggcct

SEQ ID NO: 29 - RNA, 23 nt, synthetic construct
  source 1..23: mol_type = other RNA
tgagaagggaggcatcctcggcc

SEQ ID NO: 30 - RNA, 23 nt, synthetic construct
  source 1..23: mol_type = other RNA
tcttggtggcgtgcttcatgtaa

SEQ ID NO: 31 - RNA, 23 nt, synthetic construct
  source 1..23: mol_type = other RNA
tggtcttggtggcgtgcttcatg

SEQ ID NO: 32 - RNA, 23 nt, synthetic construct
  source 1..23: mol_type = other RNA
tgcggtcttggtggcgtgcttca

SEQ ID NO: 33 - RNA, 23 nt, synthetic construct
  source 1..23: mol_type = other RNA
tggcggtcttggtggcgtgcttc

SEQ ID NO: 34 - RNA, 23 nt, synthetic construct
  source 1..23: mol_type = other RNA
ttccttggcggtcttggtggcgt

SEQ ID NO: 35 - RNA, 23 nt, synthetic construct
  source 1..23: mol_type = other RNA
tcatccttggcggtcttggtggc

SEQ ID NO: 36 - RNA, 23 nt, synthetic construct
  source 1..23: mol_type = other RNA
ttgcatccttggcggtcttggtg

SEQ ID NO: 37 - RNA, 23 nt, synthetic construct
  source 1..23: mol_type = other RNA
tgtgcatccttggcggtcttggt

SEQ ID NO: 38 - RNA, 23 nt, synthetic construct
  source 1..23: mol_type = other RNA
tcgctgctcagtgcatccttggc

SEQ ID NO: 39 - RNA, 23 nt, synthetic construct
  source 1..23: mol_type = other RNA
tacgctgctcagtgcatccttgg

SEQ ID NO: 40 - RNA, 23 nt, synthetic construct
  source 1..23: mol_type = other RNA
tcacgctgctcagtgcatccttg

SEQ ID NO: 41 - RNA, 23 nt, synthetic construct
  source 1..23: mol_type = other RNA
ttgcacgctgctcagtgcatcct

SEQ ID NO: 42 - RNA, 23 nt, synthetic construct
  source 1..23: mol_type = other RNA
ttgggactcctgcacgctgctca

SEQ ID NO: 43 - RNA, 23 nt, synthetic construct
  source 1..23: mol_type = other RNA
ttcggtcacccagcccctggcct

SEQ ID NO: 44 - RNA, 23 nt, synthetic construct
  source 1..23: mol_type = other RNA
tatcggtcacccagcccctggcc

SEQ ID NO: 45 - RNA, 23 nt, synthetic construct
  source 1..23: mol_type = other RNA
tagccatcggtcacccagcccct

SEQ ID NO: 46 - RNA, 23 nt, synthetic construct
  source 1..23: mol_type = other RNA
tgaagccatcggtcacccagccc

SEQ ID NO: 47 - RNA, 23 nt, synthetic construct
  source 1..23: mol_type = other RNA
tctgaagccatcggtcacccagc

SEQ ID NO: 48 - RNA, 23 nt, synthetic construct
  source 1..23: mol_type = other RNA
tactgaagccatcggtcacccag

SEQ ID NO: 49 - RNA, 23 nt, synthetic construct
  source 1..23: mol_type = other RNA
taactgaagccatcggtcaccca

SEQ ID NO: 50 - RNA, 23 nt, synthetic construct
  source 1..23: mol_type = other RNA
tgaactgaagccatcggtcaccc

SEQ ID NO: 51 - RNA, 23 nt, synthetic construct
  source 1..23: mol_type = other RNA
tggaactgaagccatcggtcacc

SEQ ID NO: 52 - RNA, 23 nt, synthetic construct
  source 1..23: mol_type = other RNA
tttcagggaactgaagccatcgg

SEQ ID NO: 53 - RNA, 23 nt, synthetic construct
  source 1..23: mol_type = other RNA
tagtagtctttcagggaactgaa

SEQ ID NO: 54 - RNA, 23 nt, synthetic construct
  source 1..23: mol_type = other RNA
tcggtgctccagtagtctttcag

SEQ ID NO: 55 - RNA, 23 nt, synthetic construct
  source 1..23: mol_type = other RNA
taacggtgctccagtagtctttc

SEQ ID NO: 56 - RNA, 23 nt, synthetic construct
  source 1..23: mol_type = other RNA
ttaacggtgctccagtagtcttt

SEQ ID NO: 57 - RNA, 23 nt, synthetic construct
  source 1..23: mol_type = other RNA
tttaacggtgctccagtagtctt

SEQ ID NO: 58 - RNA, 23 nt, synthetic construct
  source 1..23: mol_type = other RNA
tcttaacggtgctccagtagtct

SEQ ID NO: 59 - RNA, 23 nt, synthetic construct
  source 1..23: mol_type = other RNA
tccttaacggtgctccagtagtc

SEQ ID NO: 60 - RNA, 23 nt, synthetic construct
  source 1..23: mol_type = other RNA
ttccttaacggtgctccagtagt

SEQ ID NO: 61 - RNA, 23 nt, synthetic construct
  source 1..23: mol_type = other RNA
tgtccttaacggtgctccagtag

SEQ ID NO: 62 - RNA, 23 nt, synthetic construct
  source 1..23: mol_type = other RNA
ttgtccttaacggtgctccagta

SEQ ID NO: 63 - RNA, 23 nt, synthetic construct
  source 1..23: mol_type = other RNA
tttgtccttaacggtgctccagt

SEQ ID NO: 64 - RNA, 23 nt, synthetic construct
  source 1..23: mol_type = other RNA
tcttgtccttaacggtgctccag

SEQ ID NO: 65 - RNA, 23 nt, synthetic construct
  source 1..23: mol_type = other RNA
tacttgtccttaacggtgctcca

SEQ ID NO: 66 - RNA, 23 nt, synthetic construct
  source 1..23: mol_type = other RNA
tgaacttgtccttaacggtgctc

SEQ ID NO: 67 - RNA, 23 nt, synthetic construct
  source 1..23: mol_type = other RNA
tagaacttgtccttaacggtgct

SEQ ID NO: 68 - RNA, 23 nt, synthetic construct
  source 1..23: mol_type = other RNA
tagagaacttgtccttaacggtg

SEQ ID NO: 69 - RNA, 23 nt, synthetic construct
  source 1..23: mol_type = other RNA
tcagagaacttgtccttaacggt

SEQ ID NO: 70 - RNA, 23 nt, synthetic construct
  source 1..23: mol_type = other RNA
taactcagagaacttgtccttaa

SEQ ID NO: 71 - RNA, 23 nt, synthetic construct
  source 1..23: mol_type = other RNA
tgggtccaaatcccagaactcag

SEQ ID NO: 72 - RNA, 23 nt, synthetic construct
  source 1..23: mol_type = other RNA
tgaagttggtctgacctcagggt

SEQ ID NO: 73 - RNA, 23 nt, synthetic construct
  source 1..23: mol_type = other RNA
tacggctgaagttggtctgacct

SEQ ID NO: 74 - RNA, 23 nt, synthetic construct
  source 1..23: mol_type = other RNA
tccacggctgaagttggtctgac

SEQ ID NO: 75 - RNA, 23 nt, synthetic construct
  source 1..23: mol_type = other RNA
tgtattgaggtctcaggcagcca

SEQ ID NO: 76 - RNA, 23 nt, synthetic construct
  source 1..23: mol_type = other RNA
tggtattgaggtctcaggcagcc

SEQ ID NO: 77 - RNA, 23 nt, synthetic construct
  source 1..23: mol_type = other RNA
tggggtattgaggtctcaggcag

SEQ ID NO: 78 - RNA, 23 nt, synthetic construct
  source 1..23: mol_type = other RNA
ttggggtattgaggtctcaggca

SEQ ID NO: 79 - RNA, 23 nt, synthetic construct
  source 1..23: mol_type = other RNA
tacttggggtattgaggtctcag

SEQ ID NO: 80 - RNA, 23 nt, synthetic construct
  source 1..23: mol_type = other RNA
tgacttggggtattgaggtctca

SEQ ID NO: 81 - RNA, 23 nt, synthetic construct
  source 1..23: mol_type = other RNA
tggacttggggtattgaggtctc

SEQ ID NO: 82 - RNA, 23 nt, synthetic construct
  source 1..23: mol_type = other RNA
tgattgcaggacccaaggagctc

SEQ ID NO: 83 - RNA, 23 nt, synthetic construct
  source 1..23: mol_type = other RNA
tgagattgcaggacccaaggagc

SEQ ID NO: 84 - RNA, 23 nt, synthetic construct
  source 1..23: mol_type = other RNA
tggagattgcaggacccaaggag

SEQ ID NO: 85 - RNA, 23 nt, synthetic construct
  source 1..23: mol_type = other RNA
ttggagattgcaggacccaagga

SEQ ID NO: 86 - RNA, 23 nt, synthetic construct
  source 1..23: mol_type = other RNA
tctggagattgcaggacccaagg

SEQ ID NO: 87 - RNA, 23 nt, synthetic construct
  source 1..23: mol_type = other RNA
tcctggagattgcaggacccaag

SEQ ID NO: 88 - RNA, 23 nt, synthetic construct
  source 1..23: mol_type = other RNA
tccctggagattgcaggacccaa

SEQ ID NO: 89 - RNA, 23 nt, synthetic construct
  source 1..23: mol_type = other RNA
tgccctggagattgcaggaccca

SEQ ID NO: 90 - RNA, 23 nt, synthetic construct
  source 1..23: mol_type = other RNA
tcccttttaagcaacctacaggg

SEQ ID NO: 91 - RNA, 23 nt, synthetic construct
  source 1..23: mol_type = other RNA
tgtcccttttaagcaacctacag

SEQ ID NO: 92 - RNA, 23 nt, synthetic construct
  source 1..23: mol_type = other RNA
tgaatactgtcccttttaagcaa

SEQ ID NO: 93 - RNA, 23 nt, synthetic construct
  source 1..23: mol_type = other RNA
tgagaatactgtcccttttaagc

SEQ ID NO: 94 - RNA, 23 nt, synthetic construct
  source 1..23: mol_type = other RNA
ttgagaatactgtcccttttaag

SEQ ID NO: 95 - RNA, 23 nt, synthetic construct
  source 1..23: mol_type = other RNA
tctgagaatactgtcccttttaa

SEQ ID NO: 96 - RNA, 23 nt, synthetic construct
  source 1..23: mol_type = other RNA
tactgagaatactgtccctttta

SEQ ID NO: 97 - RNA, 23 nt, synthetic construct
  source 1..23: mol_type = other RNA
tcactgagaatactgtccctttt

SEQ ID NO: 98 - RNA, 23 nt, synthetic construct
  source 1..23: mol_type = other RNA
tgcactgagaatactgtcccttt

SEQ ID NO: 99 - RNA, 23 nt, synthetic construct
  source 1..23: mol_type = other RNA
tagagcactgagaatactgtccc

SEQ ID NO: 100 - RNA, 23 nt, synthetic construct
  source 1..23: mol_type = other RNA
tgagagcactgagaatactgtcc

SEQ ID NO: 101 - RNA, 23 nt, synthetic construct
  source 1..23: mol_type = other RNA
tgtaggagagcactgagaatact

SEQ ID NO: 102 - RNA, 23 nt, synthetic construct
  source 1..23: mol_type = other RNA
tcatgaggtggggtaggagagca

SEQ ID NO: 103 - RNA, 23 nt, synthetic construct
  source 1..23: mol_type = other RNA
taggcatgaggtggggtaggaga

SEQ ID NO: 104 - RNA, 23 nt, synthetic construct
  source 1..23: mol_type = other RNA
tttattgggaggccagcatgcct

SEQ ID NO: 105 - RNA, 23 nt, synthetic construct
  source 1..23: mol_type = other RNA
ttttattgggaggccagcatgcc

SEQ ID NO: 106 - RNA, 23 nt, synthetic construct
  source 1..23: mol_type = other RNA
tgctttattgggaggccagcatg

SEQ ID NO: 107 - RNA, 23 nt, synthetic construct
  source 1..23: mol_type = other RNA
tcagctttattgggaggccagca

SEQ ID NO: 108 - RNA, 23 nt, synthetic construct
  source 1..23: mol_type = other RNA
tccagctttattgggaggccagc

SEQ ID NO: 109 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcatggcacctctgttcctggag

SEQ ID NO: 110 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttacccggggctgcatggcacct

SEQ ID NO: 111 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtacccggggctgcatggcacc

SEQ ID NO: 112 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tagtacccggggctgcatggcac

SEQ ID NO: 113 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
taaggagtacccggggctgcatg

SEQ ID NO: 114 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tacaaggagtacccggggctgca

SEQ ID NO: 115 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
taacaaggagtacccggggctgc

SEQ ID NO: 116 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tacaacaaggagtacccggggct

SEQ ID NO: 117 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
taacaacaaggagtacccggggc

SEQ ID NO: 118 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgcaacaacaaggagtacccggg

SEQ ID NO: 119 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tggcaacaacaaggagtacccgg

SEQ ID NO: 120 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgggcaacaacaaggagtacccg

SEQ ID NO: 121 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tagggcaacaacaaggagtaccc

SEQ ID NO: 122 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcaggagggcaacaacaaggagt

SEQ ID NO: 123 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgccaggagggcaacaacaagga

SEQ ID NO: 124 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tagctcgggcagaggccaggagc

SEQ ID NO: 125 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgaagctcgggcagaggccagga

SEQ ID NO: 126 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctgaagctcgggcagaggccag

SEQ ID NO: 127 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttctgaagctcgggcagaggcca

SEQ ID NO: 128 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcctctgaagctcgggcagaggc

SEQ ID NO: 129 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tggcctctgaagctcgggcagag

SEQ ID NO: 130 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcggcctctgaagctcgggcaga

SEQ ID NO: 131 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttcggcctctgaagctcgggcag

SEQ ID NO: 132 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctcggcctctgaagctcgggca

SEQ ID NO: 133 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tatcctcggcctctgaagctcgg

SEQ ID NO: 134 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
taagggaggcatcctcggcctct

SEQ ID NO: 135 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgaagggaggcatcctcggcctc

SEQ ID NO: 136 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tagaagggaggcatcctcggcct

SEQ ID NO: 137 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgagaagggaggcatcctcggcc

SEQ ID NO: 138 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcttggtggcgtgcttcatgtaa

SEQ ID NO: 139 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tggtcttggtggcgtgcttcatg

SEQ ID NO: 140 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgcggtcttggtggcgtgcttca

SEQ ID NO: 141 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tggcggtcttggtggcgtgcttc

SEQ ID NO: 142 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttccttggcggtcttggtggcgt

SEQ ID NO: 143 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcatccttggcggtcttggtggc

SEQ ID NO: 144 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgcatccttggcggtcttggtg

SEQ ID NO: 145 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtgcatccttggcggtcttggt

SEQ ID NO: 146 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcgctgctcagtgcatccttggc

SEQ ID NO: 147 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tacgctgctcagtgcatccttgg

SEQ ID NO: 148 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcacgctgctcagtgcatccttg

SEQ ID NO: 149 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgcacgctgctcagtgcatcct

SEQ ID NO: 150 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgggactcctgcacgctgctca

SEQ ID NO: 151 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttcggtcacccagcccctggcct

SEQ ID NO: 152 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tatcggtcacccagcccctggcc

SEQ ID NO: 153 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tagccatcggtcacccagcccct

SEQ ID NO: 154 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgaagccatcggtcacccagccc

SEQ ID NO: 155 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctgaagccatcggtcacccagc

SEQ ID NO: 156 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tactgaagccatcggtcacccag

SEQ ID NO: 157 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
taactgaagccatcggtcaccca

SEQ ID NO: 158 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgaactgaagccatcggtcaccc

SEQ ID NO: 159 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tggaactgaagccatcggtcacc

SEQ ID NO: 160 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tttcagggaactgaagccatcgg

SEQ ID NO: 161 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tagtagtctttcagggaactgaa

SEQ ID NO: 162 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcggtgctccagtagtctttcag

SEQ ID NO: 163 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
taacggtgctccagtagtctttc

SEQ ID NO: 164 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttaacggtgctccagtagtcttt

SEQ ID NO: 165 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tttaacggtgctccagtagtctt

SEQ ID NO: 166 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcttaacggtgctccagtagtct

SEQ ID NO: 167 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tccttaacggtgctccagtagtc

SEQ ID NO: 168 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttccttaacggtgctccagtagt

SEQ ID NO: 169 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtccttaacggtgctccagtag

SEQ ID NO: 170 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgtccttaacggtgctccagta

SEQ ID NO: 171 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tttgtccttaacggtgctccagt

SEQ ID NO: 172 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcttgtccttaacggtgctccag

SEQ ID NO: 173 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tacttgtccttaacggtgctcca

SEQ ID NO: 174 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgaacttgtccttaacggtgctc

SEQ ID NO: 175 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tagaacttgtccttaacggtgct

SEQ ID NO: 176 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tagagaacttgtccttaacggtg

SEQ ID NO: 177 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcagagaacttgtccttaacggt

SEQ ID NO: 178 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
taactcagagaacttgtccttaa

SEQ ID NO: 179 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgggtccaaatcccagaactcag

SEQ ID NO: 180 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgaagttggtctgacctcagggt

SEQ ID NO: 181 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tacggctgaagttggtctgacct

SEQ ID NO: 182 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tccacggctgaagttggtctgac

SEQ ID NO: 183 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtattgaggtctcaggcagcca

SEQ ID NO: 184 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tggtattgaggtctcaggcagcc

SEQ ID NO: 185 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tggggtattgaggtctcaggcag

SEQ ID NO: 186 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttggggtattgaggtctcaggca

SEQ ID NO: 187 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tacttggggtattgaggtctcag

SEQ ID NO: 188 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgacttggggtattgaggtctca

SEQ ID NO: 189 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tggacttggggtattgaggtctc

SEQ ID NO: 190 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgattgcaggacccaaggagctc

SEQ ID NO: 191 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgagattgcaggacccaaggagc

SEQ ID NO: 192 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tggagattgcaggacccaaggag

SEQ ID NO: 193 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttggagattgcaggacccaagga

SEQ ID NO: 194 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctggagattgcaggacccaagg

SEQ ID NO: 195 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcctggagattgcaggacccaag

SEQ ID NO: 196 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tccctggagattgcaggacccaa

SEQ ID NO: 197 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgccctggagattgcaggaccca

SEQ ID NO: 198 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcccttttaagcaacctacaggg

SEQ ID NO: 199 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtcccttttaagcaacctacag

SEQ ID NO: 200 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgaatactgtcccttttaagcaa

SEQ ID NO: 201 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgagaatactgtcccttttaagc

SEQ ID NO: 202 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgagaatactgtcccttttaag

SEQ ID NO: 203 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctgagaatactgtcccttttaa

SEQ ID NO: 204 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tactgagaatactgtccctttta

SEQ ID NO: 205 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcactgagaatactgtccctttt

SEQ ID NO: 206 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgcactgagaatactgtcccttt

SEQ ID NO: 207 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tagagcactgagaatactgtccc

SEQ ID NO: 208 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgagagcactgagaatactgtcc

SEQ ID NO: 209 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtaggagagcactgagaatact

SEQ ID NO: 210 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcatgaggtggggtaggagagca

SEQ ID NO: 211 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
taggcatgaggtggggtaggaga

SEQ ID NO: 212 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tttattgggaggccagcatgcct

SEQ ID NO: 213 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttttattgggaggccagcatgcc

SEQ ID NO: 214 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgctttattgggaggccagcatg

SEQ ID NO: 215 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcagctttattgggaggccagca

SEQ ID NO: 216 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tccagctttattgggaggccagc

SEQ ID NO: 217 - RNA, 21 nt, synthetic construct
  source 1..21: mol_type = other RNA
ccaggaacagaggtgccatga

SEQ ID NO: 218 - RNA, 21 nt, synthetic construct
  source 1..21: mol_type = other RNA
gtgccatgcagccccgggtaa

SEQ ID NO: 219 - RNA, 21 nt, synthetic construct
  source 1..21: mol_type = other RNA
tgccatgcagccccgggtaca

SEQ ID NO: 220 - RNA, 21 nt, synthetic construct
  source 1..21: mol_type = other RNA
gccatgcagccccgggtacta

SEQ ID NO: 221 - RNA, 21 nt, synthetic construct
  source 1..21: mol_type = other RNA
tgcagccccgggtactcctta

SEQ ID NO: 222 - RNA, 21 nt, synthetic construct
  source 1..21: mol_type = other RNA
cagccccgggtactccttgta

SEQ ID NO: 223 - RNA, 21 nt, synthetic construct
  source 1..21: mol_type = other RNA
agccccgggtactccttgtta

SEQ ID NO: 224 - RNA, 21 nt, synthetic construct
  source 1..21: mol_type = other RNA
ccccgggtactccttgttgta

SEQ ID NO: 225 - RNA, 21 nt, synthetic construct
  source 1..21: mol_type = other RNA
cccgggtactccttgttgtta

SEQ ID NO: 226 - RNA, 21 nt, synthetic construct
  source 1..21: mol_type = other RNA
cgggtactccttgttgttgca

SEQ ID NO: 227 - RNA, 21 nt, synthetic construct
  source 1..21: mol_type = other RNA
gggtactccttgttgttgcca

SEQ ID NO: 228 - RNA, 21 nt, synthetic construct
  source 1..21: mol_type = other RNA
ggtactccttgttgttgccca

SEQ ID NO: 229 - RNA, 21 nt, synthetic construct
  source 1..21: mol_type = other RNA
gtactccttgttgttgcccta

SEQ ID NO: 230 - RNA, 21 nt, synthetic construct
  source 1..21: mol_type = other RNA
tccttgttgttgccctcctga

SEQ ID NO: 231 - RNA, 21 nt, synthetic construct
  source 1..21: mol_type = other RNA
cttgttgttgccctcctggca

SEQ ID NO: 232 - RNA, 21 nt, synthetic construct
  source 1..21: mol_type = other RNA
tcctggcctctgcccgagcta

SEQ ID NO: 233 - RNA, 21 nt, synthetic construct
  source 1..21: mol_type = other RNA
ctggcctctgcccgagcttca

SEQ ID NO: 234 - RNA, 21 nt, synthetic construct
  source 1..21: mol_type = other RNA
ggcctctgcccgagcttcaga

SEQ ID NO: 235 - RNA, 21 nt, synthetic construct
  source 1..21: mol_type = other RNA
gcctctgcccgagcttcagaa

SEQ ID NO: 236 - RNA, 21 nt, synthetic construct
  source 1..21: mol_type = other RNA
ctctgcccgagcttcagagga

SEQ ID NO: 237 - RNA, 21 nt, synthetic construct
  source 1..21: mol_type = other RNA
ctgcccgagcttcagaggcca

SEQ ID NO: 238 - RNA, 21 nt, synthetic construct
  source 1..21: mol_type = other RNA
tgcccgagcttcagaggccga

SEQ ID NO: 239 - RNA, 21 nt, synthetic construct
  source 1..21: mol_type = other RNA
gcccgagcttcagaggccgaa

SEQ ID NO: 240 - RNA, 21 nt, synthetic construct
  source 1..21: mol_type = other RNA
cccgagcttcagaggccgaga

SEQ ID NO: 241 - RNA, 21 nt, synthetic construct
  source 1..21: mol_type = other RNA
gagcttcagaggccgaggata

SEQ ID NO: 242 - RNA, 21 nt, synthetic construct
  source 1..21: mol_type = other RNA
aggccgaggatgcctccctta

SEQ ID NO: 243 - RNA, 21 nt, synthetic construct
  source 1..21: mol_type = other RNA
ggccgaggatgcctcccttca

SEQ ID NO: 244 - RNA, 21 nt, synthetic construct
  source 1..21: mol_type = other RNA
gccgaggatgcctcccttcta

SEQ ID NO: 245 - RNA, 21 nt, synthetic construct
  source 1..21: mol_type = other RNA
ccgaggatgcctcccttctca

SEQ ID NO: 246 - RNA, 21 nt, synthetic construct
  source 1..21: mol_type = other RNA
acatgaagcacgccaccaaga

SEQ ID NO: 247 - RNA, 21 nt, synthetic construct
  source 1..21: mol_type = other RNA
tgaagcacgccaccaagacca

SEQ ID NO: 248 - RNA, 21 nt, synthetic construct
  source 1..21: mol_type = other RNA
aagcacgccaccaagaccgca

SEQ ID NO: 249 - RNA, 21 nt, synthetic construct
  source 1..21: mol_type = other RNA
agcacgccaccaagaccgcca

SEQ ID NO: 250 - RNA, 21 nt, synthetic construct
  source 1..21: mol_type = other RNA
gccaccaagaccgccaaggaa

SEQ ID NO: 251 - RNA, 21 nt, synthetic construct
  source 1..21: mol_type = other RNA
caccaagaccgccaaggatga

SEQ ID NO: 252 - RNA, 21 nt, synthetic construct
  source 1..21: mol_type = other RNA
ccaagaccgccaaggatgcaa

SEQ ID NO: 253 - RNA, 21 nt, synthetic construct
  source 1..21: mol_type = other RNA
caagaccgccaaggatgcaca

SEQ ID NO: 254 - RNA, 21 nt, synthetic construct
  source 1..21: mol_type = other RNA
caaggatgcactgagcagcga

SEQ ID NO: 255 - RNA, 21 nt, synthetic construct
  source 1..21: mol_type = other RNA
aaggatgcactgagcagcgta

SEQ ID NO: 256 - RNA, 21 nt, synthetic construct
  source 1..21: mol_type = other RNA
aggatgcactgagcagcgtga

SEQ ID NO: 257 - RNA, 21 nt, synthetic construct
  source 1..21: mol_type = other RNA
gatgcactgagcagcgtgcaa

SEQ ID NO: 258 - RNA, 21 nt, synthetic construct
  source 1..21: mol_type = other RNA
agcagcgtgcaggagtcccaa

SEQ ID NO: 259 - RNA, 21 nt, synthetic construct
  source 1..21: mol_type = other RNA
gccaggggctgggtgaccgaa

SEQ ID NO: 260 - RNA, 21 nt, synthetic construct
  source 1..21: mol_type = other RNA
ccaggggctgggtgaccgata

SEQ ID NO: 261 - RNA, 21 nt, synthetic construct
  source 1..21: mol_type = other RNA
gggctgggtgaccgatggcta

SEQ ID NO: 262 - RNA, 21 nt, synthetic construct
  source 1..21: mol_type = other RNA
gctgggtgaccgatggcttca

SEQ ID NO: 263 - RNA, 21 nt, synthetic construct
  source 1..21: mol_type = other RNA
tgggtgaccgatggcttcaga

SEQ ID NO: 264 - RNA, 21 nt, synthetic construct
  source 1..21: mol_type = other RNA
gggtgaccgatggcttcagta

SEQ ID NO: 265 - RNA, 21 nt, synthetic construct
  source 1..21: mol_type = other RNA
ggtgaccgatggcttcagtta

SEQ ID NO: 266 - RNA, 21 nt, synthetic construct
  source 1..21: mol_type = other RNA
gtgaccgatggcttcagttca

SEQ ID NO: 267 - RNA, 21 nt, synthetic construct
  source 1..21: mol_type = other RNA
tgaccgatggcttcagttcca

SEQ ID NO: 268 - RNA, 21 nt, synthetic construct
  source 1..21: mol_type = other RNA
gatggcttcagttccctgaaa

SEQ ID NO: 269 - RNA, 21 nt, synthetic construct
  source 1..21: mol_type = other RNA
cagttccctgaaagactacta

SEQ ID NO: 270 - RNA, 21 nt, synthetic construct
  source 1..21: mol_type = other RNA
gaaagactactggagcaccga

SEQ ID NO: 271 - RNA, 21 nt, synthetic construct
  source 1..21: mol_type = other RNA
aagactactggagcaccgtta

SEQ ID NO: 272 - RNA, 21 nt, synthetic construct
  source 1..21: mol_type = other RNA
agactactggagcaccgttaa

SEQ ID NO: 273 - RNA, 21 nt, synthetic construct
  source 1..21: mol_type = other RNA
gactactggagcaccgttaaa

SEQ ID NO: 274 - RNA, 21 nt, synthetic construct
  source 1..21: mol_type = other RNA
actactggagcaccgttaaga

SEQ ID NO: 275 - RNA, 21 nt, synthetic construct
  source 1..21: mol_type = other RNA
ctactggagcaccgttaagga

SEQ ID NO: 276 - RNA, 21 nt, synthetic construct
  source 1..21: mol_type = other RNA
tactggagcaccgttaaggaa

SEQ ID NO: 277 - RNA, 21 nt, synthetic construct
  source 1..21: mol_type = other RNA
actggagcaccgttaaggaca

SEQ ID NO: 278 - RNA, 21 nt, synthetic construct
  source 1..21: mol_type = other RNA
ctggagcaccgttaaggacaa

SEQ ID NO: 279 - RNA, 21 nt, synthetic construct
  source 1..21: mol_type = other RNA
tggagcaccgttaaggacaaa

SEQ ID NO: 280 - RNA, 21 nt, synthetic construct
  source 1..21: mol_type = other RNA
ggagcaccgttaaggacaaga

SEQ ID NO: 281 - RNA, 21 nt, synthetic construct
  source 1..21: mol_type = other RNA
gagcaccgttaaggacaagta

SEQ ID NO: 282 - RNA, 21 nt, synthetic construct
  source 1..21: mol_type = other RNA
gcaccgttaaggacaagttca

SEQ ID NO: 283 - RNA, 21 nt, synthetic construct
  source 1..21: mol_type = other RNA
caccgttaaggacaagttcta

SEQ ID NO: 284 - RNA, 21 nt, synthetic construct
  source 1..21: mol_type = other RNA
ccgttaaggacaagttctcta

SEQ ID NO: 285 - RNA, 21 nt, synthetic construct
  source 1..21: mol_type = other RNA
cgttaaggacaagttctctga

SEQ ID NO: 286 - RNA, 21 nt, synthetic construct
  source 1..21: mol_type = other RNA
aaggacaagttctctgagtta

SEQ ID NO: 287 - RNA, 21 nt, synthetic construct
  source 1..21: mol_type = other RNA
gagttctgggatttggaccca

SEQ ID NO: 288 - RNA, 21 nt, synthetic construct
  source 1..21: mol_type = other RNA
cctgaggtcagaccaacttca

SEQ ID NO: 289 - RNA, 21 nt, synthetic construct
  source 1..21: mol_type = other RNA
gtcagaccaacttcagccgta

SEQ ID NO: 290 - RNA, 21 nt, synthetic construct
  source 1..21: mol_type = other RNA
cagaccaacttcagccgtgga

SEQ ID NO: 291 - RNA, 21 nt, synthetic construct
  source 1..21: mol_type = other RNA
gctgcctgagacctcaataca

SEQ ID NO: 292 - RNA, 21 nt, synthetic construct
  source 1..21: mol_type = other RNA
ctgcctgagacctcaatacca

SEQ ID NO: 293 - RNA, 21 nt, synthetic construct
  source 1..21: mol_type = other RNA
gcctgagacctcaatacccca

SEQ ID NO: 294 - RNA, 21 nt, synthetic construct
  source 1..21: mol_type = other RNA
cctgagacctcaataccccaa

SEQ ID NO: 295 - RNA, 21 nt, synthetic construct
  source 1..21: mol_type = other RNA
gagacctcaataccccaagta

SEQ ID NO: 296 - RNA, 21 nt, synthetic construct
  source 1..21: mol_type = other RNA
agacctcaataccccaagtca

SEQ ID NO: 297 - RNA, 21 nt, synthetic construct
  source 1..21: mol_type = other RNA
gacctcaataccccaagtcca

SEQ ID NO: 298 - RNA, 21 nt, synthetic construct
  source 1..21: mol_type = other RNA
gctccttgggtcctgcaatca

SEQ ID NO: 299 - RNA, 21 nt, synthetic construct
  source 1..21: mol_type = other RNA
tccttgggtcctgcaatctca

SEQ ID NO: 300 - RNA, 21 nt, synthetic construct
  source 1..21: mol_type = other RNA
ccttgggtcctgcaatctcca

SEQ ID NO: 301 - RNA, 21 nt, synthetic construct
  source 1..21: mol_type = other RNA
cttgggtcctgcaatctccaa

SEQ ID NO: 302 - RNA, 21 nt, synthetic construct
  source 1..21: mol_type = other RNA
ttgggtcctgcaatctccaga

SEQ ID NO: 303 - RNA, 21 nt, synthetic construct
  source 1..21: mol_type = other RNA
tgggtcctgcaatctccagga

SEQ ID NO: 304 - RNA, 21 nt, synthetic construct
  source 1..21: mol_type = other RNA
gggtcctgcaatctccaggga

SEQ ID NO: 305 - RNA, 21 nt, synthetic construct
  source 1..21: mol_type = other RNA
ggtcctgcaatctccagggca

SEQ ID NO: 306 - RNA, 21 nt, synthetic construct
  source 1..21: mol_type = other RNA
ctgtaggttgcttaaaaggga

SEQ ID NO: 307 - RNA, 21 nt, synthetic construct
  source 1..21: mol_type = other RNA
gtaggttgcttaaaagggaca

SEQ ID NO: 308 - RNA, 21 nt, synthetic construct
  source 1..21: mol_type = other RNA
gcttaaaagggacagtattca

SEQ ID NO: 309 - RNA, 21 nt, synthetic construct
  source 1..21: mol_type = other RNA
ttaaaagggacagtattctca

SEQ ID NO: 310 - RNA, 21 nt, synthetic construct
  source 1..21: mol_type = other RNA
taaaagggacagtattctcaa

SEQ ID NO: 311 - RNA, 21 nt, synthetic construct
  source 1..21: mol_type = other RNA
aaaagggacagtattctcaga

SEQ ID NO: 312 - RNA, 21 nt, synthetic construct
  source 1..21: mol_type = other RNA
aaagggacagtattctcagta

SEQ ID NO: 313 - RNA, 21 nt, synthetic construct
  source 1..21: mol_type = other RNA
aagggacagtattctcagtga

SEQ ID NO: 314 - RNA, 21 nt, synthetic construct
  source 1..21: mol_type = other RNA
agggacagtattctcagtgca

SEQ ID NO: 315 - RNA, 21 nt, synthetic construct
  source 1..21: mol_type = other RNA
gacagtattctcagtgctcta

SEQ ID NO: 316 - RNA, 21 nt, synthetic construct
  source 1..21: mol_type = other RNA
acagtattctcagtgctctca

SEQ ID NO: 317 - RNA, 21 nt, synthetic construct
  source 1..21: mol_type = other RNA
tattctcagtgctctcctaca

SEQ ID NO: 318 - RNA, 21 nt, synthetic construct
  source 1..21: mol_type = other RNA
ctctcctaccccacctcatga

SEQ ID NO: 319 - RNA, 21 nt, synthetic construct
  source 1..21: mol_type = other RNA
tcctaccccacctcatgccta

SEQ ID NO: 320 - RNA, 21 nt, synthetic construct
  source 1..21: mol_type = other RNA
gcatgctggcctcccaataaa

SEQ ID NO: 321 - RNA, 21 nt, synthetic construct
  source 1..21: mol_type = other RNA
catgctggcctcccaataaaa

SEQ ID NO: 322 - RNA, 21 nt, synthetic construct
  source 1..21: mol_type = other RNA
tgctggcctcccaataaagca

SEQ ID NO: 323 - RNA, 21 nt, synthetic construct
  source 1..21: mol_type = other RNA
ctggcctcccaataaagctga

SEQ ID NO: 324 - RNA, 21 nt, synthetic construct
  source 1..21: mol_type = other RNA
tggcctcccaataaagctgga

SEQ ID NO: 325 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ccaggaacagaggtgccatga

SEQ ID NO: 326 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gtgccatgcagccccgggtaa

SEQ ID NO: 327 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgccatgcagccccgggtaca

SEQ ID NO: 328 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gccatgcagccccgggtacta

SEQ ID NO: 329 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgcagccccgggtactcctta

SEQ ID NO: 330 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cagccccgggtactccttgta

SEQ ID NO: 331 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
agccccgggtactccttgtta

SEQ ID NO: 332 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ccccgggtactccttgttgta

SEQ ID NO: 333 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cccgggtactccttgttgtta

SEQ ID NO: 334 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cgggtactccttgttgttgca

SEQ ID NO: 335 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gggtactccttgttgttgcca

SEQ ID NO: 336 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ggtactccttgttgttgccca

SEQ ID NO: 337 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gtactccttgttgttgcccta

SEQ ID NO: 338 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tccttgttgttgccctcctga

SEQ ID NO: 339 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cttgttgttgccctcctggca

SEQ ID NO: 340 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tcctggcctctgcccgagcta

SEQ ID NO: 341 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctggcctctgcccgagcttca

SEQ ID NO: 342 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ggcctctgcccgagcttcaga

SEQ ID NO: 343 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcctctgcccgagcttcagaa

SEQ ID NO: 344 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctctgcccgagcttcagagga

SEQ ID NO: 345 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctgcccgagcttcagaggcca

SEQ ID NO: 346 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgcccgagcttcagaggccga

SEQ ID NO: 347 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcccgagcttcagaggccgaa

SEQ ID NO: 348 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cccgagcttcagaggccgaga

SEQ ID NO: 349 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gagcttcagaggccgaggata

SEQ ID NO: 350 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aggccgaggatgcctccctta

SEQ ID NO: 351 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ggccgaggatgcctcccttca

SEQ ID NO: 352 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gccgaggatgcctcccttcta

SEQ ID NO: 353 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ccgaggatgcctcccttctca

SEQ ID NO: 354 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
acatgaagcacgccaccaaga

SEQ ID NO: 355 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgaagcacgccaccaagacca

SEQ ID NO: 356 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aagcacgccaccaagaccgca

SEQ ID NO: 357 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
agcacgccaccaagaccgcca

SEQ ID NO: 358 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gccaccaagaccgccaaggaa

SEQ ID NO: 359 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
caccaagaccgccaaggatga

SEQ ID NO: 360 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ccaagaccgccaaggatgcaa

SEQ ID NO: 361 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
caagaccgccaaggatgcaca

SEQ ID NO: 362 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
caaggatgcactgagcagcga

SEQ ID NO: 363 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aaggatgcactgagcagcgta

SEQ ID NO: 364 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aggatgcactgagcagcgtga

SEQ ID NO: 365 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gatgcactgagcagcgtgcaa

SEQ ID NO: 366 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
agcagcgtgcaggagtcccaa

SEQ ID NO: 367 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gccaggggctgggtgaccgaa

SEQ ID NO: 368 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ccaggggctgggtgaccgata

SEQ ID NO: 369 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gggctgggtgaccgatggcta

SEQ ID NO: 370 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gctgggtgaccgatggcttca

SEQ ID NO: 371 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgggtgaccgatggcttcaga

SEQ ID NO: 372 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gggtgaccgatggcttcagta

SEQ ID NO: 373 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ggtgaccgatggcttcagtta

SEQ ID NO: 374 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gtgaccgatggcttcagttca

SEQ ID NO: 375 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgaccgatggcttcagttcca

SEQ ID NO: 376 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gatggcttcagttccctgaaa

SEQ ID NO: 377 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cagttccctgaaagactacta

SEQ ID NO: 378 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gaaagactactggagcaccga

SEQ ID NO: 379 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aagactactggagcaccgtta

SEQ ID NO: 380 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
agactactggagcaccgttaa

SEQ ID NO: 381 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gactactggagcaccgttaaa

SEQ ID NO: 382 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 9: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
actactggagcaccgttaaga

SEQ ID NO: 383 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctactggagcaccgttaagga

SEQ ID NO: 384 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tactggagcaccgttaaggaa

SEQ ID NO: 385 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
actggagcaccgttaaggaca

SEQ ID NO: 386 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctggagcaccgttaaggacaa

SEQ ID NO: 387 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tggagcaccgttaaggacaaa

SEQ ID NO: 388 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ggagcaccgttaaggacaaga

SEQ ID NO: 389 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gagcaccgttaaggacaagta

SEQ ID NO: 390 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcaccgttaaggacaagttca

SEQ ID NO: 391 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
caccgttaaggacaagttcta

SEQ ID NO: 392 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ccgttaaggacaagttctcta

SEQ ID NO: 393 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cgttaaggacaagttctctga

SEQ ID NO: 394 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aaggacaagttctctgagtta

SEQ ID NO: 395 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gagttctgggatttggaccca

SEQ ID NO: 396 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cctgaggtcagaccaacttca

SEQ ID NO: 397 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gtcagaccaacttcagccgta

SEQ ID NO: 398 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cagaccaacttcagccgtgga

SEQ ID NO: 399 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gctgcctgagacctcaataca

SEQ ID NO: 400 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctgcctgagacctcaatacca

SEQ ID NO: 401 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcctgagacctcaatacccca

SEQ ID NO: 402 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cctgagacctcaataccccaa

SEQ ID NO: 403 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gagacctcaataccccaagta

SEQ ID NO: 404 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
agacctcaataccccaagtca

SEQ ID NO: 405 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gacctcaataccccaagtcca

SEQ ID NO: 406 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gctccttgggtcctgcaatca

SEQ ID NO: 407 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tccttgggtcctgcaatctca

SEQ ID NO: 408 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ccttgggtcctgcaatctcca

SEQ ID NO: 409 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cttgggtcctgcaatctccaa

SEQ ID NO: 410 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ttgggtcctgcaatctccaga

SEQ ID NO: 411 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgggtcctgcaatctccagga

SEQ ID NO: 412 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gggtcctgcaatctccaggga

SEQ ID NO: 413 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ggtcctgcaatctccagggca

SEQ ID NO: 414 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctgtaggttgcttaaaaggga

SEQ ID NO: 415 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gtaggttgcttaaaagggaca

SEQ ID NO: 416 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcttaaaagggacagtattca

SEQ ID NO: 417 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ttaaaagggacagtattctca

SEQ ID NO: 418 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
taaaagggacagtattctcaa

SEQ ID NO: 419 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aaaagggacagtattctcaga

SEQ ID NO: 420 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aaagggacagtattctcagta

SEQ ID NO: 421 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aagggacagtattctcagtga

SEQ ID NO: 422 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
agggacagtattctcagtgca

SEQ ID NO: 423 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gacagtattctcagtgctcta

SEQ ID NO: 424 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
acagtattctcagtgctctca

SEQ ID NO: 425 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tattctcagtgctctcctaca

SEQ ID NO: 426 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctctcctaccccacctcatga

SEQ ID NO: 427 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tcctaccccacctcatgccta

SEQ ID NO: 428 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcatgctggcctcccaataaa

SEQ ID NO: 429 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
catgctggcctcccaataaaa

SEQ ID NO: 430 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgctggcctcccaataaagca

SEQ ID NO: 431 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctggcctcccaataaagctga

SEQ ID NO: 432 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tggcctcccaataaagctgga

SEQ ID NO: 433 - RNA, 23 nt, synthetic construct
  source 1..23: mol_type = other RNA
tagtacccggggctgcatggcac

SEQ ID NO: 434 - RNA, 23 nt, synthetic construct
  source 1..23: mol_type = other RNA
tacaaggagtacccggggctgca

SEQ ID NO: 435 - RNA, 23 nt, synthetic construct
  source 1..23: mol_type = other RNA
tggaactgaagccatcggtcacc

SEQ ID NO: 436 - RNA, 23 nt, synthetic construct
  source 1..23: mol_type = other RNA
tttcagggaactgaagccatcgg

SEQ ID NO: 437 - RNA, 23 nt, synthetic construct
  source 1..23: mol_type = other RNA
ttaacggtgctccagtagtcttt

SEQ ID NO: 438 - RNA, 23 nt, synthetic construct
  source 1..23: mol_type = other RNA
ttgtccttaacggtgctccagta

SEQ ID NO: 439 - RNA, 23 nt, synthetic construct
  source 1..23: mol_type = other RNA
tgaatactgtcccttttaagcaa

SEQ ID NO: 440 - RNA, 23 nt, synthetic construct
  source 1..23: mol_type = other RNA
tgagaatactgtcccttttaagc

SEQ ID NO: 441 - RNA, 23 nt, synthetic construct
  source 1..23: mol_type = other RNA
ttgagaatactgtcccttttaag

SEQ ID NO: 442 - RNA, 23 nt, synthetic construct
  source 1..23: mol_type = other RNA
tctgagaatactgtcccttttaa

SEQ ID NO: 443 - RNA, 23 nt, synthetic construct
  source 1..23: mol_type = other RNA
tactgagaatactgtccctttta

SEQ ID NO: 444 - RNA, 23 nt, synthetic construct
  source 1..23: mol_type = other RNA
tcactgagaatactgtccctttt

SEQ ID NO: 445 - RNA, 23 nt, synthetic construct
  source 1..23: mol_type = other RNA
tagtacccggggctgcatggcac

SEQ ID NO: 446 - RNA, 23 nt, synthetic construct
  source 1..23: mol_type = other RNA
tacaaggagtacccggggctgca

SEQ ID NO: 447 - RNA, 23 nt, synthetic construct
  source 1..23: mol_type = other RNA
  misc_feature 1..6: note = RNA
  misc_feature 7: note = DNA
  misc_feature 8..23: note = RNA
tggaactgaagccatcggtcacc

SEQ ID NO: 448 - RNA, 23 nt, synthetic construct
  source 1..23: mol_type = other RNA
tttcagggaactgaagccatcgg

SEQ ID NO: 449 - RNA, 23 nt, synthetic construct
  source 1..23: mol_type = other RNA
ttaacggtgctccagtagtcttt

SEQ ID NO: 450 - RNA, 23 nt, synthetic construct
  source 1..23: mol_type = other RNA
  misc_feature 1..6: note = RNA
  misc_feature 7: note = DNA
  misc_feature 8..23: note = RNA
ttgtccttaacggtgctccagta

SEQ ID NO: 451 - RNA, 23 nt, synthetic construct
  source 1..23: mol_type = other RNA
  misc_feature 1..4: note = RNA
  misc_feature 5: note = DNA
  misc_feature 6..23: note = RNA
tgaatactgtcccttttaagcaa

SEQ ID NO: 452 - RNA, 23 nt, synthetic construct
  source 1..23: mol_type = other RNA
  misc_feature 1..6: note = RNA
  misc_feature 7: note = DNA
  misc_feature 8..23: note = RNA
tgagaatactgtcccttttaagc

SEQ ID NO: 453 - RNA, 23 nt, synthetic construct
  source 1..23: mol_type = other RNA
ttgagaatactgtcccttttaag

SEQ ID NO: 454 - RNA, 23 nt, synthetic construct
  source 1..23: mol_type = other RNA
tctgagaatactgtcccttttaa

SEQ ID NO: 455 - RNA, 23 nt, synthetic construct
  source 1..23: mol_type = other RNA
tactgagaatactgtccctttta

SEQ ID NO: 456 - RNA, 23 nt, synthetic construct
  source 1..23: mol_type = other RNA
  misc_feature 1..4: note = RNA
  misc_feature 5: note = DNA
  misc_feature 6..23: note = RNA
tcactgagaatactgtccctttt

SEQ ID NO: 457 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tagtacccggggctgcatggcac

SEQ ID NO: 458 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tacaaggagtacccggggctgca

SEQ ID NO: 459 - RNA, 23 nt, synthetic construct
  source 1..23: mol_type = other RNA
tggaactgaagccatcggtcacc

SEQ ID NO: 460 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tttcagggaactgaagccatcgg

SEQ ID NO: 461 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttaacggtgctccagtagtcttt

SEQ ID NO: 462 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgtccttaacggtgctccagta

SEQ ID NO: 463 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgaatactgtcccttttaagcaa

SEQ ID NO: 464 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgagaatactgtcccttttaagc

SEQ ID NO: 465 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgagaatactgtcccttttaag

SEQ ID NO: 466 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctgagaatactgtcccttttaa

SEQ ID NO: 467 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tactgagaatactgtccctttta

SEQ ID NO: 468 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcactgagaatactgtccctttt

SEQ ID NO: 469 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..4: mod_base = OTHER; note = 2'O-Methyl ribonucleotide
  misc_feature 5: note = DNA
  modified_base 6: mod_base = OTHER; note = 2'O-Methyl ribonucleotide
  misc_feature 7: note = DNA
  modified_base 8..13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tagtacccggggctgcatggcac

SEQ ID NO: 470 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..4: mod_base = OTHER; note = 2'O-Methyl ribonucleotide
  misc_feature 5: note = DNA
  modified_base 6: mod_base = OTHER; note = 2'O-Methyl ribonucleotide
  misc_feature 7: note = DNA
  modified_base 8..13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tacaaggagtacccggggctgca

SEQ ID NO: 471 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..4: mod_base = OTHER; note = 2'O-Methyl ribonucleotide
  misc_feature 5: note = DNA
  modified_base 6: mod_base = OTHER; note = 2'O-Methyl ribonucleotide
  misc_feature 7: note = DNA
  modified_base 8..13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tggaactgaagccatcggtcacc

SEQ ID NO: 472 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..4: mod_base = OTHER; note = 2'O-Methyl ribonucleotide
  misc_feature 5: note = DNA
  modified_base 6: mod_base = OTHER; note = 2'O-Methyl ribonucleotide
  misc_feature 7: note = DNA
  modified_base 8..13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tttcagggaactgaagccatcgg

SEQ ID NO: 473 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..4: mod_base = OTHER; note = 2'O-Methyl ribonucleotide
  misc_feature 5: note = DNA
  modified_base 6: mod_base = OTHER; note = 2'O-Methyl ribonucleotide
  misc_feature 7: note = DNA
  modified_base 8..13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttaacggtgctccagtagtcttt

SEQ ID NO: 474 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..4: mod_base = OTHER; note = 2'O-Methyl ribonucleotide
  misc_feature 5: note = DNA
  modified_base 6: mod_base = OTHER; note = 2'O-Methyl ribonucleotide
  misc_feature 7: note = DNA
  modified_base 8..13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgtccttaacggtgctccagta

SEQ ID NO: 475 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..4: mod_base = OTHER; note = 2'O-Methyl ribonucleotide
  misc_feature 5: note = DNA
  modified_base 6: mod_base = OTHER; note = 2'O-Methyl ribonucleotide
  misc_feature 7: note = DNA
  modified_base 8..13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgaatactgtcccttttaagcaa

SEQ ID NO: 476 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..4: mod_base = OTHER; note = 2'O-Methyl ribonucleotide
  misc_feature 5: note = DNA
  modified_base 6: mod_base = OTHER; note = 2'O-Methyl ribonucleotide
  misc_feature 7: note = DNA
  modified_base 8..13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgagaatactgtcccttttaagc

SEQ ID NO: 477 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..4: mod_base = OTHER; note = 2'O-Methyl ribonucleotide
  misc_feature 5: note = DNA
  modified_base 6: mod_base = OTHER; note = 2'O-Methyl ribonucleotide
  misc_feature 7: note = DNA
  modified_base 8..13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgagaatactgtcccttttaag

SEQ ID NO: 478 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..4: mod_base = OTHER; note = 2'O-Methyl ribonucleotide
  misc_feature 5: note = DNA
  modified_base 6: mod_base = OTHER; note = 2'O-Methyl ribonucleotide
  misc_feature 7: note = DNA
  modified_base 8..13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctgagaatactgtcccttttaa

SEQ ID NO: 479 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..4: mod_base = OTHER; note = 2'O-Methyl ribonucleotide
  misc_feature 5: note = DNA
  modified_base 6: mod_base = OTHER; note = 2'O-Methyl ribonucleotide
  misc_feature 7: note = DNA
  modified_base 8..13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tactgagaatactgtccctttta

SEQ ID NO: 480 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..4: mod_base = OTHER; note = 2'O-Methyl ribonucleotide
  misc_feature 5: note = DNA
  modified_base 6: mod_base = OTHER; note = 2'O-Methyl ribonucleotide
  misc_feature 7: note = DNA
  modified_base 8..13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcactgagaatactgtccctttt

SEQ ID NO: 481 - RNA, 21 nt, synthetic construct
  source 1..21: mol_type = other RNA
gccatgcagccccgggtacta

SEQ ID NO: 482 - RNA, 21 nt, synthetic construct
  source 1..21: mol_type = other RNA
cagccccgggtactccttgta

SEQ ID NO: 483 - RNA, 21 nt, synthetic construct
  source 1..21: mol_type = other RNA
tgaccgatggcttcagttcca

SEQ ID NO: 484 - RNA, 21 nt, synthetic construct
  source 1..21: mol_type = other RNA
gatggcttcagttccctgaaa

SEQ ID NO: 485 - RNA, 21 nt, synthetic construct
  source 1..21: mol_type = other RNA
agactactggagcaccgttaa

SEQ ID NO: 486 - RNA, 21 nt, synthetic construct
  source 1..21: mol_type = other RNA
ctggagcaccgttaaggacaa

SEQ ID NO: 487 - RNA, 21 nt, synthetic construct
  source 1..21: mol_type = other RNA
gcttaaaagggacagtattca

SEQ ID NO: 488 - RNA, 21 nt, synthetic construct
  source 1..21: mol_type = other RNA
ttaaaagggacagtattctca

SEQ ID NO: 489 - RNA, 21 nt, synthetic construct
  source 1..21: mol_type = other RNA
taaaagggacagtattctcaa

SEQ ID NO: 490 - RNA, 21 nt, synthetic construct
  source 1..21: mol_type = other RNA
aaaagggacagtattctcaga

SEQ ID NO: 491 - RNA, 21 nt, synthetic construct
  source 1..21: mol_type = other RNA
aaagggacagtattctcagta

SEQ ID NO: 492 - RNA, 21 nt, synthetic construct
  source 1..21: mol_type = other RNA
aagggacagtattctcagtga

SEQ ID NO: 493 - RNA, 21 nt, synthetic construct
  source 1..21: mol_type = other RNA
gccatgcagccccgggtacta

SEQ ID NO: 494 - RNA, 21 nt, synthetic construct
  source 1..21: mol_type = other RNA
cagccccgggtactccttgta

SEQ ID NO: 495 - RNA, 21 nt, synthetic construct
  source 1..21: mol_type = other RNA
tgaccgatggcttcagttcca

SEQ ID NO: 496 - RNA, 21 nt, synthetic construct
  source 1..21: mol_type = other RNA
gatggcttcagttccctgaaa

SEQ ID NO: 497 - RNA, 21 nt, synthetic construct
  source 1..21: mol_type = other RNA
agactactggagcaccgttaa

SEQ ID NO: 498 - RNA, 21 nt, synthetic construct
  source 1..21: mol_type = other RNA
ctggagcaccgttaaggacaa

SEQ ID NO: 499 - RNA, 21 nt, synthetic construct
  source 1..21: mol_type = other RNA
gcttaaaagggacagtattca

SEQ ID NO: 500 - RNA, 21 nt, synthetic construct
  source 1..21: mol_type = other RNA
ttaaaagggacagtattctca

SEQ ID NO: 501 - RNA, 21 nt, synthetic construct
  source 1..21: mol_type = other RNA
taaaagggacagtattctcaa

SEQ ID NO: 502 - RNA, 21 nt, synthetic construct
  source 1..21: mol_type = other RNA
aaaagggacagtattctcaga

SEQ ID NO: 503 - RNA, 21 nt, synthetic construct
  source 1..21: mol_type = other RNA
aaagggacagtattctcagta

SEQ ID NO: 504 - RNA, 21 nt, synthetic construct
  source 1..21: mol_type = other RNA
aagggacagtattctcagtga

SEQ ID NO: 505 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gccatgcagccccgggtacta

SEQ ID NO: 506 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cagccccgggtactccttgta

SEQ ID NO: 507 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgaccgatggcttcagttcca

SEQ ID NO: 508 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gatggcttcagttccctgaaa

SEQ ID NO: 509 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
agactactggagcaccgttaa

SEQ ID NO: 510 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctggagcaccgttaaggacaa

SEQ ID NO: 511 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcttaaaagggacagtattca

SEQ ID NO: 512 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ttaaaagggacagtattctca

SEQ ID NO: 513 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
taaaagggacagtattctcaa

SEQ ID NO: 514 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aaaagggacagtattctcaga

SEQ ID NO: 515 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aaagggacagtattctcagta

SEQ ID NO: 516 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aagggacagtattctcagtga

SEQ ID NO: 517 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gccatgcagccccgggtacta

SEQ ID NO: 518 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cagccccgggtactccttgta

SEQ ID NO: 519 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgaccgatggcttcagttcca

SEQ ID NO: 520 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gatggcttcagttccctgaaa

SEQ ID NO: 521 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
agactactggagcaccgttaa

SEQ ID NO: 522 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctggagcaccgttaaggacaa

SEQ ID NO: 523 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcttaaaagggacagtattca

SEQ ID NO: 524 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ttaaaagggacagtattctca

SEQ ID NO: 525 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
taaaagggacagtattctcaa

SEQ ID NO: 526 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aaaagggacagtattctcaga

SEQ ID NO: 527 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aaagggacagtattctcagta

SEQ ID NO: 528 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aagggacagtattctcagtga

SEQ ID NO: 529 - RNA, 23 nt, synthetic construct
  source 1..23: mol_type = other RNA
tttcagggaactgaagccatcgg

SEQ ID NO: 530 - RNA, 23 nt, synthetic construct
  source 1..23: mol_type = other RNA
tgaatactgtcccttttaagcaa

SEQ ID NO: 531 - RNA, 23 nt, synthetic construct
  source 1..23: mol_type = other RNA
tagaatactgtcccttttaagca

SEQ ID NO: 532 - RNA, 23 nt, synthetic construct
  source 1..23: mol_type = other RNA
ttgagaatactgtcccttttaag

SEQ ID NO: 533 - RNA, 23 nt, synthetic construct
  source 1..23: mol_type = other RNA
tctgagaatactgtcccttttaa

SEQ ID NO: 534 - RNA, 23 nt, synthetic construct
  source 1..23: mol_type = other RNA
tcactgagaatactgtccctttt

SEQ ID NO: 535 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tttcagggaactgaagccatcgg

SEQ ID NO: 536 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgaatactgtcccttttaagcaa

SEQ ID NO: 537 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tagaatactgtcccttttaagca

SEQ ID NO: 538 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgagaatactgtcccttttaag

SEQ ID NO: 539 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctgagaatactgtcccttttaa

SEQ ID NO: 540 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcactgagaatactgtccctttt

SEQ ID NO: 541 - RNA, 21 nt, synthetic construct
  source 1..21: mol_type = other RNA
gatggcttcagttccctgaaa

SEQ ID NO: 542 - RNA, 21 nt, synthetic construct
  source 1..21: mol_type = other RNA
gcttaaaagggacagtattca

SEQ ID NO: 543 - RNA, 21 nt, synthetic construct
  source 1..21: mol_type = other RNA
cttaaaagggacagtattcta

SEQ ID NO: 544 - RNA, 21 nt, synthetic construct
  source 1..21: mol_type = other RNA
taaaagggacagtattctcaa

SEQ ID NO: 545 - RNA, 21 nt, synthetic construct
  source 1..21: mol_type = other RNA
aaaagggacagtattctcaga

SEQ ID NO: 546 - RNA, 21 nt, synthetic construct
  source 1..21: mol_type = other RNA
aagggacagtattctcagtga

SEQ ID NO: 547 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gatggcttcagttccctgaaa

SEQ ID NO: 548 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcttaaaagggacagtattca

SEQ ID NO: 549 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cttaaaagggacagtattcta

SEQ ID NO: 550 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
taaaagggacagtattctcaa

SEQ ID NO: 551 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aaaagggacagtattctcaga

SEQ ID NO: 552 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aagggacagtattctcagtga

SEQ ID NO: 553 - RNA, 23 nt, synthetic construct
  source 1..23: mol_type = other RNA
tctgagaatactgtcccttttaa

SEQ ID NO: 554 - RNA, 23 nt, synthetic construct
  source 1..23: mol_type = other RNA
tctgagaatactgtcccttttaa

SEQ ID NO: 555 - RNA, 23 nt, synthetic construct
  source 1..23: mol_type = other RNA
tctgagaatactgtccctttcaa

SEQ ID NO: 556 - RNA, 23 nt, synthetic construct
  source 1..23: mol_type = other RNA
tctgagaatactgtccctttgaa

SEQ ID NO: 557 - RNA, 23 nt, synthetic construct
  source 1..23: mol_type = other RNA
tctgagaatactgtcccttgcaa

SEQ ID NO: 558 - RNA, 23 nt, synthetic construct
  source 1..23: mol_type = other RNA
tctgagaatactgtcccttcgaa

SEQ ID NO: 559 - RNA, 23 nt, synthetic construct
  source 1..23: mol_type = other RNA
tctgagaatactgtcccttttaa

SEQ ID NO: 560 - RNA, 23 nt, synthetic construct
  source 1..23: mol_type = other RNA
tctgagaatactgtccctttgaa

SEQ ID NO: 561 - RNA, 23 nt, synthetic construct
  source 1..23: mol_type = other RNA
tctgagaatactgtcccttttaa

SEQ ID NO: 562 - RNA, 23 nt, synthetic construct
  source 1..23: mol_type = other RNA
tctgagaatactgtcccttttaa

SEQ ID NO: 563 - RNA, 23 nt, synthetic construct
  source 1..23: mol_type = other RNA
tctgagaatactgtcccttttaa

SEQ ID NO: 564 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctgagaatactgtcccttttaa

SEQ ID NO: 565 - RNA, 24 nt, synthetic construct
  source 1..24: mol_type = other RNA
  modified_base 1: mod_base = OTHER; note = 5'-vinylphosphonate modified nucleotide
  modified_base 2: mod_base = OTHER; note = 2'O-Methyl ribonucleotide
  modified_base 2..4: mod_base = OTHER; note = Phosphorothioate linkage
  modified_base 3: mod_base = OTHER; note = 2'fluoro ribonucleotide
  modified_base 4..7: mod_base = OTHER; note = 2'O-Methyl ribonucleotide
  modified_base 8: mod_base = OTHER; note = 2'fluoro ribonucleotide
  modified_base 9..12: mod_base = OTHER; note = 2'O-Methyl ribonucleotide
  modified_base 13: mod_base = OTHER; note = 2'fluoro ribonucleotide
  modified_base 14: mod_base = OTHER; note = 2'O-Methyl ribonucleotide
  modified_base 15: mod_base = OTHER; note = 2'fluoro ribonucleotide
  modified_base 16: mod_base = OTHER; note = 2'O-Methyl ribonucleotide
  modified_base 17: mod_base = OTHER; note = 2'fluoro ribonucleotide
  modified_base 18..24: mod_base = OTHER; note = 2'O-Methyl ribonucleotide
  modified_base 22..24: mod_base = OTHER; note = Phosphorothioate linkage
ntctgagaatactgtcccttttaa

SEQ ID NO: 566 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctgagaatactgtccctttcaa

SEQ ID NO: 567 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctgagaatactgtccctttgaa

SEQ ID NO: 568 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctgagaatactgtcccttgcaa

SEQ ID NO: 569 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctgagaatactgtcccttcgaa

SEQ ID NO: 570 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctgagaatactgtcccttttaa

SEQ ID NO: 571 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctgagaatactgtccctttgaa

SEQ ID NO: 572 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..4: mod_base = OTHER; note = 2'O-Methyl ribonucleotide
  misc_feature 5: note = DNA
  modified_base 6: mod_base = OTHER; note = 2'O-Methyl ribonucleotide
  misc_feature 7: note = DNA
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctgagaatactgtcccttttaa

SEQ ID NO: 573 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 14..15: mod_base = OTHER; note = Phosphorothioate linkage
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctgagaatactgtcccttttaa

SEQ ID NO: 574 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 17..18: mod_base = OTHER; note = Phosphorothioate linkage
  modified_base 21..23: mod_base = OTHER; note = Phosphorothioate linkage
tctgagaatactgtcccttttaa

SEQ ID NO: 575 - RNA, 21 nt, synthetic construct
  source 1..21: mol_type = other RNA
aaaagggacagtattctcaga

SEQ ID NO: 576 - RNA, 21 nt, synthetic construct
  source 1..21: mol_type = other RNA
aaaagggacagtattctcaga

SEQ ID NO: 577 - RNA, 21 nt, synthetic construct
  source 1..21: mol_type = other RNA
gaaagggacagtattctcaga

SEQ ID NO: 578 - RNA, 21 nt, synthetic construct
  source 1..21: mol_type = other RNA
caaagggacagtattctcaga

SEQ ID NO: 579 - RNA, 21 nt, synthetic construct
  source 1..21: mol_type = other RNA
gcaagggacagtattctcaga

SEQ ID NO: 580 - RNA, 21 nt, synthetic construct
  source 1..21: mol_type = other RNA
cgaagggacagtattctcaga

SEQ ID NO: 581 - RNA, 21 nt, synthetic construct
  source 1..21: mol_type = other RNA
aaaagggacagtattctcaga

SEQ ID NO: 582 - RNA, 21 nt, synthetic construct
  source 1..21: mol_type = other RNA
caaagggacagtattctcaga

SEQ ID NO: 583 - RNA, 21 nt, synthetic construct
  source 1..21: mol_type = other RNA
aaaagggacagtattctcaga

SEQ ID NO: 584 - RNA, 21 nt, synthetic construct
  source 1..21: mol_type = other RNA
aaaagggacagtattctcaga

SEQ ID NO: 585 - RNA, 21 nt, synthetic construct
  source 1..21: mol_type = other RNA
aaaagggacagtattctcaga

SEQ ID NO: 586 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aaaagggacagtattctcaga

SEQ ID NO: 587 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aaaagggacagtattctcaga

SEQ ID NO: 588 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gaaagggacagtattctcaga

SEQ ID NO: 589 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
caaagggacagtattctcaga

SEQ ID NO: 590 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcaagggacagtattctcaga

SEQ ID NO: 591 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cgaagggacagtattctcaga

SEQ ID NO: 592 - RNA, 22 nt, synthetic construct
  source 1..22: mol_type = other RNA
  modified_base 1: mod_base = OTHER; note = Inverted abasic nucleotide
  modified_base 2..7: mod_base = OTHER; note = 2'O-Methyl ribonucleotide
  modified_base 2..4: mod_base = OTHER; note = Phosphorothioate linkage
  modified_base 8: mod_base = OTHER; note = 2'fluoro ribonucleotide
  modified_base 9: mod_base = OTHER; note = 2'O-Methyl ribonucleotide
  modified_base 10: mod_base = OTHER; note = 2'fluoro ribonucleotide
  modified_base 11: mod_base = OTHER; note = 2'O-Methyl ribonucleotide
  modified_base 12: mod_base = OTHER; note = 2'fluoro ribonucleotide
  modified_base 13..22: mod_base = OTHER; note = 2'O-Methyl ribonucleotide
naaaagggacagtattctcaga

SEQ ID NO: 593 - RNA, 22 nt, synthetic construct
  source 1..22: mol_type = other RNA
  modified_base 1: mod_base = OTHER; note = Inverted abasic nucleotide
  modified_base 2..7: mod_base = OTHER; note = 2'O-Methyl ribonucleotide
  modified_base 2..4: mod_base = OTHER; note = Phosphorothioate linkage
  modified_base 8: mod_base = OTHER; note = 2'fluoro ribonucleotide
  modified_base 9: mod_base = OTHER; note = 2'O-Methyl ribonucleotide
  modified_base 10: mod_base = OTHER; note = 2'fluoro ribonucleotide
  modified_base 11: mod_base = OTHER; note = 2'O-Methyl ribonucleotide
  modified_base 12: mod_base = OTHER; note = 2'fluoro ribonucleotide
  modified_base 13..22: mod_base = OTHER; note = 2'O-Methyl ribonucleotide
ncaaagggacagtattctcaga

SEQ ID NO: 594 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aaaagggacagtattctcaga

SEQ ID NO: 595 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aaaagggacagtattctcaga

SEQ ID NO: 596 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aaaagggacagtattctcaga

SEQ ID NO: 597 - protein, 5 aa, synthetic construct
YIGSR

SEQ ID NO: 598 - protein, 4 aa, synthetic construct
  SITE 1: note = N-formyl-methionyl
  SITE 2: note = leucyl
MLFK